(19) United States
(12) Patent Application Publication
(10) Pub. No.: US 2010/0273651 A1
(43) Pub. Date: Oct. 28, 2010

(54) Azolylmethyloxiranes, use Thereof and Agents Containing the Same

(75) Inventors: Jochen Dietz, Karlsruhe (DE); Thomas Grote, Wachenheim (DE); Bernd Mueller, Frankenthal (DE); Jan Klaas Lohmann, Ludwigshafen (DE); Jens Renner, Bad Duerkheim (DE); Sarah Ulmschneider, Bad Duerkheim (DE); Alice Glaettli, Frankfurt (DE); Marianna Vrettou, Mannheim (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/808,799

(22) PCT Filed: Dec. 15, 2008
(86) PCT No.: PCT/EP08/67545

§ 371 Date: Jun. 17, 2010

(30) Foreign Application Priority Data
Dec. 19, 2007 (EP) 07123713.5

(51) Int. Cl. A01C 1/06; C07D 249/12; A01N 43/653; A01P 3/00; A01P 7/04; A01N 43/00; C07D 303/08

(52) U.S. Cl. 504/100; 548/264.2; 514/384; 548/263.2; 548/264.4; 504/139; 549/563

(57) ABSTRACT

The present invention relates to azolylmethyloxiranes of the formula I

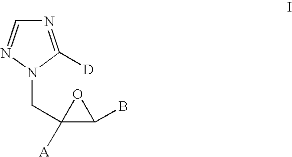

in which the variables A, B and D have the meanings as defined in the description and the claims.

The present invention relates to azolylmethyloxiranes of the formula I

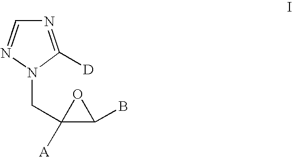

in which the variables have the following meanings:
A is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, naphthyl, phenyl; where A is unsubstituted or substituted by one, two, three or four identical or different substituents L;
B is phenyl which contains a substituent L in the 2-position and one, two or three further independently selected substituents L; where L is as defined below:
   L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-halo-alkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₈-cycloalkenyloxy, hydroxyimino-C₁-C₈-alkyl, C₁-C₈-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl, phenyloxy or a five- or six-membered saturated, partially unsaturated or aromatic heterocycle which contains one, two, three or four heteroatoms from the group consisting of O, N and S; where n, A¹, A², A³, A⁴ are as defined below:
   n is 0, 1 or 2;
   A¹ is hydrogen, hydroxyl, C₁-C₈-alkyl, C₁-C₈-haloalkyl, amino, C₁-C₈-alkylamino or di-C₁-C₈-alkylamino,
   A² is one of the groups mentioned for A₁ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
   A³,A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
   where the aliphatic and/or alicyclic and/or aromatic groups of the radical definitions of L for their part may carry one, two, three or four identical or different groups R^{L}:
   R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino;
D is S—R, where
   R is hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C(═O)R³, C(═S)R³, SO₂R⁴ or CN; where
   R³ is C₁-C₈-alkyl, C₁-C₈-haloalkyl, C₁-C₈-alkoxy, C₁-C₈-haloalkoxy or NA³A⁴; and
   R⁴ is C₁-C₈-alkyl, phenyl-C₁-C₈-alkyl or phenyl, where the phenyl groups are in each case unsubstituted or substituted by one, two or three groups independently selected from the group consisting of halogen and C₁-C₄-alkyl;
   is a group DI

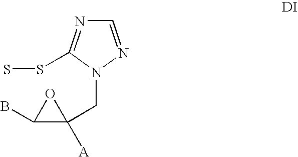

where A and B are as defined above;
is a group DII

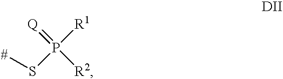

where # is the point of attachment to the triazolyl ring and Q, R¹ and R² are as defined below:
Q is O or S;
R¹, R² independently of one another are C₁-C₈-alkyl, C₁-C₈-alkoxy, C₁-C₈-alkoxy-C₁-C₈-alkoxy, C₁-C₈-haloalkoxy, C₁-C₈-alkylthio, C₂-C₈-alkenylthio, C₂-C₈-alkynyl-thio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR⁵R⁶, where R⁵ is H or C₁-C₈-alkyl and R⁶ is C₁-C₈-alkyl, phenyl-C₁-C₄-alkyl or phenyl or R⁵ and R⁶ together are an alkylene chain having four or five carbon atoms or form a radical of the formula —CH₂—CH₂—O—CH₂—CH₂— or —CH₂—CH₂—NR⁷—CH₂—CH₂— in which R⁷ is hydrogen or C₁-C₄-alkyl; where the aromatic groups in the radicals mentioned above are in each case independently of one another unsubstituted or substituted by one, two or three groups selected from the group consisting of halogen and C₁-C₄-alkyl;
or
is a group SM, where M is as defined below:
M is an alkali metal cation, an equivalent of an alkaline earth metal cation, an equivalent of a copper, zinc, iron or nickel cation or an ammonium cation of the formula (E)

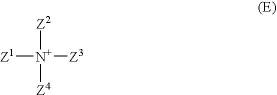

in which
Z¹ and Z² independently are hydrogen or C₁-C₈-alkyl;
Z³ and Z⁴ independently are hydrogen, C₁-C₈-alkyl, benzyl or phenyl; where the phenyl groups are in each case unsubstituted or substituted by one, two or three groups independently selected from the group consisting of halogen and C₁-C₄-alkyl;
   and an agriculturally acceptable salt thereof.

In a preferred embodiment, the invention relates to azolylmethyloxiranes of the formula I:

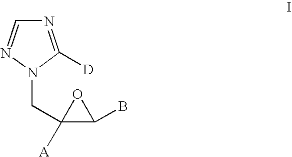

in which the variables have the following meanings:
A is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, naphthyl, phenyl; where A is unsubstituted or substituted by one, two, three or four identical or different substituents L;
B is phenyl which contains a substituent L in the 2-position and a substituent L* in the 4-position,
   or
   phenyl which contains a substituent L in the 2-position and a further independently selected substituent L in the 3-, 5- or 6-position,
   or
   phenyl which contains a substituent L in the 2-position and two or three further independently selected substituents L;
   where L and L* are as defined below:
      L L* or chlorine;
      L* is fluorine, bromine, iodine, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkylenoxy, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl, phenyloxy or a five- or six-membered saturated, partially unsaturated or aromatic heterocycle which contains one, two, three or four heteroatoms from the group consisting of O, N and S; where n, A¹, A², A³, A⁴ are as defined below:
   n is 0, 1 or 2;
   A¹ is hydrogen, hydroxyl, C₁-C₈-alkyl, C₁-C₈-haloalkyl, amino, C₁-C₈-alkylamino or di-C₁-C₈-alkylamino,
   A² is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkoxy or C₃-C₈-halocycloalkoxy;
   A³,A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halocycloalkenyl;
   where the aliphatic and/or alicyclic and/or aromatic groups of the radical definitions of L and L* for their part may carry one, two, three or four identical or different groups R^{L}:
   R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₁-C₈-alkylcarbonyl, C₁-C₈-alkylcarbonyloxy, C₁-C₈-alkoxycarbonyl, amino, C₁-C₈-alkylamino, di-C₁-C₈-alkylamino;
D is S—R, where
   R is hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C(═O)R³, C(═S)R³, SO₂R⁴ or CN; where R³ is C₁-C₈-alkyl, C₁-C₈-haloalkyl, C₁-C₈-alkoxy, C₁-C₈-haloalkoxy or NA³A⁴; and
   R⁴ is C₁-C₈-alkyl, phenyl-C₁-C₈-alkyl or phenyl, where the phenyl groups are in each case unsubstituted or substituted by one, two or three groups independently selected from the group consisting of halogen and C₁-C₄-alkyl;
   is a group DI

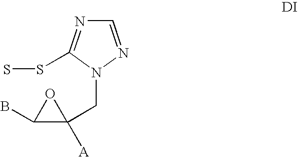

where A and B are as defined above;
is a group DII

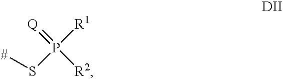

where # is the point of attachment to the triazolyl ring and Q, R¹ and R² are as defined below:
Q is O or S;
R¹, R² independently of one another are C₁-C₈-alkyl, C₁-C₈-haloalkyl, C₁-C₈-alkoxy, C₁-C₈-alkoxy-C₁-C₈-alkoxy, C₁-C₈-haloalkoxy, C₁-C₈-alkoxy-C₁-C₈-alkyl, C₁-C₈-alkylthio, C₂-C₈-alkenylthio, C₂-C₈-alkynyl-thio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR⁵R⁶, where R⁵ is H or C₁-C₈-alkyl and R⁶ is C₁-C₈-alkyl, phenyl-C₁-C₄-alkyl or phenyl or R⁵ and R⁶ together are an alkylene chain having four or five carbon atoms or form a radical of the formula —CH₂—CH₂—O—CH₂—CH₂— or —CH₂—CH₂—NR⁷—CH₂—CH₂— in which R⁷ is hydrogen or C₁-C₄-alkyl; where the aromatic groups in the radicals mentioned above are in each case independently of one another unsubstituted or substituted by one, two or three groups selected from the group consisting of halogen and C₁-C₄-alkyl;
or
is a group SM, where M is as defined below:
M is an alkali metal cation, an equivalent of an alkaline earth metal cation, an equivalent of a copper, zinc, iron or nickel cation or an ammonium cation of the formula (E)

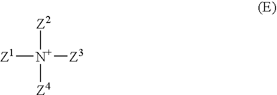

in which
Z¹ and Z² independently are hydrogen or C₁-C₈-alkyl;
Z³ and Z⁴ independently are hydrogen, C₁-C₈-alkyl, benzyl or phenyl; where the phenyl groups are in each case unsubstituted or substituted by one, two or three groups independently selected from the group consisting of halogen and C₁-C₄-alkyl;
and agriculturally acceptable salts thereof.

The compounds of the formula I can be present in the “thiol” form of the formula Ia or in the “thiono” form of the formula Ib

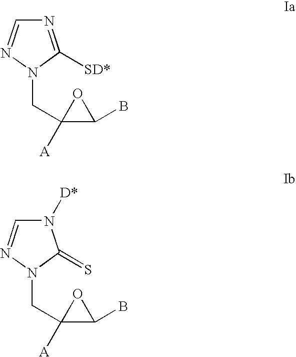

in which D* is:
R, where R has the meaning defined above;
a group DII*

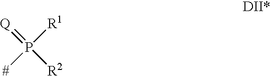

where # is the point of attachment to the sulfur atom in formula Ia or azolyl ring in formula Ib and Q, R¹ and R² have the meaning defined above; or
   a group M, where M has the meaning defined above,
      and in which the remaining substituents have the meaning defined above.

However, for the sake of simplicity, in each case generally only the “thiol” form is shown here.

The invention furthermore relates to the preparation of the compounds I, to the intermediates for preparing the compounds I and to their preparation, and also to the use of the compounds according to the invention for controlling phytopathogenic fungi, and to compositions comprising them.

Triazolylmethyloxiranes having a substituted triazole group are known, for example, from WO 96/38440, WO 97/41107, WO 97/42178, WO 97/43269, WO 97/44331, WO 97/443332, WO 99/05149 and WO 99/21853.

However, in particular at low application rates, the fungicidal action of the compounds known from the prior art is sometimes unsatisfactory. Accordingly, it was an object of the present invention to provide novel compounds which preferably have improved properties, such as improved fungicidal action and/or better toxicological properties. Surprisingly, this object was achieved with the compounds of the formula I described here.

Owing to the basic character of their nitrogen atoms, the compounds I are capable of forming salts or adducts with inorganic or organic acids or with metal ions. This also applies to most of the precursors described herein of compounds I, the salts and adducts of which are also provided by the present invention.

Examples of inorganic acids are hydrohalic acids, such as hydrogen fluoride, hydrogen chloride, hydrogen bromide and hydrogen iodide, carbonic acid, sulfuric acid, phosphoric acid and nitric acid.

Suitable organic acids are, for example, formic acid and alkanoic acids, such as acetic acid, trifluoroacetic acid, trichloroacetic acid and propionic acid, and also glycolic acid, thiocyanic acid, lactic acid, succinic acid, citric acid, benzoic acid and other arylcarboxylic acids, cinnamic acid, oxalic acid, alkylsulfonic acids (sulfonic acids having straight-chain or branched alkyl radicals of 1 to 20 carbon atoms), arylsulfonic acids or aryldisulfonic acids (aromatic radicals, such as phenyl and naphthyl, which carry one or two sulfonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphoric acid radicals), where the alkyl or aryl radicals may carry further substituents, for example p-toluenesulfonic acid, salicylic acid, p-aminosalicylic acid, 2-phenoxybenzoic acid, 2-acetoxybenzoic acid etc.

Suitable metal ions are in particular the ions of the elements of the second main group, in particular calcium and magnesium, of the third and fourth main group, in particular aluminum, tin and lead and also of the elements of transition groups one to eight, in particular chromium, manganese, iron, cobalt, nickel, copper, zinc and others. Particular preference is given to the metal ions of the elements of transition groups of the fourth period. The metals can be present in the various valencies that they can assume.

The compounds of the formula I according to the invention can be prepared by different routes analogously to processes known per se of the prior art (see, for example, the prior art cited at the outset and Pflanzenschutz-Nachrichten Bayer 57/2004, 2, pages 145-162). The compounds according to the invention can be prepared, for example, according to the syntheses shown in the schemes below.

The compounds according to the invention can be prepared in an advantageous manner from compounds of the formula II

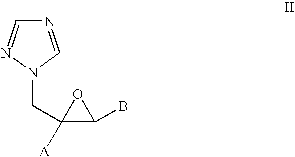

in which A and B are as defined herein by reaction with a strong base and sulfur powder. This results in the formation of compounds of the formula I in which D is SH (compounds I-1):

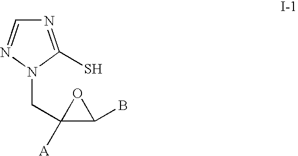

Suitable bases are all bases known to the person skilled in the art as being suitable for such reactions. Preference is given to using strong alkali metal bases such as, for example, n-butyllithium, lithium diisopropylamide, sodium hydride, sodium amide or potassium tert-butoxide. It may be preferred to carry out the reaction in the presence of an additive such as, for example, tetramethylethylenediamine (TMEDA).

Suitable solvents are all inert organic solvents customary for such reactions, where preferably ethers such as tetrahydrofuran, dioxane, diethyl ether and 1,2-dimethoxyethane or liquid ammonia or strongly polar solvents such as dimethyl sulfoxide may be used.

Sulfur is preferably used as a powder. For the hydrolysis, use is made of water, if appropriate in the presence of an organic or inorganic acid such as, for example, acetic acid, dilute sulfuric acid or dilute hydrochloric acid.

The reaction temperature is preferably between −70° C. and +20° C., in particular between −70° C. and 0° C. The reaction is generally carried out under atmospheric pressure.

In general, 1 to 3 equivalents, preferably 1 to 2.5 equivalents, of a strong base and then an equivalent amount or an excess of sulfur are employed per mole of the compound of the formula II. The reaction can be carried out under an atmosphere of protective gas such as, for example, under nitrogen or argon. Work-up is carried out according to procedures generally known to the person skilled in the art. Usually, the reaction mixture is extracted with a suitable organic solvent, and the residue is, if appropriate, purified by recrystallization and/or chromatography.

It is also possible to prepare compounds I by direct reaction with sulfur, preferably sulfur powder, without the use of a strong base such as butyllithium.

A further alternative of preparing, starting with compound II, compounds I according to the invention is to react compounds II with sulfur in the presence of an aprotic polar solvent, such as, for example, an amide (such as dimethylformamide (DMF)) or N-alkylpyrrolidone (such as N-octylpyrrolidone, N-dodecylpyrrolidone or N-methylpyrrolidone (NMP)). The corresponding processes are described, for example, in WO 99/19307, WO 97/06151, WO 97/05119 and WO 96/41804.

The reaction is generally carried out at temperatures in the range of from 140° C. to 160° C. The reaction components are usually employed in amounts such that from about 6 to 15 mol of sulfur are used per mole of the compound II. Sulfur is generally used in the form of a powder. During the reaction, air is passed over the reaction mixture.

Moreover, the compounds according to the invention can be prepared in an advantageous manner from compounds of the formula II by reaction with disulfides or thiocyanogen:

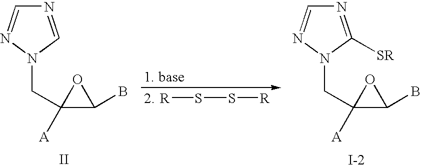

in which A and B are defined as described herein, and R is C₁-C₈-alkyl, C₁-C₈-haloalkyl, C₂-C₈-alkenyl, C₂-C₈-haloalkenyl, C₂-C₈-alkynyl, C₂-C₈-haloalkynyl or CN.

Suitable bases are all bases known to the person skilled in the art as being suitable for such reactions. Preference is given to using strong alkali metal bases, such as, for example, n-butyllithium, lithiumdiisopropylamide, sodium hydride, sodium amide or potassium tert-butoxide. It may be preferred to carry out the reaction in the presence of an additive, such as, for example, tetramethylethylenediamine (TMEDA). The disulfides are commercially available or can be synthesized by known preparation processes. A specific disulfide is thiocyanogen NC—S—S—CN.

Suitable solvents are all inert organic solvents customary for such reactions; and preference is given to using ethers, such as tetrahydrofuran, dioxane, diethylether and 1,2-dimethoxyethane, or liquid ammonia or strongly polar solvents, such as dimethyl sulfoxide.

The reaction temperature is preferably between −70° C. and +20° C., in particular between −70° C. and 0° C. The reaction was generally carried out under atmospheric pressure.

In general, from 1 to 3 equivalents, preferably from 1 to 2.5 equivalents, of a strong base and then an equivalent amount or an excess of disulfide are employed per mole of the compound of the formula II. The reaction can be carried out under an atmosphere of protective gas, such as, for example, under nitrogen or argon. Work-up was carried out by procedures generally known to the person skilled in the art. Usually, the reaction mixture is extracted with a suitable organic solvent and the residue is, if appropriate, purified by recrystallization and/or chromatography.

By further reaction of compounds I-1 with R—X, where R is as defined herein at a different location and X is a leaving group such as, for example, halogen, such as Cl, Br or I, or trifluoro-C₁-C₆-alkylsulfonate, it is possible to prepare various compounds of the formula I according to the invention. To prepare compounds where D=SR where R═C₁-C₆-alkyl, preferably methyl or ethyl, a compound 1-1 is reacted with the corresponding alkyl halide (see also WO 96/38440).

Compounds of the formula I in which D is S—CN can be synthesized analogously to the process described in WO 99/44331.

Compounds of the formula I in which D is a group DI can be synthesized analogously to the process described in WO 97/43269.

Compounds of the formula I in which D is a group S—C(═O)R³ where R³═C₁-C₈-alkyl, C₁-C₈-haloalkyl, C₁-C₈-alkoxy or C₁-C₈-haloalkoxy can be synthesized analogously to the process described in WO 97/42178.

Compounds of the formula I in which D is a group SM can be synthesized analogously to the process described in WO 97/41107.

The compounds of the formula II can be synthesized analogously to the prior art cited at the outset. Some compounds of the formula II are described in the patent applications PCT/EP2007/063213, PCT/EP2007/056124, PCT/EP2007/055870, PCT/EP200/055932. On the other hand, some compounds of the formula II are novel, and they and their salts and adducts also form part of the subject matter of the present invention. Compounds II also have fungicidal activity, and accordingly the present invention also relates to the use of the compounds II and/or the salts and adducts thereof as fungicides. Preferred compounds of the formula II are specified further below in Tables 1c to 162c.

Compounds of the formula III

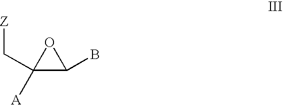

in which Z is a leaving group X (compounds 111.1, see below) or OH (compounds 111.2, see below) and A and B are as defined below are important starting compounds needed to obtain, finally, the compounds according to the invention.

Thus, compounds II can be prepared, for example, from compounds 111.1

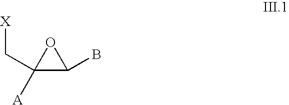

where X is a leaving group such as, for example, halogen (for example Cl or Br) or OSO₂R, where R is C₁-C₆-alkyl, C₁-C₆-haloalkyl, aryl or substituted aryl; OSO₂R is in particular a mesylate, triflate, phenyl or toluenesulfonate group. To obtain compounds of the formula II, compounds of the formula III.1 are reacted with 1,2,4-triazole and a base such as, for example, sodium hydride, for example in DMF. See also, for example, EP 0 421 125 A2.

Some of the compounds of the formula III.1 are novel. Accordingly, the invention also provides compounds of the formula III.1 where A and B are as defined or as preferably defined for formula I and X is a leaving group, in particular halogen (for example Cl or Br) or OSO₂R where R is C₁-C₆-alkyl, C₁-C₆-haloalkyl, aryl or substituted aryl, except for the compounds 2-bromomethyl-2-(4-chlorophenyl)-3-(2,4-dichlorophenyl)oxiran, 2-bromomethyl-2-(4-bromophenyl)-3-(2,4-dichlorophenyl)oxiran, 2-bromomethyl-2-(tert-butyl)-3-(2,4-dichlorophenyl)oxiran, 2-bromomethyl-2-(methyl)-3-(2,4-dichlorophenyl)-oxiran, 2-bromomethyl-2-(4-(tert-butyl)phenyl)-3-(2,4-dichlorophenyl)oxiran, 2-bromo-methyl-2-(2-methoxyphenyl)-3-(2,4-dichlorophenyl)oxiran, 2-bromomethyl-2-(4-(tert-butoxyphenyl)-3-(2,4-dichlorophenyl)oxiran, 2-bromomethyl-2-(2-naphthyl)-3-(2,4-dichlorophenyl)oxiran, 2-bromomethyl-2-(4-fluorophenyl)-3-(2,4-dichlorophenyl)oxiran, 2-bronnomethyl-2-(4-chlorophenyl)-3-(2-chloro-4-fluorophenypoxiran, 2-bromomethyl-2-(phenyl)-3-(2-chloro-4-fluorophenyl)oxiran, 2-bromomethyl-2-(4-fluorophenyl)-3-(2-chloro-4-fluorophenyl)oxiran, 2-bromomethyl-2-(4-chlorophenyl)-3-(2-chloro-6-fluorophenyl)oxiran, 2-bromomethyl-2-(phenyl)-3-(2-chloro-6-fluorophenyl)oxiran, 2-bromomethyl-2-(phenyl)-3-(2,4-dichlorophenyl)oxiran.

The invention furthermore provides compounds of the formula III.1 where A and B are as defined or as preferably defined for formula I and X is a leaving group, in particular halogen (for example Cl or Br) or OSO₂R, where R is C₁-C₆-alkyl, C₁-C₆-haloalkyl, aryl or substituted aryl, except for the compounds 2-bromomethyl-2-(4-chlorophenyl)-3-(2-chloro-4-fluorophenyl)oxirane, 2-bromomethyl-2-(phenyl)-3-(2-chloro-4-fluorophenyl)oxirane, 2-bromomethyl-2-(4-fluorophenyl)-3-(2-chloro-4-fluorophenypoxirane, 2-bromonnethyl-2-(4-chlorophenyl)-3-(2-chloro-6-fluorophenyl)oxirane, 2-bromomethyl-2-(phenyl)-3-(2-chloro-6-fluorophenyl)oxirane, 2-bromomethyl-2-(phenyl)-3-(2,4-dichlorophenyl)oxirane.

According to a further embodiment of the compounds III.1, A is phenyl which contains one, two or three independently selected substituents L, with the exceptions just mentioned.

A=phenyl or 4-chlorophenyl; and
B=2-chloro-6-fluorophenyl.

According to a further embodiment of the compounds 111.1, A is phenyl which contains one, two or three independently selected substituents L, with the exceptions just mentioned.

According to a further embodiment, compounds 111.1 in which A and B are as defined below are also excluded:

A=phenyl; and
B=2-chloro-5-NO₂-phenyl or 2,4-dimethylphenyl;
A=cyclohexyl, 2-chlorophenyl or 2,4-dichlorophenyl; and
B=2,4-dichlorophenyl;
A=4-fluorophenyl
B=2-chloro-5-NO₂-phenyl, 2-chloro-4-methoxyphenyl, 2,4-difluorophenyl, 2,6-difluorophenyl, 2-fluoro-3-fluorophenyl or 2,4-dimethylphenyl.

According to a further embodiment of the compounds 111.1, A is phenyl which contains one, two or three independently selected substituents L, with the exceptions mentioned. According to one aspect, the phenyl ring A contains two or three substituents L. According to a further aspect, the phenyl ring A contains a substituent L in the 4-position which is selected from the group consisting of C₁-C₄-alkyl, C₁-C₄-haloalkyl, C₁-C₄-alkoxy, C₁-C₄-haloalkoxy and C₁-C₄-haloalkylthio and no further substituent L or one or two further independently selected substituents L.

According to a further embodiment of the compounds III.1, X is Cl.

According to yet a further embodiment of the compounds III.1, X is OSO₂R, where R is C₁-C₆-alkyl, C₁-C₆-haloalkyl, aryl or substituted aryl; in particular, OSO₂R is a mesylate, triflate, phenyl or toluenesulfonate group.

According to yet a further embodiment of the compounds III.1, Z is Br and B is phenyl which contains one substituent L in the 2-position and exactly one further substituent L, where L is in each case independently selected from the group consisting of F, Br, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkylenoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ᵣ,A¹, C(═O)A², C(═S)A², NA³A⁴, phenyl, phenyloxy or a five- or six-membered saturated, partially unsaturated or aromatic heterocycle which contains one, two, three or four heteroatoms from the group consisting of O, N and S; where n, A¹, A², A³, A⁴ are as defined herein.

According to yet a further embodiment of the compounds 111.1, Z is mesylate or tosylate and B is phenyl which contains one substituent L in the 2-position and exactly one further substituent L, where L is in each case independently selected from the group consisting of F, Br, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₈-cycloalkenyloxy, hydroxyimino-C₁-C₈-alkyl, C₁-C₆-alkylene, oxy-C₂-C₄-alkylene, oxy-C₁-C₃-alkylenoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl, phenyloxy or a five- or six-membered saturated, partially unsaturated or aromatic heterocycle which contains one, two, three or four heteroatoms from the group consisting of O, N and S; where n, A¹, A², A³, A⁴ are as defined herein.

A and B in III.1 have in particular the meanings as specified herein for formula I, taking into account the compounds which are excluded.

One way of preparing compounds III.1 consists in converting the double bond in compounds of the formula IVa

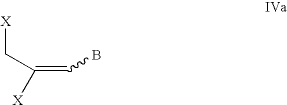

into the epoxide. X is as defined or as preferably defined for formula III.1, and A and B are as defined or as preferably defined for formula I. Suitable epoxidation methods are known to the person skilled in the art. It is possible, for example, to use hydrogen peroxide/maleic anhydride for this purpose.

In formula IVa, the double bond can be present either in (E) or in (Z) configuration. This is indicated by the zig-zag bond between B and the double bond. The present invention furthermore provides compounds of the formula IVa in which A and B are as defined or as preferably defined for formula I, except for the compounds 1-(2,4-dichlorophenyl)-2-(4-chlorophenyl)-3-bromoprop-1-ene, 1-(2,4-dichlorophenyl)-2-(4-bromophenyl)-3-bromoprop-1-ene, 1-(2,4-dichlorophenyl)-2-(tert-butyl)-3-bromoprop-1-ene, 1-(2,4-dichlorophenyl)-2-(methyl)-3-bromoprop-1-ene, 1-(2,4-dichlorophenyl)-2-(4-tert-butylphenyl)-3-bromoprop-1-ene, 1-(2,4-dichlorophenyl)-2-(2-methoxyphenyl)-3-bromoprop-1-ene, 1-(2,4-dichlorophenyl)-2-(4-tert-butoxyphenyl)-3-bromoprop-1-ene, 1-(2,4-dichlorophenyl)-2-(2-naphthyl)-3-bromoprop-1-ene, 1-(2,4-dichlorophenyl)-2-(4-fluorophenyl)-3-bromoprop-1-ene, 1-(2-chloro-4-fluorophenyl)-2-(4-chlorophenyl)-3-bromoprop-1-ene, 1-(2-chloro-4-fluorophenyl)-2-(phenyl)-3-bromoprop-1-ene, 1-(2-chloro-4-fluorophenyl)-2-(4-fluorophenyl)-3-bromoprop-1-ene, 1-(2-chloro-6-fluorophenyl)-2-(4-chlorophenyl)-3-bromoprop-1-ene, 1-(2-chloro-6-fluorophenyl)-2-(phenyl)-3-bromoprop-1-ene, 1-(2,4-dichlorophenyl)-2-(tert-butyl)-3-bromoprop-1-ene and 1-(2,4-dichlorophenyl)-2-(phenyl)-3-bromoprop-1-ene. X has the meaning as described above for formula III.1.

According to one embodiment of the compounds IVa, B is not 2,4-dichlorophenyl. According to a further embodiment, B is not 2,4-dichlorophenyl, not 2-chloro-4-fluorophenyl and not 2-chloro-6-fluorophenyl.

According to a further embodiment of the compounds IVa, A is phenyl which contains one, two or three independently selected substituents L, with the exceptions just mentioned. According to one aspect, the phenyl ring A contains two or three substituents L. According to a further aspect, the phenyl ring A contains one substituent L in the 4-position selected from the group consisting of C₁-C₄-alkyl, C₁-C₄-haloalkyl, C₁-C₄-alkoxy, C₁-C₄-haloalkoxy and C₁-C₄-haloalkylthio and no further substituent L or one or two further independently selected substituents L.

A and B and X in IVa have in particular the meanings as specified herein for formula I or III.1, taking into account the compounds which are excluded.

Compounds IVa can be obtained from compounds IVc

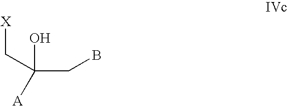

by reacting compounds IVc, for example, with acetic acid/H₂SO₄ in a suitable organic solvent such as, for example, an ether, such as Et₂O or dioxane, to form the double bond. Suitable methods are known to the person skilled in the art. X is as defined for formula III and A and B are as defined for formula I.

Some of the compounds of the formula IVc are novel. Accordingly, the invention also provides compounds of the formula IVc in which A and B are as defined or as preferably defined for formula I.

A and B and X in IVc have in particular the meanings as specified herein for formula I or III.1.

Compounds IVc can be obtained, for example, via a Grignard reaction according to the scheme below:

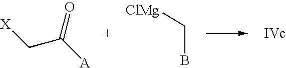

See also EP 409049.

Compounds of the formula III.1 can also be obtained from compounds of the formula III.2

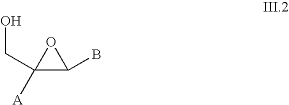

by introducing the leaving group X using methods known to the person skilled in the art. Accordingly, a compound of the formula III.2, for example with R—SO₂Y, where R is as defined for formula III.1 and Y is halogen, where R—SO₂Y is, for example, mesyl chloride, is reacted in the presence of a base (for example NEt₃) (see also EP 386557). To obtain compounds III.1 in which X is halogen, the corresponding compound III.2 can be reacted with C(Hal)₄ (Hal=Br or Cl) with PPh₃ in, for example, CH₂Cl₂, to give a compound III.1. Alternatively, a compound III.2 can be reacted with SOCl₂/pyridine (see also WO 2005/056548).

Some compounds of the formula III.2 are novel. Accordingly, the present invention also provides compounds of the formula III.2 in which A and B are as defined or as preferably defined for formula I.

According to a further embodiment of the compounds III.1, A is phenyl which contains one, two or three independently selected substituents L. According to one aspect, the phenyl ring A contains two or three substituents L. According to a further aspect, the phenyl ring A contains one substituent L in the 4-position selected from the group consisting of C₁-C₄-alkyl, C₁-C₄-haloalkyl, C₁-C₄-alkoxy, C₁-C₄-haloalkoxy and C₁-C₄-haloalkylthio and no further substituent L or one or two further independently selected substituents L.

According to one embodiment, compounds III.2 are excluded in which A and B are as defined below:

A=phenyl, 4-chlorophenyl, 4-bromophenyl, 4-fluorophenyl, 2-methoxyphenyl, 4-(tert-butyl)phenyl, 4-(tert-butoxy)phenyl, tert-butyl, methyl or 2-naphthyl; and
B=2,4-dichlorophenyl;
A=phenyl, 4-chlorophenyl or 4-fluorophenyl; and
B=2-chloro-4-fluorophenyl;
A=phenyl or 4-chlorophenyl; and
B=2-chloro-6-fluorophenyl.

According to a further embodiment, compounds III.2 in which A and B are as defined below are also excluded:

A=phenyl; and
B=2-chloro-5-NO₂-phenyl or 2,4-dimethylphenyl;
A=cyclohexyl, 2-chlorophenyl or 2,4-dichlorophenyl; and
B=2,4-dichlorophenyl;
A=4-fluorophenyl; and
B=2-chloro-5-NO₂-phenyl, 2-chloro-4-methoxyphenyl, 2,4-difluorophenyl, 2,6-difluorophenyl, 2-fluoro-3-fluorophenyl or 2,4-dimethylphenyl.

According to one aspect, A is phenyl which contains one, two or three substituents L.

A and B in III.2 have in particular the meanings as specified herein for formula III.1.

Compounds of the formula III.2 can be obtained from α,β-disubstituted acroleins of the type of the formula V

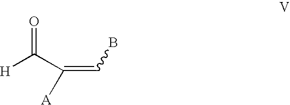

by initial epoxidation, for example with H₂O₂ in the presence of a base such as, for example, NaOH or by reaction with a peracid (for example MCPBA=m-chloroperoxy-benzoic acid) or tert-butyl hydroperoxide). This gives compounds of the formula Va

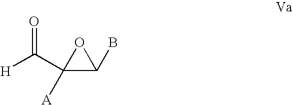

By reducing the aldehyde group in compounds Va, for example with NaBH₄ (see also EP 0 386 557A1), compounds III.2 are formed. Processes for epoxidation and reduction of the aldehyde group are well known to the person skilled in the art.

In formula V, the double bond can be present either in the (E) or in the (Z) configuration. This is indicated by the zig-zag bond between B and the double bond. Some of the compounds of the formula V are novel. Accordingly, the invention also provides compounds of the formula V in which A and B are as defined or as preferably defined for formula I.

The compounds V can be synthesized, for example, analogously to the procedure described in DE3601927, i.e. by reacting compounds of the type of the formula VI

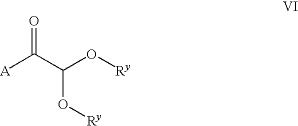

with appropriate phosphorus compounds of the Wittig or Horner-Emmons type with subsequent acidic cleavage of the acetal. In this case, R^{y} is in each case independently C₁-C₄-alkyl.

An alternative preparation of compounds V consists in the oxidation of compounds of the formula VII.

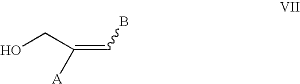

Suitable oxidizing agents and conditions are known to the person skilled in the art, for example a reaction according to Swern (Australian Journal of Chemistry, 57(6), 537-548; 2004), reactions with hypervalent iodine compounds (Organic Letters, 5(17), 2989-2992; 2003), with chromium compounds such as, for example, pyridinium dichromate (Tetrahedron, 45(1), 239-58; 1989) or with manganese oxides such as, for example, MnO₂ (Journal of the American Chemical Society, 107(13), 3963-71; 1985). The oxidation may also be carried out via a Dess-Martin oxidation in a solvent such as, for example, CH₂Cl₂.

In formula VII, the double bond may be present either in the (E) or in the (Z) configuration. This is indicated by the zig-zag bond between B and the double bond. Some compounds of the formula VII are novel. Accordingly, the invention furthermore provides compounds of the formula VII in which A and B are as defined or as preferably defined for formula I.

Starting with compounds VII, it is also possible to prepare compounds III.2 directly by epoxidation in the presence of a transition metal alkoxide such as, for example, V(O)(OR)₃ or Ti(OR)₄ (R═C₁-C₆-alkyl) and an oxidizing agent such as, for example, tBuOOH analogously to U.S. Pat. No. 5,399,708.

Compounds of the formula VII can be prepared from α,β-unsaturated acrylic esters of the formula VIII:

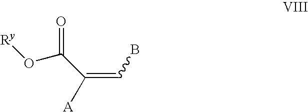

To this end, esters of the formula VIII are reduced to the alcohol VII. Suitable reducing procedures are well known to the person skilled in the art.

In formula VIII, the double bond may be present either in the (E) or in the (Z) configuration. This is indicated by the zig-zag bond between B and the double bond. Some compounds of the formula VIII are novel, and these compounds also form part of the subject matter of the present invention.

Compounds of the formula VIII can also be reduced in one step to the acrolein of the formula V, for example using metal hydrides such as, for example, diisobutylaluminum hydride at low temperatures. To this end, it is possible to use in particular aluminum hydrides, preferably lithium alanate (European Journal of Medicinal Chemistry, 40(6), 529-541; 2005) or dialkylaluminum hydrides such as, for example, DIBAL-H (Synlett, (18), 3182-3184; 2006).

The acrylic esters of the formula VIII are obtainable from glyoxalic esters of the formula IX by reaction with phosphorus compounds, for example of the Horner-Emmons type or Wittig compounds.

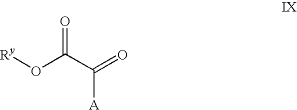

Such reactions are described, inter alia, Tetrahedron, 46(13-14), 4951-94; 1990, Tetrahedron Letters, 47(16), 2675-2678; 2006, Synthesis, (12), 1797-1802; 2003, WO9929645 or Synthetic Communications, 20(12), 1781-91; 1990.

The synthesis of glyoxalic esters is described, inter alia, in Journal of Organic Chemistry, 52(22), 5026-30; 1987 by reaction of Grignard compounds (starting with commercially available halogen compounds and magnesium) X¹MgA with oxalic esters of the formula

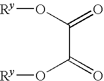

Suitable phosphorus compounds (of the Horner-Emmons type and Wittig type) can be prepared by known standard methods, for example from a compound of the type below:

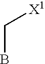

B is as defined above, X¹ is a leaving group such as, for example, a halide, preferably chlorine or bromine. The conversion of such halides into the desired Horner-Emmons or Wittig reagents can be carried out as described, for example, in Chemistry of Materials, 13(9), 3009-3017; 2001, European Journal of Organic Chemistry, (7), 1247-1257; 2005 or WO1992/05145.

The alkyl halides are either commercially available or can be prepared by standard methods, for example by halogenation of the corresponding methyl compound. Suitable halogenating agents for this reaction are N-bromosuccinimide (Chemistry-A European Journal, 12(21), 5632-5641; 2006) or N-chlorosuccinimide (Tetrahedron Letters, 47(37), 6607-6609; 2006).

According to one alternative, compounds of the formula V can also be prepared via an aldol synthesis according to the scheme below:

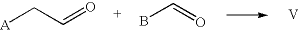

A further alternative of preparing compounds of the formula II consists in the epoxidation of a compound of the formula IVb.

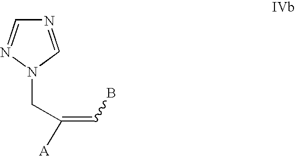

Suitable epoxidation methods are known to the person skilled in the art, see also the preparation of the compounds III.2 from compounds V.

In formula IVb, the double bond can be present either in the (E) or in the (Z) configuration. This is indicated by the zig-zag bond between B and the double bond. The present invention furthermore provides compounds of the formula IVb in which A and B are as defined or preferred for formula I.

According to a further embodiment of the compounds IVb, A is phenyl which contains one, two or three independently selected substituents L. According to one aspect, the phenyl ring A contains two or three substituents L. According to a further aspect, the phenyl ring A contains one substituent L in the 4-position selected from the group consisting of C₁-C₄-alkyl, C₁-C₄-haloalkyl, C₁-C₄-alkoxy, C₁-C₄-haloalkoxy and C₁-C₄-haloalkylthio and no further substituent L or one or two further independently selected substituents L.

According to one embodiment, compounds IVb are excluded in which A and B are as defined below:

A=phenyl, 4-chlorophenyl, 4-bromophenyl, 4-fluorophenyl, 2-methoxyphenyl, 4-(tert-butyl)phenyl, 4-(tert-butoxy)phenyl, tert-butyl, methyl or 2-naphthyl; and
B=2,4-dichlorophenyl;
A=phenyl, 4-chlorophenyl or 4-fluorophenyl; and
B=2-chloro-4-fluorophenyl;
A=phenyl or 4-chlorophenyl; and
B=2-chloro-6-fluorophenyl.

According to a further embodiment, compounds Vb in which A and B are as defined below are also excluded:

A=phenyl; and
B=2-chloro-5-NO₂-phenyl or 2,4-dimethyl phenyl;
or
A=cyclohexyl, 2-chlorophenyl or 2,4-dichlorophenyl; and
B=2,4-dichlorophenyl
or
A=4-fluorophenyl
B=2-chloro-5-NO₂-phenyl, 2-chloro-4-methoxyphenyl, 2,4-difluorophenyl, 2,6-difluorophenyl, 2-fluoro-3-fluorophenyl or 2,4-dimethylphenyl

According to one aspect, A is phenyl which contains one, two or three substituents L.

A and B in IVb have in particular the meanings as specified herein for formula III.1.

Compounds of the formula IVb can be obtained by reacting a compound of the formula IVa, as described above, with 1,2,4-triazole and a base. The reaction conditions can be chosen as described above for the preparation of compounds II from compounds III.

A further alternative of preparing compounds of the formula I consists in initially converting compounds of the formula III (see above) with hydrazine into compounds of the formula IIIa.

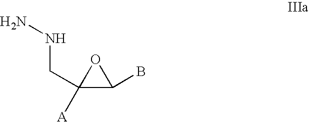

The present invention also provides compounds of the formula IIIa in which A and B are as defined or preferred for formula I.

Compounds of the formula IIIa can then be reacted with a thiocyanate YSCN in which Y is an alkali metal or ammonium, preferably sodium, potassium or ammonium, particularly preferably using NH₄SCN. This gives thiosemicarbazide compounds IIIb

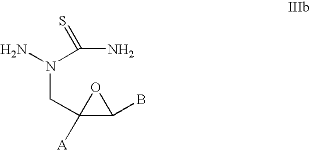

The triazolyl ring can be formed by reaction with formic acid, and what is obtained is the thione form of the corresponding compound of the formula I (D=SH) according to the invention, which, if appropriate, may be reacted further (see above). See also DE 19744400 (WO99/18088).

The present invention furthermore provides compounds of the formula IIIb in which A and B are as defined or preferred for formula I.

According to a further alternative, compounds IIIa can be reacted with formaldehyde ((CH₂O)ₙ) and a thiocyanate (YSCN, see above), which gives compounds of the formula IIIc

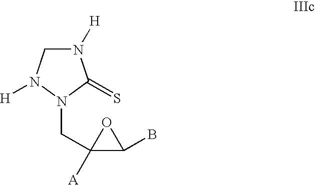

The triazolyl ring and thus the corresponding compound of the formula I is then formed by oxidation with, for example, iron(III) chloride in aqueous HCl (see also DE19961603 or WO 00/146158) or with oxygen in the presence of KOH and sulfur (see also WO 99/18087).

The present invention furthermore provides compounds of the formula IIIc in which A and B are as defined or preferred for formula I.

Yet another synthesis alternative consists in reacting a compound IIIa with a carbonyl compound (R^{x1}R^{x2})C═O (R^{x1}═C₁-C₄-alkyl or phenyl, R^{x2}=hydrogen or C₁-C₄-alkyl or R^{x1} and R^{x2} together form a —(CH₂)₅— chain), for example using acetone ((CH₃)₂CO) and a thiocyanate YSCN in which Y is sodium, potassium or ammonium, to give compounds IIId,

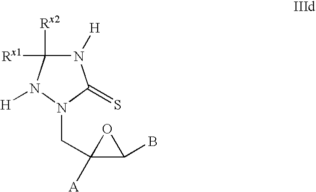

and then converting the compound formed in this manner with formic acid, if appropriate in the presence of a catalyst (for example HCl, H₂SO₄, p-toluenesulfonic acid, metal oxides such as, for example, amorphous TiO₂) into the corresponding triazole compound I. In this case, R^{x1} and R^{x2} are preferably both methyl (compounds IIId-1). See also DE19744401 and WO 99/18086.

The present invention furthermore provides compounds of the formula IIId in which A and B are as defined or preferred for formula I.

In some of the definitions of the symbols in the formulae given herein, collective terms are used which are generally representative of the following substituents:

halogen: fluorine, chlorine, bromine and iodine;
alkyl and the alkyl moieties of composite groups such as, for example, alkylamino: saturated straight-chain or branched hydrocarbon radicals having 1 to 4, 6, 8 or 12 carbon atoms, for example C₁-Cₛ-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-tri-methylpropyl, 1-ethyl-1-methylpropyl and 1-ethyl-2-methylpropyl;
haloalkyl: alkyl as mentioned above, where some or all of the hydrogen atoms in these groups are replaced by halogen atoms as mentioned above; in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl
alkenyl and also the alkenyl moieties in composite groups, such as alkenyloxy: unsaturated straight-chain or branched hydrocarbon radicals having 2 to 4, 2 to 6 or 2 to 8 carbon atoms and one double bond in any position. According to the invention, it may be preferred to use small alkenyl groups, such as (C₂-C₄)-alkenyl; on the other hand, it may also be preferred to employ larger alkenyl groups, such as (C₅-C₈)-alkenyl. Examples of alkenyl groups are, for example, C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: alkenyl as defined above, where some or all of the hydrogen atoms in these groups are replaced by halogen atoms as described above under haloalkyl, in particular by fluorine, chlorine or bromine;
alkadienyl: unsaturated straight-chain or branched hydrocarbon radicals having 4 to 6 or 4 to 8 carbon atoms and two double bonds in any position;
alkynyl and the alkynyl moieties in composite groups: straight-chain or branched hydrocarbon groups having 2 to 4, 2 to 6 or 2 to 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
haloalkynyl: alkynyl as defined above, where some or all of the hydrogen atoms in these groups are replaced by halogen atoms as described above under haloalkyl, in particular by fluorine, chlorine or bromine;
cycloalkyl and also the cycloalkyl moieties in composite groups: mono- or bicyclic saturated hydrocarbon groups having 3 to 8, in particular 3 to 6, carbon ring members, for example C₃-C₆-cycloalkyl, such as cyclopropyl, cyclobutyl, cyclopentyl, cyclohexyl;
halocycloalkyl: cycloalkyl as defined above, where some or all of the hydrogen atoms in these groups are replaced by halogen atoms as described above under haloalkyl, in particular by fluorine, chlorine or bromine;
cycloalkenyl: monocyclic monounsaturated hydrocarbon groups having preferably 3 to 8 or 4 to 6, in particular 5 to 6, carbon ring members, such as cyclopenten-1-yl, cyclopenten-3-yl, cyclohexen-1-yl, cyclohexen-3-yl, cyclohexen-4-yl and the like;
halocycloalkenyl: cycloalkenyl as defined above, where some or all of the hydrogen atoms in these groups are replaced by halogen atoms as described above under haloalkyl, in particular by fluorine, chlorine or bromine;
alkoxy: an alkyl group as defined above which is attached via an oxygen, preferably having 1 to 8, more preferably 2 to 6, carbon atoms. Examples are: methoxy, ethoxy, n-propoxy, 1-methylethoxy, butoxy, 1-methylpropoxy, 2-methylpropoxy or 1,1-dimethylethoxy, and also for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;
haloalkoxy: alkoxy as defined above, where some or all of the hydrogen atoms in these groups are replaced by halogen atoms as described above under haloalkyl, in particular by fluorine, chlorine or bromine. Examples are OCH₂F, OCHF₂, OCF₃, OCH₂Cl, OCHCl₂, OCCl₃, chlorofluoromethoxy, dichlorofluoromethoxy, chlorodifluoro-methoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy; and also 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy.
alkylene: divalent unbranched chains of CH₂ groups. Preference is given to (C₁-C₆)-alkylene, more preference to (C₂-C₄)-alkylene; furthermore, it may be preferred to use (C₁-C₃)-alkylene groups. Examples of preferred alkylene radicals are CH₂, CH₂CH₂, CH₂CH₂CH₂, CH₂(CH₂)₂CH₂, CH₂(CH₂)₃CH₂ and CH₂(CH₂)₄CH₂;
a 3-, 4-, 5-, 6-, 7-, 8-, 9- or 10-membered saturated or partially unsaturated heterocycle which contains 1, 2, 3 or 4 heteroatoms from the group consisting of O, N and S, where the heterocycle in question may be attached via a carbon atom or, if present, via a nitrogen atom. According to the invention, it may be preferred for the heterocycle in question to be attached via carbon, on the other hand, it may also be preferred for the heterocycle to be attached via nitrogen. In particular:
a three- or four-membered saturated heterocycle (hereinbelow also referred to as heterocyclyl) which contains one or two heteroatoms from the group consisting of O, N and S as ring members;
a five- or six-membered saturated or partially unsaturated heterocycle which contains one, two, three or four heteroatoms from the group consisting of O, N and S as ring members: for example monocyclic saturated or partially unsaturated heterocycles which, in addition to carbon ring members, contain one, two or three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydro-oxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexa-hydropyridazinyl, 2-hexahydropyrimidinyl, 4-hexahydropyrimidinyl, 5-hexahydro-pyrimidinyl, 2-piperazinyl, 1,3,5-hexahydrotriazin-2-yl and 1,2,4-hexahydrotriazin-3-yl and also the corresponding-ylidene radicals;
a seven-membered saturated or partially unsaturated heterocycle which contains one, two, three or four heteroatoms from the group consisting of O, N and S as ring members: for example mono- and bicyclic heterocycles having 7 ring members which, in addition to carbon ring members, contain one, two or three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetra-hydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding ylidene radicals;
a 5-, 6-, 7-, 8-, 9- or 10-membered aromatic heterocycle which contains 1, 2, 3 or 4 heteroatoms from the group consisting of O, N and S: in particular a five- or six-membered aromatic mono- or bicyclic heterocycle which contains one, two, three or four heteroatoms from the group consisting of O, N and S: the heterocycle in question may be attached via a carbon atom or, if present, via a nitrogen atom. According to the invention, it may be preferred for the heterocycle in question to be attached via carbon, on the other hand, it may also be preferred for the heterocycle to be attached via nitrogen. The heterocycle is in particular:
   5-membered heteroaryl which contains one, two, three or four nitrogen atoms or one, two or three nitrogen atoms and/or one sulfur or oxygen atom, where the heteroaryl may be attached via carbon or nitrogen, if present: 5-membered heteroaryl groups which, in addition to carbon atoms, may contain one to four nitrogen atoms or one, two or three nitrogen atoms and/or one sulfur or oxygen atom as ring members, for example furyl, thienyl, pyrrolyl, pyrazolyl, imidazolyl, triazolyl (1,2,3-; 1,2,4-triazolyl), tetrazolyl, oxazolyl, isoxazolyl, 1,3,4-oxadiazolyl, thiazolyl, isothiazolyl and thiadiazolyl, in particular 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
   6-membered heteroaryl which contains one, two, three or four, preferably one, two or three, nitrogen atoms, where the heteroaryl may be attached via carbon or nitrogen, if present: 6-ring heteroaryl groups which, in addition to carbon atoms, may contain one to four or one, two or three nitrogen atoms as ring members, for example pyridinyl, pyrimidinyl, pyrazinyl, pyridazinyl, 1,2,3-triazinyl, 1,2,4-triazinyl, 1,3,5-triazinyl, in particular 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The novel compounds according to the invention contain chiral centers and are generally obtained in the form of racemates or as diastereomer mixtures of erythro and threo forms. The erythro and threo diastereomers of the compounds according to the invention can be separated and isolated in pure form, for example, on the basis of their different solubilities or by column chromatography. Using known methods, such uniform pairs of diastereomers can be used to obtain uniform enantiomers. Suitable for use as antimicrobial agents are both the uniform diastereomers or enantiomers and mixtures thereof obtained in the synthesis. This applies correspondingly to the fungicidal compositions.

Accordingly, the invention provides both the pure enantiomers or diastereomers and mixtures thereof. This applies to the compounds according to the invention of the formula I and, if appropriate, correspondingly to their precursors. The scope of the present invention includes in particular the (R) and (S) isomers and the racemates of the compounds according to the invention, in particular of the formula I or II, which have centers of chirality. Suitable compounds according to the invention, in particular of the formula I or II, also include all possible stereoisomers (cis/trans isomers) and mixtures thereof.

The compounds according to the invention, in particular of the formula I or II, may be present in various crystal modifications which may differ in their biological activity. They are included in the scope of the present invention.

In the compounds according to the invention I, particular preference is given to the following meanings of the substituents, in each case on their own or in combination. According to the invention, B is phenyl which contains one substituent L in the 2-position and one, two or three further independently selected substituents L. According to a preferred embodiment R is a group B-1

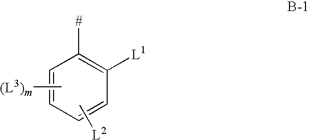

in which # denotes the point of attachment of the phenyl ring to the oxirane ring; and
L¹ is selected from the group consisting of halogen, C₁-C₄-alkyl, C₁-C₄-haloalkyl, C₁-C₄-alkoxy, C₁-C₄-haloalkoxy and C₁-C₄-haloalkylthio, preferably selected from the group consisting of F, Cl, methyl, ethyl, methoxy, ethoxy, CF₃, CHF₂, OCF₃, OCHF₂ and SCF₃;
L² is selected from the group consisting of halogen, C₁-C₄-alkyl, C₁-C₄-haloalkyl, C₁-C₄-alkoxy, C₁-C₄-haloalkoxy and C₁-C₄-haloalkylthio, preferably selected from the group consisting of F, Cl, methyl, ethyl, methoxy, ethoxy, CF₃, CHF₂, OCF₃, OCHF₂ and SCF₃;
L³ is independently selected from the group consisting of halogen, C₁-C₄-alkyl, C₁-C₄-haloalkyl, C₁-C₄-alkoxy, C₁-C₄-haloalkoxy and C₁-C₄-haloalkylthio, preferably selected from the group consisting of F, Cl, methyl, ethyl, methoxy, ethoxy, CF₃, CHF₂, OCF₃, OCHF₂ and SCF₃; and
m is 0, 1 or 2.

According to a preferred embodiment, L¹ is F. According to another preferred embodiment, L¹ is Cl. According to a further preferred embodiment, L¹ is methyl. According to yet a further preferred embodiment, L¹ is methoxy. According to yet a further preferred embodiment, L¹ is CF₃. According to yet a further preferred embodiment, L¹ is OCF₃ or OCHF₂. According to a preferred embodiment, in the compounds of the formula I according to the invention, B is thus phenyl which contains a substituent selected from the group consisting of F, Cl, CH₃, OCH₃, CF₃, CHF₂, OCF₃ and OCHF₂ in the 2-position and one or two further independently selected substituents L.

According to a preferred embodiment, L² is F. According to another preferred embodiment, L² is Cl. According to a further preferred embodiment, L² is methyl.

According to yet a further preferred embodiment, L² is methoxy. According to yet a further preferred embodiment, L² is CF₃. According to yet a further preferred embodiment, L² is OCF₃ or OCHF₂.

According to a preferred embodiment, L³ is F. According to another preferred embodiment, L³ is Cl. According to a further preferred embodiment, L³ is methyl. According to yet a further preferred embodiment, L³ is methoxy. According to yet a further preferred embodiment, L³ is CF₃. According to yet a further preferred embodiment, L³ is OCF₃ or OCHF₂.

According to a preferred embodiment, n=0; i.e. B is a disubstituted phenyl ring. According to a preferred aspect, B is a 2,3-disubstituted phenyl ring. According to a further preferred aspect, the pheny ring B is 2,4-disubstituted. According to yet a further preferred aspect, the phenyl ring B is 2,5-disubstituted. According to yet a further preferred aspect, the phenyl ring is 2,6-disubstituted.

B is phenyl which contains a substituent L in the 2-position and exactly one further substituent L, where L is in each case independently selected from the group consisting of F, Br, cyano, nitro, cyanato (OCN), C₁-C₈-alkyl, C₁-C₈-haloalkyl, phenyl-C₁-Cₒ-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈-alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkylenoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)^{,N}, C(═O)A², C(═S)A², wok-, phenyl, phenyloxy and a five- or six-membered saturated, partially unsaturated or aromatic heterocycle which contains one, two, three or four heteroatoms from the group consisting of O, N and S; where n, A¹, A², A³, A⁴ are as defined herein.

According to a further preferred embodiment, m=1; i.e. B is a trisubstituted phenyl ring. According to a preferred aspect, the phenyl ring B is 2,3,5-trisubstituted. According to another preferred further aspect, the phenyl ring B is 2,3,4-trisubstituted. According to yet a further preferred embodiment, the phenyl ring B is 2,4,5-trisubstituted.

Preference is given to the compounds of the formula I in which B is phenyl which contains a substituent L¹ in the 2-position and a substituent L* in the 4-position:

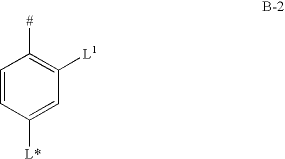

where # is the point of attachment of the phenyl ring to the oxirane ring; and L¹ has the meaning of L.

The meanings mentioned as being preferred for L are also to be considered as being preferred for L*, with the proviso that it is not chlorine.

Preference is furthermore given to the compounds of the formula I in which B is phenyl which contains a substituent L¹ in the 2-position and a further substituent L in the 3-, 5- or 6-position:

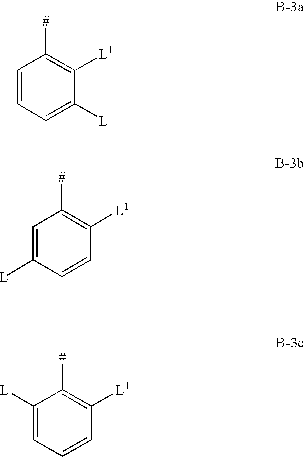

where # is the point of attachment of the phenyl ring to the oxirane ring; and L¹ has the meaning of L.

Preference is furthermore given to the compounds of the formula I in which B is phenyl which contains a substituent L¹ in the 2-position and two further substituents L:

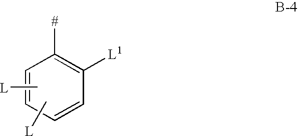

where # is the point of attachment of the phenyl ring to the oxirane ring; and L¹ has the meaning of L.

Preference is furthermore given to the compounds of the formula I in which B is phenyl which contains a substituent L¹ in the 2-position and three further substituents L:

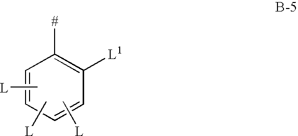

where # is the point of attachment of the phenyl ring to the oxirane ring; and L¹ has the meaning of L.

According to a preferred embodiment, B is a phenyl B-6:

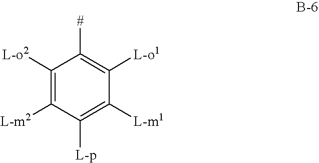

where # is the point of attachment of the phenyl ring to the oxirane ring; and where

### L-o¹ L,

L-m¹ L or hydrogen,
L-p L* or hydrogen,
L-m² L or hydrogen,
L-o² L or hydrogen,
with the proviso that L-m¹, L-p, L-m² and L-o² are not all simultaneously hydrogen. L-o¹ is preferably F, Cl, CH₃, C₂H₅, CF₃, OCH₃, OC₂H₅, OCF₃, OCHF₂,SCF₃; particularly preferably F, Cl, CH₃, OCH₃, CF₃, CHF₂, OCF₃ and OCHF₂; particularly preferably F, Cl, CH₃.
L-m¹ is preferably hydrogen, F, Cl, CH₃, C₂H₅, CF₃, OCH₃, OC₂H₅, OCF₃, OCHF₂, SCF₃;
particularly preferably hydrogen, F, Cl, CH₃, OCH₃, CF₃, CHF₂, OCF₃ and OCHF₂;

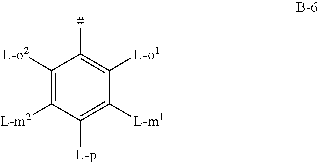

where # is the point of attachment of the phenyl ring to the oxirane ring; and where

### L-o¹ L,

L-m¹ L or hydrogen,
L-p L* or hydrogen,
L-m² L or hydrogen,
L-o² L or hydrogen,
with the proviso that L-m¹, L-p, L-m² and L-o² are not all simultaneously hydrogen.
L-o¹ is preferably F, Cl, CH₃, C₂H₅, CF₃, OCH₃, OC₂H₅, OCF₃, OCHF₂, SCF₃; particularly preferably F, Cl, CH₃, OCH₃, CF₃, CHF₂, OCF₃ and OCHF₂; particularly preferably F, Cl, CH₃.
L-m¹ is preferably hydrogen, F, Cl, CH₃, C₂H₅, CF₃, OCH₃, OC₂H₅, OCF₃, OCHF₂, SCF₃; particularly preferably hydrogen, F, Cl, CH₃, OCH₃, CF₃, CHF₂, OCF₃ and OCHF₂;
particularly preferably hydrogen, F, Cl, CH₃.
L-p is preferably hydrogen, F, CH₃, C₂H₅, CF₃, OCH₃, OC₂H₅, OCF₃, OCHF₂,SCF₃; particularly preferably hydrogen, F, CH₃, OCH₃, CF₃, CHF₂, OCF₃ and OCHF₂;
particularly preferably F, CH₃.
L-m² is preferably hydrogen, F, Cl, CH₃, C₂H₅, CF₃, OCH₃, OC₂H₅, OCF₃, OCHF₂, SCF₃; particularly preferably hydrogen, F, Cl, CH₃; particularly preferably hydrogen.
L-o² is preferably hydrogen, F, Cl, CH₃, C₂H₅, CF₃, OCH₃, OC₂H₅, OCF₃, OCHF₂, SCF₃;
particularly preferably hydrogen, F, Cl, CH₃;
particularly preferably hydrogen.

Particular preference is given to the meanings of B-6, in which L-m² and L-o² are hydrogen.

The meanings given as being preferred for L are also to be considered as being preferred for L*, with the proviso that it is not chlorine.

According to a particularly preferred embodiment, the respective independently selected substituents L are selected from the group consisting of F, Cl, CH₃, OCH₃, CF₃, CHF₂, OCF₃ and OCHF₂, with the proviso that L in the 4-position of B is not chlorine.

According to a further preferred embodiment, A is C₁-C₈-haloalkyl, in particular CF₃.

According to a further embodiment, A is unsubstituted phenyl.

According to yet a further embodiment, A is phenyl which contains one, two, three or four, in particular one, two or three, independently selected substituents L.

According to a further embodiment, the phenyl ring is monosubstituted by a substituent L, where L, according to a specific aspect of this embodiment, is located in the para-position to the point of attachment of the phenyl ring to the oxirane ring.

According to a further embodiment, the phenyl ring is disubstituted by two independently selected substituents L. According to a further embodiment of the invention, A is a phenyl ring which contains a substituent L in the para-position and furthermore has a further independently selected substituent L. According to one aspect, the phenyl ring is 2,4-disubstituted. According to a further aspect, the phenyl ring is 3,4-disubstituted.

According to a further embodiment of the invention, A is a phenyl ring which contains a substituent L in the para-position and furthermore contains two further independently selected substituents L. According to one aspect, the phenyl ring is 2,3,4-trisubstituted. According to yet a further aspect, the phenyl ring is 2,4,6-trisubstituted.

Unless indicated otherwise, L independently has the following meanings:

According to one embodiment, L is independently selected from the group consisting of halogen, cyano, nitro, cyanato (OCN), C₁-C₄-alkyl, C₁-C₄-haloalkyl, C₁-C₄-alkoxy, C₁-C₄-haloalkoxy, C₃-C₆-cycloalkyl, C₃-C₆-halocycloalkyl, S-A¹, C(═O)A², C(═S)A², NA³A⁴; where A¹, A², A³, A⁴ are as defined below:
A¹ is hydrogen, hydroxyl, C₁-C₄-alkyl, C₁-C₄-haloalkyl;
A² is one of the groups mentioned for A₁ or C₁-C₄-alkoxy, C₁-C₄-haloalkoxy, C₃-C₆-cycloalkyl, C₃-C₆-halocycloalkyl, C₃-C₆-cycloalkoxy or C₃-C₆-halocycloalkoxy;
A³,A⁴ independently of one another are hydrogen, C₁-C₄-alkyl, C₁-C₄-haloalkyl;
where the aliphatic and/or alicyclic groups of the radical definitions of L for their part may carry one, two, three or four identical or different groups R^{L}:
R^{L} is halogen, cyano, nitro, C₁-C₄-alkyl, C₁-C₄-haloalkyl, C₁-C₄-alkoxy, C₁-C₄-haloalkoxy, C₃-C₆-cycloalkyl, C₃-C₆-halocycloalkyl, amino, C₁-C₈-alkylamino, di-C₁-C₈-alkylamino.

Furthermore preferably, L is independently selected from the group consisting of halogen, NO₂, amino, C₁-C₄-alkyl, C₁-C₄-haloalkyl, C₁-C₄-alkoxy, C₁-C₄-haloalkoxy, C₁-C₄-alkylamino, di-C₁-C₄-alkylamino, thio and C₁-C₄-alkylthio.

Furthermore preferably, L is independently selected from the group consisting of halogen, C₁-C₄-alkyl, C₁-C₄-haloalkyl, C₁-C₄-alkoxy, C₁-C₄-haloalkoxy and C₁-C₄-haloalkylthio.

According to a further preferred embodiment, L is independently selected from the group consisting of F, Cl, Br, CH₃, C₂H₅, i-C₃H₇, t-C₄H₅, OCH₃, OC₂H₆, CF₃, CCl₃, CHF₂, CClF₂, OCF₃, OCHF₂ and SCF₃, in particular selected from the group consisting of F, Cl, CH₃, C₂H₅, OCH₃, OC₂H₆, CF₃, CHF₂, OCF₃, OCHF₂ and SCF₃, in particular selected from the group consisting of F, Cl, CH₃, C₂H₅, CF₃, OCH₃, OC₂H₆, OCF₃, OCHF₂ and SCF₃. According to one aspect, L is independently selected from the group consisting of F, Cl, CH₃, OCH₃, CF₃, OCF₃ and OCHF₂. It may be preferred for L to be independently F or Cl.

The meanings described above of the variables A, B and L for compounds I apply, unless indicated otherwise, correspondingly to the precursors of the compounds according to the invention.

According to one embodiment of the invention, D is a group SR, where R is hydrogen (compounds I-1). According to a further embodiment, D is a group SR, where R is C₁-C₄-alkyl, in particular methyl or ethyl, preferably methyl.

According to a further embodiment of the invention, D is a group SR where R is C(═O)R³ and R³ is NA³A⁴, where A³ and A⁴ independently of one another are hydrogen or C₁-C₈-alkyl.

According to a further embodiment of the invention, D is a group SR, where R is C(═O)R³ and R³ is hydrogen, C₁-C₄-alkyl, C₁-C₄-haloalkyl, C₁-C₄-alkoxy, C₁-C₄-haloalkoxy, phenyl or benzyl. According to a specific aspect thereof, R³ is hydrogen. According to a further aspect thereof, R³ is C₁-C₄-alkyl, in particular methyl or ethyl, preferably methyl. According to yet a further aspect, R³ is C₁-C₄-haloalkyl, in particular trifluoromethyl. According to yet a further aspect, R³ is C₁-C₄-alkoxy, in particular methoxy or ethoxy.

According to a further embodiment of the invention, D is a group SR, where R is C(═O)R³ and R³ is (C₁-C₄)alkylamino, di(C₁-C₄)alkylamino or phenylamino. According to one aspect thereof, R³ is methylamino, dimethylamino, ethylamino, diethylamino or phenylamino.

According to a further embodiment of the invention, D is a group SR, where R is CN.

According to a further embodiment of the invention, D is a group SR, where R is SO₂R⁴ and R⁴ is C₁-C₄-alkyl, phenyl-C₁-C₄-alkyl or phenyl, where the phenyl groups are in each case unsubstituted or substituted by one, two or three groups independently selected from the group consisting of halogen and C₁-C₄-alkyl.

According to a further embodiment of the invention, D is a group SM, where M is an alkali metal cation, an equivalent of an alkaline earth metal cation, an equivalent of a copper, zinc, iron or nickel cation or an ammonium cation of the formula (E)

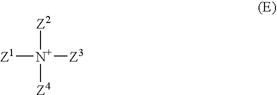

in which
Z¹ and Z² independently of one another are hydrogen or C₁-C₄-alkyl; and
Z³ and Z⁴ independently of one another are hydrogen, C₁-C₄-alkyl, benzyl or phenyl.

According to one embodiment, M is Na, 1/2 Cu, 1/3 Fe, HN(CH₃)₃, HN(C₂H₅)₃, N(CH₃)₄ or H₂N(C₃H₇)₂, in particular Na, 1/2 Cu, HN(CH₃)₃ or HN(C₂H₅)₃, especially Na, 1/2 Cu, HN(CH₃)₃ or HN(C₂H₅)₃.

According to a further embodiment of the invention, D is a group DI (compounds I-2) where A and R ara independently as defined or preferred herein:

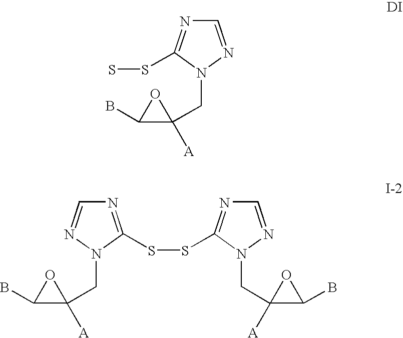

Preferably, both As and both Bs in the compounds I-2 have the same meaning.

According to a further embodiment of the invention, D is a group DII where # is the point of attachment to the triazolyl ring and Q, R¹ and R² are as defined or preferred herein:

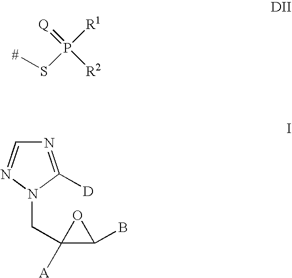

In particular with a view to their use, preference is given to the compounds I according to the invention compiled in Tables 1a to 162a and Tables 1b to 162b below. The groups mentioned for a substituent in the tables are furthermore per se, independently of the combination in which they are mentioned, a particularly preferred aspect of the substituent in question.

Table 1a
Compounds I in which B is 2,3-difluorophenyl and the combination of A and D corresponds in each case to one row of Table A (compounds I.1aA-1 to I.1aA-2295)

Table 2a
Compounds I in which B is 2,4-difluorophenyl and the combination of A and D corresponds in each case to one row of Table A (compounds I.2aA-1 to I.2aA-2295)

Table 3a

Compounds I in which B is 2,5-difluorophenyl and the combination of A and D corresponds in each case to one row of Table A (compounds I.3aA-1 to I.3aA-2295)

Table 4a
Compounds I in which B is 2,6-difluorophenyl and the combination of A and D corresponds in each case to one row of Table A (compounds I.4aA-1 to I.4aA-2295)

Table 5a
Compounds I in which B is 2-fluoro-3-chlorophenyl and the combination of A and D corresponds in each case to one row of Table A (compounds I.5aA-1 to I.5aA-2295)

Table 6a Compounds I in which B is 2-fluoro-4-chlorophenyl and the combination of A and D corresponds in each case to one row of Table A (compounds I.6aA-1 to I.6aA-2295)

Table 7a
Compounds I in which B is 2-fluoro-5-chlorophenyl and the combination of A and D corresponds in each case to one row of Table A (compounds I.7aA-1 to I.7aA-2295)

Table 8a
Compounds I in which B is 2-fluoro-6-chlorophenyl and the combination of A and D corresponds in each case to one row of Table A (compounds I.8aA-1 to I.8aA-2295)

Table 9a
Compounds I in which B is 2-chloro-3-fluorophenyl and the combination of A and D corresponds in each case to one row of Table A (compounds I.9aA-1 to I.9aA-2295)

Table 10a
Compounds I in which B is 2-chloro-4-fluorophenyl and the combination of A and D corresponds in each case to one row of Table A (compounds I.10aA-1 to I.10aA-2295)

Table 11a
Compounds I in which B is 2-chloro-5-fluorophenyl and the combination of A and D corresponds in each case to one row of Table A (compounds I.11aA-1 to I.11aA-2295)

Table 12a
Compounds I in which B is 2-methyl-3-fluorophenyl and the combination of A and D corresponds in each case to one row of Table A (compounds I.12aA-1 to I.12aA-2295)

Table 13a
Compounds I in which B is 2-methyl-4-fluorophenyl and the combination of A and D corresponds in each case to one row of Table A (compounds I.13aA-1 to I.13aA-2295)

Table 14a

Compounds I in which B is 2-methyl-5-fluorophenyl and the combination of A and D corresponds in each case to one row of Table A (compounds I.14aA-1 to I.14aA-2295)

Table 15a Compounds I in which B is 2-methyl-6-fluorophenyl and the combination of A and D corresponds in each case to one row of Table A (compounds I.15aA-1 to I.15aA-2295)

Table 16a
Compounds I in which B is 2-fluoro-3-methylphenyl and the combination of A and D corresponds in each case to one row of Table A (compounds I.16aA-1 to I.16aA-2295)

Table 17a
Compounds I in which B is 2-fluoro-4-methylphenyl and the combination of A and D corresponds in each case to one row of Table A (compounds I.17aA-1 to I.17aA-2295)

Table 18a Compounds I in which B is 2-fluoro-5-methylphenyl and the combination of A and D corresponds in each case to one row of Table A (compounds I.18aA-1 to I.18aA-2295)

Table 19a
Compounds I in which B is 2-ethyl-3-fluorophenyl and the combination of A and D corresponds in each case to one row of Table A (compounds I.19aA-1 to I.19aA-2295)

Table 20a
Compounds I in which B is 2-ethyl-4-fluorophenyl and the combination of A and D corresponds in each case to one row of Table A (compounds I.20aA-1 to I.20aA-2295)

Table 21a
Compounds I in which B is 2-ethyl-5-fluorophenyl and the combination of A and D corresponds in each case to one row of Table A (compounds I.21aA-1 to I.21aA-2295)

Table 22a
Compounds I in which B is 2-ethyl-6-fluorophenyl and the combination of A and D corresponds in each case to one row of Table A (compounds I.22aA-1 to I.22aA-2295)

Table 23a
Compounds I in which B is 2-fluoro-3-ethylphenyl and the combination of A and D corresponds in each case to one row of Table A (compounds I.23aA-1 to I.23aA-2295)

Table 24a
Compounds I in which B is 2-fluoro-4-ethylphenyl and the combination of A and D corresponds in each case to one row of Table A (compounds I.24aA-1 to I.24aA-2295)

Table 25a
Compounds I in which B is 2-methoxy-3-fluorophenyl and the combination of A and D corresponds in each case to one row of Table A (compounds I.25aA-1 to I.25aA-2295)

Table 26a
Compounds I in which B is 2-methoxy-4-fluorophenyl and the combination of A and D corresponds in each case to one row of Table A (compounds I.26aA-1 to I.26aA-2295)

Table 27a
Compounds I in which B is 2-methoxy-5-fluorophenyl and the combination of A and D corresponds in each case to one row of Table A (compounds I.27aA-1 to I.27aA-2295)

Table 28a
Compounds I in which B is 2-methoxy-6-fluorophenyl and the combination of A and D corresponds in each case to one row of Table A (compounds I.28aA-1 to I.28aA-2295)

Table 29a
Compounds I in which B is 2-fluoro-3-methoxyphenyl and the combination of A and D corresponds in each case to one row of Table A (compounds I.29aA-1 to I.29aA-2295)

Table 30a
Compounds I in which B is 2-fluoro-4-methoxyphenyl and the combination of A and D corresponds in each case to one row of Table A (compounds I.30aA-1 to I.30aA-2295)

Table 31a
Compounds I in which B is 2-fluoro-5-methoxyphenyl and the combination of A and D corresponds in each case to one row of Table A (compounds I.31aA-1 to I.31aA-2295)

Table 32a
Compounds I in which B is 2-(trifluoromethyl)-3-fluorophenyl and the combination of A and D corresponds in each case to one row of Table A (compounds I.32aA-1 to I.32aA-2295)

Table 33a
Compounds I in which B is 2,4,5-trimethoxyphenyl and the combination of A and D corresponds in each case to one row of Table A (compounds I.33aA-1 to I.33aA-2295)

Table 34a
Compounds I in which B is 2,4,6-trimethoxyphenyl and the combination of A and D corresponds in each case to one row of Table A (compounds I.34aA-1 to I.34aA-2295)

Table 35a
Compounds I in which B is 2-fluoro-5-ethylphenyl and the combination of A and D corresponds in each case to one row of Table A (compounds I.35aA-1 to I.35aA-2295)

Table 36a
Compounds I in which B is 2-(trifluoromethyl)-4-fluorophenyl and the combination of A and D corresponds in each case to one row of Table A (compounds I.36aA-1 to I.36aA-2295)

Table 37a
Compounds I in which B is 2-(trifluoromethyl)-5-fluorophenyl and the combination of A and D corresponds in each case to one row of Table A (compounds I.37aA-1 to I.37aA-2295)

Table 38a
Compounds I in which B is 2-(trifluoromethyl)-6-fluorophenyl and the combination of A and D corresponds in each case to one row of Table A (compounds I.38aA-1 to I.38aA-2295)

Table 39a
Compounds I in which B is 2-fluoro-3-(trifluoromethyl)phenyl and the combination of A and D corresponds in each case to one row of Table A (compounds I.39aA-1 to 1.39aA-2295)

Table 40a
Compounds I in which B is 2-fluoro-4-(trifluoromethyl)phenyl and the combination of A and D corresponds in each case to one row of Table A (compounds I.40aA-1 to I.40aA-2295)

Table 41a
Compounds I in which B is 2-fluoro-5-(trifluoromethyl)phenyl and the combination of A and D corresponds in each case to one row of Table A (compounds I.41 aA-1 to I.41 aA-2295)

Table 42a
Compounds I in which B is 2-(trifluoromethoxy)-3-fluorophenyl and the combination of A and D corresponds in each case to one row of Table A (compounds I.42aA-1 to I.42aA-2295)

Table 43a
Compounds I in which B is 2-(trifluoromethoxy)-4-fluorophenyl and the combination of A and D corresponds in each case to one row of Table A (compounds I.43aA-1 to I.43aA-2295)

Table 44a
Compounds I in which B is 2-(trifluoromethoxy)-5-fluorophenyl and the combination of A and D corresponds in each case to one row of Table A (compounds I.44aA-1 to I.44aA-2295)

Table 45a
Compounds I in which B is 2-(trifluoromethoxy)-6-fluorophenyl and the combination of A and D corresponds in each case to one row of Table A (compounds I.45aA-1 to I.45aA-2295)

Table 46a
Compounds I in which B is 2-fluoro-3-(trifluoromethoxy)phenyl and the combination of A and D corresponds in each case to one row of Table A (compounds I.46aA-1 to I.46aA-2295)

Table 47a
Compounds I in which B is 2-fluoro-4-(trifluoromethoxy)phenyl and the combination of A and D corresponds in each case to one row of Table A (compounds I.47aA-1 to I.47aA-2295)

Table 48a
Compounds I in which B is 2-fluoro-5-(trifluoromethoxy)phenyl and the combination of A and D corresponds in each case to one row of Table A (compounds I.48aA-1 to I.48aA-2295)

Table 49a
Compounds I in which B is 2-(difluoromethoxy)-3-fluorophenyl and the combination of A and D corresponds in each case to one row of Table A (compounds I.49aA-1 to I.49aA-2295)

Table 50a
Compounds I in which B is 2-(difluoromethoxy)-4-fluorophenyl and the combination of A and D corresponds in each case to one row of Table A (compounds I.50aA-1 to I.50aA-2295)

Table 51a
Compounds I in which B is 2-(difluoromethoxy)-5-fluorophenyl and the combination of A and D corresponds in each case to one row of Table A (compounds I.51aA-1 to I.51aA-2295)

Table 52a
Compounds I in which B is 2-(difluoromethoxy)-6-fluorophenyl and the combination of A and D corresponds in each case to one row of Table A (compounds I.52aA-1 to I.52aA-2295)

Table 53a
Compounds I in which B is 2-fluoro-3-(difluoromethoxy)phenyl and the combination of A and D corresponds in each case to one row of Table A (compounds I.53aA-1 to I.53aA-2295)

Table 54a
Compounds I in which B is 2-fluoro-4-(difluoromethoxy)phenyl and the combination of A and D corresponds in each case to one row of Table A (compounds I.54aA-1 to I.54aA-2295)

Table 55a
Compounds I in which B is 2-fluoro-5-(difluoromethoxy)phenyl and the combination of A and D corresponds in each case to one row of Table A (compounds I.55aA-1 to I.55aA-2295)

Table 56a
Compounds I in which B is 2-(trifluoromethylthio)-3-fluorophenyl and the combination of A and D corresponds in each case to one row of Table A (compounds I.56aA-1 to I.56aA-2295)

Table 57a
Compounds I in which B is 2-(trifluoromethylthio)-4-fluorophenyl and the combination of A and D corresponds in each case to one row of Table A (compounds I.57aA-1 to I.57aA-2295)

Table 58a
Compounds I in which B is 2-(trifluoromethylthio)-5-fluorophenyl and the combination of A and D corresponds in each case to one row of Table A (compounds I.58aA-1 to I.58aA-2295)

Table 59a
Compounds I in which B is 2-(trifluoromethylthio)-6-fluorophenyl and the combination of A and D corresponds in each case to one row of Table A (compounds I.59aA-1 to I.59aA-2295)

Table 60a
Compounds I in which B is 2-fluoro-3-(trifluoromethylthio)phenyl and the combination of A and D corresponds in each case to one row of Table A (compounds I.60aA-1 to I.60aA-2295)

Table 61a
Compounds I in which B is 2-fluoro-4-(trifluoromethylthio)phenyl and the combination of A and D corresponds in each case to one row of Table A (compounds I.61aA-1 to I.61aA-2295)

Table 62a
Compounds I in which B is 2-fluoro-5-(trifluoromethylthio)phenyl and the combination of A and D corresponds in each case to one row of Table A (compounds I.622A-1 to I.62aA-2295)

Table 63a
Compounds I in which B is 2,3,4-trifluorophenyl and the combination of A and D corresponds in each case to one row of Table A (compounds I.63aA-1 to I.63aA-2295)

Table 64a
Compounds I in which B is 2,3,5-trifluorophenyl and the combination of A and D corresponds in each case to one row of Table A (compounds I.64aA-1 to I.64aA-2295)

Table 65a
Compounds I in which B is 2,3,6-trifluorophenyl and the combination of A and D corresponds in each case to one row of Table A (compounds I.65aA-1 to I.65aA-2295)

Table 66a
Compounds I in which B is 2,4,5-trifluorophenyl and the combination of A and D corresponds in each case to one row of Table A (compounds I.66aA-1 to I.66aA-2295)

Table 67a
Compounds I in which B is 2,4,6-trifluorophenyl and the combination of A and D corresponds in each case to one row of Table A (compounds I.67aA-1 to I.67aA-2295)

Table 68a
Compounds I in which B is 2,3-dichlorophenyl and the combination of A and D corresponds in each case to one row of Table A (compounds I.68aA-1 to I.68aA-2295)

Table 69a
Compounds I in which B is 2,4-dichlorophenyl and the combination of A and D corresponds in each case to one row of Table A (compounds I.69aA-1 to I.69aA-2295)

Table 70a
Compounds I in which B is 2,5-dichlorophenyl and the combination of A and D corresponds in each case to one row of Table A (compounds I.70aA-1 to I.70aA-2295)

Table 71a
Compounds I in which B is 2,6-dichlorophenyl and the combination of A and D corresponds in each case to one row of Table A (compounds I.71aA-1 to I.71aA-2295)

Table 72a
Compounds I in which B is 2,3-dimethylphenyl and the combination of A and D corresponds in each case to one row of Table A (compounds I.72aA-1 to I.72aA-2295)

Table 73a
Compounds I in which B is 2,4-dimethylphenyl and the combination of A and D corresponds in each case to one row of Table A (compounds I.73aA-1 to I.73aA-2295)

Table 74a
Compounds I in which B is 2,5-dimethylphenyl and the combination of A and D corresponds in each case to one row of Table A (compounds I.74aA-1 to I.74aA-2295)

Table 75a
Compounds I in which B is 2,6-dimethylphenyl and the combination of A and D corresponds in each case to one row of Table A (compounds I.75aA-1 to I.75aA-2295)

Table 76a
Compounds I in which B is 2,3-diethylphenyl and the combination of A and D corresponds in each case to one row of Table A (compounds I.76aA-1 to I.76aA-2295)

Table 77a
Compounds I in which B is 2,4-diethylphenyl and the combination of A and D corresponds in each case to one row of Table A (compounds I.77aA-1 to I.77aA-2295)

Table 78a
Compounds I in which B is 2,5-diethylphenyl and the combination of A and D corresponds in each case to one row of Table A (compounds I.78aA-1 to I.78aA-2295)

Table 79a
Compounds I in which B is 2,6-diethylphenyl and the combination of A and D corresponds in each case to one row of Table A (compounds I.79aA-1 to I.79aA-2295)

Table 80a
Compounds I in which B is 2,3-dimethoxyphenyl and the combination of A and D corresponds in each case to one row of Table A (compounds I.80aA-1 to I.80aA-2295)

Table 81a
Compounds I in which B is 2,4-dimethoxyphenyl and the combination of A and D corresponds in each case to one row of Table A (compounds I.81aA-1 to I.81aA-2295)

Table 82a
Compounds I in which B is 2,5-dimethoxyphenyl and the combination of A and D corresponds in each case to one row of Table A (compounds I.82aA-1 to I.82aA-2295)

Table 83a
Compounds I in which B is 2,6-dimethoxyphenyl and the combination of A and D corresponds in each case to one row of Table A (compounds I.83aA-1 to I.83aA-2295)

Table 84a
Compounds I in which B is 2,3-di(trifluoromethyl)phenyl and the combination of A and D corresponds in each case to one row of Table A (compounds I.84aA-1 to I.84aA-2295)

Table 85a
Compounds I in which B is 2,4-di(trifluoromethyl)phenyl and the combination of A and D corresponds in each case to one row of Table A (compounds I.85aA-1 to I.85aA-2295)

Table 86a
Compounds I in which B is 2,5-di(trifluoromethyl)phenyl and the combination of A and D corresponds in each case to one row of Table A (compounds I.86aA-1 to I.86aA-2295)

Table 87a
Compounds I in which B is 2,6-di(trifluoromethyl)phenyl and the combination of A and D corresponds in each case to one row of Table A (compounds I.87aA-1 to I.87aA-2295)

Table 88a
Compounds I in which B is 2,3-di(trifluoromethoxy)phenyl and the combination of A and D corresponds in each case to one row of Table A (compounds I.88aA-1 to I.88aA-2295)

Table 89a
Compounds I in which B is 2,4-di(trifluoromethoxy)phenyl and the combination of A and D corresponds in each case to one row of Table A (compounds I.89aA-1 to I.89aA-2295)

Table 90a
Compounds I in which B is 2,5-di(trifluoromethoxy)phenyl and the combination of A and D corresponds in each case to one row of Table A (compounds I.90aA-1 to I.90aA-2295)

Table 91a
Compounds I in which B is 2,6-di(trifluoromethoxy)phenyl and the combination of A and D corresponds in each case to one row of Table A (compounds I.91aA-1 to I.91 aA-2295)

Table 92a
Compounds I in which B is 2,3-di(difluoromethoxy)phenyl and the combination of A and D corresponds in each case to one row of Table A (compounds I.92aA-1 to I.92aA-2295)

Table 93a
Compounds I in which B is 2,4-di(difluoromethoxy)phenyl and the combination of A and D corresponds in each case to one row of Table A (compounds I.93aA-1 to I.93aA-2295)

Table 94a
Compounds I in which B is 2,5-di(difluoromethoxy)phenyl and the combination of A and D corresponds in each case to one row of Table A (compounds I.94aA-1 to I.94aA-2295)

Table 95a
Compounds I in which B is 2,6-di(difluoromethoxy)phenyl and the combination of A and D corresponds in each case to one row of Table A (compounds I.95aA-1 to I.95aA-2295)

Table 96a
Compounds I in which B is 2,3-di(trifluoromethylthio)phenyl and the combination of A and D corresponds in each case to one row of Table A (compounds I.96aA-1 to I.96aA-2295)

Table 97a
Compounds I in which B is 2,4-di(trifluoromethylthio)phenyl and the combination of A and D corresponds in each case to one row of Table A (compounds I.97aA-1 to I.97aA-2295)

Table 98a
Compounds I in which B is 2,5-di(trifluoromethylthio)phenyl and the combination of A and D corresponds in each case to one row of Table A (compounds I.98aA-1 to I.98aA-2295)

Table 99a
Compounds I in which B is 2,6-di(trifluoromethylthio)phenyl and the combination of A and D corresponds in each case to one row of Table A (compounds I.99aA-1 to I.99aA-2295)

Table 100a
Compounds I in which B is 2-methyl-3-chlorophenyl and the combination of A and D corresponds in each case to one row of Table A (compounds I.100aA-1 to I.100aA-2295)

Table 101a
Compounds I in which B is 2-methyl-4-chlorophenyl and the combination of A and D corresponds in each case to one row of Table A (compounds I.101aA-1 to I.101 aA-2295)

Table 102a
Compounds I in which B is 2-methyl-5-chlorophenyl and the combination of A and D corresponds in each case to one row of Table A (compounds I.102aA-1 to I.102aA-2295)

Table 103a
Compounds I in which B is 2-methyl-6-chlorophenyl and the combination of A and D corresponds in each case to one row of Table A (compounds I.103aA-1 to 1.103aA-2295)

Table 104a
Compounds I in which B is 2-chloro-3-methylphenyl and the combination of A and D corresponds in each case to one row of Table A (compounds I.104aA-1 to I.104aA-2295)

Table 105a
Compounds I in which B is 2-chloro-4-methylphenyl and the combination of A and D corresponds in each case to one row of Table A (compounds I.105aA-1 to I.105aA-2295)

Table 106a
Compounds I in which B is 2-chloro-5-methylphenyl and the combination of A and D corresponds in each case to one row of Table A (compounds I.106aA-1 to I.106aA-2295)

Table 107a
Compounds I in which B is 2-chloro-3-ethylphenyl and the combination of A and D corresponds in each case to one row of Table A (compounds I.107aA-1 to I.107aA-2295)

Table 108a
Compounds I in which B is 2-chloro-4-ethylphenyl and the combination of A and D corresponds in each case to one row of Table A (compounds I.108aA-1 to I.108aA-2295)

Table 109a
Compounds I in which B is 2-chloro-5-ethylphenyl and the combination of A and D corresponds in each case to one row of Table A (compounds I.109aA-1 to I.109aA-2295)

Table 110a
Compounds I in which B is 2-ethyl-3-chlorophenyl and the combination of A and D corresponds in each case to one row of Table A (compounds I.110aA-1 to I.110aA-2295)

Table 111a
Compounds I in which B is 2-ethyl-4-chlorophenyl and the combination of A and D corresponds in each case to one row of Table A (compounds I.111aA-1 to I.111aA-2295)

Table 112a
Compounds I in which B is 2-ethyl-5-chlorophenyl and the combination of A and D corresponds in each case to one row of Table A (compounds I.112aA-1 to I.112aA-2295)

Table 113a
Compounds I in which B is 2-ethyl-6-chlorophenyl and the combination of A and D corresponds in each case to one row of Table A (compounds I.113aA-1 to I.113aA-2295)

Table 114a
Compounds I in which B is 2-methoxy-3-chlorophenyl and the combination of A and D corresponds in each case to one row of Table A (compounds I.114aA-1 to I.114aA-2295)

Table 115a
Compounds I in which B is 2-methoxy-4-chlorophenyl and the combination of A and D corresponds in each case to one row of Table A (compounds I.115aA-1 to I.115aA-2295)

Table 116a
Compounds I in which B is 2-methoxy-5-chlorophenyl and the combination of A and D corresponds in each case to one row of Table A (compounds I.116aA-1 to I.116aA-2295)

Table 117a
Compounds I in which B is 2-methoxy-6-chlorophenyl and the combination of A and D corresponds in each case to one row of Table A (compounds I.117aA-1 to I.117aA-2295)

Table 118a
Compounds I in which B is 2-chloro-3-methoxyphenyl and the combination of A and D corresponds in each case to one row of Table A (compounds I.118aA-1 to I.118aA-2295)

Table 119a
Compounds I in which B is 2-chloro-4-methoxyphenyl and the combination of A and D corresponds in each case to one row of Table A (compounds I.119aA-1 to I.119aA-2295)

Table 120a
Compounds I in which B is 2-chloro-5-methoxyphenyl and the combination of A and D corresponds in each case to one row of Table A (compounds I.120aA-1 to I.120aA-2295)

Table 121a
Compounds I in which B is 2-(trifluoromethyl)-3-chlorophenyl and the combination of A and D corresponds in each case to one row of Table A (compounds I.121aA-1 to I.121aA-2295)

Table 122a
Compounds I in which B is 2-(trifluoromethyl)-4-chlorophenyl and the combination of A and D corresponds in each case to one row of Table A (compounds I.122aA-1 to I.122aA-2295)

Table 123a
Compounds I in which B is 2-(trifluoromethyl)-5-chlorophenyl and the combination of A and D corresponds in each case to one row of Table A (compounds I.123aA-1 to I.123aA-2295)

Table 124a
Compounds I in which B is 2-(trifluoromethyl)-6-chlorophenyl and the combination of A and D corresponds in each case to one row of Table A (compounds I.124aA-1 to I.124aA-2295)

Table 125a
Compounds I in which B is 2-chloro-3-(trifluoromethyl)phenyl and the combination of A and D corresponds in each case to one row of Table A (compounds I.125aA-1 to I.125aA-2295)

Table 126a
Compounds I in which B is 2-chloro-4-(trifluoromethyl)phenyl and the combination of A and D corresponds in each case to one row of Table A (compounds I.126aA-1 to I.126aA-2295)

Table 127a
Compounds I in which B is 2-chloro-5-(trifluoromethyl)phenyl and the combination of A and D corresponds in each case to one row of Table A (compounds I.127aA-1 to I.127aA-2295)

Table 128a
Compounds I in which B is 2-(trifluoromethoxy)-3-chlorophenyl and the combination of A and D corresponds in each case to one row of Table A (compounds I.128aA-1 to I.128aA-2295)

Table 129a
Compounds I in which B is 2-(trifluoromethoxy)-4-chlorophenyl and the combination of A and D corresponds in each case to one row of Table A (compounds I.129aA-1 to I.129aA-2295)

Table 130a
Compounds I in which B is 2-(trifluoromethoxy)-5-chlorophenyl and the combination of A and D corresponds in each case to one row of Table A (compounds I.130aA-1 to I.130aA-2295)

Table 131a
Compounds I in which B is 2-(trifluoromethoxy)-6-chlorophenyl and the combination of A and D corresponds in each case to one row of Table A (compounds I.131aA-1 to I.131aA-2295)

Table 132a
Compounds I in which B is 2-chloro-3-(trifluoromethoxy)phenyl and the combination of A and D corresponds in each case to one row of Table A (compounds I.132aA-1 to I.132aA-2295)

Table 133a
Compounds I in which B is 2-chloro-4-(trifluoromethoxy)phenyl and the combination of A and D corresponds in each case to one row of Table A (compounds I.133aA-1 to I.133aA-2295)

Table 134a
Compounds I in which B is 2-chloro-5-(trifluoromethoxy)phenyl and the combination of A and D corresponds in each case to one row of Table A (compounds I.134aA-1 to I.134aA-2295)

Table 135a
Compounds I in which B is 2-(difluoromethoxy)-3-chlorophenyl and the combination of A and D corresponds in each case to one row of Table A (compounds I.135aA-1 to I.135aA-2295)

Table 136a
Compounds I in which B is 2-(difluoromethoxy)-4-chlorophenyl and the combination of A and D corresponds in each case to one row of Table A (compounds I.136aA-1 to I.1362A-2295)

Table 137a
Compounds I in which B is 2-(difluoromethoxy)-5-chlorophenyl and the combination of A and D corresponds in each case to one row of Table A (compounds I.137aA-1 to I.137aA-2295)

Table 138a
Compounds I in which B is 2-(difluoromethoxy)-6-chlorophenyl and the combination of A and D corresponds in each case to one row of Table A (compounds I.138aA-1 to I.138aA-2295)

Table 139a
Compounds I in which B is 3-(difluoromethoxy)-4-chlorophenyl and the combination of A and D corresponds in each case to one row of Table A (compounds I.139aA-1 to I.139aA-2295)

Table 140a
Compounds I in which B is 2-chloro-3-(difluoromethoxy)phenyl and the combination of A and D corresponds in each case to one row of Table A (compounds I.140aA-1 to I.140aA-2295)

Table 141a
Compounds I in which B is 2-chloro-4-(difluoromethoxy)phenyl and the combination of A and D corresponds in each case to one row of Table A (compounds I.141aA-1 to I.141aA-2295)

Table 142a
Compounds I in which B is 2-chloro-5-(difluoromethoxy)phenyl and the combination of A and D corresponds in each case to one row of Table A (compounds I.142aA-1 to I.142aA-2295)

Table 143a
Compounds I in which B is 2-(trifluoromethylthio)-3-chlorophenyl and the combination of A and D corresponds in each case to one row of Table A (compounds I.143aA-1 to I.143aA-2295)

Table 144a
Compounds I in which B is 2-(trifluoromethylthio)-4-chlorophenyl and the combination of A and D corresponds in each case to one row of Table A (compounds I.144aA-1 to I.144aA-2295)

Table 145a
Compounds I in which B is 2-(trifluoromethylthio)-5-chlorophenyl and the combination of A and D corresponds in each case to one row of Table A (compounds I.145aA-1 to I.145aA-2295)

Table 146a
Compounds I in which B is 2-(trifluoromethylthio)-6-chlorophenyl and the combination of A and D corresponds in each case to one row of Table A (compounds I.146aA-1 to I.146aA-2295)

Table 147a
Compounds I in which B is 2-chloro-3-(trifluoromethylthio)phenyl and the combination of A and D corresponds in each case to one row of Table A (compounds I.147aA-1 to I.147aA-2295)

Table 148a
Compounds I in which B is 2-chloro-4-(trifluoromethylthio)phenyl and the combination of A and D corresponds in each case to one row of Table A (compounds I.148aA-1 to I.148aA-2295)

Table 149a
Compounds I in which B is 2-chloro-5-(trifluoromethylthio)phenyl and the combination of A and D corresponds in each case to one row of Table A (compounds I.149aA-1 to I.149aA-2295)

Table 150a
Compounds I in which B is 2,3,4-trichlorophenyl and the combination of A and D corresponds in each case to one row of Table A (compounds I.150aA-1 to I.150aA-2295)

Table 151a
Compounds I in which B is 2,3,5-trichlorophenyl and the combination of A and D corresponds in each case to one row of Table A (compounds I.151aA-1 to I.151aA-2295)

Table 152a
Compounds I in which B is 2,3,6-trichlorophenyl and the combination of A and D corresponds in each case to one row of Table A (compounds I.152aA-1 to I.152aA-2295)

Table 153a
Compounds I in which B is 2,4,5-trichlorophenyl and the combination of A and D corresponds in each case to one row of Table A (compounds I.153aA-1 to I.153aA-2295)

Table 154a
Compounds I in which B is 2,4,6-trichlorophenyl and the combination of A and D corresponds in each case to one row of Table A (compounds I.154aA-1 to I.154aA-2295)

Table 155a
Compounds I in which B is 2,3,4-trimethylphenyl and A corresponds in each case to one group of one row of Table A (compounds I.155aA-1 to I.155aA-2295)

Table 156a
Compounds I in which B is 2,3,5-trimethylphenyl and A corresponds in each case to one group of one row of Table A (compounds I.156aA-1 to I.156aA-2295)

Table 157a
Compounds I in which B is 2,3,6-trimethylphenyl and A corresponds in each case to one group of one row of Table A (compounds I.157aA-1 to I.157aA-2295)

Table 158a
Compounds I in which B is 2,4,5-trimethylphenyl and A corresponds in each case to one group of one row of Table A (compounds I.158aA-1 to I.158aA-2295)

Table 159a
Compounds I in which B is 2,4,6-trimethylphenyl and A corresponds in each case to one group of one row of Table A (compounds I.159aA-1 to I.159aA-2295)

Table 160a
Compounds I in which B is 2,3,4-trimethoxyphenyl and A corresponds in each case to one group of one row of Table A (compounds I.160aA-1 to I.160aA-2295)

Table 161a
Compounds I in which B is 2,3,5-trimethoxyphenyl and A corresponds in each case to one group of one row of Table A (compounds I.161aA-1 to I.161aA-2295)

Table 162a
Compounds I in which B is 2,3,6-trimethoxyphenyl and A corresponds in each case to one group of one row of Table A (compounds I.162aA-1 to I.162aA-2295)

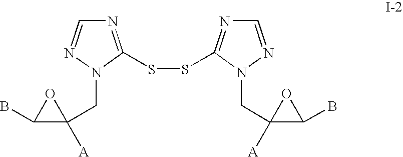

Table 1b
Compounds I-2 in which both B are 2,3-difluorophenyl and A corresponds in each case to one row of Table B (compounds I-2.1bB-1 to I-2.1bB-255)

Table 2b
Compounds I-2 in which both B are 2,4-difluorophenyl and A corresponds in each case to one row of Table B (compounds I-2.2bB-1 to I-2.2bB-255)

Table 3b
Compounds I-2 in which both B are 2,5-difluorophenyl and A corresponds in each case to one row of Table B (compounds I-2.3bB-1 to I-2.3bB-255)

Table 4b

Compounds I-2 in which both B are 2,6-difluorophenyl and A corresponds in each case to one row of Table B (compounds I-2.4bB-1 to I-2.4bB-255)

Table 5b
Compounds I-2 in which both B are 2-fluoro-3-chlorophenyl and A corresponds in each case to one row of Table B (compounds I-2.5bB-1 to I-2.5bB-255)

Table 6b
Compounds I-2 in which both B are 2-fluoro-4-chlorophenyl and A corresponds in each case to one row of Table B (compounds I-2.6bB-1 to I-2.6bB-255)

Table 7b
Compounds I-2 in which both B are 2-fluoro-5-chlorophenyl and A corresponds in each case to one row of Table B (compounds I-2.7bB-1 to I-2.7bB-255)

Table 8b
Compounds I-2 in which both B are 2-fluoro-6-chlorophenyl and A corresponds in each case to one row of Table B (compounds I-2.8bB-1 to I-2.8bB-255)

Table 9b
Compounds I-2 in which both B are 2-chloro-3-fluorophenyl and A corresponds in each case to one row of Table B (compounds I-2.9bB-1 to I-2.9bB-255)

Table 10b
Compounds I-2 in which both B are 2-chloro-4-fluorophenyl and A corresponds in each case to one row of Table B (compounds I-2.10bB-1 to I-2.10bB-255)

Table 11b
Compounds I-2 in which both B are 2-chloro-5-fluorophenyl and A corresponds in each case to one row of Table B (compounds I-2.11bB-1 to I-2.11bB-255)

Table 12b
Compounds I-2 in which both B are 2-methyl-3-fluorophenyl and A corresponds in each case to one row of Table B (compounds I-2.12bB-1 to I-2.12bB-255)

Table 13b
Compounds I-2 in which both B are 2-methyl-4-fluorophenyl and A corresponds in each case to one row of Table B (compounds I-2.13bB-1 to I-2.13bB-255)

Table 14b
Compounds I-2 in which both B are 2-methyl-5-fluorophenyl and A corresponds in each case to one row of Table B (compounds I-2.14bB-1 to I-2.14bB-255)

Table 15b
Compounds I-2 in which both B are 2-methyl-6-fluorophenyl and A corresponds in each case to one row of Table B (compounds I-2.15bB-1 to I-2.15bB-255)

Table 16b
Compounds I-2 in which both B are 2-fluoro-3-methylphenyl and A corresponds in each case to one row of Table B (compounds I-2.16bB-1 to I-2.16bB-255)

Table 17b
Compounds I-2 in which both B are 2-fluoro-4-methylphenyl and A corresponds in each case to one row of Table B (compounds I-2.17bB-1 to I-2.17bB-255)

Table 18b
Compounds I-2 in which both B are 2-fluoro-5-methylphenyl and A corresponds in each case to one row of Table B (compounds I-2.18bB-1 to I-2.18bB-255)

Table 19b
Compounds I-2 in which both B are 2-ethyl-3-fluorophenyl and A corresponds in each case to one row of Table B (compounds I-2.19bB-1 to I-2.19bB-255)

Table 20b
Compounds I-2 in which both B are 2-ethyl-4-fluorophenyl and A corresponds in each case to one row of Table B (compounds I-2.20bB-1 to I-2.20bB-255)

Table 21b
Compounds I-2 in which both B are 2-ethyl-5-fluorophenyl and A corresponds in each case to one row of Table B (compounds I-2.21 bB-1 to I-2.21 bB-255)

Table 22b
Compounds I-2 in which both B are 2-ethyl-6-fluorophenyl and A corresponds in each case to one row of Table B (compounds I-2.22bB-1 to I-2.22bB-255)

Table 23b
Compounds I-2 in which both B are 2-fluoro-3-ethylphenyl and A corresponds in each case to one row of Table B (compounds I-2.23bB-1 to I-2.23bB-255)

Table 24b
Compounds I-2 in which both B are 2-fluoro-4-ethylphenyl and A corresponds in each case to one row of Table B (compounds I-2.24bB-1 to I-2.24bB-255)

Table 25b
Compounds I-2 in which both B are 2-methoxy-3-fluorophenyl and A corresponds in each case to one row of Table B (compounds I-2.25bB-1 to I-2.25bB-255)

Table 26b
Compounds I-2 in which both B are 2-methoxy-4-fluorophenyl and A corresponds in each case to one row of Table B (compounds I-2.26bB-1 to I-2.26bB-255)

Table 27b
Compounds I-2 in which both B are 2-methoxy-5-fluorophenyl and A corresponds in each case to one row of Table B (compounds I-2.27bB-1 to I-2.27bB-255)

Table 28b
Compounds I-2 in which both B are 2-methoxy-6-fluorophenyl and A corresponds in each case to one row of Table B (compounds I-2.28bB-1 to I-2.28bB-255)

Table 29b
Compounds I-2 in which both B are 2-fluoro-3-methoxyphenyl and A corresponds in each case to one row of Table B (compounds I-2.29bB-1 to I-2.29bB-255)

Table 30b
Compounds I-2 in which both B are 2-fluoro-4-methoxyphenyl and A corresponds in each case to one row of Table B (compounds I-2.30bB-1 to I-2.30bB-255)

Table 31b
Compounds I-2 in which both B are 2-fluoro-5-methoxyphenyl and A corresponds in each case to one row of Table B (compounds I-2.31bB-1 to I-2.31bB-255)

Table 32b
Compounds I-2 in which both B are 2-(trifluoromethyl)-3-fluorophenyl and A corresponds in each case to one row of Table B (compounds I-2.32bB-1 to I-2.32bB-255)

Table 33b
Compounds I-2 in which both B are 2,4,5-trimethoxyphenyl and A corresponds in each case to one row of Table B (compounds I-2.33bB-1 to I-2.33bB-255)

Table 34b
Compounds I-2 in which both B are 2,4,6-trimethoxyphenyl and A corresponds in each case to one row of Table B (compounds I-2.34bB-1 to I-2.34bB-255)

Table 35b
Compounds I-2 in which both B are 2-fluoro-5-ethylphenyl and A corresponds in each case to one row of Table B (compounds I-2.35bB-1 to I-2.35bB-255)

Table 36b
Compounds I-2 in which both B are 2-(trifluoromethyl)-4-fluorophenyl and A corresponds in each case to one row of Table B (compounds I-2.36bB-1 to I-2.36bB-255)

Table 37b
Compounds I-2 in which both B are 2-(trifluoromethyl)-5-fluorophenyl and A corresponds in each case to one row of Table B (compounds I-2.37bB-1 to I-2.37bB-255)

Table 38b
Compounds I-2 in which both B are 2-(trifluoromethyl)-6-fluorophenyl and A corresponds in each case to one row of Table B (compounds I-2.38bB-1 to I-2.38bB-255)

Table 39b
Compounds I-2 in which both B are 2-fluoro-3-(trifluoromethyl)phenyl and A corresponds in each case to one row of Table B (compounds I-2.39bB-1 to I-2.39bB-255)

Table 40b
Compounds I-2 in which both B are 2-fluoro-4-(trifluoromethyl)phenyl and A corresponds in each case to one row of Table B (compounds I-2.40bB-1 to I-2.40bB-255)

Table 41b
Compounds I-2 in which both B are 2-fluoro-5-(trifluoromethyl)phenyl and A corresponds in each case to one row of Table B (compounds I-2.41 bB-1 to I-2.41 bB-255)

Table 42b
Compounds I-2 in which both B are 2-(trifluoromethoxy)-3-fluorophenyl and A corresponds in each case to one row of Table B (compounds I-2.42bB-1 to I-2.42bB-255)

Table 43b
Compounds I-2 in which both B are 2-(trifluoromethoxy)-4-fluorophenyl and A corresponds in each case to one row of Table B (compounds I-2.43bB-1 to I-2.43bB-255)

Table 44b
Compounds I-2 in which both B are 2-(trifluoromethoxy)-5-fluorophenyl and A corresponds in each case to one row of Table B (compounds I-2.44bB-1 to I-2.44bB-255)

Table 45b
Compounds I-2 in which both B are 2-(trifluoromethoxy)-6-fluorophenyl and A corresponds in each case to one row of Table B (compounds I-2.45bB-1 to I-2.45bB-255)

Table 46b
Compounds I-2 in which both B are 2-fluoro-3-(trifluoromethoxy)phenyl and A corresponds in each case to one row of Table B (compounds I-2.46bB-1 to I-2.46bB-255)

Table 47b
Compounds I-2 in which both B are 2-fluoro-4-(trifluoromethoxy)phenyl and A corresponds in each case to one row of Table B (compounds I-2.47bB-1 to I-2.47bB-255)

Table 48b
Compounds I-2 in which both B are 2-fluoro-5-(trifluoromethoxy)phenyl and A corresponds in each case to one row of Table B (compounds I-2.48bB-1 to I-2.48bB-255)

Table 49b
Compounds I-2 in which both B are 2-(difluoromethoxy)-3-fluorophenyl and A corresponds in each case to one row of Table B (compounds I-2.49bB-1 to I-2.49bB-255)

Table 50b
Compounds I-2 in which both B are 2-(difluoromethoxy)-4-fluorophenyl and A corresponds in each case to one row of Table B (compounds I-2.50bB-1 to I-2.50bB-255)

Table 51b
Compounds I-2 in which both B are 2-(difluoromethoxy)-5-fluorophenyl and A corresponds in each case to one row of Table B (compounds I-2.51 bB-1 to I-2.51 bB-255)

Table 52b
Compounds I-2 in which both B are 2-(difluoromethoxy)-6-fluorophenyl and A corresponds in each case to one row of Table B (compounds I-2.52bB-1 to I-2.52bB-255)

Table 53b
Compounds I-2 in which both B are 2-fluoro-3-(difluoromethoxy)phenyl and A corresponds in each case to one row of Table B (compounds I-2.53bB-1 to I-2.53bB-255)

Table 54b
Compounds I-2 in which both B are 2-fluoro-4-(difluoromethoxy)phenyl and A corresponds in each case to one row of Table B (compounds I-2.54bB-1 to I-2.54bB-255)

Table 55b
Compounds I-2 in which both B are 2-fluoro-5-(difluoromethoxy)phenyl and A corresponds in each case to one row of Table B (compounds I-2.55bB-1 to I-2.55bB-255)

Table 56b
Compounds I-2 in which both B are 2-(trifluoromethylthio)-3-fluorophenyl and A corresponds in each case to one row of Table B (compounds I-2.56bB-1 to I-2.56bB-255)

Table 57b
Compounds I-2 in which both B are 2-(trifluoromethylthio)-4-fluorophenyl and A corresponds in each case to one row of Table B (compounds I-2.57bB-1 to I-2.57bB-255)

Table 58b
Compounds I-2 in which both B are 2-(trifluoromethylthio)-5-fluorophenyl and A corresponds in each case to one row of Table B (compounds I-2.58bB-1 to I-2.58bB-255)

Table 59b
Compounds I-2 in which both B are 2-(trifluoromethylthio)-6-fluorophenyl and A corresponds in each case to one row of Table B (compounds I-2.59bB-1 to I-2.59bB-255)

Table 60b
Compounds I-2 in which both B are 2-fluoro-3-(trifluoromethylthio)phenyl and A corresponds in each case to one row of Table B (compounds I-2.60bB-1 to I-2.60bB-255)

Table 61b
Compounds I-2 in which both B are 2-fluoro-4-(trifluoromethylthio)phenyl and A corresponds in each case to one row of Table B (compounds I-2.61bB-1 to I-2.61bB-255)

Table 62b
Compounds I-2 in which both B are 2-fluoro-5-(trifluoromethylthio)phenyl and A corresponds in each case to one row of Table B (compounds I-2.62bB-1 to I-2.62bB-255)

Table 63b
Compounds I-2 in which both B are 2,3,4-trifluorophenyl and A corresponds in each case to one row of Table B (compounds I-2.63bB-1 to I-2.63bB-255)

Table 64b
Compounds I-2 in which both B are 2,3,5-trifluorophenyl and A corresponds in each case to one row of Table B (compounds I-2.64bB-1 to I-2.64bB-255)

Table 65b
Compounds I-2 in which both B are 2,3,6-trifluorophenyl and A corresponds in each case to one row of Table B (compounds I-2.65bB-1 to I-2.65bB-255)

Table 66b
Compounds I-2 in which both B are 2,4,5-trifluorophenyl and A corresponds in each case to one row of Table B (compounds I-2.66bB-1 to I-2.66bB-255)

Table 67b
Compounds I-2 in which both B are 2,4,6-trifluorophenyl and A corresponds in each case to one row of Table B (compounds I-2.67bB-1 to I-2.67bB-255)

Table 68b
Compounds I-2 in which both B are 2,3-dichlorophenyl and A corresponds in each case to one row of Table B (compounds I-2.68bB-1 to I-2.68bB-255)

Table 69b
Compounds I-2 in which both B are 2,4-dichlorophenyl and A corresponds in each case to one row of Table B (compounds I-2.69bB-1 to I-2.69bB-255)

Table 70b
Compounds I-2 in which both B are 2,5-dichlorophenyl and A corresponds in each case to one row of Table B (compounds I-2.70bB-1 to I-2.70bB-255)

Table 71b
Compounds I-2 in which both B are 2,6-dichlorophenyl and A corresponds in each case to one row of Table B (compounds I-2.71 bB-1 to I-2.71 bB-255)

Table 72b
Compounds I-2 in which both B are 2,3-dimethylphenyl and A corresponds in each case to one row of Table B (compounds I-2.72bB-1 to I-2.72bB-255)

Table 73b
Compounds I-2 in which both B are 2,4-dimethylphenyl and A corresponds in each case to one row of Table B (compounds I-2.73bB-1 to I-2.73bB-255)

Table 74b
Compounds I-2 in which both B are 2,5-dimethylphenyl and A corresponds in each case to one row of Table B (compounds I-2.74bB-1 to I-2.74bB-255)

Table 75b
Compounds I-2 in which both B are 2,6-dimethylphenyl and A corresponds in each case to one row of Table B (compounds I-2.75bB-1 to I-2.75bB-255)

Table 76b
Compounds I-2 in which both B are 2,3-diethylphenyl and A corresponds in each case to one row of Table B (compounds I-2.76bB-1 to I-2.76bB-255)

Table 77b
Compounds I-2 in which both B are 2,4-diethylphenyl and A corresponds in each case to one row of Table B (compounds I-2.77bB-1 to I-2.77bB-255)

Table 78b
Compounds I-2 in which both B are 2,5-diethylphenyl and A corresponds in each case to one row of Table B (compounds I-2.78bB-1 to I-2.78bB-255)

Table 79b
Compounds I-2 in which both B are 2,6-diethylphenyl and A corresponds in each case to one row of Table B (compounds I-2.79bB-1 to I-2.79bB-255)

Table 80b
Compounds I-2 in which both B are 2,3-dimethoxyphenyl and A corresponds in each case to one row of Table B (compounds I-2.80bB-1 to I-2.80bB-255)

Table 81b
Compounds I-2 in which both B are 2,4-dimethoxyphenyl and A corresponds in each case to one row of Table B (compounds I-2.81bB-1 to I-2.81bB-255)

Table 82b
Compounds I-2 in which both B are 2,5-dimethoxyphenyl and A corresponds in each case to one row of Table B (compounds I-2.82bB-1 to I-2.82bB-255)

Table 83b
Compounds I-2 in which both B are 2,6-dimethoxyphenyl and A corresponds in each case to one row of Table B (compounds I-2.83bB-1 to I-2.83bB-255)

Table 84b
Compounds I-2 in which both B are 2,3-di(trifluoromethyl)phenyl and A corresponds in each case to one row of Table B (compounds I-2.84bB-1 to I-2.84bB-255)

Table 85b
Compounds I-2 in which both B are 2,4-di(trifluoromethyl)phenyl and A corresponds in each case to one row of Table B (compounds I-2.85bB-1 to I-2.85bB-255)

Table 86b
Compounds I-2 in which both B are 2,5-di(trifluoromethyl)phenyl and A corresponds in each case to one row of Table B (compounds I-2.86bB-1 to I-2.86bB-255)

Table 87b
Compounds I-2 in which both B are 2,6-di(trifluoromethyl)phenyl and A corresponds in each case to one row of Table B (compounds I-2.87bB-1 to I-2.87bB-255)

Table 88b
Compounds I-2 in which both B are 2,3-di(trifluoromethoxy)phenyl and A corresponds in each case to one row of Table B (compounds I-2.88bB-1 to I-2.88bB-255)

Table 89b
Compounds I-2 in which both B are 2,4-di(trifluoromethoxy)phenyl and A corresponds in each case to one row of Table B (compounds I-2.89bB-1 to I-2.89bB-255)

Table 90b
Compounds I-2 in which both B are 2,5-di(trifluoromethoxy)phenyl and A corresponds in each case to one row of Table B (compounds I-2.90bB-1 to I-2.90bB-255)

Table 91b
Compounds I-2 in which both B are 2,6-di(trifluoromethoxy)phenyl and A corresponds in each case to one row of Table B (compounds I-2.91 bB-1 to I-2.91 bB-255)

Table 92b
Compounds I-2 in which both B are 2,3-di(difluoromethoxy)phenyl and A corresponds in each case to one row of Table B (compounds I-2.92bB-1 to I-2.92bB-255)

Table 93b
Compounds I-2 in which both B are 2,4-di(difluoromethoxy)phenyl and A corresponds in each case to one row of Table B (compounds I-2.93bB-1 to I-2.93bB-255)

Table 94b
Compounds I-2 in which both B are 2,5-di(difluoromethoxy)phenyl and A corresponds in each case to one row of Table B (compounds I-2.94bB-1 to I-2.94bB-255)

Table 95b
Compounds I-2 in which both B are 2,6-di(difluoromethoxy)phenyl and A corresponds in each case to one row of Table B (compounds I-2.95bB-1 to I-2.95bB-255)

Table 96b
Compounds I-2 in which both B are 2,3-di(trifluoromethylthio)phenyl and A corresponds in each case to one row of Table B (compounds I-2.96bB-1 to I-2.96bB-255)

Table 97b
Compounds I-2 in which both B are 2,4-di(trifluoromethylthio)phenyl and A corresponds in each case to one row of Table B (compounds I-2.97bB-1 to I-2.97bB-255)

Table 98b
Compounds I-2 in which both B are 2,5-di(trifluoromethylthio)phenyl and A corresponds in each case to one row of Table B (compounds I-2.98bB-1 to I-2.98bB-255)

Table 99b
Compounds I-2 in which both B are 2,6-di(trifluoromethylthio)phenyl and A corresponds in each case to one row of Table B (compounds I-2.99bB-1 to I-2.99bB-255)

Table 100b
Compounds I-2 in which both B are 2-methyl-3-chlorophenyl and A corresponds in each case to one row of Table B (compounds I-2.100bB-1 to I-2.100bB-255)

Table 101b
Compounds I-2 in which both B are 2-methyl-4-chlorophenyl and A corresponds in each case to one row of Table B (compounds I-2.101bB-1 to I-2.101bB-255)

Table 102b
Compounds I-2 in which both B are 2-methyl-5-chlorophenyl and A corresponds in each case to one row of Table B (compounds I-2.102bB-1 to I-2.102bB-255)

Table 103b
Compounds I-2 in which both B are 2-methyl-6-chlorophenyl and A corresponds in each case to one row of Table B (compounds I-2.103bB-1 to I-2.103bB-255)

Table 104b
Compounds I-2 in which both B are 2-chloro-3-methylphenyl and A corresponds in each case to one row of Table B (compounds I-2.104bB-1 to I-2.104bB-255)

Table 105b
Compounds I-2 in which both B are 2-chloro-4-methylphenyl and A corresponds in each case to one row of Table B (compounds I-2.105bB-1 to I-2.105bB-255)

Table 106b
Compounds I-2 in which both B are 2-chloro-5-methylphenyl and A corresponds in each case to one row of Table B (compounds I-2.106bB-1 to I-2.106bB-255)

Table 107b
Compounds I-2 in which both B are 2-chloro-3-ethylphenyl and A corresponds in each case to one row of Table B (compounds I-2.107bB-1 to I-2.107bB-255)

Table 108b
Compounds I-2 in which both B are 2-chloro-4-ethylphenyl and A corresponds in each case to one row of Table B (compounds I-2.108bB-1 to I-2.108bB-255)

Table 109b
Compounds I-2 in which both B are 2-chloro-5-ethylphenyl and A corresponds in each case to one row of Table B (compounds I-2.109bB-1 to I-2.109bB-255)

Table 110b
Compounds I-2 in which both B are 2-ethyl-3-chlorophenyl and A corresponds in each case to one row of Table B (compounds I-2.110bB-1 to I-2.110bB-255)

Table 111b
Compounds I-2 in which both B are 2-ethyl-4-chlorophenyl and A corresponds in each case to one row of Table B (compounds I-2.111bB-1 to I-2.111 bB-255)

Table 112b
Compounds I-2 in which both B are 2-ethyl-5-chlorophenyl and A corresponds in each case to one row of Table B (compounds I-2.112bB-1 to I-2.112bB-255)

Table 113b
Compounds I-2 in which both B are 2-ethyl-6-chlorophenyl and A corresponds in each case to one row of Table B (compounds I-2.113bB-1 to I-2.113bB-255)

Table 114b
Compounds I-2 in which both B are 2-methoxy-3-chlorophenyl and A corresponds in each case to one row of Table B (compounds I-2.114bB-1 to I-2.114bB-255)

Table 115b
Compounds I-2 in which both B are 2-methoxy-4-chlorophenyl and A corresponds in each case to one row of Table B (compounds I-2.115bB-1 to I-2.115bB-255)

Table 116b
Compounds I-2 in which both B are 2-methoxy-5-chlorophenyl and A corresponds in each case to one row of Table B (compounds I-2.116bB-1 to I-2.116bB-255)

Table 117b
Compounds I-2 in which both B are 2-methoxy-6-chlorophenyl and A corresponds in each case to one row of Table B (compounds I-2.117bB-1 to I-2.117bB-255)

Table 118b
Compounds I-2 in which both B are 2-chloro-3-methoxyphenyl and A corresponds in each case to one row of Table B (compounds I-2.118bB-1 to I-2.118bB-255)

Table 119b
Compounds I-2 in which both B are 2-chloro-4-methoxyphenyl and A corresponds in each case to one row of Table B (compounds I-2.119bB-1 to I-2.119bB-255)

Table 120b
Compounds I-2 in which both B are 2-chloro-5-methoxyphenyl and A corresponds in each case to one row of Table B (compounds I-2.120bB-1 to I-2.120bB-255)

Table 121b
Compounds I-2 in which both B are 2-(trifluoromethyl)-3-chlorophenyl and A corresponds in each case to one row of Table B (compounds I-2.121bB-1 to I-2.121bB-255)

Table 122b
Compounds I-2 in which both B are 2-(trifluoromethyl)-4-chlorophenyl and A corresponds in each case to one row of Table B (compounds I-2.122bB-1 to I-2.122bB-255)

Table 123b
Compounds I-2 in which both B are 2-(trifluoromethyl)-5-chlorophenyl and A corresponds in each case to one row of Table B (compounds I-2.123bB-1 to I-2.123bB-255)

Table 124b
Compounds I-2 in which both B are 2-(trifluoromethyl)-6-chlorophenyl and A corresponds in each case to one row of Table B (compounds I-2.124bB-1 to I-2.124bB-255)

Table 125b
Compounds I-2 in which both B are 2-chloro-3-(trifluoromethyl)phenyl and A corresponds in each case to one row of Table B (compounds I-2.125bB-1 to I-2.125bB-255)

Table 126b
Compounds I-2 in which both B are 2-chloro-4-(trifluoromethyl)phenyl and A corresponds in each case to one row of Table B (compounds I-2.126bB-1 to I-2.126bB-255)

Table 127b
Compounds I-2 in which both B are 2-chloro-5-(trifluoromethyl)phenyl and A corresponds in each case to one row of Table B (compounds I-2.127bB-1 to I-2.127bB-255)

Table 128b
Compounds I-2 in which both B are 2-(trifluoromethoxy)-3-chlorophenyl and A corresponds in each case to one row of Table B (compounds I-2.128bB-1 to 1-2.128bB-255)

Table 129b
Compounds I-2 in which both B are 2-(trifluoromethoxy)-4-chlorophenyl and A corresponds in each case to one row of Table B (compounds I-2.129bB-1 to I-2.129bB-255)

Table 130b
Compounds I-2 in which both B are 2-(trifluoromethoxy)-5-chlorophenyl and A corresponds in each case to one row of Table B (compounds I-2.130bB-1 to I-2.130bB-255)

Table 131b
Compounds I-2 in which both B are 2-(trifluoromethoxy)-6-chlorophenyl and A corresponds in each case to one row of Table B (compounds I-2.131bB-1 to I-2.131 bB-255)

Table 132b
Compounds I-2 in which both B are 2-chloro-3-(trifluoromethoxy)phenyl and A corresponds in each case to one row of Table B (compounds I-2.132bB-1 to I-2.132bB-255)

Table 133b
Compounds I-2 in which both B are 2-chloro-4-(trifluoromethoxy)phenyl and A corresponds in each case to one row of Table B (compounds I-2.133bB-1 to I-2.133bB-255)

Table 134b
Compounds I-2 in which both B are 2-chloro-5-(trifluoromethoxy)phenyl and A corresponds in each case to one row of Table B (compounds I-2.134bB-1 to I-2.134bB-255)

Table 135b
Compounds I-2 in which both B are 2-(difluoromethoxy)-3-chlorophenyl and A corresponds in each case to one row of Table B (compounds I-2.135bB-1 to I-2.135bB-255)

Table 136b
Compounds I-2 in which both B are 2-(difluoromethoxy)-4-chlorophenyl and A corresponds in each case to one row of Table B (compounds I-2.136bB-1 to I-2.136bB-255)

Table 137b
Compounds I-2 in which both B are 2-(difluoromethoxy)-5-chlorophenyl and A corresponds in each case to one row of Table B (compounds I-2.137bB-1 to I-2.137bB-255)

Table 138b
Compounds I-2 in which both B are 2-(difluoromethoxy)-6-chlorophenyl and A corresponds in each case to one row of Table B (compounds I-2.138bB-1 to I-2.138bB-255)

Table 139b
Compounds I-2 in which both B are 3-(difluoromethoxy)-4-chlorophenyl and A corresponds in each case to one row of Table B (compounds I-2.139bB-1 to I-2.139bB-255)

Table 140b
Compounds I-2 in which both B are 2-chloro-3-(difluoromethoxy)phenyl and A corresponds in each case to one row of Table B (compounds I-2.140bB-1 to I-2.140bB-255)

Table 141b
Compounds I-2 in which both B are 2-chloro-4-(difluoromethoxy)phenyl and A corresponds in each case to one row of Table B (compounds I-2.141bB-1 to I-2.141bB-255)

Table 142b
Compounds I-2 in which both B are 2-chloro-5-(difluoromethoxy)phenyl and A corresponds in each case to one row of Table B (compounds I-2.142bB-1 to I-2.142bB-255)

Table 143b
Compounds I-2 in which both B are 2-(trifluoromethylthio)-3-chlorophenyl and A corresponds in each case to one row of Table B (compounds I-2.143bB-1 to I-2.143bB-255)

Table 144b
Compounds I-2 in which both B are 2-(trifluoromethylthio)-4-chlorophenyl and A corresponds in each case to one row of Table B (compounds I-2.144bB-1 to I-2.144bB-255)

Table 145b
Compounds I-2 in which both B are 2-(trifluoromethylthio)-5-chlorophenyl and A corresponds in each case to one row of Table B (compounds I-2.145bB-1 to I-2.145bB-255)

Table 146b
Compounds I-2 in which both B are 2-(trifluoromethylthio)-6-chlorophenyl and A corresponds in each case to one row of Table B (compounds I-2.146bB-1 to I-2.146bB-255)

Table 147b
Compounds I-2 in which both B are 2-chloro-3-(trifluoromethylthio)phenyl and A corresponds in each case to one row of Table B (compounds I-2.147bB-1 to I-2.147bB-255)

Table 148b
Compounds I-2 in which both B are 2-chloro-4-(trifluoromethylthio)phenyl and A corresponds in each case to one row of Table B (compounds I-2.148bB-1 to I-2.148bB-255)

Table 149b
Compounds I-2 in which both B are 2-chloro-5-(trifluoromethylthio)phenyl and A corresponds in each case to one row of Table B (compounds I-2.149bB-1 to I-2.149bB-255)

Table 150b
Compounds I-2 in which both B are 2,3,4-trichlorophenyl and A corresponds in each case to one row of Table B (compounds I-2.150bB-1 to I-2.150bB-255)

Table 151b
Compounds I-2 in which both B are 2,3,5-trichlorophenyl and A corresponds in each case to one row of Table B (compounds I-2.151bB-1 to I-2.151bB-255)

Table 152b
Compounds I-2 in which both B are 2,3,6-trichlorophenyl and A corresponds in each case to one row of Table B (compounds I-2.152bB-1 to I-2.152bB-255)

Table 153b
Compounds I-2 in which both B are 2,4,5-trichlorophenyl and A corresponds in each case to one row of Table B (compounds I-2.153bB-1 to I-2.153bB-255)

Table 154b
Compounds I-2 in which both B are 2,4,6-trichlorophenyl and A corresponds in each case to one row of Table B (compounds I-2.154bB-1 to I-2.154bB-255)

Table 155b
Compounds I-2 in which both B are 2,3,4-trimethylphenyl and A corresponds in each case to one row of Table B (compounds I-2.155bB-1 to I-2.155bB-255)

Table 156b
Compounds I-2 in which both B are 2,3,5-trimethylphenyl and A corresponds in each case to one row of Table B (compounds I-2.156bB-1 to I-2.156bB-255)

Table 157b
Compounds I-2 in which both B are 2,3,6-trimethylphenyl and A corresponds in each case to one row of Table B (compounds I-2.157bB-1 to I-2.157bB-255)

Table 158b
Compounds I-2 in which both B are 2,4,5-trimethylphenyl and A corresponds in each case to one row of Table B (compounds I-2.158bB-1 to I-2.158bB-255)

Table 159b
Compounds I-2 in which both B are 2,4,6-trimethylphenyl and A corresponds in each case to one row of Table B (compounds I-2.159bB-1 to I-2.159bB-255)

Table 160b
Compounds I-2 in which both B are 2,3,4-trimethoxyphenyl and A corresponds in each case to one row of Table B (compounds I-2.160bB-1 to I-2.160bB-255)

Table 161b
Compounds I-2 in which both B are 2,3,5-trimethoxyphenyl and A corresponds in each case to one row of Table B (compounds I-2.161bB-1 to I-2.161bB-255)

Table 162b
Compounds I-2 in which both B are 2,3,6-trimethoxyphenyl and A corresponds in each case to one row of Table B (compounds I-2.162bB-1 to I-2.162bB-255)

TABLE A

| Row | A | D |
|---|---|---|
| A-1 | Phenyl | SH |
| A-2 | 2-Chlorophenyl | SH |
| A-3 | 3-Chlorophenyl | SH |
| A-4 | 4-Chlorophenyl | SH |
| A-5 | 2-Fluorophenyl | SH |
| A-6 | 3-Fluorophenyl | SH |
| A-7 | 4-Fluorophenyl | SH |
| A-8 | 2-Methylphenyl | SH |
| A-9 | 3-Methylphenyl | SH |
| A-10 | 4-Methylphenyl | SH |
| A-11 | 2-Ethylphenyl | SH |
| A-12 | 3-Ethylphenyl | SH |
| A-13 | 4-Ethylphenyl | SH |
| A-14 | 2-Methoxyphenyl | SH |
| A-15 | 3-Methoxyphenyl | SH |
| A-16 | 4-Methoxyphenyl | SH |
| A-17 | 2-Trifluoromethylphenyl | SH |
| A-18 | 3-Trifluoromethylphenyl | SH |
| A-19 | 4-Trifluoromethylphenyl | SH |
| A-20 | 2-Trifluoromethoxyphenyl | SH |
| A-21 | 3-Trifluoromethoxyphenyl | SH |
| A-22 | 4-Trifluoromethoxyphenyl | SH |
| A-23 | 2-Difluoromethoxyphenyl | SH |
| A-24 | 3-Difluoromethoxyphenyl | SH |
| A-25 | 4-Difluoromethoxyphenyl | SH |
| A-26 | 2-Trifluoromethylthiophenyl | SH |
| A-27 | 3-Trifluoromethylthiophenyl | SH |
| A-28 | 4-Trifluoromethylthiophenyl | SH |
| A-29 | 2,3-Dichlorophenyl | SH |
| A-30 | 2,4-Dichlorophenyl | SH |
| A-31 | 2,5-Dichlorophenyl | SH |
| A-32 | 2,6-Dichlorophenyl | SH |
| A-33 | 3,4-Dichlorophenyl | SH |
| A-34 | 3,5-Dichlorophenyl | SH |
| A-35 | 2,3-Difluorophenyl | SH |
| A-36 | 2,4-Difluorophenyl | SH |
| A-37 | 2,5-Difluorophenyl | SH |
| A-38 | 2,6-Difluorophenyl | SH |
| A-39 | 3,4-Difluorophenyl | SH |
| A-40 | 3,5-Difluorophenyl | SH |
| A-41 | 2,3-Dimethylphenyl | SH |
| A-42 | 2,4-Dimethylphenyl | SH |
| A-43 | 2,5-Dimethylphenyl | SH |
| A-44 | 2,6-Dimethylphenyl | SH |
| A-45 | 3,4-Dimethylphenyl | SH |
| A-46 | 3,5-Dimethylphenyl | SH |
| A-47 | 2,3-Diethylphenyl | SH |
| A-48 | 2,4-Diethylphenyl | SH |
| A-49 | 2,5-Diethylphenyl | SH |
| A-50 | 2,6-Diethylphenyl | SH |
| A-51 | 3,5-Diethylphenyl | SH |
| A-52 | 3,4-Diethylphenyl | SH |
| A-53 | 2,3-Dimethoxyphenyl | SH |
| A-54 | 2,4-Dimethoxyphenyl | SH |
| A-55 | 2,5-Dimethoxyphenyl | SH |
| A-56 | 2,6-Dimethoxyphenyl | SH |
| A-57 | 3,4-Dimethoxyphenyl | SH |
| A-58 | 3,5-Dimethoxyphenyl | SH |
| A-59 | 2,3-Di(trifluoromethyl)phenyl | SH |
| A-60 | 2,4-Di(trifluoromethyl)phenyl | SH |
| A-61 | 2,5-Di(trifluoromethyl)phenyl | SH |
| A-62 | 2,6-Di(trifluoromethyl)phenyl | SH |
| A-63 | 3,4-Di(trifluoromethyl)phenyl | SH |
| A-64 | 3,5-Di(trifluoromethyl)phenyl | SH |
| A-65 | 2,3-Di(trifluoromethoxy)phenyl | SH |
| A-66 | 2,4-Di(trifluoromethoxy)phenyl | SH |
| A-67 | 2,5-Di(trifluoromethoxy)phenyl | SH |
| A-68 | 2,6-Di(trifluoromethoxy)phenyl | SH |
| A-69 | 3,4-Di(trifluoromethoxy)phenyl | SH |
| A-70 | 3,5-Di(trifluoromethoxy)phenyl | SH |
| A-71 | 2,3-Di(difluoromethoxy)phenyl | SH |
| A-72 | 2,4-Di(difluoromethoxy)phenyl | SH |
| A-73 | 2,5-Di(difluoromethoxy)phenyl | SH |
| A-74 | 2,6-Di(difluoromethoxy)phenyl | SH |
| A-75 | 3,4-Di(difluoromethoxy)phenyl | SH |
| A-76 | 3,5-Di(difluoromethoxy)phenyl | SH |
| A-77 | 2,3-Di(trifluoromethylthio)phenyl | SH |
| A-78 | 2,4-Di(trifluoromethylthio)phenyl | SH |
| A-79 | 2,5-Di(trifluoromethylthio)phenyl | SH |
| A-80 | 2,6-Di(trifluoromethylthio)phenyl | SH |
| A-81 | 3,4-Di(trifluoromethylthio)phenyl | SH |
| A-82 | 3,5-Di(trifluoromethylthio)phenyl | SH |
| A-83 | 2-Fluoro-3-chlorophenyl | SH |
| A-84 | 2-Fluoro-4-chlorophenyl | SH |
| A-85 | 2-Fluoro-5-chlorophenyl | SH |
| A-86 | 2-Fluoro-6-chlorophenyl | SH |
| A-87 | 3-Fluoro-4-chlorophenyl | SH |
| A-88 | 3-Fluoro-5-chlorophenyl | SH |
| A-89 | 2-Chloro-3-fluorophenyl | SH |
| A-90 | 2-Chloro-4-fluorophenyl | SH |
| A-91 | 2-Chloro-5-fluorophenyl | SH |
| A-92 | 3-Chloro-4-fluorophenyl | SH |
| A-93 | 2-Methyl-3-chlorophenyl | SH |
| A-94 | 2-Methyl-4-chlorophenyl | SH |
| A-95 | 2-Methyl-5-chlorophenyl | SH |
| A-96 | 2-Methyl-6-chlorophenyl | SH |
| A-97 | 3-Methyl-4-chlorophenyl | SH |
| A-98 | 3-Methyl-5-chlorophenyl | SH |
| A-99 | 2-Chloro-3-methylphenyl | SH |
| A-100 | 2-Chloro-4-methylphenyl | SH |
| A-101 | 2-Chloro-5-methylphenyl | SH |
| A-102 | 3-Chloro-4-methylphenyl | SH |
| A-103 | 2-Methyl-3-fluorophenyl | SH |
| A-104 | 2-Methyl-4-fluorophenyl | SH |
| A-105 | 2-Methyl-5-fluorophenyl | SH |
| A-106 | 2-Methyl-6-fluorophenyl | SH |
| A-107 | 3-Methyl-4-fluorophenyl | SH |
| A-108 | 3-Methyl-5-fluorophenyl | SH |
| A-109 | 2-Fluoro-3-methylphenyl | SH |
| A-110 | 2-Fluoro-4-methylphenyl | SH |
| A-111 | 2-Fluoro-5-methylphenyl | SH |
| A-112 | 3-Fluoro-4-methylphenyl | SH |
| A-113 | 2-Chloro-3-ethylphenyl | SH |
| A-114 | 2-Chloro-4-ethylphenyl | SH |
| A-115 | 2-Chloro-5-ethylphenyl | SH |
| A-116 | 3-Chloro-4-ethylphenyl | SH |
| A-117 | 2-Ethyl-3-chlorophenyl | SH |
| A-118 | 2-Ethyl-4-chlorophenyl | SH |
| A-119 | 2-Ethyl-5-chlorophenyl | SH |
| A-120 | 2-Ethyl-6-chlorophenyl | SH |
| A-121 | 2-Ethyl-3-fluorophenyl | SH |
| A-122 | 2-Ethyl-4-fluorophenyl | SH |
| A-123 | 2-Ethyl-5-fluorophenyl | SH |
| A-124 | 2-Ethyl-6-fluorophenyl | SH |
| A-125 | 3-Ethyl-4-fluorophenyl | SH |
| A-126 | 3-Ethyl-5-fluorophenyl | SH |
| A-127 | 2-Fluoro-3-ethylphenyl | SH |
| A-128 | 2-Fluoro-4-ethylphenyl | SH |
| A-129 | 2-Fluoro-5-ethylphenyl | SH |
| A-130 | 3-Fluoro-4-ethylphenyl | SH |
| A-131 | 2-Methoxy-3-chlorophenyl | SH |
| A-132 | 2-Methoxy-4-chlorophenyl | SH |
| A-133 | 2-Methoxy-5-chlorophenyl | SH |
| A-134 | 2-Methoxy-6-chlorophenyl | SH |
| A-135 | 3-Methoxy-4-chlorophenyl | SH |
| A-136 | 3-Methoxy-5-chlorophenyl | SH |
| A-137 | 2-Chloro-3-methoxyphenyl | SH |
| A-138 | 2-Chloro-4-methoxyphenyl | SH |
| A-139 | 2-Chloro-5-methoxyphenyl | SH |
| A-140 | 3-Chloro-4-methoxyphenyl | SH |
| A-141 | 2-Methoxy-3-fluorophenyl | SH |
| A-142 | 2-Methoxy-4-fluorophenyl | SH |
| A-143 | 2-Methoxy-5-fluorophenyl | SH |
| A-144 | 2-Methoxy-6-fluorophenyl | SH |
| A-145 | 3-Methoxy-4-fluorophenyl | SH |
| A-146 | 3-Methoxy-5-fluorophenyl | SH |
| A-147 | 2-Fluoro-3-methoxyphenyl | SH |
| A-148 | 2-Fluoro-4-methoxyphenyl | SH |
| A-149 | 2-Fluoro-5-methoxyphenyl | SH |
| A-150 | 3-Fluoro-4-methoxyphenyl | SH |
| A-151 | 3-Fluoro-5-methoxyphenyl | SH |
| A-152 | 2-(trifluoromethyl)-3-chlorophenyl | SH |
| A-153 | 2-(trifluoromethyl)-4-chlorophenyl | SH |
| A-154 | 2-(trifluoromethyl)-5-chlorophenyl | SH |
| A-155 | 2-(trifluoromethyl)-6-chlorophenyl | SH |
| A-156 | 3-(trifluoromethyl)-4-chlorophenyl | SH |
| A-157 | 3-(trifluoromethyl)-5-chlorophenyl | SH |
| A-158 | 2-Chloro-3-(trifluoromethyl)phenyl | SH |
| A-159 | 2-Chloro-4-(trifluoromethyl)phenyl | SH |
| A-160 | 2-Chloro-5-(trifluoromethyl)phenyl | SH |
| A-161 | 3-Chloro-4-(trifluoromethyl)phenyl | SH |
| A-162 | 2-(trifluoromethyl)-3-fluorophenyl | SH |
| A-163 | 2-(trifluoromethyl)-4-fluorophenyl | SH |
| A-164 | 2-(trifluoromethyl)-5-fluorophenyl | SH |
| A-165 | 2-(trifluoromethyl)-6-fluorophenyl | SH |
| A-166 | 3-(trifluoromethyl)-4-fluorophenyl | SH |
| A-167 | 3-(trifluoromethyl)-5-fluorophenyl | SH |
| A-168 | 2-Fluoro-3-(trifluoromethyl)phenyl | SH |
| A-169 | 2-Fluoro-4-(trifluoromethyl)phenyl | SH |
| A-170 | 2-Fluoro-5-(trifluoromethyl)phenyl | SH |
| A-171 | 3-Fluoro-4-(trifluoromethyl)phenyl | SH |
| A-172 | 2-(trifluoromethoxy)-3-chlorophenyl | SH |
| A-173 | 2-(trifluoromethoxy)-4-chlorophenyl | SH |
| A-174 | 2-(trifluoromethoxy)-5-chlorophenyl | SH |
| A-175 | 2-(trifluoromethoxy)-6-chlorophenyl | SH |
| A-176 | 3-(trifluoromethoxy)-4-chlorophenyl | SH |
| A-177 | 3-(trifluoromethoxy)-5-chlorophenyl | SH |
| A-178 | 2-Chloro-3-(trifluoromethoxy)phenyl | SH |
| A-179 | 2-Chloro-4-(trifluoromethoxy)phenyl | SH |
| A-180 | 2-Chloro-5-(trifluoromethoxy)phenyl | SH |
| A-181 | 3-Chloro-4-(trifluoromethoxy)phenyl | SH |
| A-182 | 2-(Trifluoromethoxy)-3-fluorophenyl | SH |
| A-183 | 2-(Trifluoromethoxy)-4-fluorophenyl | SH |
| A-184 | 2-(Trifluoromethoxy)-5-fluorophenyl | SH |
| A-185 | 2-(Trifluoromethoxy)-6-fluorophenyl | SH |
| A-186 | 3-(Trifluoromethoxy)-4-fluorophenyl | SH |
| A-187 | 3-(Trifluoromethoxy)-5-fluorophenyl | SH |
| A-188 | 2-Fluoro-3-(trifluoromethoxy)phenyl | SH |
| A-189 | 2-Fluoro-4-(trifluoromethoxy)phenyl | SH |
| A-190 | 2-Fluoro-5-(trifluoromethoxy)phenyl | SH |
| A-191 | 3-Fluoro-4-(trifluoromethoxy)phenyl | SH |
| A-192 | 2-(Difluoromethoxy)-3-chlorophenyl | SH |
| A-193 | 2-(Difluoromethoxy)-4-chlorophenyl | SH |
| A-194 | 2-(Difluoromethoxy)-5-chlorophenyl | SH |
| A-195 | 2-(Difluoromethoxy)-6-chlorophenyl | SH |
| A-196 | 3-(Difluoromethoxy)-4-chlorophenyl | SH |
| A-197 | 3-(Difluoromethoxy)-5-chlorophenyl | SH |
| A-198 | 2-Chloro-3-(difluoromethoxy)phenyl | SH |
| A-199 | 2-Chloro-4-(difluoromethoxy)phenyl | SH |
| A-200 | 2-Chloro-5-(difluoromethoxy)phenyl | SH |
| A-201 | 3-Chloro-4-(difluoromethoxy)phenyl | SH |
| A-202 | 2-(Difluoromethoxy)-3-fluorophenyl | SH |
| A-203 | 2-(Difluoromethoxy)-4-fluorophenyl | SH |
| A-204 | 2-(Difluoromethoxy)-5-fluorophenyl | SH |
| A-205 | 2-(Difluoromethoxy)-6-fluorophenyl | SH |
| A-206 | 3-(Difluoromethoxy)-4-fluorophenyl | SH |
| A-207 | 3-(Difluoromethoxy)-5-fluorophenyl | SH |
| A-208 | 2-Fluoro-3-(difluoromethoxy)phenyl | SH |
| A-209 | 2-Fluoro-4-(difluoromethoxy)phenyl | SH |
| A-210 | 2-Fluoro-5-(difluoromethoxy)phenyl | SH |
| A-211 | 3-Fluoro-4-(difluoromethoxy)phenyl | SH |
| A-212 | 2-(Trifluoromethylthio)-3-chlorophenyl | SH |
| A-213 | 2-(Trifluoromethylthio)-4-chlorophenyl | SH |
| A-214 | 2-(Trifluoromethylthio)-5-chlorophenyl | SH |
| A-215 | 2-(Trifluoromethylthio)-6-chlorophenyl | SH |
| A-216 | 3-(Trifluoromethylthio)-4-chlorophenyl | SH |
| A-217 | 3-(Trifluoromethylthio)-5-chlorophenyl | SH |
| A-218 | 2-Chloro-3-(trifluoromethylthio)phenyl | SH |
| A-219 | 2-Chloro-4-(trifluoromethylthio)phenyl | SH |
| A-220 | 2-Chloro-5-(trifluoromethylthio)phenyl | SH |
| A-221 | 3-Chloro-4-(trifluoromethylthio)phenyl | SH |
| A-222 | 2-(Trifluoromethylthio)-3-fluorophenyl | SH |
| A-223 | 2-(Trifluoromethylthio)-4-fluorophenyl | SH |
| A-224 | 2-(Trifluoromethylthio)-5-fluorophenyl | SH |
| A-225 | 2-(Trifluoromethylthio)-6-fluorophenyl | SH |
| A-226 | 3-(Trifluoromethylthio)-4-fluorophenyl | SH |
| A-227 | 3-(Trifluoromethylthio)-5-fluorophenyl | SH |
| A-228 | 2-Fluoro-3-(trifluoromethylthio)phenyl | SH |
| A-229 | 2-Fluoro-4-(trifluoromethylthio)phenyl | SH |
| A-230 | 2-Fluoro-5-(trifluoromethylthio)phenyl | SH |
| A-231 | 3-Fluoro-4-(trifluoromethylthio)phenyl | SH |
| A-232 | 2,3,4-Trichlorophenyl | SH |
| A-233 | 2,3,5-Trichlorophenyl | SH |
| A-234 | 2,3,6-Trichlorophenyl | SH |
| A-235 | 2,4,5-Trichlorophenyl | SH |
| A-236 | 2,4,6-Trichlorophenyl | SH |
| A-237 | 3,4,5-Trichlorophenyl | SH |
| A-238 | 2,3,4-Trifluorophenyl | SH |
| A-239 | 2,3,5-Trifluorophenyl | SH |
| A-240 | 2,3,6-Trifluorophenyl | SH |
| A-241 | 2,4,5-Trifluorophenyl | SH |
| A-242 | 2,4,6-Trifluorophenyl | SH |
| A-243 | 3,4,5-Trifluorophenyl | SH |
| A-244 | 2,3,4-Trimethylphenyl | SH |
| A-245 | 2,3,5-Trimethylphenyl | SH |
| A-246 | 2,3,6-Trimethylphenyl | SH |
| A-247 | 2,4,5-Trimethylphenyl | SH |
| A-248 | 2,4,6-Trimethylphenyl | SH |
| A-249 | 3,4,5-Trimethylphenyl | SH |
| A-250 | 2,3,4-Trimethoxyphenyl | SH |
| A-251 | 2,3,5-Trimethoxyphenyl | SH |
| A-252 | 2,3,6-Trimethoxyphenyl | SH |
| A-253 | 2,4,5-Trimethoxyphenyl | SH |
| A-254 | 2,4,6-Trimethoxyphenyl | SH |
| A-255 | 3,4,5-Trimethoxyphenyl | SH |
| A-256 | Phenyl | SCH₃ |
| A-257 | 2-Chlorophenyl | SCH₃ |
| A-258 | 3-Chlorophenyl | SCH₃ |
| A-259 | 4-Chlorophenyl | SCH₃ |
| A-260 | 2-fluorophenyl | SCH₃ |
| A-261 | 3-fluorophenyl | SCH₃ |
| A-262 | 4-fluorophenyl | SCH₃ |
| A-263 | 2-Methylphenyl | SCH₃ |
| A-264 | 3-Methylphenyl | SCH₃ |
| A-265 | 4-Methylphenyl | SCH₃ |
| A-266 | 2-Ethylphenyl | SCH₃ |
| A-267 | 3-Ethylphenyl | SCH₃ |
| A-268 | 4-Ethylphenyl | SCH₃ |
| A-269 | 2-Methoxyphenyl | SCH₃ |
| A-270 | 3-Methoxyphenyl | SCH₃ |
| A-271 | 4-Methoxyphenyl | SCH₃ |
| A-272 | 2-Trifluoromethylphenyl | SCH₃ |
| A-273 | 3-Trifluoromethylphenyl | SCH₃ |
| A-274 | 4-Trifluoromethylphenyl | SCH₃ |
| A-275 | 2-Trifluoromethoxyphenyl | SCH₃ |
| A-276 | 3-Trifluoromethoxyphenyl | SCH₃ |
| A-277 | 4-Trifluoromethoxyphenyl | SCH₃ |
| A-278 | 2-Difluoromethoxyphenyl | SCH₃ |
| A-279 | 3-Difluoromethoxyphenyl | SCH₃ |
| A-280 | 4-Difluoromethoxyphenyl | SCH₃ |
| A-281 | 2-Trifluoromethylthiophenyl | SCH₃ |
| A-282 | 3-Trifluoromethylthiophenyl | SCH₃ |
| A-283 | 4-Trifluoromethylthiophenyl | SCH₃ |
| A-284 | 2,3-Dichlorophenyl | SCH₃ |
| A-285 | 2,4-Dichlorophenyl | SCH₃ |
| A-286 | 2,5-Dichlorophenyl | SCH₃ |
| A-287 | 2,6-Dichlorophenyl | SCH₃ |
| A-288 | 3,4-Dichlorophenyl | SCH₃ |
| A-289 | 3,5-Dichlorophenyl | SCH₃ |
| A-290 | 2,3-Difluorophenyl | SCH₃ |
| A-291 | 2,4-Difluorophenyl | SCH₃ |
| A-292 | 2,5-Difluorophenyl | SCH₃ |
| A-293 | 2,6-Difluorophenyl | SCH₃ |
| A-294 | 3,4-Difluorophenyl | SCH₃ |
| A-295 | 3,5-Difluorophenyl | SCH₃ |
| A-296 | 2,3-Dimethylphenyl | SCH₃ |
| A-297 | 2,4-Dimethylphenyl | SCH₃ |
| A-298 | 2,5-Dimethylphenyl | SCH₃ |
| A-299 | 2,6-Dimethylphenyl | SCH₃ |
| A-300 | 3,4-Dimethylphenyl | SCH₃ |
| A-301 | 3,5-Dimethylphenyl | SCH₃ |
| A-302 | 2,3-Diethylphenyl | SCH₃ |
| A-303 | 2,4-Diethylphenyl | SCH₃ |
| A-304 | 2,5-Diethylphenyl | SCH₃ |
| A-305 | 2,6-Diethylphenyl | SCH₃ |
| A-306 | 3,5-Diethylphenyl | SCH₃ |
| A-307 | 3,4-Diethylphenyl | SCH₃ |
| A-308 | 2,3-Dimethoxyphenyl | SCH₃ |
| A-309 | 2,4-Dimethoxyphenyl | SCH₃ |
| A-310 | 2,5-Dimethoxyphenyl | SCH₃ |
| A-311 | 2,6-Dimethoxyphenyl | SCH₃ |
| A-312 | 3,4-Dimethoxyphenyl | SCH₃ |
| A-313 | 3,5-Dimethoxyphenyl | SCH₃ |
| A-314 | 2,3-Di(trifluoromethyl)phenyl | SCH₃ |
| A-315 | 2,4-Di(trifluoromethyl)phenyl | SCH₃ |
| A-316 | 2,5-Di(trifluoromethyl)phenyl | SCH₃ |
| A-317 | 2,6-Di(trifluoromethyl)phenyl | SCH₃ |
| A-318 | 3,4-Di(trifluoromethyl)phenyl | SCH₃ |
| A-319 | 3,5-Di(trifluoromethyl)phenyl | SCH₃ |
| A-320 | 2,3-Di(trifluoromethoxy)phenyl | SCH₃ |
| A-321 | 2,4-Di(trifluoromethoxy)phenyl | SCH₃ |
| A-322 | 2,5-Di(trifluoromethoxy)phenyl | SCH₃ |
| A-323 | 2,6-Di(trifluoromethoxy)phenyl | SCH₃ |
| A-324 | 3,4-Di(trifluoromethoxy)phenyl | SCH₃ |
| A-325 | 3,5-Di(trifluoromethoxy)phenyl | SCH₃ |
| A-326 | 2,3-Di(difluoromethoxy)phenyl | SCH₃ |
| A-327 | 2,4-Di(difluoromethoxy)phenyl | SCH₃ |
| A-328 | 2,5-Di(difluoromethoxy)phenyl | SCH₃ |
| A-329 | 2,6-Di(difluoromethoxy)phenyl | SCH₃ |
| A-330 | 3,4-Di(difluoromethoxy)phenyl | SCH₃ |
| A-331 | 3,5-Di(difluoromethoxy)phenyl | SCH₃ |
| A-332 | 2,3-Di(trifluoromethylthio)phenyl | SCH₃ |
| A-333 | 2,4-Di(trifluoromethylthio)phenyl | SCH₃ |
| A-334 | 2,5-Di(trifluoromethylthio)phenyl | SCH₃ |
| A-335 | 2,6-Di(trifluoromethylthio)phenyl | SCH₃ |
| A-336 | 3,4-Di(trifluoromethylthio)phenyl | SCH₃ |
| A-337 | 3,5-Di(trifluoromethylthio)phenyl | SCH₃ |
| A-338 | 2-Fluoro-3-chlorophenyl | SCH₃ |
| A-339 | 2-Fluoro-4-chlorophenyl | SCH₃ |
| A-340 | 2-Fluoro-5-chlorophenyl | SCH₃ |
| A-341 | 2-Fluoro-6-chlorophenyl | SCH₃ |
| A-342 | 3-Fluoro-4-chlorophenyl | SCH₃ |
| A-343 | 3-Fluoro-5-chlorophenyl | SCH₃ |
| A-344 | 2-Chloro-3-fluorophenyl | SCH₃ |
| A-345 | 2-Chloro-4-fluorophenyl | SCH₃ |
| A-346 | 2-Chloro-5-fluorophenyl | SCH₃ |
| A-347 | 3-Chloro-4-fluorophenyl | SCH₃ |
| A-348 | 2-Methyl-3-chlorophenyl | SCH₃ |
| A-349 | 2-Methyl-4-chlorophenyl | SCH₃ |
| A-350 | 2-Methyl-5-chlorophenyl | SCH₃ |
| A-351 | 2-Methyl-6-chlorophenyl | SCH₃ |
| A-352 | 3-Methyl-4-chlorophenyl | SCH₃ |
| A-353 | 3-Methyl-5-chlorophenyl | SCH₃ |
| A-354 | 2-Chloro-3-methylphenyl | SCH₃ |
| A-355 | 2-Chloro-4-methylphenyl | SCH₃ |
| A-356 | 2-Chloro-5-methylphenyl | SCH₃ |
| A-357 | 3-Chloro-4-methylphenyl | SCH₃ |
| A-358 | 2-Methyl-3-fluorophenyl | SCH₃ |
| A-359 | 2-Methyl-4-fluorophenyl | SCH₃ |
| A-360 | 2-Methyl-5-fluorophenyl | SCH₃ |
| A-361 | 2-Methyl-6-fluorophenyl | SCH₃ |
| A-362 | 3-Methyl-4-fluorophenyl | SCH₃ |
| A-363 | 3-Methyl-5-fluorophenyl | SCH₃ |
| A-364 | 2-Fluoro-3-methylphenyl | SCH₃ |
| A-365 | 2-Fluoro-4-methylphenyl | SCH₃ |
| A-366 | 2-Fluoro-5-methylphenyl | SCH₃ |
| A-367 | 3-Fluoro-4-methylphenyl | SCH₃ |
| A-368 | 2-Chloro-3-ethylphenyl | SCH₃ |
| A-369 | 2-Chloro-4-ethylphenyl | SCH₃ |
| A-370 | 2-Chloro-5-ethylphenyl | SCH₃ |
| A-371 | 3-Chloro-4-ethylphenyl | SCH₃ |
| A-372 | 2-Ethyl-3-chlorophenyl | SCH₃ |
| A-373 | 2-Ethyl-4-chlorophenyl | SCH₃ |
| A-374 | 2-Ethyl-5-chlorophenyl | SCH₃ |
| A-375 | 2-Ethyl-6-chlorophenyl | SCH₃ |
| A-376 | 2-Ethyl-3-fluorophenyl | SCH₃ |
| A-377 | 2-Ethyl-4-fluorophenyl | SCH₃ |
| A-378 | 2-Ethyl-5-fluorophenyl | SCH₃ |
| A-379 | 2-Ethyl-6-fluorophenyl | SCH₃ |
| A-380 | 3-Ethyl-4-fluorophenyl | SCH₃ |
| A-381 | 3-Ethyl-5-fluorophenyl | SCH₃ |
| A-382 | 2-Fluoro-3-ethylphenyl | SCH₃ |
| A-383 | 2-Fluoro-4-ethylphenyl | SCH₃ |
| A-384 | 2-Fluoro-5-ethylphenyl | SCH₃ |
| A-385 | 3-Fluoro-4-ethylphenyl | SCH₃ |
| A-386 | 2-Methoxy-3-chlorophenyl | SCH₃ |
| A-387 | 2-Methoxy-4-chlorophenyl | SCH₃ |
| A-388 | 2-Methoxy-5-chlorophenyl | SCH₃ |
| A-389 | 2-Methoxy-6-chlorophenyl | SCH₃ |
| A-390 | 3-Methoxy-4-chlorophenyl | SCH₃ |
| A-391 | 3-Methoxy-5-chlorophenyl | SCH₃ |
| A-392 | 2-Chloro-3-methoxyphenyl | SCH₃ |
| A-393 | 2-Chloro-4-methoxyphenyl | SCH₃ |
| A-394 | 2-Chloro-5-methoxyphenyl | SCH₃ |
| A-395 | 3-Chloro-4-methoxyphenyl | SCH₃ |
| A-396 | 2-Methoxy-3-fluorophenyl | SCH₃ |
| A-397 | 2-Methoxy-4-fluorophenyl | SCH₃ |
| A-398 | 2-Methoxy-5-fluorophenyl | SCH₃ |
| A-399 | 2-Methoxy-6-fluorophenyl | SCH₃ |
| A-400 | 3-Methoxy-4-fluorophenyl | SCH₃ |
| A-401 | 3-Methoxy-5-fluorophenyl | SCH₃ |
| A-402 | 2-Fluoro-3-methoxyphenyl | SCH₃ |
| A-403 | 2-Fluoro-4-methoxyphenyl | SCH₃ |
| A-404 | 2-Fluoro-5-methoxyphenyl | SCH₃ |
| A-405 | 3-Fluoro-4-methoxyphenyl | SCH₃ |
| A-406 | 3-Fluoro-5-methoxyphenyl | SCH₃ |
| A-407 | 2-(Trifluoromethyl)-3-chlorophenyl | SCH₃ |
| A-408 | 2-(Trifluoromethyl)-4-chlorophenyl | SCH₃ |
| A-409 | 2-(Trifluoromethyl)-5-chlorophenyl | SCH₃ |
| A-410 | 2-(Trifluoromethyl)-6-chlorophenyl | SCH₃ |
| A-411 | 3-(Trifluoromethyl)-4-chlorophenyl | SCH₃ |
| A-412 | 3-(Trifluoromethyl)-5-chlorophenyl | SCH₃ |
| A-413 | 2-Chloro-3-(trifluoromethyl)phenyl | SCH₃ |
| A-414 | 2-Chloro-4-(trifluoromethyl)phenyl | SCH₃ |
| A-415 | 2-Chloro-5-(trifluoromethyl)phenyl | SCH₃ |
| A-416 | 3-Chloro-4-(trifluoromethyl)phenyl | SCH₃ |
| A-417 | 2-(Trifluoromethyl)-3-fluorophenyl | SCH₃ |
| A-418 | 2-(Trifluoromethyl)-4-fluorophenyl | SCH₃ |
| A-419 | 2-(Trifluoromethyl)-5-fluorophenyl | SCH₃ |
| A-420 | 2-(Trifluoromethyl)-6-fluorophenyl | SCH₃ |
| A-421 | 3-(Trifluoromethyl)-4-fluorophenyl | SCH₃ |
| A-422 | 3-(Trifluoromethyl)-5-fluorophenyl | SCH₃ |
| A-423 | 2-Fluoro-3-(trifluoromethyl)phenyl | SCH₃ |
| A-424 | 2-Fluoro-4-(trifluoromethyl)phenyl | SCH₃ |
| A-425 | 2-Fluoro-5-(trifluoromethyl)phenyl | SCH₃ |
| A-426 | 3-Fluoro-4-(trifluoromethyl)phenyl | SCH₃ |
| A-427 | 2-(Trifluoromethoxy)-3-chlorophenyl | SCH₃ |
| A-428 | 2-(Trifluoromethoxy)-4-chlorophenyl | SCH₃ |
| A-429 | 2-(Trifluoromethoxy)-5-chlorophenyl | SCH₃ |
| A-430 | 2-(Trifluoromethoxy)-6-chlorophenyl | SCH₃ |
| A-431 | 3-(Trifluoromethoxy)-4-chlorophenyl | SCH₃ |
| A-432 | 3-(Trifluoromethoxy)-5-chlorophenyl | SCH₃ |
| A-433 | 2-Chloro-3-(trifluoromethoxy)phenyl | SCH₃ |
| A-434 | 2-Chloro-4-(trifluoromethoxy)phenyl | SCH₃ |
| A-435 | 2-Chloro-5-(trifluoromethoxy)phenyl | SCH₃ |
| A-436 | 3-Chloro-4-(trifluoromethoxy)phenyl | SCH₃ |
| A-437 | 2-(Trifluoromethoxy)-3-fluorophenyl | SCH₃ |
| A-438 | 2-(Trifluoromethoxy)-4-fluorophenyl | SCH₃ |
| A-439 | 2-(Trifluoromethoxy)-5-fluorophenyl | SCH₃ |
| A-440 | 2-(Trifluoromethoxy)-6-fluorophenyl | SCH₃ |
| A-441 | 3-(Trifluoromethoxy)-4-fluorophenyl | SCH₃ |
| A-442 | 3-(Trifluoromethoxy)-5-fluorophenyl | SCH₃ |
| A-443 | 2-Fluoro-3-(trifluoromethoxy)phenyl | SCH₃ |
| A-444 | 2-Fluoro-4-(trifluoromethoxy)phenyl | SCH₃ |
| A-445 | 2-Fluoro-5-(trifluoromethoxy)phenyl | SCH₃ |
| A-446 | 3-Fluoro-4-(trifluoromethoxy)phenyl | SCH₃ |
| A-447 | 2-(Difluoromethoxy)-3-chlorophenyl | SCH₃ |
| A-448 | 2-(Difluoromethoxy)-4-chlorophenyl | SCH₃ |
| A-449 | 2-(Difluoromethoxy)-5-chlorophenyl | SCH₃ |
| A-450 | 2-(Difluoromethoxy)-6-chlorophenyl | SCH₃ |
| A-451 | 3-(Difluoromethoxy)-4-chlorophenyl | SCH₃ |
| A-452 | 3-(Difluoromethoxy)-5-chlorophenyl | SCH₃ |
| A-453 | 2-Chloro-3-(difluoromethoxy)phenyl | SCH₃ |
| A-454 | 2-Chloro-4-(difluoromethoxy)phenyl | SCH₃ |
| A-455 | 2-Chloro-5-(difluoromethoxy)phenyl | SCH₃ |
| A-456 | 3-Chloro-4-(difluoromethoxy)phenyl | SCH₃ |
| A-457 | 2-(Difluoromethoxy)-3-fluorophenyl | SCH₃ |
| A-458 | 2-(Difluoromethoxy)-4-fluorophenyl | SCH₃ |
| A-459 | 2-(Difluoromethoxy)-5-fluorophenyl | SCH₃ |
| A-460 | 2-(Difluoromethoxy)-6-fluorophenyl | SCH₃ |
| A-461 | 3-(Difluoromethoxy)-4-fluorophenyl | SCH₃ |
| A-462 | 3-(Difluoromethoxy)-5-fluorophenyl | SCH₃ |
| A-463 | 2-Fluoro-3-(difluoromethoxy)phenyl | SCH₃ |
| A-464 | 2-Fluoro-4-(difluoromethoxy)phenyl | SCH₃ |
| A-465 | 2-Fluoro-5-(difluoromethoxy)phenyl | SCH₃ |
| A-466 | 3-Fluoro-4-(difluoromethoxy)phenyl | SCH₃ |
| A-467 | 2-(Trifluoromethylthio)-3-chlorophenyl | SCH₃ |
| A-468 | 2-(Trifluoromethylthio)-4-chlorophenyl | SCH₃ |
| A-469 | 2-(Trifluoromethylthio)-5-chlorophenyl | SCH₃ |
| A-470 | 2-(Trifluoromethylthio)-6-chlorophenyl | SCH₃ |
| A-471 | 3-(Trifluoromethylthio)-4-chlorophenyl | SCH₃ |
| A-472 | 3-(Trifluoromethylthio)-5-chlorophenyl | SCH₃ |
| A-473 | 2-Chloro-3-(trifluoromethylthio)phenyl | SCH₃ |
| A-474 | 2-Chloro-4-(trifluoromethylthio)phenyl | SCH₃ |
| A-475 | 2-Chloro-5-(trifluoromethylthio)phenyl | SCH₃ |
| A-476 | 3-Chloro-4-(trifluoromethylthio)phenyl | SCH₃ |
| A-477 | 2-(Trifluoromethylthio)-3-fluorophenyl | SCH₃ |
| A-478 | 2-(Trifluoromethylthio)-4-fluorophenyl | SCH₃ |
| A-479 | 2-(Trifluoromethylthio)-5-fluorophenyl | SCH₃ |
| A-480 | 2-(Trifluoromethylthio)-6-fluorophenyl | SCH₃ |
| A-481 | 3-(Trifluoromethylthio)-4-fluorophenyl | SCH₃ |
| A-482 | 3-(Trifluoromethylthio)-5-fluorophenyl | SCH₃ |
| A-483 | 2-Fluoro-3-(trifluoromethylthio)phenyl | SCH₃ |
| A-484 | 2-Fluoro-4-(trifluoromethylthio)phenyl | SCH₃ |
| A-485 | 2-Fluoro-5-(trifluoromethylthio)phenyl | SCH₃ |
| A-486 | 3-Fluoro-4-(trifluoromethylthio)phenyl | SCH₃ |
| A-487 | 2,3,4-Trichlorophenyl | SCH₃ |
| A-488 | 2,3,5-Trichlorophenyl | SCH₃ |
| A-489 | 2,3,6-Trichlorophenyl | SCH₃ |
| A-490 | 2,4,5-Trichlorophenyl | SCH₃ |
| A-491 | 2,4,6-Trichlorophenyl | SCH₃ |
| A-492 | 3,4,5-Trichlorophenyl | SCH₃ |
| A-493 | 2,3,4-Trifluorophenyl | SCH₃ |
| A-494 | 2,3,5-Trifluorophenyl | SCH₃ |
| A-495 | 2,3,6-Trifluorophenyl | SCH₃ |
| A-496 | 2,4,5-Trifluorophenyl | SCH₃ |
| A-497 | 2,4,6-Trifluorophenyl | SCH₃ |
| A-498 | 3,4,5-Trifluorophenyl | SCH₃ |
| A-499 | 2,3,4-Trimethylphenyl | SCH₃ |
| A-500 | 2,3,5-Trimethylphenyl | SCH₃ |
| A-501 | 2,3,6-Trimethylphenyl | SCH₃ |
| A-502 | 2,4,5-Trimethylphenyl | SCH₃ |
| A-503 | 2,4,6-Trimethylphenyl | SCH₃ |
| A-504 | 3,4,5-Trimethylphenyl | SCH₃ |
| A-505 | 2,3,4-Trimethoxyphenyl | SCH₃ |
| A-506 | 2,3,5-Trimethoxyphenyl | SCH₃ |
| A-507 | 2,3,6-Trimethoxyphenyl | SCH₃ |
| A-508 | 2,4,5-Trimethoxyphenyl | SCH₃ |
| A-509 | 2,4,6-Trimethoxyphenyl | SCH₃ |
| A-510 | 3,4,5-Trimethoxyphenyl | SCH₃ |
| A-511 | Phenyl | SC₂H₅ |
| A-512 | 2-Chlorophenyl | SC₂H₅ |
| A-513 | 3-Chlorophenyl | SC₂H₅ |
| A-514 | 4-Chlorophenyl | SC₂H₅ |
| A-515 | 2-Fluorophenyl | SC₂H₅ |
| A-516 | 3-Fluorophenyl | SC₂H₅ |
| A-517 | 4-Fluorophenyl | SC₂H₅ |
| A-518 | 2-Methylphenyl | SC₂H₅ |
| A-519 | 3-Methylphenyl | SC₂H₅ |
| A-520 | 4-Methylphenyl | SC₂H₅ |
| A-521 | 2-Ethylphenyl | SC₂H₅ |
| A-522 | 3-Ethylphenyl | SC₂H₅ |
| A-523 | 4-Ethylphenyl | SC₂H₅ |
| A-524 | 2-Methoxyphenyl | SC₂H₅ |
| A-525 | 3-Methoxyphenyl | SC₂H₅ |
| A-526 | 4-Methoxyphenyl | SC₂H₅ |
| A-527 | 2-Trifluoromethylphenyl | SC₂H₅ |
| A-528 | 3-Trifluoromethylphenyl | SC₂H₅ |
| A-529 | 4-Trifluoromethylphenyl | SC₂H₅ |
| A-530 | 2-Trifluoromethoxyphenyl | SC₂H₅ |
| A-531 | 3-Trifluoromethoxyphenyl | SC₂H₅ |
| A-532 | 4-Trifluoromethoxyphenyl | SC₂H₅ |
| A-533 | 2-Difluoromethoxyphenyl | SC₂H₅ |
| A-534 | 3-Difluoromethoxyphenyl | SC₂H₅ |
| A-535 | 4-Difluoromethoxyphenyl | SC₂H₅ |
| A-536 | 2-Trifluoromethylthiophenyl | SC₂H₅ |
| A-537 | 3-Trifluoromethylthiophenyl | SC₂H₅ |
| A-538 | 4-Trifluoromethylthiophenyl | SC₂H₅ |
| A-539 | 2,3-Dichlorophenyl | SC₂H₅ |
| A-540 | 2,4-Dichlorophenyl | SC₂H₅ |
| A-541 | 2,5-Dichlorophenyl | SC₂H₅ |
| A-542 | 2,6-Dichlorophenyl | SC₂H₅ |
| A-543 | 3,4-Dichlorophenyl | SC₂H₅ |
| A-544 | 3,5-Dichlorophenyl | SC₂H₅ |
| A-545 | 2,3-Difluorophenyl | SC₂H₅ |
| A-546 | 2,4-Difluorophenyl | SC₂H₅ |
| A-547 | 2,5-Difluorophenyl | SC₂H₅ |
| A-548 | 2,6-Difluorophenyl | SC₂H₅ |
| A-549 | 3,4-Difluorophenyl | SC₂H₅ |
| A-550 | 3,5-Difluorophenyl | SC₂H₅ |
| A-551 | 2,3-Dimethylphenyl | SC₂H₅ |
| A-552 | 2,4-Dimethylphenyl | SC₂H₅ |
| A-553 | 2,5-Dimethylphenyl | SC₂H₅ |
| A-554 | 2,6-Dimethylphenyl | SC₂H₅ |
| A-555 | 3,4-Dimethylphenyl | SC₂H₅ |
| A-556 | 3,5-Dimethylphenyl | SC₂H₅ |
| A-557 | 2,3-Diethylphenyl | SC₂H₅ |
| A-558 | 2,4-Diethylphenyl | SC₂H₅ |
| A-559 | 2,5-Diethylphenyl | SC₂H₅ |
| A-560 | 2,6-Diethylphenyl | SC₂H₅ |
| A-561 | 3,5-Diethylphenyl | SC₂H₅ |
| A-562 | 3,4-Diethylphenyl | SC₂H₅ |
| A-563 | 2,3-Dimethoxyphenyl | SC₂H₅ |
| A-564 | 2,4-Dimethoxyphenyl | SC₂H₅ |
| A-565 | 2,5-Dimethoxyphenyl | SC₂H₅ |
| A-566 | 2,6-Dimethoxyphenyl | SC₂H₅ |
| A-567 | 3,4-Dimethoxyphenyl | SC₂H₅ |
| A-568 | 3,5-Dimethoxyphenyl | SC₂H₅ |
| A-569 | 2,3-Di(trifluoromethyl)phenyl | SC₂H₅ |
| A-570 | 2,4-Di(trifluoromethyl)phenyl | SC₂H₅ |
| A-571 | 2,5-Di(trifluoromethyl)phenyl | SC₂H₅ |
| A-572 | 2,6-Di(trifluoromethyl)phenyl | SC₂H₅ |
| A-573 | 3,4-Di(trifluoromethyl)phenyl | SC₂H₅ |
| A-574 | 3,5-Di(trifluoromethyl)phenyl | SC₂H₅ |
| A-575 | 2,3-Di(trifluoromethoxy)phenyl | SC₂H₅ |
| A-576 | 2,4-Di(trifluoromethoxy)phenyl | SC₂H₅ |
| A-577 | 2,5-Di(trifluoromethoxy)phenyl | SC₂H₅ |
| A-578 | 2,6-Di(trifluoromethoxy)phenyl | SC₂H₅ |
| A-579 | 3,4-Di(trifluoromethoxy)phenyl | SC₂H₅ |
| A-580 | 3,5-Di(trifluoromethoxy)phenyl | SC₂H₅ |
| A-581 | 2,3-Di(difluoromethoxy)phenyl | SC₂H₅ |
| A-582 | 2,4-Di(difluoromethoxy)phenyl | SC₂H₅ |
| A-583 | 2,5-Di(difluoromethoxy)phenyl | SC₂H₅ |
| A-584 | 2,6-Di(difluoromethoxy)phenyl | SC₂H₅ |
| A-585 | 3,4-Di(difluoromethoxy)phenyl | SC₂H₅ |
| A-586 | 3,5-Di(difluoromethoxy)phenyl | SC₂H₅ |
| A-587 | 2,3-Di(trifluoromethylthio)phenyl | SC₂H₅ |
| A-588 | 2,4-Di(trifluoromethylthio)phenyl | SC₂H₅ |
| A-589 | 2,5-Di(trifluoromethylthio)phenyl | SC₂H₅ |
| A-590 | 2,6-Di(trifluoromethylthio)phenyl | SC₂H₅ |
| A-591 | 3,4-Di(trifluoromethylthio)phenyl | SC₂H₅ |
| A-592 | 3,5-Di(trifluoromethylthio)phenyl | SC₂H₅ |
| A-593 | 2-Fluoro-3-chlorophenyl | SC₂H₅ |
| A-594 | 2-Fluoro-4-chlorophenyl | SC₂H₅ |
| A-595 | 2-Fluoro-5-chlorophenyl | SC₂H₅ |
| A-596 | 2-Fluoro-6-chlorophenyl | SC₂H₅ |
| A-597 | 3-Fluoro-4-chlorophenyl | SC₂H₅ |
| A-598 | 3-Fluoro-5-chlorophenyl | SC₂H₅ |
| A-599 | 2-Chloro-3-fluorophenyl | SC₂H₅ |
| A-600 | 2-Chloro-4-fluorophenyl | SC₂H₅ |
| A-601 | 2-Chloro-5-fluorophenyl | SC₂H₅ |
| A-602 | 3-Chloro-4-fluorophenyl | SC₂H₅ |
| A-603 | 2-Methyl-3-chlorophenyl | SC₂H₅ |
| A-604 | 2-Methyl-4-chlorophenyl | SC₂H₅ |
| A-605 | 2-Methyl-5-chlorophenyl | SC₂H₅ |
| A-606 | 2-Methyl-6-chlorophenyl | SC₂H₅ |
| A-607 | 3-Methyl-4-chlorophenyl | SC₂H₅ |
| A-608 | 3-Methyl-5-chlorophenyl | SC₂H₅ |
| A-609 | 2-Chloro-3-methylphenyl | SC₂H₅ |
| A-610 | 2-Chloro-4-methylphenyl | SC₂H₅ |
| A-611 | 2-Chloro-5-methylphenyl | SC₂H₅ |
| A-612 | 3-Chloro-4-methylphenyl | SC₂H₅ |
| A-613 | 2-Methyl-3-fluorophenyl | SC₂H₅ |
| A-614 | 2-Methyl-4-fluorophenyl | SC₂H₅ |
| A-615 | 2-Methyl-5-fluorophenyl | SC₂H₅ |
| A-616 | 2-Methyl-6-fluorophenyl | SC₂H₅ |
| A-617 | 3-Methyl-4-fluorophenyl | SC₂H₅ |
| A-618 | 3-Methyl-5-fluorophenyl | SC₂H₅ |
| A-619 | 2-Fluoro-3-methylphenyl | SC₂H₅ |
| A-620 | 2-Fluoro-4-methylphenyl | SC₂H₅ |
| A-621 | 2-Fluoro-5-methylphenyl | SC₂H₅ |
| A-622 | 3-Fluoro-4-methylphenyl | SC₂H₅ |
| A-623 | 2-Chloro-3-ethylphenyl | SC₂H₅ |
| A-624 | 2-Chloro-4-ethylphenyl | SC₂H₅ |
| A-625 | 2-Chloro-5-ethylphenyl | SC₂H₅ |
| A-626 | 3-Chloro-4-ethylphenyl | SC₂H₅ |
| A-627 | 2-Ethyl-3-chlorophenyl | SC₂H₅ |
| A-628 | 2-Ethyl-4-chlorophenyl | SC₂H₅ |
| A-629 | 2-Ethyl-5-chlorophenyl | SC₂H₅ |
| A-630 | 2-Ethyl-6-chlorophenyl | SC₂H₅ |
| A-631 | 2-Ethyl-3-fluorophenyl | SC₂H₅ |
| A-632 | 2-Ethyl-4-fluorophenyl | SC₂H₅ |
| A-633 | 2-Ethyl-5-fluorophenyl | SC₂H₅ |
| A-634 | 2-Ethyl-6-fluorophenyl | SC₂H₅ |
| A-635 | 3-Ethyl-4-fluorophenyl | SC₂H₅ |
| A-636 | 3-Ethyl-5-fluorophenyl | SC₂H₅ |
| A-637 | 2-Fluoro-3-ethylphenyl | SC₂H₅ |
| A-638 | 2-Fluoro-4-ethylphenyl | SC₂H₅ |
| A-639 | 2-Fluoro-5-ethylphenyl | SC₂H₅ |
| A-640 | 3-Fluoro-4-ethylphenyl | SC₂H₅ |
| A-641 | 2-Methoxy-3-chlorophenyl | SC₂H₅ |
| A-642 | 2-Methoxy-4-chlorophenyl | SC₂H₅ |
| A-643 | 2-Methoxy-5-chlorophenyl | SC₂H₅ |
| A-644 | 2-Methoxy-6-chlorophenyl | SC₂H₅ |
| A-645 | 3-Methoxy-4-chlorophenyl | SC₂H₅ |
| A-646 | 3-Methoxy-5-chlorophenyl | SC₂H₅ |
| A-647 | 2-Chloro-3-methoxyphenyl | SC₂H₅ |
| A-648 | 2-Chloro-4-methoxyphenyl | SC₂H₅ |
| A-649 | 2-Chloro-5-methoxyphenyl | SC₂H₅ |
| A-650 | 3-Chloro-4-methoxyphenyl | SC₂H₅ |
| A-651 | 2-Methoxy-3-fluorophenyl | SC₂H₅ |
| A-652 | 2-Methoxy-4-fluorophenyl | SC₂H₅ |
| A-653 | 2-Methoxy-5-fluorophenyl | SC₂H₅ |
| A-654 | 2-Methoxy-6-fluorophenyl | SC₂H₅ |
| A-655 | 3-Methoxy-4-fluorophenyl | SC₂H₅ |
| A-656 | 3-Methoxy-5-fluorophenyl | SC₂H₅ |
| A-657 | 2-Fluoro-3-methoxyphenyl | SC₂H₅ |
| A-658 | 2-Fluoro-4-methoxyphenyl | SC₂H₅ |
| A-659 | 2-Fluoro-5-methoxyphenyl | SC₂H₅ |
| A-660 | 3-Fluoro-4-methoxyphenyl | SC₂H₅ |
| A-661 | 3-Fluoro-5-methoxyphenyl | SC₂H₅ |
| A-662 | 2-(Trifluoromethyl)-3-chlorophenyl | SC₂H₅ |
| A-663 | 2-(Trifluoromethyl)-4-chlorophenyl | SC₂H₅ |
| A-664 | 2-(Trifluoromethyl)-5-chlorophenyl | SC₂H₅ |
| A-665 | 2-(Trifluoromethyl)-6-chlorophenyl | SC₂H₅ |
| A-666 | 3-(Trifluoromethyl)-4-chlorophenyl | SC₂H₅ |
| A-667 | 3-(Trifluoromethyl)-5-chlorophenyl | SC₂H₅ |
| A-668 | 2-Chloro-3-(trifluoromethyl)phenyl | SC₂H₅ |
| A-669 | 2-Chloro-4-(trifluoromethyl)phenyl | SC₂H₅ |
| A-670 | 2-Chloro-5-(trifluoromethyl)phenyl | SC₂H₅ |
| A-671 | 3-Chloro-4-(trifluoromethyl)phenyl | SC₂H₅ |
| A-672 | 2-(Trifluoromethyl)-3-fluorophenyl | SC₂H₅ |
| A-673 | 2-(Trifluoromethyl)-4-fluorophenyl | SC₂H₅ |
| A-674 | 2-(Trifluoromethyl)-5-fluorophenyl | SC₂H₅ |
| A-675 | 2-(Trifluoromethyl)-6-fluorophenyl | SC₂H₅ |
| A-676 | 3-(Trifluoromethyl)-4-fluorophenyl | SC₂H₅ |
| A-677 | 3-(Trifluoromethyl)-5-fluorophenyl | SC₂H₅ |
| A-678 | 2-Fluoro-3-(trifluoromethyl)phenyl | SC₂H₅ |
| A-679 | 2-Fluoro-4-(trifluoromethyl)phenyl | SC₂H₅ |
| A-680 | 2-Fluoro-5-(trifluoromethyl)phenyl | SC₂H₅ |
| A-681 | 3-Fluoro-4-(trifluoromethyl)phenyl | SC₂H₅ |
| A-682 | 2-(Trifluoromethoxy)-3-chlorophenyl | SC₂H₅ |
| A-683 | 2-(Trifluoromethoxy)-4-chlorophenyl | SC₂H₅ |
| A-684 | 2-(Trifluoromethoxy)-5-chlorophenyl | SC₂H₅ |
| A-685 | 2-(Trifluoromethoxy)-6-chlorophenyl | SC₂H₅ |
| A-686 | 3-(Trifluoromethoxy)-4-chlorophenyl | SC₂H₅ |
| A-687 | 3-(Trifluoromethoxy)-5-chlorophenyl | SC₂H₅ |
| A-688 | 2-Chloro-3-(trifluoromethoxy)phenyl | SC₂H₅ |
| A-689 | 2-Chloro-4-(trifluoromethoxy)phenyl | SC₂H₅ |
| A-690 | 2-Chloro-5-(trifluoromethoxy)phenyl | SC₂H₅ |
| A-691 | 3-Chloro-4-(trifluoromethoxy)phenyl | SC₂H₅ |
| A-692 | 2-(Trifluoromethoxy)-3-fluorophenyl | SC₂H₅ |
| A-693 | 2-(Trifluoromethoxy)-4-fluorophenyl | SC₂H₅ |
| A-694 | 2-(Trifluoromethoxy)-5-fluorophenyl | SC₂H₅ |
| A-695 | 2-(Trifluoromethoxy)-6-fluorophenyl | SC₂H₅ |
| A-696 | 3-(Trifluoromethoxy)-4-fluorophenyl | SC₂H₅ |
| A-697 | 3-(Trifluoromethoxy)-5-fluorophenyl | SC₂H₅ |
| A-698 | 2-Fluoro-3-(trifluoromethoxy)phenyl | SC₂H₅ |
| A-699 | 2-Fluoro-4-(trifluoromethoxy)phenyl | SC₂H₅ |
| A-700 | 2-Fluoro-5-(trifluoromethoxy)phenyl | SC₂H₅ |
| A-701 | 3-Fluoro-4-(trifluoromethoxy)phenyl | SC₂H₅ |
| A-702 | 2-(Difluoromethoxy)-3-chlorophenyl | SC₂H₅ |
| A-703 | 2-(Difluoromethoxy)-4-chlorophenyl | SC₂H₅ |
| A-704 | 2-(Difluoromethoxy)-5-chlorophenyl | SC₂H₅ |
| A-705 | 2-(Difluoromethoxy)-6-chlorophenyl | SC₂H₅ |
| A-706 | 3-(Difluoromethoxy)-4-chlorophenyl | SC₂H₅ |
| A-707 | 3-(Difluoromethoxy)-5-chlorophenyl | SC₂H₅ |
| A-708 | 2-Chloro-3-(difluoromethoxy)phenyl | SC₂H₅ |
| A-709 | 2-Chloro-4-(difluoromethoxy)phenyl | SC₂H₅ |
| A-710 | 2-Chloro-5-(difluoromethoxy)phenyl | SC₂H₅ |
| A-711 | 3-Chloro-4-(difluoromethoxy)phenyl | SC₂H₅ |
| A-712 | 2-(Difluoromethoxy)-3-fluorophenyl | SC₂H₅ |
| A-713 | 2-(Difluoromethoxy)-4-fluorophenyl | SC₂H₅ |
| A-714 | 2-(Difluoromethoxy)-5-fluorophenyl | SC₂H₅ |
| A-715 | 2-(Difluoromethoxy)-6-fluorophenyl | SC₂H₅ |
| A-716 | 3-(Difluoromethoxy)-4-fluorophenyl | SC₂H₅ |
| A-717 | 3-(Difluoromethoxy)-5-fluorophenyl | SC₂H₅ |
| A-718 | 2-Fluoro-3-(difluoromethoxy)phenyl | SC₂H₅ |
| A-719 | 2-Fluoro-4-(difluoromethoxy)phenyl | SC₂H₅ |
| A-720 | 2-Fluoro-5-(difluoromethoxy)phenyl | SC₂H₅ |
| A-721 | 3-Fluoro-4-(difluoromethoxy)phenyl | SC₂H₅ |
| A-722 | 2-(Trifluoromethylthio)-3-chlorophenyl | SC₂H₅ |
| A-723 | 2-(Trifluoromethylthio)-4-chlorophenyl | SC₂H₅ |
| A-724 | 2-(Trifluoromethylthio)-5-chlorophenyl | SC₂H₅ |
| A-725 | 2-(Trifluoromethylthio)-6-chlorophenyl | SC₂H₅ |
| A-726 | 3-(Trifluoromethylthio)-4-chlorophenyl | SC₂H₅ |
| A-727 | 3-(Trifluoromethylthio)-5-chlorophenyl | SC₂H₅ |
| A-728 | 2-Chloro-3-(trifluoromethylthio)phenyl | SC₂H₅ |
| A-729 | 2-Chloro-4-(trifluoromethylthio)phenyl | SC₂H₅ |
| A-730 | 2-Chloro-5-(trifluoromethylthio)phenyl | SC₂H₅ |
| A-731 | 3-Chloro-4-(trifluoromethylthio)phenyl | SC₂H₅ |
| A-732 | 2-(Trifluoromethylthio)-3-fluorophenyl | SC₂H₅ |
| A-733 | 2-(Trifluoromethylthio)-4-fluorophenyl | SC₂H₅ |
| A-734 | 2-(Trifluoromethylthio)-5-fluorophenyl | SC₂H₅ |
| A-735 | 2-(Trifluoromethylthio)-6-fluorophenyl | SC₂H₅ |
| A-736 | 3-(Trifluoromethylthio)-4-fluorophenyl | SC₂H₅ |
| A-737 | 3-(Trifluoromethylthio)-5-fluorophenyl | SC₂H₅ |
| A-738 | 2-Fluoro-3-(trifluoromethylthio)phenyl | SC₂H₅ |
| A-739 | 2-Fluoro-4-(trifluoromethylthio)phenyl | SC₂H₅ |
| A-740 | 2-Fluoro-5-(trifluoromethylthio)phenyl | SC₂H₅ |
| A-741 | 3-Fluoro-4-(trifluoromethylthio)phenyl | SC₂H₅ |
| A-742 | 2,3,4-Trichlorophenyl | SC₂H₅ |
| A-743 | 2,3,5-Trichlorophenyl | SC₂H₅ |
| A-744 | 2,3,6-Trichlorophenyl | SC₂H₅ |
| A-745 | 2,4,5-Trichlorophenyl | SC₂H₅ |
| A-746 | 2,4,6-Trichlorophenyl | SC₂H₅ |
| A-747 | 3,4,5-Trichlorophenyl | SC₂H₅ |
| A-748 | 2,3,4-Trifluorophenyl | SC₂H₅ |
| A-749 | 2,3,5-Trifluorophenyl | SC₂H₅ |
| A-750 | 2,3,6-Trifluorophenyl | SC₂H₅ |
| A-751 | 2,4,5-Trifluorophenyl | SC₂H₅ |
| A-752 | 2,4,6-Trifluorophenyl | SC₂H₅ |
| A-753 | 3,4,5-Trifluorophenyl | SC₂H₅ |
| A-754 | 2,3,4-Trimethylphenyl | SC₂H₅ |
| A-755 | 2,3,5-Trimethylphenyl | SC₂H₅ |
| A-756 | 2,3,6-Trimethylphenyl | SC₂H₅ |
| A-757 | 2,4,5-Trimethylphenyl | SC₂H₅ |
| A-758 | 2,4,6-Trimethylphenyl | SC₂H₅ |
| A-759 | 3,4,5-Trimethylphenyl | SC₂H₅ |
| A-760 | 2,3,4-Trimethoxyphenyl | SC₂H₅ |
| A-761 | 2,3,5-Trimethoxyphenyl | SC₂H₅ |
| A-762 | 2,3,6-Trimethoxyphenyl | SC₂H₅ |
| A-763 | 2,4,5-Trimethoxyphenyl | SC₂H₅ |
| A-764 | 2,4,6-Trimethoxyphenyl | SC₂H₅ |
| A-765 | 3,4,5-Trimethoxyphenyl | SC₂H₅ |
| A-766 | Phenyl | SNa |
| A-767 | 2-Chlorophenyl | SNa |
| A-768 | 3-Chlorophenyl | SNa |
| A-769 | 4-Chlorophenyl | SNa |
| A-770 | 2-Fluorophenyl | SNa |
| A-771 | 3-Fluorophenyl | SNa |
| A-772 | 4-Fluorophenyl | SNa |
| A-773 | 2-Methylphenyl | SNa |
| A-774 | 3-Methylphenyl | SNa |
| A-775 | 4-Methylphenyl | SNa |
| A-776 | 2-Ethylphenyl | SNa |
| A-777 | 3-Ethylphenyl | SNa |
| A-778 | 4-Ethylphenyl | SNa |
| A-779 | 2-Methoxyphenyl | SNa |
| A-780 | 3-Methoxyphenyl | SNa |
| A-781 | 4-Methoxyphenyl | SNa |
| A-782 | 2-Trifluoromethylphenyl | SNa |
| A-783 | 3-Trifluoromethylphenyl | SNa |
| A-784 | 4-Trifluoromethylphenyl | SNa |
| A-785 | 2-Trifluoromethoxyphenyl | SNa |
| A-786 | 3-Trifluoromethoxyphenyl | SNa |
| A-787 | 4-Trifluoromethoxyphenyl | SNa |
| A-788 | 2-Difluoromethoxyphenyl | SNa |
| A-789 | 3-Difluoromethoxyphenyl | SNa |
| A-790 | 4-Difluoromethoxyphenyl | SNa |
| A-791 | 2-Trifluoromethylthiophenyl | SNa |
| A-792 | 3-Trifluoromethylthiophenyl | SNa |
| A-793 | 4-Trifluoromethylthiophenyl | SNa |
| A-794 | 2,3-Dichlorophenyl | SNa |
| A-795 | 2,4-Dichlorophenyl | SNa |
| A-796 | 2,5-Dichlorophenyl | SNa |
| A-797 | 2,6-Dichlorophenyl | SNa |
| A-798 | 3,4-Dichlorophenyl | SNa |
| A-799 | 3,5-Dichlorophenyl | SNa |
| A-800 | 2,3-Difluorophenyl | SNa |
| A-801 | 2,4-Difluorophenyl | SNa |
| A-802 | 2,5-Difluorophenyl | SNa |
| A-803 | 2,6-Difluorophenyl | SNa |
| A-804 | 3,4-Difluorophenyl | SNa |
| A-805 | 3,5-Difluorophenyl | SNa |
| A-806 | 2,3-Dimethylphenyl | SNa |
| A-807 | 2,4-Dimethylphenyl | SNa |
| A-808 | 2,5-Dimethylphenyl | SNa |
| A-809 | 2,6-Dimethylphenyl | SNa |
| A-810 | 3,4-Dimethylphenyl | SNa |
| A-811 | 3,5-Dimethylphenyl | SNa |
| A-812 | 2,3-Diethylphenyl | SNa |
| A-813 | 2,4-Diethylphenyl | SNa |
| A-814 | 2,5-Diethylphenyl | SNa |
| A-815 | 2,6-Diethylphenyl | SNa |
| A-816 | 3,5-Diethylphenyl | SNa |
| A-817 | 3,4-Diethylphenyl | SNa |
| A-818 | 2,3-Dimethoxyphenyl | SNa |
| A-819 | 2,4-Dimethoxyphenyl | SNa |
| A-820 | 2,5-Dimethoxyphenyl | SNa |
| A-821 | 2,6-Dimethoxyphenyl | SNa |
| A-822 | 3,4-Dimethoxyphenyl | SNa |
| A-823 | 3,5-Dimethoxyphenyl | SNa |
| A-824 | 2,3-Di(trifluoromethyl)phenyl | SNa |
| A-825 | 2,4-Di(trifluoromethyl)phenyl | SNa |
| A-826 | 2,5-Di(trifluoromethyl)phenyl | SNa |
| A-827 | 2,6-Di(trifluoromethyl)phenyl | SNa |
| A-828 | 3,4-Di(trifluoromethyl)phenyl | SNa |
| A-829 | 3,5-Di(trifluoromethyl)phenyl | SNa |
| A-830 | 2,3-Di(trifluoromethoxy)phenyl | SNa |
| A-831 | 2,4-Di(trifluoromethoxy)phenyl | SNa |
| A-832 | 2,5-Di(trifluoromethoxy)phenyl | SNa |
| A-833 | 2,6-Di(trifluoromethoxy)phenyl | SNa |
| A-834 | 3,4-Di(trifluoromethoxy)phenyl | SNa |
| A-835 | 3,5-Di(trifluoromethoxy)phenyl | SNa |
| A-836 | 2,3-Di(difluoromethoxy)phenyl | SNa |
| A-837 | 2,4-Di(difluoromethoxy)phenyl | SNa |
| A-838 | 2,5-Di(difluoromethoxy)phenyl | SNa |
| A-839 | 2,6-Di(difluoromethoxy)phenyl | SNa |
| A-840 | 3,4-Di(difluoromethoxy)phenyl | SNa |
| A-841 | 3,5-Di(difluoromethoxy)phenyl | SNa |
| A-842 | 2,3-Di(trifluoromethylthio)phenyl | SNa |
| A-843 | 2,4-Di(trifluoromethylthio)phenyl | SNa |
| A-844 | 2,5-Di(trifluoromethylthio)phenyl | SNa |
| A-845 | 2,6-Di(trifluoromethylthio)phenyl | SNa |
| A-846 | 3,4-Di(trifluoromethylthio)phenyl | SNa |
| A-847 | 3,5-Di(trifluoromethylthio)phenyl | SNa |
| A-848 | 2-Fluoro-3-chlorophenyl | SNa |
| A-849 | 2-Fluoro-4-chlorophenyl | SNa |
| A-850 | 2-Fluoro-5-chlorophenyl | SNa |
| A-851 | 2-Fluoro-6-chlorophenyl | SNa |
| A-852 | 3-Fluoro-4-chlorophenyl | SNa |
| A-853 | 3-Fluoro-5-chlorophenyl | SNa |
| A-854 | 2-Chloro-3-fluorophenyl | SNa |
| A-855 | 2-Chloro-4-fluorophenyl | SNa |
| A-856 | 2-Chloro-5-fluorophenyl | SNa |
| A-857 | 3-Chloro-4-fluorophenyl | SNa |
| A-858 | 2-Methyl-3-chlorophenyl | SNa |
| A-859 | 2-Methyl-4-chlorophenyl | SNa |
| A-860 | 2-Methyl-5-chlorophenyl | SNa |
| A-861 | 2-Methyl-6-chlorophenyl | SNa |
| A-862 | 3-Methyl-4-chlorophenyl | SNa |
| A-863 | 3-Methyl-5-chlorophenyl | SNa |
| A-864 | 2-Chloro-3-methylphenyl | SNa |
| A-865 | 2-Chloro-4-methylphenyl | SNa |
| A-866 | 2-Chloro-5-methylphenyl | SNa |
| A-867 | 3-Chloro-4-methylphenyl | SNa |
| A-868 | 2-Methyl-3-fluorophenyl | SNa |
| A-869 | 2-Methyl-4-fluorophenyl | SNa |
| A-870 | 2-Methyl-5-fluorophenyl | SNa |
| A-871 | 2-Methyl-6-fluorophenyl | SNa |
| A-872 | 3-Methyl-4-fluorophenyl | SNa |
| A-873 | 3-Methyl-5-fluorophenyl | SNa |
| A-874 | 2-Fluoro-3-methylphenyl | SNa |
| A-875 | 2-Fluoro-4-methylphenyl | SNa |
| A-876 | 2-Fluoro-5-methylphenyl | SNa |
| A-877 | 3-Fluoro-4-methylphenyl | SNa |
| A-878 | 2-Chloro-3-ethylphenyl | SNa |
| A-879 | 2-Chloro-4-ethylphenyl | SNa |
| A-880 | 2-Chloro-5-ethylphenyl | SNa |
| A-881 | 3-Chloro-4-ethylphenyl | SNa |
| A-882 | 2-Ethyl-3-chlorophenyl | SNa |
| A-883 | 2-Ethyl-4-chlorophenyl | SNa |
| A-884 | 2-Ethyl-5-chlorophenyl | SNa |
| A-885 | 2-Ethyl-6-chlorophenyl | SNa |
| A-886 | 2-Ethyl-3-fluorophenyl | SNa |
| A-887 | 2-Ethyl-4-fluorophenyl | SNa |
| A-888 | 2-Ethyl-5-fluorophenyl | SNa |
| A-889 | 2-Ethyl-6-fluorophenyl | SNa |
| A-890 | 3-Ethyl-4-fluorophenyl | SNa |
| A-891 | 3-Ethyl-5-fluorophenyl | SNa |
| A-892 | 2-Fluoro-3-ethylphenyl | SNa |
| A-893 | 2-Fluoro-4-ethylphenyl | SNa |
| A-894 | 2-Fluoro-5-ethylphenyl | SNa |
| A-895 | 3-Fluoro-4-ethylphenyl | SNa |
| A-896 | 2-Methoxy-3-chlorophenyl | SNa |
| A-897 | 2-Methoxy-4-chlorophenyl | SNa |
| A-898 | 2-Methoxy-5-chlorophenyl | SNa |
| A-899 | 2-Methoxy-6-chlorophenyl | SNa |
| A-900 | 3-Methoxy-4-chlorophenyl | SNa |
| A-901 | 3-Methoxy-5-chlorophenyl | SNa |
| A-902 | 2-Chloro-3-methoxyphenyl | SNa |
| A-903 | 2-Chloro-4-methoxyphenyl | SNa |
| A-904 | 2-Chloro-5-methoxyphenyl | SNa |
| A-905 | 3-Chloro-4-methoxyphenyl | SNa |
| A-906 | 2-Methoxy-3-fluorophenyl | SNa |
| A-907 | 2-Methoxy-4-fluorophenyl | SNa |
| A-908 | 2-Methoxy-5-fluorophenyl | SNa |
| A-909 | 2-Methoxy-6-fluorophenyl | SNa |
| A-910 | 3-Methoxy-4-fluorophenyl | SNa |
| A-911 | 3-Methoxy-5-fluorophenyl | SNa |
| A-912 | 2-Fluoro-3-methoxyphenyl | SNa |
| A-913 | 2-Fluoro-4-methoxyphenyl | SNa |
| A-914 | 2-Fluoro-5-methoxyphenyl | SNa |
| A-915 | 3-Fluoro-4-methoxyphenyl | SNa |
| A-916 | 3-Fluoro-5-methoxyphenyl | SNa |
| A-917 | 2-(Trifluoromethyl)-3-chlorophenyl | SNa |
| A-918 | 2-(Trifluoromethyl)-4-chlorophenyl | SNa |
| A-919 | 2-(Trifluoromethyl)-5-chlorophenyl | SNa |
| A-920 | 2-(Trifluoromethyl)-6-chlorophenyl | SNa |
| A-921 | 3-(Trifluoromethyl)-4-chlorophenyl | SNa |
| A-922 | 3-(Trifluoromethyl)-5-chlorophenyl | SNa |
| A-923 | 2-Chloro-3-(trifluoromethyl)phenyl | SNa |
| A-924 | 2-Chloro-4-(trifluoromethyl)phenyl | SNa |
| A-925 | 2-Chloro-5-(trifluoromethyl)phenyl | SNa |
| A-926 | 3-Chloro-4-(trifluoromethyl)phenyl | SNa |
| A-927 | 2-(Trifluoromethyl)-3-fluorophenyl | SNa |
| A-928 | 2-(Trifluoromethyl)-4-fluorophenyl | SNa |
| A-929 | 2-(Trifluoromethyl)-5-fluorophenyl | SNa |
| A-930 | 2-(Trifluoromethyl)-6-fluorophenyl | SNa |
| A-931 | 3-(Trifluoromethyl)-4-fluorophenyl | SNa |
| A-932 | 3-(Trifluoromethyl)-5-fluorophenyl | SNa |
| A-933 | 2-Fluoro-3-(trifluoromethyl)phenyl | SNa |
| A-934 | 2-Fluoro-4-(trifluoromethyl)phenyl | SNa |
| A-935 | 2-Fluoro-5-(trifluoromethyl)phenyl | SNa |
| A-936 | 3-Fluoro-4-(trifluoromethyl)phenyl | SNa |
| A-937 | 2-(Trifluoromethoxy)-3-chlorophenyl | SNa |
| A-938 | 2-(Trifluoromethoxy)-4-chlorophenyl | SNa |
| A-939 | 2-(Trifluoromethoxy)-5-chlorophenyl | SNa |
| A-940 | 2-(Trifluoromethoxy)-6-chlorophenyl | SNa |
| A-941 | 3-(Trifluoromethoxy)-4-chlorophenyl | SNa |
| A-942 | 3-(Trifluoromethoxy)-5-chlorophenyl | SNa |
| A-943 | 2-Chloro-3-(trifluoromethoxy)phenyl | SNa |
| A-944 | 2-Chloro-4-(trifluoromethoxy)phenyl | SNa |
| A-945 | 2-Chloro-5-(trifluoromethoxy)phenyl | SNa |
| A-946 | 3-Chloro-4-(trifluoromethoxy)phenyl | SNa |
| A-947 | 2-(Trifluoromethoxy)-3-fluorophenyl | SNa |
| A-948 | 2-(Trifluoromethoxy)-4-fluorophenyl | SNa |
| A-949 | 2-(Trifluoromethoxy)-5-fluorophenyl | SNa |
| A-950 | 2-(Trifluoromethoxy)-6-fluorophenyl | SNa |
| A-951 | 3-(Trifluoromethoxy)-4-fluorophenyl | SNa |
| A-952 | 3-(Trifluoromethoxy)-5-fluorophenyl | SNa |
| A-953 | 2-Fluoro-3-(trifluoromethoxy)phenyl | SNa |
| A-954 | 2-Fluoro-4-(trifluoromethoxy)phenyl | SNa |
| A-955 | 2-Fluoro-5-(trifluoromethoxy)phenyl | SNa |
| A-956 | 3-Fluoro-4-(trifluoromethoxy)phenyl | SNa |
| A-957 | 2-(Difluoromethoxy)-3-chlorophenyl | SNa |
| A-958 | 2-(Difluoromethoxy)-4-chlorophenyl | SNa |
| A-959 | 2-(Difluoromethoxy)-5-chlorophenyl | SNa |
| A-960 | 2-(Difluoromethoxy)-6-chlorophenyl | SNa |
| A-961 | 3-(Difluoromethoxy)-4-chlorophenyl | SNa |
| A-962 | 3-(Difluoromethoxy)-5-chlorophenyl | SNa |
| A-963 | 2-Chloro-3-(difluoromethoxy)phenyl | SNa |
| A-964 | 2-Chloro-4-(difluoromethoxy)phenyl | SNa |
| A-965 | 2-Chloro-5-(difluoromethoxy)phenyl | SNa |
| A-966 | 3-Chloro-4-(difluoromethoxy)phenyl | SNa |
| A-967 | 2-(Difluoromethoxy)-3-fluorophenyl | SNa |
| A-968 | 2-(Difluoromethoxy)-4-fluorophenyl | SNa |
| A-969 | 2-(Difluoromethoxy)-5-fluorophenyl | SNa |
| A-970 | 2-(Difluoromethoxy)-6-fluorophenyl | SNa |
| A-971 | 3-(Difluoromethoxy)-4-fluorophenyl | SNa |
| A-972 | 3-(Difluoromethoxy)-5-fluorophenyl | SNa |
| A-973 | 2-Fluoro-3-(difluoromethoxy)phenyl | SNa |
| A-974 | 2-Fluoro-4-(difluoromethoxy)phenyl | SNa |
| A-975 | 2-Fluoro-5-(difluoromethoxy)phenyl | SNa |
| A-976 | 3-Fluoro-4-(difluoromethoxy)phenyl | SNa |
| A-977 | 2-(Trifluoromethylthio)-3-chlorophenyl | SNa |
| A-978 | 2-(Trifluoromethylthio)-4-chlorophenyl | SNa |
| A-979 | 2-(Trifluoromethylthio)-5-chlorophenyl | SNa |
| A-980 | 2-(Trifluoromethylthio)-6-chlorophenyl | SNa |
| A-981 | 3-(Trifluoromethylthio)-4-chlorophenyl | SNa |
| A-982 | 3-(Trifluoromethylthio)-5-chlorophenyl | SNa |
| A-983 | 2-Chloro-3-(trifluoromethylthio)phenyl | SNa |
| A-984 | 2-Chloro-4-(trifluoromethylthio)phenyl | SNa |
| A-985 | 2-Chloro-5-(trifluoromethylthio)phenyl | SNa |
| A-986 | 3-Chloro-4-(trifluoromethylthio)phenyl | SNa |
| A-987 | 2-(Trifluoromethylthio)-3-fluorophenyl | SNa |
| A-988 | 2-(Trifluoromethylthio)-4-fluorophenyl | SNa |
| A-989 | 2-(Trifluoromethylthio)-5-fluorophenyl | SNa |
| A-990 | 2-(Trifluoromethylthio)-6-fluorophenyl | SNa |
| A-991 | 3-(Trifluoromethylthio)-4-fluorophenyl | SNa |
| A-992 | 3-(Trifluoromethylthio)-5-fluorophenyl | SNa |
| A-993 | 2-Fluoro-3-(trifluoromethylthio)phenyl | SNa |
| A-994 | 2-Fluoro-4-(trifluoromethylthio)phenyl | SNa |
| A-995 | 2-Fluoro-5-(trifluoromethylthio)phenyl | SNa |
| A-996 | 3-Fluoro-4-(trifluoromethylthio)phenyl | SNa |
| A-997 | 2,3,4-Trichlorophenyl | SNa |
| A-998 | 2,3,5-Trichlorophenyl | SNa |
| A-999 | 2,3,6-Trichlorophenyl | SNa |
| A-1000 | 2,4,5-Trichlorophenyl | SNa |
| A-1001 | 2,4,6-Trichlorophenyl | SNa |
| A-1002 | 3,4,5-Trichlorophenyl | SNa |
| A-1003 | 2,3,4-Trifluorophenyl | SNa |
| A-1004 | 2,3,5-Trifluorophenyl | SNa |
| A-1005 | 2,3,6-Trifluorophenyl | SNa |
| A-1006 | 2,4,5-Trifluorophenyl | SNa |
| A-1007 | 2,4,6-Trifluorophenyl | SNa |
| A-1008 | 3,4,5-Trifluorophenyl | SNa |
| A-1009 | 2,3,4-Trimethylphenyl | SNa |
| A-1010 | 2,3,5-Trimethylphenyl | SNa |
| A-1011 | 2,3,6-Trimethylphenyl | SNa |
| A-1012 | 2,4,5-Trimethylphenyl | SNa |
| A-1013 | 2,4,6-Trimethylphenyl | SNa |
| A-1014 | 3,4,5-Trimethylphenyl | SNa |
| A-1015 | 2,3,4-Trimethoxyphenyl | SNa |
| A-1016 | 2,3,5-Trimethoxyphenyl | SNa |
| A-1017 | 2,3,6-Trimethoxyphenyl | SNa |
| A-1018 | 2,4,5-Trimethoxyphenyl | SNa |
| A-1019 | 2,4,6-Trimethoxyphenyl | SNa |
| A-1020 | 3,4,5-Trimethoxyphenyl | SNa |
| A-1021 | Phenyl | S(1/2Cu) |
| A-1022 | 2-Chlorophenyl | S(1/2Cu) |
| A-1023 | 3-Chlorophenyl | S(1/2Cu) |
| A-1024 | 4-Chlorophenyl | S(1/2Cu) |
| A-1025 | 2-Fluorophenyl | S(1/2Cu) |
| A-1026 | 3-Fluorophenyl | S(1/2Cu) |
| A-1027 | 4-Fluorophenyl | S(1/2Cu) |
| A-1028 | 2-Methylphenyl | S(1/2Cu) |
| A-1029 | 3-Methylphenyl | S(1/2Cu) |
| A-1030 | 4-Methylphenyl | S(1/2Cu) |
| A-1031 | 2-Ethylphenyl | S(1/2Cu) |
| A-1032 | 3-Ethylphenyl | S(1/2Cu) |
| A-1033 | 4-Ethylphenyl | S(1/2Cu) |
| A-1034 | 2-Methoxyphenyl | S(1/2Cu) |
| A-1035 | 3-Methoxyphenyl | S(1/2Cu) |
| A-1036 | 4-Methoxyphenyl | S(1/2Cu) |
| A-1037 | 2-Trifluoromethylphenyl | S(1/2Cu) |
| A-1038 | 3-Trifluoromethylphenyl | S(1/2Cu) |
| A-1039 | 4-Trifluoromethylphenyl | S(1/2Cu) |
| A-1040 | 2-Trifluoromethoxyphenyl | S(1/2Cu) |
| A-1041 | 3-Trifluoromethoxyphenyl | S(1/2Cu) |
| A-1042 | 4-Trifluoromethoxyphenyl | S(1/2Cu) |
| A-1043 | 2-Difluoromethoxyphenyl | S(1/2Cu) |
| A-1044 | 3-Difluoromethoxyphenyl | S(1/2Cu) |
| A-1045 | 4-Difluoromethoxyphenyl | S(1/2Cu) |
| A-1046 | 2-Trifluoromethylthiophenyl | S(1/2Cu) |
| A-1047 | 3-Trifluoromethylthiophenyl | S(1/2Cu) |
| A-1048 | 4-Trifluoromethylthiophenyl | S(1/2Cu) |
| A-1049 | 2,3-Dichlorophenyl | S(1/2Cu) |
| A-1050 | 2,4-Dichlorophenyl | S(1/2Cu) |
| A-1051 | 2,5-Dichlorophenyl | S(1/2Cu) |
| A-1052 | 2,6-Dichlorophenyl | S(1/2Cu) |
| A-1053 | 3,4-Dichlorophenyl | S(1/2Cu) |
| A-1054 | 3,5-Dichlorophenyl | S(1/2Cu) |
| A-1055 | 2,3-Difluorophenyl | S(1/2Cu) |
| A-1056 | 2,4-Difluorophenyl | S(1/2Cu) |
| A-1057 | 2,5-Difluorophenyl | S(1/2Cu) |
| A-1058 | 2,6-Difluorophenyl | S(1/2Cu) |
| A-1059 | 3,4-Difluorophenyl | S(1/2Cu) |
| A-1060 | 3,5-Difluorophenyl | S(1/2Cu) |
| A-1061 | 2,3-Dimethylphenyl | S(1/2Cu) |
| A-1062 | 2,4-Dimethylphenyl | S(1/2Cu) |
| A-1063 | 2,5-Dimethylphenyl | S(1/2Cu) |
| A-1064 | 2,6-Dimethylphenyl | S(1/2Cu) |
| A-1065 | 3,4-Dimethylphenyl | S(1/2Cu) |
| A-1066 | 3,5-Dimethylphenyl | S(1/2Cu) |
| A-1067 | 2,3-Diethylphenyl | S(1/2Cu) |
| A-1068 | 2,4-Diethylphenyl | S(1/2Cu) |
| A-1069 | 2,5-Diethylphenyl | S(1/2Cu) |
| A-1070 | 2,6-Diethylphenyl | S(1/2Cu) |
| A-1071 | 3,5-Diethylphenyl | S(1/2Cu) |
| A-1072 | 3,4-Diethylphenyl | S(1/2Cu) |
| A-1073 | 2,3-Dimethoxyphenyl | S(1/2Cu) |
| A-1074 | 2,4-Dimethoxyphenyl | S(1/2Cu) |
| A-1075 | 2,5-Dimethoxyphenyl | S(1/2Cu) |
| A-1076 | 2,6-Dimethoxyphenyl | S(1/2Cu) |
| A-1077 | 3,4-Dimethoxyphenyl | S(1/2Cu) |
| A-1078 | 3,5-Dimethoxyphenyl | S(1/2Cu) |
| A-1079 | 2,3-Di(trifluoromethyl)phenyl | S(1/2Cu) |
| A-1080 | 2,4-Di(trifluoromethyl)phenyl | S(1/2Cu) |
| A-1081 | 2,5-Di(trifluoromethyl)phenyl | S(1/2Cu) |
| A-1082 | 2,6-Di(trifluoromethyl)phenyl | S(1/2Cu) |
| A-1083 | 3,4-Di(trifluoromethyl)phenyl | S(1/2Cu) |
| A-1084 | 3,5-Di(trifluoromethyl)phenyl | S(1/2Cu) |
| A-1085 | 2,3-Di(trifluoromethoxy)phenyl | S(1/2Cu) |
| A-1086 | 2,4-Di(trifluoromethoxy)phenyl | S(1/2Cu) |
| A-1087 | 2,5-Di(trifluoromethoxy)phenyl | S(1/2Cu) |
| A-1088 | 2,6-Di(trifluoromethoxy)phenyl | S(1/2Cu) |
| A-1089 | 3,4-Di(trifluoromethoxy)phenyl | S(1/2Cu) |
| A-1090 | 3,5-Di(trifluoromethoxy)phenyl | S(1/2Cu) |
| A-1091 | 2,3-Di(difluoromethoxy)phenyl | S(1/2Cu) |
| A-1092 | 2,4-Di(difluoromethoxy)phenyl | S(1/2Cu) |
| A-1093 | 2,5-Di(difluoromethoxy)phenyl | S(1/2Cu) |
| A-1094 | 2,6-Di(difluoromethoxy)phenyl | S(1/2Cu) |
| A-1095 | 3,4-Di(difluoromethoxy)phenyl | S(1/2Cu) |
| A-1096 | 3,5-Di(difluoromethoxy)phenyl | S(1/2Cu) |
| A-1097 | 2,3-Di(trifluoromethylthio)phenyl | S(1/2Cu) |
| A-1098 | 2,4-Di(trifluoromethylthio)phenyl | S(1/2Cu) |
| A-1099 | 2,5-Di(trifluoromethylthio)phenyl | S(1/2Cu) |
| A-1100 | 2,6-Di(trifluoromethylthio)phenyl | S(1/2Cu) |
| A-1101 | 3,4-Di(trifluoromethylthio)phenyl | S(1/2Cu) |
| A-1102 | 3,5-Di(trifluoromethylthio)phenyl | S(1/2Cu) |
| A-1103 | 2-Fluoro-3-chlorophenyl | S(1/2Cu) |
| A-1104 | 2-Fluoro-4-chlorophenyl | S(1/2Cu) |
| A-1105 | 2-Fluoro-5-chlorophenyl | S(1/2Cu) |
| A-1106 | 2-Fluoro-6-chlorophenyl | S(1/2Cu) |
| A-1107 | 3-Fluoro-4-chlorophenyl | S(1/2Cu) |
| A-1108 | 3-Fluoro-5-chlorophenyl | S(1/2Cu) |
| A-1109 | 2-Chloro-3-fluorophenyl | S(1/2Cu) |
| A-1110 | 2-Chloro-4-fluorophenyl | S(1/2Cu) |
| A-1111 | 2-Chloro-5-fluorophenyl | S(1/2Cu) |
| A-1112 | 3-Chloro-4-fluorophenyl | S(1/2Cu) |
| A-1113 | 2-Methyl-3-chlorophenyl | S(1/2Cu) |
| A-1114 | 2-Methyl-4-chlorophenyl | S(1/2Cu) |
| A-1115 | 2-Methyl-5-chlorophenyl | S(1/2Cu) |
| A-1116 | 2-Methyl-6-chlorophenyl | S(1/2Cu) |
| A-1117 | 3-Methyl-4-chlorophenyl | S(1/2Cu) |
| A-1118 | 3-Methyl-5-chlorophenyl | S(1/2Cu) |
| A-1119 | 2-Chloro-3-methylphenyl | S(1/2Cu) |
| A-1120 | 2-Chloro-4-methylphenyl | S(1/2Cu) |
| A-1121 | 2-Chloro-5-methylphenyl | S(1/2Cu) |
| A-1122 | 3-Chloro-4-methylphenyl | S(1/2Cu) |
| A-1123 | 2-Methyl-3-fluorophenyl | S(1/2Cu) |
| A-1124 | 2-Methyl-4-fluorophenyl | S(1/2Cu) |
| A-1125 | 2-Methyl-5-fluorophenyl | S(1/2Cu) |
| A-1126 | 2-Methyl-6-fluorophenyl | S(1/2Cu) |
| A-1127 | 3-Methyl-4-fluorophenyl | S(1/2Cu) |
| A-1128 | 3-Methyl-5-fluorophenyl | S(1/2Cu) |
| A-1129 | 2-Fluoro-3-methylphenyl | S(1/2Cu) |
| A-1130 | 2-Fluoro-4-methylphenyl | S(1/2Cu) |
| A-1131 | 2-Fluoro-5-methylphenyl | S(1/2Cu) |
| A-1132 | 3-Fluoro-4-methylphenyl | S(1/2Cu) |
| A-1133 | 2-Chloro-3-ethylphenyl | S(1/2Cu) |
| A-1134 | 2-Chloro-4-ethylphenyl | S(1/2Cu) |
| A-1135 | 2-Chloro-5-ethylphenyl | S(1/2Cu) |
| A-1136 | 3-Chloro-4-ethylphenyl | S(1/2Cu) |
| A-1137 | 2-Ethyl-3-chlorophenyl | S(1/2Cu) |
| A-1138 | 2-Ethyl-4-chlorophenyl | S(1/2Cu) |
| A-1139 | 2-Ethyl-5-chlorophenyl | S(1/2Cu) |
| A-1140 | 2-Ethyl-6-chlorophenyl | S(1/2Cu) |
| A-1141 | 2-Ethyl-3-fluorophenyl | S(1/2Cu) |
| A-1142 | 2-Ethyl-4-fluorophenyl | S(1/2Cu) |
| A-1143 | 2-Ethyl-5-fluorophenyl | S(1/2Cu) |
| A-1144 | 2-Ethyl-6-fluorophenyl | S(1/2Cu) |
| A-1145 | 3-Ethyl-4-fluorophenyl | S(1/2Cu) |
| A-1146 | 3-Ethyl-5-fluorophenyl | S(1/2Cu) |
| A-1147 | 2-Fluoro-3-ethylphenyl | S(1/2Cu) |
| A-1148 | 2-Fluoro-4-ethylphenyl | S(1/2Cu) |
| A-1149 | 2-Fluoro-5-ethylphenyl | S(1/2Cu) |
| A-1150 | 3-Fluoro-4-ethylphenyl | S(1/2Cu) |
| A-1151 | 2-Methoxy-3-chlorophenyl | S(1/2Cu) |
| A-1152 | 2-Methoxy-4-chlorophenyl | S(1/2Cu) |
| A-1153 | 2-Methoxy-5-chlorophenyl | S(1/2Cu) |
| A-1154 | 2-Methoxy-6-chlorophenyl | S(1/2Cu) |
| A-1155 | 3-Methoxy-4-chlorophenyl | S(1/2Cu) |
| A-1156 | 3-Methoxy-5-chlorophenyl | S(1/2Cu) |
| A-1157 | 2-Chloro-3-methoxyphenyl | S(1/2Cu) |
| A-1158 | 2-Chloro-4-methoxyphenyl | S(1/2Cu) |
| A-1159 | 2-Chloro-5-methoxyphenyl | S(1/2Cu) |
| A-1160 | 3-Chloro-4-methoxyphenyl | S(1/2Cu) |
| A-1161 | 2-Methoxy-3-fluorophenyl | S(1/2Cu) |
| A-1162 | 2-Methoxy-4-fluorophenyl | S(1/2Cu) |
| A-1163 | 2-Methoxy-5-fluorophenyl | S(1/2Cu) |
| A-1164 | 2-Methoxy-6-fluorophenyl | S(1/2Cu) |
| A-1165 | 3-Methoxy-4-fluorophenyl | S(1/2Cu) |
| A-1166 | 3-Methoxy-5-fluorophenyl | S(1/2Cu) |
| A-1167 | 2-Fluoro-3-methoxyphenyl | S(1/2Cu) |
| A-1168 | 2-Fluoro-4-methoxyphenyl | S(1/2Cu) |
| A-1169 | 2-Fluoro-5-methoxyphenyl | S(1/2Cu) |
| A-1170 | 3-Fluoro-4-methoxyphenyl | S(1/2Cu) |
| A-1171 | 3-Fluoro-5-methoxyphenyl | S(1/2Cu) |
| A-1172 | 2-(Trifluoromethyl)-3-chlorophenyl | S(1/2Cu) |
| A-1173 | 2-(Trifluoromethyl)-4-chlorophenyl | S(1/2Cu) |
| A-1174 | 2-(Trifluoromethyl)-5-chlorophenyl | S(1/2Cu) |
| A-1175 | 2-(Trifluoromethyl)-6-chlorophenyl | S(1/2Cu) |
| A-1176 | 3-(Trifluoromethyl)-4-chlorophenyl | S(1/2Cu) |
| A-1177 | 3-(Trifluoromethyl)-5-chlorophenyl | S(1/2Cu) |
| A-1178 | 2-Chloro-3-(trifluoromethyl)phenyl | S(1/2Cu) |
| A-1179 | 2-Chloro-4-(trifluoromethyl)phenyl | S(1/2Cu) |
| A-1180 | 2-Chloro-5-(trifluoromethyl)phenyl | S(1/2Cu) |
| A-1181 | 3-Chloro-4-(trifluoromethyl)phenyl | S(1/2Cu) |
| A-1182 | 2-(Trifluoromethyl)-3-fluorophenyl | S(1/2Cu) |
| A-1183 | 2-(Trifluoromethyl)-4-fluorophenyl | S(1/2Cu) |
| A-1184 | 2-(Trifluoromethyl)-5-fluorophenyl | S(1/2Cu) |
| A-1185 | 2-(Trifluoromethyl)-6-fluorophenyl | S(1/2Cu) |
| A-1186 | 3-(Trifluoromethyl)-4-fluorophenyl | S(1/2Cu) |
| A-1187 | 3-(Trifluoromethyl)-5-fluorophenyl | S(1/2Cu) |
| A-1188 | 2-Fluoro-3-(trifluoromethyl)phenyl | S(1/2Cu) |
| A-1189 | 2-Fluoro-4-(trifluoromethyl)phenyl | S(1/2Cu) |
| A-1190 | 2-Fluoro-5-(trifluoromethyl)phenyl | S(1/2Cu) |
| A-1191 | 3-Fluoro-4-(trifluoromethyl)phenyl | S(1/2Cu) |
| A-1192 | 2-(Trifluoromethoxy)-3-chlorophenyl | S(1/2Cu) |
| A-1193 | 2-(Trifluoromethoxy)-4-chlorophenyl | S(1/2Cu) |
| A-1194 | 2-(Trifluoromethoxy)-5-chlorophenyl | S(1/2Cu) |
| A-1195 | 2-(Trifluoromethoxy)-6-chlorophenyl | S(1/2Cu) |
| A-1196 | 3-(Trifluoromethoxy)-4-chlorophenyl | S(1/2Cu) |
| A-1197 | 3-(Trifluoromethoxy)-5-chlorophenyl | S(1/2Cu) |
| A-1198 | 2-Chloro-3-(trifluoromethoxy)phenyl | S(1/2Cu) |
| A-1199 | 2-Chloro-4-(trifluoromethoxy)phenyl | S(1/2Cu) |
| A-1200 | 2-Chloro-5-(trifluoromethoxy)phenyl | S(1/2Cu) |
| A-1201 | 3-Chloro-4-(trifluoromethoxy)phenyl | S(1/2Cu) |
| A-1202 | 2-(Trifluoromethoxy)-3-fluorophenyl | S(1/2Cu) |
| A-1203 | 2-(Trifluoromethoxy)-4-fluorophenyl | S(1/2Cu) |
| A-1204 | 2-(Trifluoromethoxy)-5-fluorophenyl | S(1/2Cu) |
| A-1205 | 2-(Trifluoromethoxy)-6-fluorophenyl | S(1/2Cu) |
| A-1206 | 3-(Trifluoromethoxy)-4-fluorophenyl | S(1/2Cu) |
| A-1207 | 3-(Trifluoromethoxy)-5-fluorophenyl | S(1/2Cu) |
| A-1208 | 2-Fluoro-3-(trifluoromethoxy)phenyl | S(1/2Cu) |
| A-1209 | 2-Fluoro-4-(trifluoromethoxy)phenyl | S(1/2Cu) |
| A-1210 | 2-Fluoro-5-(trifluoromethoxy)phenyl | S(1/2Cu) |
| A-1211 | 3-Fluoro-4-(trifluoromethoxy)phenyl | S(1/2Cu) |
| A-1212 | 2-(Difluoromethoxy)-3-chlorophenyl | S(1/2Cu) |
| A-1213 | 2-(Difluoromethoxy)-4-chlorophenyl | S(1/2Cu) |
| A-1214 | 2-(Difluoromethoxy)-5-chlorophenyl | S(1/2Cu) |
| A-1215 | 2-(Difluoromethoxy)-6-chlorophenyl | S(1/2Cu) |
| A-1216 | 3-(Difluoromethoxy)-4-chlorophenyl | S(1/2Cu) |
| A-1217 | 3-(Difluoromethoxy)-5-chlorophenyl | S(1/2Cu) |
| A-1218 | 2-Chloro-3-(difluoromethoxy)phenyl | S(1/2Cu) |
| A-1219 | 2-Chloro-4-(difluoromethoxy)phenyl | S(1/2Cu) |
| A-1220 | 2-Chloro-5-(difluoromethoxy)phenyl | S(1/2Cu) |
| A-1221 | 3-Chloro-4-(difluoromethoxy)phenyl | S(1/2Cu) |
| A-1222 | 2-(Difluoromethoxy)-3-fluorophenyl | S(1/2Cu) |
| A-1223 | 2-(Difluoromethoxy)-4-fluorophenyl | S(1/2Cu) |
| A-1224 | 2-(Difluoromethoxy)-5-fluorophenyl | S(1/2Cu) |
| A-1225 | 2-(Difluoromethoxy)-6-fluorophenyl | S(1/2Cu) |
| A-1226 | 3-(Difluoromethoxy)-4-fluorophenyl | S(1/2Cu) |
| A-1227 | 3-(Difluoromethoxy)-5-fluorophenyl | S(1/2Cu) |
| A-1228 | 2-Fluoro-3-(difluoromethoxy)phenyl | S(1/2Cu) |
| A-1229 | 2-Fluoro-4-(difluoromethoxy)phenyl | S(1/2Cu) |
| A-1230 | 2-Fluoro-5-(difluoromethoxy)phenyl | S(1/2Cu) |
| A-1231 | 3-Fluoro-4-(difluoromethoxy)phenyl | S(1/2Cu) |
| A-1232 | 2-(Trifluoromethylthio)-3-chlorophenyl | S(1/2Cu) |
| A-1233 | 2-(Trifluoromethylthio)-4-chlorophenyl | S(1/2Cu) |
| A-1234 | 2-(Trifluoromethylthio)-5-chlorophenyl | S(1/2Cu) |
| A-1235 | 2-(Trifluoromethylthio)-6-chlorophenyl | S(1/2Cu) |
| A-1236 | 3-(Trifluoromethylthio)-4-chlorophenyl | S(1/2Cu) |
| A-1237 | 3-(Trifluoromethylthio)-5-chlorophenyl | S(1/2Cu) |
| A-1238 | 2-Chloro-3-(trifluoromethylthio)phenyl | S(1/2Cu) |
| A-1239 | 2-Chloro-4-(trifluoromethylthio)phenyl | S(1/2Cu) |
| A-1240 | 2-Chloro-5-(trifluoromethylthio)phenyl | S(1/2Cu) |
| A-1241 | 3-Chloro-4-(trifluoromethylthio)phenyl | S(1/2Cu) |
| A-1242 | 2-(Trifluoromethylthio)-3-fluorophenyl | S(1/2Cu) |
| A-1243 | 2-(Trifluoromethylthio)-4-fluorophenyl | S(1/2Cu) |
| A-1244 | 2-(Trifluoromethylthio)-5-fluorophenyl | S(1/2Cu) |
| A-1245 | 2-(Trifluoromethylthio)-6-fluorophenyl | S(1/2Cu) |
| A-1246 | 3-(Trifluoromethylthio)-4-fluorophenyl | S(1/2Cu) |
| A-1247 | 3-(Trifluoromethylthio)-5-fluorophenyl | S(1/2Cu) |
| A-1248 | 2-Fluoro-3-(trifluoromethylthio)phenyl | S(1/2Cu) |
| A-1249 | 2-Fluoro-4-(trifluoromethylthio)phenyl | S(1/2Cu) |
| A-1250 | 2-Fluoro-5-(trifluoromethylthio)phenyl | S(1/2Cu) |
| A-1251 | 3-Fluoro-4-(trifluoromethylthio)phenyl | S(1/2Cu) |
| A-1252 | 2,3,4-Trichlorophenyl | S(1/2Cu) |
| A-1253 | 2,3,5-Trichlorophenyl | S(1/2Cu) |
| A-1254 | 2,3,6-Trichlorophenyl | S(1/2Cu) |
| A-1255 | 2,4,5-Trichlorophenyl | S(1/2Cu) |
| A-1256 | 2,4,6-Trichlorophenyl | S(1/2Cu) |
| A-1257 | 3,4,5-Trichlorophenyl | S(1/2Cu) |
| A-1258 | 2,3,4-Trifluorophenyl | S(1/2Cu) |
| A-1259 | 2,3,5-Trifluorophenyl | S(1/2Cu) |
| A-1260 | 2,3,6-Trifluorophenyl | S(1/2Cu) |
| A-1261 | 2,4,5-Trifluorophenyl | S(1/2Cu) |
| A-1262 | 2,4,6-Trifluorophenyl | S(1/2Cu) |
| A-1263 | 3,4,5-Trifluorophenyl | S(1/2Cu) |
| A-1264 | 2,3,4-Trimethylphenyl | S(1/2Cu) |
| A-1265 | 2,3,5-Trimethylphenyl | S(1/2Cu) |
| A-1266 | 2,3,6-Trimethylphenyl | S(1/2Cu) |
| A-1267 | 2,4,5-Trimethylphenyl | S(1/2Cu) |
| A-1268 | 2,4,6-Trimethylphenyl | S(1/2Cu) |
| A-1269 | 3,4,5-Trimethylphenyl | S(1/2Cu) |
| A-1270 | 2,3,4-Trimethoxyphenyl | S(1/2Cu) |
| A-1271 | 2,3,5-Trimethoxyphenyl | S(1/2Cu) |
| A-1272 | 2,3,6-Trimethoxyphenyl | S(1/2Cu) |
| A-1273 | 2,4,5-Trimethoxyphenyl | S(1/2Cu) |
| A-1274 | 2,4,6-Trimethoxyphenyl | S(1/2Cu) |
| A-1275 | 3,4,5-Trimethoxyphenyl | S(1/2Cu) |
| A-1276 | Phenyl | S(HNEt₃) |
| A-1277 | 2-Chlorophenyl | S(HNEt₃) |
| A-1278 | 3-Chlorophenyl | S(HNEt₃) |
| A-1279 | 4-Chlorophenyl | S(HNEt₃) |
| A-1280 | 2-Fluorophenyl | S(HNEt₃) |
| A-1281 | 3-Fluorophenyl | S(HNEt₃) |
| A-1282 | 4-Fluorophenyl | S(HNEt₃) |
| A-1283 | 2-Methylphenyl | S(HNEt₃) |
| A-1284 | 3-Methylphenyl | S(HNEt₃) |
| A-1285 | 4-Methylphenyl | S(HNEt₃) |
| A-1286 | 2-Ethylphenyl | S(HNEt₃) |
| A-1287 | 3-Ethylphenyl | S(HNEt₃) |
| A-1288 | 4-Ethylphenyl | S(HNEt₃) |
| A-1289 | 2-Methoxyphenyl | S(HNEt₃) |
| A-1290 | 3-Methoxyphenyl | S(HNEt₃) |
| A-1291 | 4-Methoxyphenyl | S(HNEt₃) |
| A-1292 | 2-Trifluoromethylphenyl | S(HNEt₃) |
| A-1293 | 3-Trifluoromethylphenyl | S(HNEt₃) |
| A-1294 | 4-Trifluoromethylphenyl | S(HNEt₃) |
| A-1295 | 2-Trifluoromethoxyphenyl | S(HNEt₃) |
| A-1296 | 3-Trifluoromethoxyphenyl | S(HNEt₃) |
| A-1297 | 4-Trifluoromethoxyphenyl | S(HNEt₃) |
| A-1298 | 2-Difluoromethoxyphenyl | S(HNEt₃) |
| A-1299 | 3-Difluoromethoxyphenyl | S(HNEt₃) |
| A-1300 | 4-Difluoromethoxyphenyl | S(HNEt₃) |
| A-1301 | 2-Trifluoromethylthiophenyl | S(HNEt₃) |
| A-1302 | 3-Trifluoromethylthiophenyl | S(HNEt₃) |
| A-1303 | 4-Trifluoromethylthiophenyl | S(HNEt₃) |
| A-1304 | 2,3-Dichlorophenyl | S(HNEt₃) |
| A-1305 | 2,4-Dichlorophenyl | S(HNEt₃) |
| A-1306 | 2,5-Dichlorophenyl | S(HNEt₃) |
| A-1307 | 2,6-Dichlorophenyl | S(HNEt₃) |
| A-1308 | 3,4-Dichlorophenyl | S(HNEt₃) |
| A-1309 | 3,5-Dichlorophenyl | S(HNEt₃) |
| A-1310 | 2,3-Difluorophenyl | S(HNEt₃) |
| A-1311 | 2,4-Difluorophenyl | S(HNEt₃) |
| A-1312 | 2,5-Difluorophenyl | S(HNEt₃) |
| A-1313 | 2,6-Difluorophenyl | S(HNEt₃) |
| A-1314 | 3,4-Difluorophenyl | S(HNEt₃) |
| A-1315 | 3,5-Difluorophenyl | S(HNEt₃) |
| A-1316 | 2,3-Dimethylphenyl | S(HNEt₃) |
| A-1317 | 2,4-Dimethylphenyl | S(HNEt₃) |
| A-1318 | 2,5-Dimethylphenyl | S(HNEt₃) |
| A-1319 | 2,6-Dimethylphenyl | S(HNEt₃) |
| A-1320 | 3,4-Dimethylphenyl | S(HNEt₃) |
| A-1321 | 3,5-Dimethylphenyl | S(HNEt₃) |
| A-1322 | 2,3-Diethylphenyl | S(HNEt₃) |
| A-1323 | 2,4-Diethylphenyl | S(HNEt₃) |
| A-1324 | 2,5-Diethylphenyl | S(HNEt₃) |
| A-1325 | 2,6-Diethylphenyl | S(HNEt₃) |
| A-1326 | 3,5-Diethylphenyl | S(HNEt₃) |
| A-1327 | 3,4-Diethylphenyl | S(HNEt₃) |
| A-1328 | 2,3-Dimethoxyphenyl | S(HNEt₃) |
| A-1329 | 2,4-Dimethoxyphenyl | S(HNEt₃) |
| A-1330 | 2,5-Dimethoxyphenyl | S(HNEt₃) |
| A-1331 | 2,6-Dimethoxyphenyl | S(HNEt₃) |
| A-1332 | 3,4-Dimethoxyphenyl | S(HNEt₃) |
| A-1333 | 3,5-Dimethoxyphenyl | S(HNEt₃) |
| A-1334 | 2,3-Di(trifluoromethyl)phenyl | S(HNEt₃) |
| A-1335 | 2,4-Di(trifluoromethyl)phenyl | S(HNEt₃) |
| A-1336 | 2,5-Di(trifluoromethyl)phenyl | S(HNEt₃) |
| A-1337 | 2,6-Di(trifluoromethyl)phenyl | S(HNEt₃) |
| A-1338 | 3,4-Di(trifluoromethyl)phenyl | S(HNEt₃) |
| A-1339 | 3,5-Di(trifluoromethyl)phenyl | S(HNEt₃) |
| A-1340 | 2,3-Di(trifluoromethoxy)phenyl | S(HNEt₃) |
| A-1341 | 2,4-Di(trifluoromethoxy)phenyl | S(HNEt₃) |
| A-1342 | 2,5-Di(trifluoromethoxy)phenyl | S(HNEt₃) |
| A-1343 | 2,6-Di(trifluoromethoxy)phenyl | S(HNEt₃) |
| A-1344 | 3,4-Di(trifluoromethoxy)phenyl | S(HNEt₃) |
| A-1345 | 3,5-Di(trifluoromethoxy)phenyl | S(HNEt₃) |
| A-1346 | 2,3-Di(difluoromethoxy)phenyl | S(HNEt₃) |
| A-1347 | 2,4-Di(difluoromethoxy)phenyl | S(HNEt₃) |
| A-1348 | 2,5-Di(difluoromethoxy)phenyl | S(HNEt₃) |
| A-1349 | 2,6-Di(difluoromethoxy)phenyl | S(HNEt₃) |
| A-1350 | 3,4-Di(difluoromethoxy)phenyl | S(HNEt₃) |
| A-1351 | 3,5-Di(difluoromethoxy)phenyl | S(HNEt₃) |
| A-1352 | 2,3-Di(trifluoromethylthio)phenyl | S(HNEt₃) |
| A-1353 | 2,4-Di(trifluoromethylthio)phenyl | S(HNEt₃) |
| A-1354 | 2,5-Di(trifluoromethylthio)phenyl | S(HNEt₃) |
| A-1355 | 2,6-Di(trifluoromethylthio)phenyl | S(HNEt₃) |
| A-1356 | 3,4-Di(trifluoromethylthio)phenyl | S(HNEt₃) |
| A-1357 | 3,5-Di(trifluoromethylthio)phenyl | S(HNEt₃) |
| A-1358 | 2-Fluoro-3-chlorophenyl | S(HNEt₃) |
| A-1359 | 2-Fluoro-4-chlorophenyl | S(HNEt₃) |
| A-1360 | 2-Fluoro-5-chlorophenyl | S(HNEt₃) |
| A-1361 | 2-Fluoro-6-chlorophenyl | S(HNEt₃) |
| A-1362 | 3-Fluoro-4-chlorophenyl | S(HNEt₃) |
| A-1363 | 3-Fluoro-5-chlorophenyl | S(HNEt₃) |
| A-1364 | 2-Chloro-3-fluorophenyl | S(HNEt₃) |
| A-1365 | 2-Chloro-4-fluorophenyl | S(HNEt₃) |
| A-1366 | 2-Chloro-5-fluorophenyl | S(HNEt₃) |
| A-1367 | 3-Chloro-4-fluorophenyl | S(HNEt₃) |
| A-1368 | 2-Methyl-3-chlorophenyl | S(HNEt₃) |
| A-1369 | 2-Methyl-4-chlorophenyl | S(HNEt₃) |
| A-1370 | 2-Methyl-5-chlorophenyl | S(HNEt₃) |
| A-1371 | 2-Methyl-6-chlorophenyl | S(HNEt₃) |
| A-1372 | 3-Methyl-4-chlorophenyl | S(HNEt₃) |
| A-1373 | 3-Methyl-5-chlorophenyl | S(HNEt₃) |
| A-1374 | 2-Chloro-3-methylphenyl | S(HNEt₃) |
| A-1375 | 2-Chloro-4-methylphenyl | S(HNEt₃) |
| A-1376 | 2-Chloro-5-methylphenyl | S(HNEt₃) |
| A-1377 | 3-Chloro-4-methylphenyl | S(HNEt₃) |
| A-1378 | 2-Methyl-3-fluorophenyl | S(HNEt₃) |
| A-1379 | 2-Methyl-4-fluorophenyl | S(HNEt₃) |
| A-1380 | 2-Methyl-5-fluorophenyl | S(HNEt₃) |
| A-1381 | 2-Methyl-6-fluorophenyl | S(HNEt₃) |
| A-1382 | 3-Methyl-4-fluorophenyl | S(HNEt₃) |
| A-1383 | 3-Methyl-5-fluorophenyl | S(HNEt₃) |
| A-1384 | 2-Fluoro-3-methylphenyl | S(HNEt₃) |
| A-1385 | 2-Fluoro-4-methylphenyl | S(HNEt₃) |
| A-1386 | 2-Fluoro-5-methylphenyl | S(HNEt₃) |
| A-1387 | 3-Fluoro-4-methylphenyl | S(HNEt₃) |
| A-1388 | 2-Chloro-3-ethylphenyl | S(HNEt₃) |
| A-1389 | 2-Chloro-4-ethylphenyl | S(HNEt₃) |
| A-1390 | 2-Chloro-5-ethylphenyl | S(HNEt₃) |
| A-1391 | 3-Chloro-4-ethylphenyl | S(HNEt₃) |
| A-1392 | 2-Ethyl-3-chlorophenyl | S(HNEt₃) |
| A-1393 | 2-Ethyl-4-chlorophenyl | S(HNEt₃) |
| A-1394 | 2-Ethyl-5-chlorophenyl | S(HNEt₃) |
| A-1395 | 2-Ethyl-6-chlorophenyl | S(HNEt₃) |
| A-1396 | 2-Ethyl-3-fluorophenyl | S(HNEt₃) |
| A-1397 | 2-Ethyl-4-fluorophenyl | S(HNEt₃) |
| A-1398 | 2-Ethyl-5-fluorophenyl | S(HNEt₃) |
| A-1399 | 2-Ethyl-6-fluorophenyl | S(HNEt₃) |
| A-1400 | 3-Ethyl-4-fluorophenyl | S(HNEt₃) |
| A-1401 | 3-Ethyl-5-fluorophenyl | S(HNEt₃) |
| A-1402 | 2-Fluoro-3-ethylphenyl | S(HNEt₃) |
| A-1403 | 2-Fluoro-4-ethylphenyl | S(HNEt₃) |
| A-1404 | 2-Fluoro-5-ethylphenyl | S(HNEt₃) |
| A-1405 | 3-Fluoro-4-ethylphenyl | S(HNEt₃) |
| A-1406 | 2-Methoxy-3-chlorophenyl | S(HNEt₃) |
| A-1407 | 2-Methoxy-4-chlorophenyl | S(HNEt₃) |
| A-1408 | 2-Methoxy-5-chlorophenyl | S(HNEt₃) |
| A-1409 | 2-Methoxy-6-chlorophenyl | S(HNEt₃) |
| A-1410 | 3-Methoxy-4-chlorophenyl | S(HNEt₃) |
| A-1411 | 3-Methoxy-5-chlorophenyl | S(HNEt₃) |
| A-1412 | 2-Chloro-3-methoxyphenyl | S(HNEt₃) |
| A-1413 | 2-Chloro-4-methoxyphenyl | S(HNEt₃) |
| A-1414 | 2-Chloro-5-methoxyphenyl | S(HNEt₃) |
| A-1415 | 3-Chloro-4-methoxyphenyl | S(HNEt₃) |
| A-1416 | 2-Methoxy-3-fluorophenyl | S(HNEt₃) |
| A-1417 | 2-Methoxy-4-fluorophenyl | S(HNEt₃) |
| A-1418 | 2-Methoxy-5-fluorophenyl | S(HNEt₃) |
| A-1419 | 2-Methoxy-6-fluorophenyl | S(HNEt₃) |
| A-1420 | 3-Methoxy-4-fluorophenyl | S(HNEt₃) |
| A-1421 | 3-Methoxy-5-fluorophenyl | S(HNEt₃) |
| A-1422 | 2-Fluoro-3-methoxyphenyl | S(HNEt₃) |
| A-1423 | 2-Fluoro-4-methoxyphenyl | S(HNEt₃) |
| A-1424 | 2-Fluoro-5-methoxyphenyl | S(HNEt₃) |
| A-1425 | 3-Fluoro-4-methoxyphenyl | S(HNEt₃) |
| A-1426 | 3-Fluoro-5-methoxyphenyl | S(HNEt₃) |
| A-1427 | 2-(Trifluoromethyl)-3-chlorophenyl | S(HNEt₃) |
| A-1428 | 2-(Trifluoromethyl)-4-chlorophenyl | S(HNEt₃) |
| A-1429 | 2-(Trifluoromethyl)-5-chlorophenyl | S(HNEt₃) |
| A-1430 | 2-(Trifluoromethyl)-6-chlorophenyl | S(HNEt₃) |
| A-1431 | 3-(Trifluoromethyl)-4-chlorophenyl | S(HNEt₃) |
| A-1432 | 3-(Trifluoromethyl)-5-chlorophenyl | S(HNEt₃) |
| A-1433 | 2-Chloro-3-(trifluoromethyl)phenyl | S(HNEt₃) |
| A-1434 | 2-Chloro-4-(trifluoromethyl)phenyl | S(HNEt₃) |
| A-1435 | 2-Chloro-5-(trifluoromethyl)phenyl | S(HNEt₃) |
| A-1436 | 3-Chloro-4-(trifluoromethyl)phenyl | S(HNEt₃) |
| A-1437 | 2-(Trifluoromethyl)-3-fluorophenyl | S(HNEt₃) |
| A-1438 | 2-(Trifluoromethyl)-4-fluorophenyl | S(HNEt₃) |
| A-1439 | 2-(Trifluoromethyl)-5-fluorophenyl | S(HNEt₃) |
| A-1440 | 2-(Trifluoromethyl)-6-fluorophenyl | S(HNEt₃) |
| A-1441 | 3-(Trifluoromethyl)-4-fluorophenyl | S(HNEt₃) |
| A-1442 | 3-(Trifluoromethyl)-5-fluorophenyl | S(HNEt₃) |
| A-1443 | 2-Fluoro-3-(trifluoromethyl)phenyl | S(HNEt₃) |
| A-1444 | 2-Fluoro-4-(trifluoromethyl)phenyl | S(HNEt₃) |
| A-1445 | 2-Fluoro-5-(trifluoromethyl)phenyl | S(HNEt₃) |
| A-1446 | 3-Fluoro-4-(trifluoromethyl)phenyl | S(HNEt₃) |
| A-1447 | 2-(Trifluoromethoxy)-3-chlorophenyl | S(HNEt₃) |
| A-1448 | 2-(Trifluoromethoxy)-4-chlorophenyl | S(HNEt₃) |
| A-1449 | 2-(Trifluoromethoxy)-5-chlorophenyl | S(HNEt₃) |
| A-1450 | 2-(Trifluoromethoxy)-6-chlorophenyl | S(HNEt₃) |
| A-1451 | 3-(Trifluoromethoxy)-4-chlorophenyl | S(HNEt₃) |
| A-1452 | 3-(Trifluoromethoxy)-5-chlorophenyl | S(HNEt₃) |
| A-1453 | 2-Chloro-3-(trifluoromethoxy)phenyl | S(HNEt₃) |
| A-1454 | 2-Chloro-4-(trifluoromethoxy)phenyl | S(HNEt₃) |
| A-1455 | 2-Chloro-5-(trifluoromethoxy)phenyl | S(HNEt₃) |
| A-1456 | 3-Chloro-4-(trifluoromethoxy)phenyl | S(HNEt₃) |
| A-1457 | 2-(Trifluoromethoxy)-3-fluorophenyl | S(HNEt₃) |
| A-1458 | 2-(Trifluoromethoxy)-4-fluorophenyl | S(HNEt₃) |
| A-1459 | 2-(Trifluoromethoxy)-5-fluorophenyl | S(HNEt₃) |
| A-1460 | 2-(Trifluoromethoxy)-6-fluorophenyl | S(HNEt₃) |
| A-1461 | 3-(Trifluoromethoxy)-4-fluorophenyl | S(HNEt₃) |
| A-1462 | 3-(Trifluoromethoxy)-5-fluorophenyl | S(HNEt₃) |
| A-1463 | 2-Fluoro-3-(trifluoromethoxy)phenyl | S(HNEt₃) |
| A-1464 | 2-Fluoro-4-(trifluoromethoxy)phenyl | S(HNEt₃) |
| A-1465 | 2-Fluoro-5-(trifluoromethoxy)phenyl | S(HNEt₃) |
| A-1466 | 3-Fluoro-4-(trifluoromethoxy)phenyl | S(HNEt₃) |
| A-1467 | 2-(Difluoromethoxy)-3-chlorophenyl | S(HNEt₃) |
| A-1468 | 2-(Difluoromethoxy)-4-chlorophenyl | S(HNEt₃) |
| A-1469 | 2-(Difluoromethoxy)-5-chlorophenyl | S(HNEt₃) |
| A-1470 | 2-(Difluoromethoxy)-6-chlorophenyl | S(HNEt₃) |
| A-1471 | 3-(Difluoromethoxy)-4-chlorophenyl | S(HNEt₃) |
| A-1472 | 3-(Difluoromethoxy)-5-chlorophenyl | S(HNEt₃) |
| A-1473 | 2-Chloro-3-(difluoromethoxy)phenyl | S(HNEt₃) |
| A-1474 | 2-Chloro-4-(difluoromethoxy)phenyl | S(HNEt₃) |
| A-1475 | 2-Chloro-5-(difluoromethoxy)phenyl | S(HNEt₃) |
| A-1476 | 3-Chloro-4-(difluoromethoxy)phenyl | S(HNEt₃) |
| A-1477 | 2-(Difluoromethoxy)-3-fluorophenyl | S(HNEt₃) |
| A-1478 | 2-(Difluoromethoxy)-4-fluorophenyl | S(HNEt₃) |
| A-1479 | 2-(Difluoromethoxy)-5-fluorophenyl | S(HNEt₃) |
| A-1480 | 2-(Difluoromethoxy)-6-fluorophenyl | S(HNEt₃) |
| A-1481 | 3-(Difluoromethoxy)-4-fluorophenyl | S(HNEt₃) |
| A-1482 | 3-(Difluoromethoxy)-5-fluorophenyl | S(HNEt₃) |
| A-1483 | 2-Fluoro-3-(difluoromethoxy)phenyl | S(HNEt₃) |
| A-1484 | 2-Fluoro-4-(difluoromethoxy)phenyl | S(HNEt₃) |
| A-1485 | 2-Fluoro-5-(difluoromethoxy)phenyl | S(HNEt₃) |
| A-1486 | 3-Fluoro-4-(difluoromethoxy)phenyl | S(HNEt₃) |
| A-1487 | 2-(Trifluoromethylthio)-3-chlorophenyl | S(HNEt₃) |
| A-1488 | 2-(Trifluoromethylthio)-4-chlorophenyl | S(HNEt₃) |
| A-1489 | 2-(Trifluoromethylthio)-5-chlorophenyl | S(HNEt₃) |
| A-1490 | 2-(Trifluoromethylthio)-6-chlorophenyl | S(HNEt₃) |
| A-1491 | 3-(Trifluoromethylthio)-4-chlorophenyl | S(HNEt₃) |
| A-1492 | 3-(Trifluoromethylthio)-5-chlorophenyl | S(HNEt₃) |
| A-1493 | 2-Chloro-3-(trifluoromethylthio)phenyl | S(HNEt₃) |
| A-1494 | 2-Chloro-4-(trifluoromethylthio)phenyl | S(HNEt₃) |
| A-1495 | 2-Chloro-5-(trifluoromethylthio)phenyl | S(HNEt₃) |
| A-1496 | 3-Chloro-4-(trifluoromethylthio)phenyl | S(HNEt₃) |
| A-1497 | 2-(Trifluoromethylthio)-3-fluorophenyl | S(HNEt₃) |
| A-1498 | 2-(Trifluoromethylthio)-4-fluorophenyl | S(HNEt₃) |
| A-1499 | 2-(Trifluoromethylthio)-5-fluorophenyl | S(HNEt₃) |
| A-1500 | 2-(Trifluoromethylthio)-6-fluorophenyl | S(HNEt₃) |
| A-1501 | 3-(Trifluoromethylthio)-4-fluorophenyl | S(HNEt₃) |
| A-1502 | 3-(Trifluoromethylthio)-5-fluorophenyl | S(HNEt₃) |
| A-1503 | 2-Fluoro-3-(trifluoromethylthio)phenyl | S(HNEt₃) |
| A-1504 | 2-Fluoro-4-(trifluoromethylthio)phenyl | S(HNEt₃) |
| A-1505 | 2-Fluoro-5-(trifluoromethylthio)phenyl | S(HNEt₃) |
| A-1506 | 3-Fluoro-4-(trifluoromethylthio)phenyl | S(HNEt₃) |
| A-1507 | 2,3,4-Trichlorophenyl | S(HNEt₃) |
| A-1508 | 2,3,5-Trichlorophenyl | S(HNEt₃) |
| A-1509 | 2,3,6-Trichlorophenyl | S(HNEt₃) |
| A-1510 | 2,4,5-Trichlorophenyl | S(HNEt₃) |
| A-1511 | 2,4,6-Trichlorophenyl | S(HNEt₃) |
| A-1512 | 3,4,5-Trichlorophenyl | S(HNEt₃) |
| A-1513 | 2,3,4-Trifluorophenyl | S(HNEt₃) |
| A-1514 | 2,3,5-Trifluorophenyl | S(HNEt₃) |
| A-1515 | 2,3,6-Trifluorophenyl | S(HNEt₃) |
| A-1516 | 2,4,5-Trifluorophenyl | S(HNEt₃) |
| A-1517 | 2,4,6-Trifluorophenyl | S(HNEt₃) |
| A-1518 | 3,4,5-Trifluorophenyl | S(HNEt₃) |
| A-1519 | 2,3,4-Trimethylphenyl | S(HNEt₃) |
| A-1520 | 2,3,5-Trimethylphenyl | S(HNEt₃) |
| A-1521 | 2,3,6-Trimethylphenyl | S(HNEt₃) |
| A-1522 | 2,4,5-Trimethylphenyl | S(HNEt₃) |
| A-1523 | 2,4,6-Trimethylphenyl | S(HNEt₃) |
| A-1524 | 3,4,5-Trimethylphenyl | S(HNEt₃) |
| A-1525 | 2,3,4-Trimethoxyphenyl | S(HNEt₃) |
| A-1526 | 2,3,5-Trimethoxyphenyl | S(HNEt₃) |
| A-1527 | 2,3,6-Trimethoxyphenyl | S(HNEt₃) |
| A-1528 | 2,4,5-Trimethoxyphenyl | S(HNEt₃) |
| A-1529 | 2,4,6-Trimethoxyphenyl | S(HNEt₃) |
| A-1530 | 3,4,5-Trimethoxyphenyl | S(HNEt₃) |
| A-1531 | Phenyl | SCN |
| A-1532 | 2-Chlorophenyl | SCN |
| A-1533 | 3-Chlorophenyl | SCN |
| A-1534 | 4-Chlorophenyl | SCN |
| A-1535 | 2-Fluorophenyl | SCN |
| A-1536 | 3-Fluorophenyl | SCN |
| A-1537 | 4-Fluorophenyl | SCN |
| A-1538 | 2-Methylphenyl | SCN |
| A-1539 | 3-Methylphenyl | SCN |
| A-1540 | 4-Methylphenyl | SCN |
| A-1541 | 2-Ethylphenyl | SCN |
| A-1542 | 3-Ethylphenyl | SCN |
| A-1543 | 4-Ethylphenyl | SCN |
| A-1544 | 2-Methoxyphenyl | SCN |
| A-1545 | 3-Methoxyphenyl | SCN |
| A-1546 | 4-Methoxyphenyl | SCN |
| A-1547 | 2-Trifluoromethylphenyl | SCN |
| A-1548 | 3-Trifluoromethylphenyl | SCN |
| A-1549 | 4-Trifluoromethylphenyl | SCN |
| A-1550 | 2-Trifluoromethoxyphenyl | SCN |
| A-1551 | 3-Trifluoromethoxyphenyl | SCN |
| A-1552 | 4-Trifluoromethoxyphenyl | SCN |
| A-1553 | 2-Difluoromethoxyphenyl | SCN |
| A-1554 | 3-Difluoromethoxyphenyl | SCN |
| A-1555 | 4-Difluoromethoxyphenyl | SCN |
| A-1556 | 2-Trifluoromethylthiophenyl | SCN |
| A-1557 | 3-Trifluoromethylthiophenyl | SCN |
| A-1558 | 4-Trifluoromethylthiophenyl | SCN |
| A-1559 | 2,3-Dichlorophenyl | SCN |
| A-1560 | 2,4-Dichlorophenyl | SCN |
| A-1561 | 2,5-Dichlorophenyl | SCN |
| A-1562 | 2,6-Dichlorophenyl | SCN |
| A-1563 | 3,4-Dichlorophenyl | SCN |
| A-1564 | 3,5-Dichlorophenyl | SCN |
| A-1565 | 2,3-Difluorophenyl | SCN |
| A-1566 | 2,4-Difluorophenyl | SCN |
| A-1567 | 2,5-Difluorophenyl | SCN |
| A-1568 | 2,6-Difluorophenyl | SCN |
| A-1569 | 3,4-Difluorophenyl | SCN |
| A-1570 | 3,5-Difluorophenyl | SCN |
| A-1571 | 2,3-Dimethylphenyl | SCN |
| A-1572 | 2,4-Dimethylphenyl | SCN |
| A-1573 | 2,5-Dimethylphenyl | SCN |
| A-1574 | 2,6-Dimethylphenyl | SCN |
| A-1575 | 3,4-Dimethylphenyl | SCN |
| A-1576 | 3,5-Dimethylphenyl | SCN |
| A-1577 | 2,3-Diethylphenyl | SCN |
| A-1578 | 2,4-Diethylphenyl | SCN |
| A-1579 | 2,5-Diethylphenyl | SCN |
| A-1580 | 2,6-Diethylphenyl | SCN |
| A-1581 | 3,5-Diethylphenyl | SCN |
| A-1582 | 3,4-Diethylphenyl | SCN |
| A-1583 | 2,3-Dimethoxyphenyl | SCN |
| A-1584 | 2,4-Dimethoxyphenyl | SCN |
| A-1585 | 2,5-Dimethoxyphenyl | SCN |
| A-1586 | 2,6-Dimethoxyphenyl | SCN |
| A-1587 | 3,4-Dimethoxyphenyl | SCN |
| A-1588 | 3,5-Dimethoxyphenyl | SCN |
| A-1589 | 2,3-Di(trifluoromethyl)phenyl | SCN |
| A-1590 | 2,4-Di(trifluoromethyl)phenyl | SCN |
| A-1591 | 2,5-Di(trifluoromethyl)phenyl | SCN |
| A-1592 | 2,6-Di(trifluoromethyl)phenyl | SCN |
| A-1593 | 3,4-Di(trifluoromethyl)phenyl | SCN |
| A-1594 | 3,5-Di(trifluoromethyl)phenyl | SCN |
| A-1595 | 2,3-Di(trifluoromethoxy)phenyl | SCN |
| A-1596 | 2,4-Di(trifluoromethoxy)phenyl | SCN |
| A-1597 | 2,5-Di(trifluoromethoxy)phenyl | SCN |
| A-1598 | 2,6-Di(trifluoromethoxy)phenyl | SCN |
| A-1599 | 3,4-Di(trifluoromethoxy)phenyl | SCN |
| A-1600 | 3,5-Di(trifluoromethoxy)phenyl | SCN |
| A-1601 | 2,3-Di(difluoromethoxy)phenyl | SCN |
| A-1602 | 2,4-Di(difluoromethoxy)phenyl | SCN |
| A-1603 | 2,5-Di(difluoromethoxy)phenyl | SCN |
| A-1604 | 2,6-Di(difluoromethoxy)phenyl | SCN |
| A-1605 | 3,4-Di(difluoromethoxy)phenyl | SCN |
| A-1606 | 3,5-Di(difluoromethoxy)phenyl | SCN |
| A-1607 | 2,3-Di(trifluoromethylthio)phenyl | SCN |
| A-1608 | 2,4-Di(trifluoromethylthio)phenyl | SCN |
| A-1609 | 2,5-Di(trifluoromethylthio)phenyl | SCN |
| A-1610 | 2,6-Di(trifluoromethylthio)phenyl | SCN |
| A-1611 | 3,4-Di(trifluoromethylthio)phenyl | SCN |
| A-1612 | 3,5-Di(trifluoromethylthio)phenyl | SCN |
| A-1613 | 2-Fluoro-3-chlorophenyl | SCN |
| A-1614 | 2-Fluoro-4-chlorophenyl | SCN |
| A-1615 | 2-Fluoro-5-chlorophenyl | SCN |
| A-1616 | 2-Fluoro-6-chlorophenyl | SCN |
| A-1617 | 3-Fluoro-4-chlorophenyl | SCN |
| A-1618 | 3-Fluoro-5-chlorophenyl | SCN |
| A-1619 | 2-Chloro-3-fluorophenyl | SCN |
| A-1620 | 2-Chloro-4-fluorophenyl | SCN |
| A-1621 | 2-Chloro-5-fluorophenyl | SCN |
| A-1622 | 3-Chloro-4-fluorophenyl | SCN |
| A-1623 | 2-Methyl-3-chlorophenyl | SCN |
| A-1624 | 2-Methyl-4-chlorophenyl | SCN |
| A-1625 | 2-Methyl-5-chlorophenyl | SCN |
| A-1626 | 2-Methyl-6-chlorophenyl | SCN |
| A-1627 | 3-Methyl-4-chlorophenyl | SCN |
| A-1628 | 3-Methyl-5-chlorophenyl | SCN |
| A-1629 | 2-Chloro-3-methylphenyl | SCN |
| A-1630 | 2-Chloro-4-methylphenyl | SCN |
| A-1631 | 2-Chloro-5-methylphenyl | SCN |
| A-1632 | 3-Chloro-4-methylphenyl | SCN |
| A-1633 | 2-Methyl-3-fluorophenyl | SCN |
| A-1634 | 2-Methyl-4-fluorophenyl | SCN |
| A-1635 | 2-Methyl-5-fluorophenyl | SCN |
| A-1636 | 2-Methyl-6-fluorophenyl | SCN |
| A-1637 | 3-Methyl-4-fluorophenyl | SCN |
| A-1638 | 3-Methyl-5-fluorophenyl | SCN |
| A-1639 | 2-Fluoro-3-methylphenyl | SCN |
| A-1640 | 2-Fluoro-4-methylphenyl | SCN |
| A-1641 | 2-Fluoro-5-methylphenyl | SCN |
| A-1642 | 3-Fluoro-4-methylphenyl | SCN |
| A-1643 | 2-Chloro-3-ethylphenyl | SCN |
| A-1644 | 2-Chloro-4-ethylphenyl | SCN |
| A-1645 | 2-Chloro-5-ethylphenyl | SCN |
| A-1646 | 3-Chloro-4-ethylphenyl | SCN |
| A-1647 | 2-Ethyl-3-chlorophenyl | SCN |
| A-1648 | 2-Ethyl-4-chlorophenyl | SCN |
| A-1649 | 2-Ethyl-5-chlorophenyl | SCN |
| A-1650 | 2-Ethyl-6-chlorophenyl | SCN |
| A-1651 | 2-Ethyl-3-fluorophenyl | SCN |
| A-1652 | 2-Ethyl-4-fluorophenyl | SCN |
| A-1653 | 2-Ethyl-5-fluorophenyl | SCN |
| A-1654 | 2-Ethyl-6-fluorophenyl | SCN |
| A-1655 | 3-Ethyl-4-fluorophenyl | SCN |
| A-1656 | 3-Ethyl-5-fluorophenyl | SCN |
| A-1657 | 2-Fluoro-3-ethylphenyl | SCN |
| A-1658 | 2-Fluoro-4-ethylphenyl | SCN |
| A-1659 | 2-Fluoro-5-ethylphenyl | SCN |
| A-1660 | 3-Fluoro-4-ethylphenyl | SCN |
| A-1661 | 2-Methoxy-3-chlorophenyl | SCN |
| A-1662 | 2-Methoxy-4-chlorophenyl | SCN |
| A-1663 | 2-Methoxy-5-chlorophenyl | SCN |
| A-1664 | 2-Methoxy-6-chlorophenyl | SCN |
| A-1665 | 3-Methoxy-4-chlorophenyl | SCN |
| A-1666 | 3-Methoxy-5-chlorophenyl | SCN |
| A-1667 | 2-Chloro-3-methoxyphenyl | SCN |
| A-1668 | 2-Chloro-4-methoxyphenyl | SCN |
| A-1669 | 2-Chloro-5-methoxyphenyl | SCN |
| A-1670 | 3-Chloro-4-methoxyphenyl | SCN |
| A-1671 | 2-Methoxy-3-fluorophenyl | SCN |
| A-1672 | 2-Methoxy-4-fluorophenyl | SCN |
| A-1673 | 2-Methoxy-5-fluorophenyl | SCN |
| A-1674 | 2-Methoxy-6-fluorophenyl | SCN |
| A-1675 | 3-Methoxy-4-fluorophenyl | SCN |
| A-1676 | 3-Methoxy-5-fluorophenyl | SCN |
| A-1677 | 2-Fluoro-3-methoxyphenyl | SCN |
| A-1678 | 2-Fluoro-4-methoxyphenyl | SCN |
| A-1679 | 2-Fluoro-5-methoxyphenyl | SCN |
| A-1680 | 3-Fluoro-4-methoxyphenyl | SCN |
| A-1681 | 3-Fluoro-5-methoxyphenyl | SCN |
| A-1682 | 2-(Trifluoromethyl)-3-chlorophenyl | SCN |
| A-1683 | 2-(Trifluoromethyl)-4-chlorophenyl | SCN |
| A-1684 | 2-(Trifluoromethyl)-5-chlorophenyl | SCN |
| A-1685 | 2-(Trifluoromethyl)-6-chlorophenyl | SCN |
| A-1686 | 3-(Trifluoromethyl)-4-chlorophenyl | SCN |
| A-1687 | 3-(Trifluoromethyl)-5-chlorophenyl | SCN |
| A-1688 | 2-Chloro-3-(trifluoromethyl)phenyl | SCN |
| A-1689 | 2-Chloro-4-(trifluoromethyl)phenyl | SCN |
| A-1690 | 2-Chloro-5-(trifluoromethyl)phenyl | SCN |
| A-1691 | 3-Chloro-4-(trifluoromethyl)phenyl | SCN |
| A-1692 | 2-(Trifluoromethyl)-3-fluorophenyl | SCN |
| A-1693 | 2-(Trifluoromethyl)-4-fluorophenyl | SCN |
| A-1694 | 2-(Trifluoromethyl)-5-fluorophenyl | SCN |
| A-1695 | 2-(Trifluoromethyl)-6-fluorophenyl | SCN |
| A-1696 | 3-(Trifluoromethyl)-4-fluorophenyl | SCN |
| A-1697 | 3-(Trifluoromethyl)-5-fluorophenyl | SCN |
| A-1698 | 2-Fluoro-3-(trifluoromethyl)phenyl | SCN |
| A-1699 | 2-Fluoro-4-(trifluoromethyl)phenyl | SCN |
| A-1700 | 2-Fluoro-5-(trifluoromethyl)phenyl | SCN |
| A-1701 | 3-Fluoro-4-(trifluoromethyl)phenyl | SCN |
| A-1702 | 2-(Trifluoromethoxy)-3-chlorophenyl | SCN |
| A-1703 | 2-(Trifluoromethoxy)-4-chlorophenyl | SCN |
| A-1704 | 2-(Trifluoromethoxy)-5-chlorophenyl | SCN |
| A-1705 | 2-(Trifluoromethoxy)-6-chlorophenyl | SCN |
| A-1706 | 3-(Trifluoromethoxy)-4-chlorophenyl | SCN |
| A-1707 | 3-(Trifluoromethoxy)-5-chlorophenyl | SCN |
| A-1708 | 2-Chloro-3-(trifluoromethoxy)phenyl | SCN |
| A-1709 | 2-Chloro-4-(trifluoromethoxy)phenyl | SCN |
| A-1710 | 2-Chloro-5-(trifluoromethoxy)phenyl | SCN |
| A-1711 | 3-Chloro-4-(trifluoromethoxy)phenyl | SCN |
| A-1712 | 2-(Trifluoromethoxy)-3-fluorophenyl | SCN |
| A-1713 | 2-(Trifluoromethoxy)-4-fluorophenyl | SCN |
| A-1714 | 2-(Trifluoromethoxy)-5-fluorophenyl | SCN |
| A-1715 | 2-(Trifluoromethoxy)-6-fluorophenyl | SCN |
| A-1716 | 3-(Trifluoromethoxy)-4-fluorophenyl | SCN |
| A-1717 | 3-(Trifluoromethoxy)-5-fluorophenyl | SCN |
| A-1718 | 2-Fluoro-3-(trifluoromethoxy)phenyl | SCN |
| A-1719 | 2-Fluoro-4-(trifluoromethoxy)phenyl | SCN |
| A-1720 | 2-Fluoro-5-(trifluoromethoxy)phenyl | SCN |
| A-1721 | 3-Fluoro-4-(trifluoromethoxy)phenyl | SCN |
| A-1722 | 2-(Difluoromethoxy)-3-chlorophenyl | SCN |
| A-1723 | 2-(Difluoromethoxy)-4-chlorophenyl | SCN |
| A-1724 | 2-(Difluoromethoxy)-5-chlorophenyl | SCN |
| A-1725 | 2-(Difluoromethoxy)-6-chlorophenyl | SCN |
| A-1726 | 3-(Difluoromethoxy)-4-chlorophenyl | SCN |
| A-1727 | 3-(Difluoromethoxy)-5-chlorophenyl | SCN |
| A-1728 | 2-Chloro-3-(difluoromethoxy)phenyl | SCN |
| A-1729 | 2-Chloro-4-(difluoromethoxy)phenyl | SCN |
| A-1730 | 2-Chloro-5-(difluoromethoxy)phenyl | SCN |
| A-1731 | 3-Chloro-4-(difluoromethoxy)phenyl | SCN |
| A-1732 | 2-(Difluoromethoxy)-3-fluorophenyl | SCN |
| A-1733 | 2-(Difluoromethoxy)-4-fluorophenyl | SCN |
| A-1734 | 2-(Difluoromethoxy)-5-fluorophenyl | SCN |
| A-1735 | 2-(Difluoromethoxy)-6-fluorophenyl | SCN |
| A-1736 | 3-(Difluoromethoxy)-4-fluorophenyl | SCN |
| A-1737 | 3-(Difluoromethoxy)-5-fluorophenyl | SCN |
| A-1738 | 2-Fluoro-3-(difluoromethoxy)phenyl | SCN |
| A-1739 | 2-Fluoro-4-(difluoromethoxy)phenyl | SCN |
| A-1740 | 2-Fluoro-5-(difluoromethoxy)phenyl | SCN |
| A-1741 | 3-Fluoro-4-(difluoromethoxy)phenyl | SCN |
| A-1742 | 2-(Trifluoromethylthio)-3-chlorophenyl | SCN |
| A-1743 | 2-(Trifluoromethylthio)-4-chlorophenyl | SCN |
| A-1744 | 2-(Trifluoromethylthio)-5-chlorophenyl | SCN |
| A-1745 | 2-(Trifluoromethylthio)-6-chlorophenyl | SCN |
| A-1746 | 3-(Trifluoromethylthio)-4-chlorophenyl | SCN |
| A-1747 | 3-(Trifluoromethylthio)-5-chlorophenyl | SCN |
| A-1748 | 2-Chloro-3-(trifluoromethylthio)phenyl | SCN |
| A-1749 | 2-Chloro-4-(trifluoromethylthio)phenyl | SCN |
| A-1750 | 2-Chloro-5-(trifluoromethylthio)phenyl | SCN |
| A-1751 | 3-Chloro-4-(trifluoromethylthio)phenyl | SCN |
| A-1752 | 2-(Trifluoromethylthio)-3-fluorophenyl | SCN |
| A-1753 | 2-(Trifluoromethylthio)-4-fluorophenyl | SCN |
| A-1754 | 2-(Trifluoromethylthio)-5-fluorophenyl | SCN |
| A-1755 | 2-(Trifluoromethylthio)-6-fluorophenyl | SCN |
| A-1756 | 3-(Trifluoromethylthio)-4-fluorophenyl | SCN |
| A-1757 | 3-(Trifluoromethylthio)-5-fluorophenyl | SCN |
| A-1758 | 2-Fluoro-3-(trifluoromethylthio)phenyl | SCN |
| A-1759 | 2-Fluoro-4-(trifluoromethylthio)phenyl | SCN |
| A-1760 | 2-Fluoro-5-(trifluoromethylthio)phenyl | SCN |
| A-1761 | 3-Fluoro-4-(trifluoromethylthio)phenyl | SCN |
| A-1762 | 2,3,4-Trichlorophenyl | SCN |
| A-1763 | 2,3,5-Trichlorophenyl | SCN |
| A-1764 | 2,3,6-Trichlorophenyl | SCN |
| A-1765 | 2,4,5-Trichlorophenyl | SCN |
| A-1766 | 2,4,6-Trichlorophenyl | SCN |
| A-1767 | 3,4,5-Trichlorophenyl | SCN |
| A-1768 | 2,3,4-Trifluorophenyl | SCN |
| A-1769 | 2,3,5-Trifluorophenyl | SCN |
| A-1770 | 2,3,6-Trifluorophenyl | SCN |
| A-1771 | 2,4,5-Trifluorophenyl | SCN |
| A-1772 | 2,4,6-Trifluorophenyl | SCN |
| A-1773 | 3,4,5-Trifluorophenyl | SCN |
| A-1774 | 2,3,4-Trimethylphenyl | SCN |
| A-1775 | 2,3,5-Trimethylphenyl | SCN |
| A-1776 | 2,3,6-Trimethylphenyl | SCN |
| A-1777 | 2,4,5-Trimethylphenyl | SCN |
| A-1778 | 2,4,6-Trimethylphenyl | SCN |
| A-1779 | 3,4,5-Trimethylphenyl | SCN |
| A-1780 | 2,3,4-Trimethoxyphenyl | SCN |
| A-1781 | 2,3,5-Trimethoxyphenyl | SCN |
| A-1782 | 2,3,6-Trimethoxyphenyl | SCN |
| A-1783 | 2,4,5-Trimethoxyphenyl | SCN |
| A-1784 | 2,4,6-Trimethoxyphenyl | SCN |
| A-1785 | 3,4,5-Trimethoxyphenyl | SCN |
| A-1786 | Phenyl | S(C═O)CH₃ |
| A-1787 | 2-Chlorophenyl | S(C═O)CH₃ |
| A-1788 | 3-Chlorophenyl | S(C═O)CH₃ |
| A-1789 | 4-Chlorophenyl | S(C═O)CH₃ |
| A-1790 | 2-Fluorophenyl | S(C═O)CH₃ |
| A-1791 | 3-Fluorophenyl | S(C═O)CH₃ |
| A-1792 | 4-Fluorophenyl | S(C═O)CH₃ |
| A-1793 | 2-Methylphenyl | S(C═O)CH₃ |
| A-1794 | 3-Methylphenyl | S(C═O)CH₃ |
| A-1795 | 4-Methylphenyl | S(C═O)CH₃ |
| A-1796 | 2-Ethylphenyl | S(C═O)CH₃ |
| A-1797 | 3-Ethylphenyl | S(C═O)CH₃ |
| A-1798 | 4-Ethylphenyl | S(C═O)CH₃ |
| A-1799 | 2-Methoxyphenyl | S(C═O)CH₃ |
| A-1800 | 3-Methoxyphenyl | S(C═O)CH₃ |
| A-1801 | 4-Methoxyphenyl | S(C═O)CH₃ |
| A-1802 | 2-Trifluoromethylphenyl | S(C═O)CH₃ |
| A-1803 | 3-Trifluoromethylphenyl | S(C═O)CH₃ |
| A-1804 | 4-Trifluoromethylphenyl | S(C═O)CH₃ |
| A-1805 | 2-Trifluoromethoxyphenyl | S(C═O)CH₃ |
| A-1806 | 3-Trifluoromethoxyphenyl | S(C═O)CH₃ |
| A-1807 | 4-Trifluoromethoxyphenyl | S(C═O)CH₃ |
| A-1808 | 2-Difluoromethoxyphenyl | S(C═O)CH₃ |
| A-1809 | 3-Difluoromethoxyphenyl | S(C═O)CH₃ |
| A-1810 | 4-Difluoromethoxyphenyl | S(C═O)CH₃ |
| A-1811 | 2-Trifluoromethylthiophenyl | S(C═O)CH₃ |
| A-1812 | 3-Trifluoromethylthiophenyl | S(C═O)CH₃ |
| A-1813 | 4-Trifluoromethylthiophenyl | S(C═O)CH₃ |
| A-1814 | 2,3-Dichlorophenyl | S(C═O)CH₃ |
| A-1815 | 2,4-Dichlorophenyl | S(C═O)CH₃ |
| A-1816 | 2,5-Dichlorophenyl | S(C═O)CH₃ |
| A-1817 | 2,6-Dichlorophenyl | S(C═O)CH₃ |
| A-1818 | 3,4-Dichlorophenyl | S(C═O)CH₃ |
| A-1819 | 3,5-Dichlorophenyl | S(C═O)CH₃ |
| A-1820 | 2,3-Difluorophenyl | S(C═O)CH₃ |
| A-1821 | 2,4-Difluorophenyl | S(C═O)CH₃ |
| A-1822 | 2,5-Difluorophenyl | S(C═O)CH₃ |
| A-1823 | 2,6-Difluorophenyl | S(C═O)CH₃ |
| A-1824 | 3,4-Difluorophenyl | S(C═O)CH₃ |
| A-1825 | 3,5-Difluorophenyl | S(C═O)CH₃ |
| A-1826 | 2,3-Dimethylphenyl | S(C═O)CH₃ |
| A-1827 | 2,4-Dimethylphenyl | S(C═O)CH₃ |
| A-1828 | 2,5-Dimethylphenyl | S(C═O)CH₃ |
| A-1829 | 2,6-Dimethylphenyl | S(C═O)CH₃ |
| A-1830 | 3,4-Dimethylphenyl | S(C═O)CH₃ |
| A-1831 | 3,5-Dimethylphenyl | S(C═O)CH₃ |
| A-1832 | 2,3-Diethylphenyl | S(C═O)CH₃ |
| A-1833 | 2,4-Diethylphenyl | S(C═O)CH₃ |
| A-1834 | 2,5-Diethylphenyl | S(C═O)CH₃ |
| A-1835 | 2,6-Diethylphenyl | S(C═O)CH₃ |
| A-1836 | 3,5-Diethylphenyl | S(C═O)CH₃ |
| A-1837 | 3,4-Diethylphenyl | S(C═O)CH₃ |
| A-1838 | 2,3-Dimethoxyphenyl | S(C═O)CH₃ |
| A-1839 | 2,4-Dimethoxyphenyl | S(C═O)CH₃ |
| A-1840 | 2,5-Dimethoxyphenyl | S(C═O)CH₃ |
| A-1841 | 2,6-Dimethoxyphenyl | S(C═O)CH₃ |
| A-1842 | 3,4-Dimethoxyphenyl | S(C═O)CH₃ |
| A-1843 | 3,5-Dimethoxyphenyl | S(C═O)CH₃ |
| A-1844 | 2,3-Di(trifluoromethyl)phenyl | S(C═O)CH₃ |
| A-1845 | 2,4-Di(trifluoromethyl)phenyl | S(C═O)CH₃ |
| A-1846 | 2,5-Di(trifluoromethyl)phenyl | S(C═O)CH₃ |
| A-1847 | 2,6-Di(trifluoromethyl)phenyl | S(C═O)CH₃ |
| A-1848 | 3,4-Di(trifluoromethyl)phenyl | S(C═O)CH₃ |
| A-1849 | 3,5-Di(trifluoromethyl)phenyl | S(C═O)CH₃ |
| A-1850 | 2,3-Di(trifluoromethoxy)phenyl | S(C═O)CH₃ |
| A-1851 | 2,4-Di(trifluoromethoxy)phenyl | S(C═O)CH₃ |
| A-1852 | 2,5-Di(trifluoromethoxy)phenyl | S(C═O)CH₃ |
| A-1853 | 2,6-Di(trifluoromethoxy)phenyl | S(C═O)CH₃ |
| A-1854 | 3,4-Di(trifluoromethoxy)phenyl | S(C═O)CH₃ |
| A-1855 | 3,5-Di(trifluoromethoxy)phenyl | S(C═O)CH₃ |
| A-1856 | 2,3-Di(difluoromethoxy)phenyl | S(C═O)CH₃ |
| A-1857 | 2,4-Di(difluoromethoxy)phenyl | S(C═O)CH₃ |
| A-1858 | 2,5-Di(difluoromethoxy)phenyl | S(C═O)CH₃ |
| A-1859 | 2,6-Di(difluoromethoxy)phenyl | S(C═O)CH₃ |
| A-1860 | 3,4-Di(difluoromethoxy)phenyl | S(C═O)CH₃ |
| A-1861 | 3,5-Di(difluoromethoxy)phenyl | S(C═O)CH₃ |
| A-1862 | 2,3-Di(trifluoromethylthio)phenyl | S(C═O)CH₃ |
| A-1863 | 2,4-Di(trifluoromethylthio)phenyl | S(C═O)CH₃ |
| A-1864 | 2,5-Di(trifluoromethylthio)phenyl | S(C═O)CH₃ |
| A-1865 | 2,6-Di(trifluoromethylthio)phenyl | S(C═O)CH₃ |
| A-1866 | 3,4-Di(trifluoromethylthio)phenyl | S(C═O)CH₃ |
| A-1867 | 3,5-Di(trifluoromethylthio)phenyl | S(C═O)CH₃ |
| A-1868 | 2-Fluoro-3-chlorophenyl | S(C═O)CH₃ |
| A-1869 | 2-Fluoro-4-chlorophenyl | S(C═O)CH₃ |
| A-1870 | 2-Fluoro-5-chlorophenyl | S(C═O)CH₃ |
| A-1871 | 2-Fluoro-6-chlorophenyl | S(C═O)CH₃ |
| A-1872 | 3-Fluoro-4-chlorophenyl | S(C═O)CH₃ |
| A-1873 | 3-Fluoro-5-chlorophenyl | S(C═O)CH₃ |
| A-1874 | 2-Chloro-3-fluorophenyl | S(C═O)CH₃ |
| A-1875 | 2-Chloro-4-fluorophenyl | S(C═O)CH₃ |
| A-1876 | 2-Chloro-5-fluorophenyl | S(C═O)CH₃ |
| A-1877 | 3-Chloro-4-fluorophenyl | S(C═O)CH₃ |
| A-1878 | 2-Methyl-3-chlorophenyl | S(C═O)CH₃ |
| A-1879 | 2-Methyl-4-chlorophenyl | S(C═O)CH₃ |
| A-1880 | 2-Methyl-5-chlorophenyl | S(C═O)CH₃ |
| A-1881 | 2-Methyl-6-chlorophenyl | S(C═O)CH₃ |
| A-1882 | 3-Methyl-4-chlorophenyl | S(C═O)CH₃ |
| A-1883 | 3-Methyl-5-chlorophenyl | S(C═O)CH₃ |
| A-1884 | 2-Chloro-3-methylphenyl | S(C═O)CH₃ |
| A-1885 | 2-Chloro-4-methylphenyl | S(C═O)CH₃ |
| A-1886 | 2-Chloro-5-methylphenyl | S(C═O)CH₃ |
| A-1887 | 3-Chloro-4-methylphenyl | S(C═O)CH₃ |
| A-1888 | 2-Methyl-3-fluorophenyl | S(C═O)CH₃ |
| A-1889 | 2-Methyl-4-fluorophenyl | S(C═O)CH₃ |
| A-1890 | 2-Methyl-5-fluorophenyl | S(C═O)CH₃ |
| A-1891 | 2-Methyl-6-fluorophenyl | S(C═O)CH₃ |
| A-1892 | 3-Methyl-4-fluorophenyl | S(C═O)CH₃ |
| A-1893 | 3-Methyl-5-fluorophenyl | S(C═O)CH₃ |
| A-1894 | 2-Fluoro-3-methylphenyl | S(C═O)CH₃ |
| A-1895 | 2-Fluoro-4-methylphenyl | S(C═O)CH₃ |
| A-1896 | 2-Fluoro-5-methylphenyl | S(C═O)CH₃ |
| A-1897 | 3-Fluoro-4-methylphenyl | S(C═O)CH₃ |
| A-1898 | 2-Chloro-3-ethylphenyl | S(C═O)CH₃ |
| A-1899 | 2-Chloro-4-ethylphenyl | S(C═O)CH₃ |
| A-1900 | 2-Chloro-5-ethylphenyl | S(C═O)CH₃ |
| A-1901 | 3-Chloro-4-ethylphenyl | S(C═O)CH₃ |
| A-1902 | 2-Ethyl-3-chlorophenyl | S(C═O)CH₃ |
| A-1903 | 2-Ethyl-4-chlorophenyl | S(C═O)CH₃ |
| A-1904 | 2-Ethyl-5-chlorophenyl | S(C═O)CH₃ |
| A-1905 | 2-Ethyl-6-chlorophenyl | S(C═O)CH₃ |
| A-1906 | 2-Ethyl-3-fluorophenyl | S(C═O)CH₃ |
| A-1907 | 2-Ethyl-4-fluorophenyl | S(C═O)CH₃ |
| A-1908 | 2-Ethyl-5-fluorophenyl | S(C═O)CH₃ |
| A-1909 | 2-Ethyl-6-fluorophenyl | S(C═O)CH₃ |
| A-1910 | 3-Ethyl-4-fluorophenyl | S(C═O)CH₃ |
| A-1911 | 3-Ethyl-5-fluorophenyl | S(C═O)CH₃ |
| A-1912 | 2-Fluoro-3-ethylphenyl | S(C═O)CH₃ |
| A-1913 | 2-Fluoro-4-ethylphenyl | S(C═O)CH₃ |
| A-1914 | 2-Fluoro-5-ethylphenyl | S(C═O)CH₃ |
| A-1915 | 3-Fluoro-4-ethylphenyl | S(C═O)CH₃ |
| A-1916 | 2-Methoxy-3-chlorophenyl | S(C═O)CH₃ |
| A-1917 | 2-Methoxy-4-chlorophenyl | S(C═O)CH₃ |
| A-1918 | 2-Methoxy-5-chlorophenyl | S(C═O)CH₃ |
| A-1919 | 2-Methoxy-6-chlorophenyl | S(C═O)CH₃ |
| A-1920 | 3-Methoxy-4-chlorophenyl | S(C═O)CH₃ |
| A-1921 | 3-Methoxy-5-chlorophenyl | S(C═O)CH₃ |
| A-1922 | 2-Chloro-3-methoxyphenyl | S(C═O)CH₃ |
| A-1923 | 2-Chloro-4-methoxyphenyl | S(C═O)CH₃ |
| A-1924 | 2-Chloro-5-methoxyphenyl | S(C═O)CH₃ |
| A-1925 | 3-Chloro-4-methoxyphenyl | S(C═O)CH₃ |
| A-1926 | 2-Methoxy-3-fluorophenyl | S(C═O)CH₃ |
| A-1927 | 2-Methoxy-4-fluorophenyl | S(C═O)CH₃ |
| A-1928 | 2-Methoxy-5-fluorophenyl | S(C═O)CH₃ |
| A-1929 | 2-Methoxy-6-fluorophenyl | S(C═O)CH₃ |
| A-1930 | 3-Methoxy-4-fluorophenyl | S(C═O)CH₃ |
| A-1931 | 3-Methoxy-5-fluorophenyl | S(C═O)CH₃ |
| A-1932 | 2-Fluoro-3-methoxyphenyl | S(C═O)CH₃ |
| A-1933 | 2-Fluoro-4-methoxyphenyl | S(C═O)CH₃ |
| A-1934 | 2-Fluoro-5-methoxyphenyl | S(C═O)CH₃ |
| A-1935 | 3-Fluoro-4-methoxyphenyl | S(C═O)CH₃ |
| A-1936 | 3-Fluoro-5-methoxyphenyl | S(C═O)CH₃ |
| A-1937 | 2-(Trifluoromethyl)-3-chlorophenyl | S(C═O)CH₃ |
| A-1938 | 2-(Trifluoromethyl)-4-chlorophenyl | S(C═O)CH₃ |
| A-1939 | 2-(Trifluoromethyl)-5-chlorophenyl | S(C═O)CH₃ |
| A-1940 | 2-(Trifluoromethyl)-6-chlorophenyl | S(C═O)CH₃ |
| A-1941 | 3-(Trifluoromethyl)-4-chlorophenyl | S(C═O)CH₃ |
| A-1942 | 3-(Trifluoromethyl)-5-chlorophenyl | S(C═O)CH₃ |
| A-1943 | 2-Chloro-3-(trifluoromethyl)phenyl | S(C═O)CH₃ |
| A-1944 | 2-Chloro-4-(trifluoromethyl)phenyl | S(C═O)CH₃ |
| A-1945 | 2-Chloro-5-(trifluoromethyl)phenyl | S(C═O)CH₃ |
| A-1946 | 3-Chloro-4-(trifluoromethyl)phenyl | S(C═O)CH₃ |
| A-1947 | 2-(Trifluoromethyl)-3-fluorophenyl | S(C═O)CH₃ |
| A-1948 | 2-(Trifluoromethyl)-4-fluorophenyl | S(C═O)CH₃ |
| A-1949 | 2-(Trifluoromethyl)-5-fluorophenyl | S(C═O)CH₃ |
| A-1950 | 2-(Trifluoromethyl)-6-fluorophenyl | S(C═O)CH₃ |
| A-1951 | 3-(Trifluoromethyl)-4-fluorophenyl | S(C═O)CH₃ |
| A-1952 | 3-(Trifluoromethyl)-5-fluorophenyl | S(C═O)CH₃ |
| A-1953 | 2-Fluoro-3-(trifluoromethyl)phenyl | S(C═O)CH₃ |
| A-1954 | 2-Fluoro-4-(trifluoromethyl)phenyl | S(C═O)CH₃ |
| A-1955 | 2-Fluoro-5-(trifluoromethyl)phenyl | S(C═O)CH₃ |
| A-1956 | 3-Fluoro-4-(trifluoromethyl)phenyl | S(C═O)CH₃ |
| A-1957 | 2-(Trifluoromethoxy)-3-chlorophenyl | S(C═O)CH₃ |
| A-1958 | 2-(Trifluoromethoxy)-4-chlorophenyl | S(C═O)CH₃ |
| A-1959 | 2-(Trifluoromethoxy)-5-chlorophenyl | S(C═O)CH₃ |
| A-1960 | 2-(Trifluoromethoxy)-6-chlorophenyl | S(C═O)CH₃ |
| A-1961 | 3-(Trifluoromethoxy)-4-chlorophenyl | S(C═O)CH₃ |
| A-1962 | 3-(Trifluoromethoxy)-5-chlorophenyl | S(C═O)CH₃ |
| A-1963 | 2-Chloro-3-(trifluoromethoxy)phenyl | S(C═O)CH₃ |
| A-1964 | 2-Chloro-4-(trifluoromethoxy)phenyl | S(C═O)CH₃ |
| A-1965 | 2-Chloro-5-(trifluoromethoxy)phenyl | S(C═O)CH₃ |
| A-1966 | 3-Chloro-4-(trifluoromethoxy)phenyl | S(C═O)CH₃ |
| A-1967 | 2-(Trifluoromethoxy)-3-fluorophenyl | S(C═O)CH₃ |
| A-1968 | 2-(Trifluoromethoxy)-4-fluorophenyl | S(C═O)CH₃ |
| A-1969 | 2-(Trifluoromethoxy)-5-fluorophenyl | S(C═O)CH₃ |
| A-1970 | 2-(Trifluoromethoxy)-6-fluorophenyl | S(C═O)CH₃ |
| A-1971 | 3-(Trifluoromethoxy)-4-fluorophenyl | S(C═O)CH₃ |
| A-1972 | 3-(Trifluoromethoxy)-5-fluorophenyl | S(C═O)CH₃ |
| A-1973 | 2-Fluoro-3-(trifluoromethoxy)phenyl | S(C═O)CH₃ |
| A-1974 | 2-Fluoro-4-(trifluoromethoxy)phenyl | S(C═O)CH₃ |
| A-1975 | 2-Fluoro-5-(trifluoromethoxy)phenyl | S(C═O)CH₃ |
| A-1976 | 3-Fluoro-4-(trifluoromethoxy)phenyl | S(C═O)CH₃ |
| A-1977 | 2-(Difluoromethoxy)-3-chlorophenyl | S(C═O)CH₃ |
| A-1978 | 2-(Difluoromethoxy)-4-chlorophenyl | S(C═O)CH₃ |
| A-1979 | 2-(Difluoromethoxy)-5-chlorophenyl | S(C═O)CH₃ |
| A-1980 | 2-(Difluoromethoxy)-6-chlorophenyl | S(C═O)CH₃ |
| A-1981 | 3-(Difluoromethoxy)-4-chlorophenyl | S(C═O)CH₃ |
| A-1982 | 3-(Difluoromethoxy)-5-chlorophenyl | S(C═O)CH₃ |
| A-1983 | 2-Chloro-3-(difluoromethoxy)phenyl | S(C═O)CH₃ |
| A-1984 | 2-Chloro-4-(difluoromethoxy)phenyl | S(C═O)CH₃ |
| A-1985 | 2-Chloro-5-(difluoromethoxy)phenyl | S(C═O)CH₃ |
| A-1986 | 3-Chloro-4-(difluoromethoxy)phenyl | S(C═O)CH₃ |
| A-1987 | 2-(Difluoromethoxy)-3-fluorophenyl | S(C═O)CH₃ |
| A-1988 | 2-(Difluoromethoxy)-4-fluorophenyl | S(C═O)CH₃ |
| A-1989 | 2-(Difluoromethoxy)-5-fluorophenyl | S(C═O)CH₃ |
| A-1990 | 2-(Difluoromethoxy)-6-fluorophenyl | S(C═O)CH₃ |
| A-1991 | 3-(Difluoromethoxy)-4-fluorophenyl | S(C═O)CH₃ |
| A-1992 | 3-(Difluoromethoxy)-5-fluorophenyl | S(C═O)CH₃ |
| A-1993 | 2-Fluoro-3-(difluoromethoxy)phenyl | S(C═O)CH₃ |
| A-1994 | 2-Fluoro-4-(difluoromethoxy)phenyl | S(C═O)CH₃ |
| A-1995 | 2-Fluoro-5-(difluoromethoxy)phenyl | S(C═O)CH₃ |
| A-1996 | 3-Fluoro-4-(difluoromethoxy)phenyl | S(C═O)CH₃ |
| A-1997 | 2-(Trifluoromethylthio)-3-chlorophenyl | S(C═O)CH₃ |
| A-1998 | 2-(Trifluoromethylthio)-4-chlorophenyl | S(C═O)CH₃ |
| A-1999 | 2-(Trifluoromethylthio)-5-chlorophenyl | S(C═O)CH₃ |
| A-2000 | 2-(Trifluoromethylthio)-6-chlorophenyl | S(C═O)CH₃ |
| A-2001 | 3-(Trifluoromethylthio)-4-chlorophenyl | S(C═O)CH₃ |
| A-2002 | 3-(Trifluoromethylthio)-5-chlorophenyl | S(C═O)CH₃ |
| A-2003 | 2-Chloro-3-(trifluoromethylthio)phenyl | S(C═O)CH₃ |
| A-2004 | 2-Chloro-4-(trifluoromethylthio)phenyl | S(C═O)CH₃ |
| A-2005 | 2-Chloro-5-(trifluoromethylthio)phenyl | S(C═O)CH₃ |
| A-2006 | 3-Chloro-4-(trifluoromethylthio)phenyl | S(C═O)CH₃ |
| A-2007 | 2-(Trifluoromethylthio)-3-fluorophenyl | S(C═O)CH₃ |
| A-2008 | 2-(Trifluoromethylthio)-4-fluorophenyl | S(C═O)CH₃ |
| A-2009 | 2-(Trifluoromethylthio)-5-fluorophenyl | S(C═O)CH₃ |
| A-2010 | 2-(Trifluoromethylthio)-6-fluorophenyl | S(C═O)CH₃ |
| A-2011 | 3-(Trifluoromethylthio)-4-fluorophenyl | S(C═O)CH₃ |
| A-2012 | 3-(Trifluoromethylthio)-5-fluorophenyl | S(C═O)CH₃ |
| A-2013 | 2-Fluoro-3-(trifluoromethylthio)phenyl | S(C═O)CH₃ |
| A-2014 | 2-Fluoro-4-(trifluoromethylthio)phenyl | S(C═O)CH₃ |
| A-2015 | 2-Fluoro-5-(trifluoromethylthio)phenyl | S(C═O)CH₃ |
| A-2016 | 3-Fluoro-4-(trifluoromethylthio)phenyl | S(C═O)CH₃ |
| A-2017 | 2,3,4-Trichlorophenyl | S(C═O)CH₃ |
| A-2018 | 2,3,5-Trichlorophenyl | S(C═O)CH₃ |
| A-2019 | 2,3,6-Trichlorophenyl | S(C═O)CH₃ |
| A-2020 | 2,4,5-Trichlorophenyl | S(C═O)CH₃ |
| A-2021 | 2,4,6-Trichlorophenyl | S(C═O)CH₃ |
| A-2022 | 3,4,5-Trichlorophenyl | S(C═O)CH₃ |
| A-2023 | 2,3,4-Trifluorophenyl | S(C═O)CH₃ |
| A-2024 | 2,3,5-Trifluorophenyl | S(C═O)CH₃ |
| A-2025 | 2,3,6-Trifluorophenyl | S(C═O)CH₃ |
| A-2026 | 2,4,5-Trifluorophenyl | S(C═O)CH₃ |
| A-2027 | 2,4,6-Trifluorophenyl | S(C═O)CH₃ |
| A-2028 | 3,4,5-Trifluorophenyl | S(C═O)CH₃ |
| A-2029 | 2,3,4-Trimethylphenyl | S(C═O)CH₃ |
| A-2030 | 2,3,5-Trimethylphenyl | S(C═O)CH₃ |
| A-2031 | 2,3,6-Trimethylphenyl | S(C═O)CH₃ |
| A-2032 | 2,4,5-Trimethylphenyl | S(C═O)CH₃ |
| A-2033 | 2,4,6-Trimethylphenyl | S(C═O)CH₃ |
| A-2034 | 3,4,5-Trimethylphenyl | S(C═O)CH₃ |
| A-2035 | 2,3,4-Trimethoxyphenyl | S(C═O)CH₃ |
| A-2036 | 2,3,5-Trimethoxyphenyl | S(C═O)CH₃ |
| A-2037 | 2,3,6-Trimethoxyphenyl | S(C═O)CH₃ |
| A-2038 | 2,4,5-Trimethoxyphenyl | S(C═O)CH₃ |
| A-2039 | 2,4,6-Trimethoxyphenyl | S(C═O)CH₃ |
| A-2040 | 3,4,5-Trimethoxyphenyl | S(C═O)CH₃ |
| A-2041 | Phenyl | S(C═O)OCH₃ |
| A-2042 | 2-Chlorophenyl | S(C═O)OCH₃ |
| A-2043 | 3-Chlorophenyl | S(C═O)OCH₃ |
| A-2044 | 4-Chlorophenyl | S(C═O)OCH₃ |
| A-2045 | 2-Fluorophenyl | S(C═O)OCH₃ |
| A-2046 | 3-Fluorophenyl | S(C═O)OCH₃ |
| A-2047 | 4-Fluorophenyl | S(C═O)OCH₃ |
| A-2048 | 2-Methylphenyl | S(C═O)OCH₃ |
| A-2049 | 3-Methylphenyl | S(C═O)OCH₃ |
| A-2050 | 4-Methylphenyl | S(C═O)OCH₃ |
| A-2051 | 2-Ethylphenyl | S(C═O)OCH₃ |
| A-2052 | 3-Ethylphenyl | S(C═O)OCH₃ |
| A-2053 | 4-Ethylphenyl | S(C═O)OCH₃ |
| A-2054 | 2-Methoxyphenyl | S(C═O)OCH₃ |
| A-2055 | 3-Methoxyphenyl | S(C═O)OCH₃ |
| A-2056 | 4-Methoxyphenyl | S(C═O)OCH₃ |
| A-2057 | 2-Trifluoromethylphenyl | S(C═O)OCH₃ |
| A-2058 | 3-Trifluoromethylphenyl | S(C═O)OCH₃ |
| A-2059 | 4-Trifluoromethylphenyl | S(C═O)OCH₃ |
| A-2060 | 2-Trifluoromethoxyphenyl | S(C═O)OCH₃ |
| A-2061 | 3-Trifluoromethoxyphenyl | S(C═O)OCH₃ |
| A-2062 | 4-Trifluoromethoxyphenyl | S(C═O)OCH₃ |
| A-2063 | 2-Difluoromethoxyphenyl | S(C═O)OCH₃ |
| A-2064 | 3-Difluoromethoxyphenyl | S(C═O)OCH₃ |
| A-2065 | 4-Difluoromethoxyphenyl | S(C═O)OCH₃ |
| A-2066 | 2-Trifluoromethylthiophenyl | S(C═O)OCH₃ |
| A-2067 | 3-Trifluoromethylthiophenyl | S(C═O)OCH₃ |
| A-2068 | 4-Trifluoromethylthiophenyl | S(C═O)OCH₃ |
| A-2069 | 2,3-Dichlorophenyl | S(C═O)OCH₃ |
| A-2070 | 2,4-Dichlorophenyl | S(C═O)OCH₃ |
| A-2071 | 2,5-Dichlorophenyl | S(C═O)OCH₃ |
| A-2072 | 2,6-Dichlorophenyl | S(C═O)OCH₃ |
| A-2073 | 3,4-Dichlorophenyl | S(C═O)OCH₃ |
| A-2074 | 3,5-Dichlorophenyl | S(C═O)OCH₃ |
| A-2075 | 2,3-Difluorophenyl | S(C═O)OCH₃ |
| A-2076 | 2,4-Difluorophenyl | S(C═O)OCH₃ |
| A-2077 | 2,5-Difluorophenyl | S(C═O)OCH₃ |
| A-2078 | 2,6-Difluorophenyl | S(C═O)OCH₃ |
| A-2079 | 3,4-Difluorophenyl | S(C═O)OCH₃ |
| A-2080 | 3,5-Difluorophenyl | S(C═O)OCH₃ |
| A-2081 | 2,3-Dimethylphenyl | S(C═O)OCH₃ |
| A-2082 | 2,4-Dimethylphenyl | S(C═O)OCH₃ |
| A-2083 | 2,5-Dimethylphenyl | S(C═O)OCH₃ |
| A-2084 | 2,6-Dimethylphenyl | S(C═O)OCH₃ |
| A-2085 | 3,4-Dimethylphenyl | S(C═O)OCH₃ |
| A-2086 | 3,5-Dimethylphenyl | S(C═O)OCH₃ |
| A-2087 | 2,3-Diethylphenyl | S(C═O)OCH₃ |
| A-2088 | 2,4-Diethylphenyl | S(C═O)OCH₃ |
| A-2089 | 2,5-Diethylphenyl | S(C═O)OCH₃ |
| A-2090 | 2,6-Diethylphenyl | S(C═O)OCH₃ |
| A-2091 | 3,5-Diethylphenyl | S(C═O)OCH₃ |
| A-2092 | 3,4-Diethylphenyl | S(C═O)OCH₃ |
| A-2093 | 2,3-Dimethoxyphenyl | S(C═O)OCH₃ |
| A-2094 | 2,4-Dimethoxyphenyl | S(C═O)OCH₃ |
| A-2095 | 2,5-Dimethoxyphenyl | S(C═O)OCH₃ |
| A-2096 | 2,6-Dimethoxyphenyl | S(C═O)OCH₃ |
| A-2097 | 3,4-Dimethoxyphenyl | S(C═O)OCH₃ |
| A-2098 | 3,5-Dimethoxyphenyl | S(C═O)OCH₃ |
| A-2099 | 2,3-Di(trifluoromethyl)phenyl | S(C═O)OCH₃ |
| A-2100 | 2,4-Di(trifluoromethyl)phenyl | S(C═O)OCH₃ |
| A-2101 | 2,5-Di(trifluoromethyl)phenyl | S(C═O)OCH₃ |
| A-2102 | 2,6-Di(trifluoromethyl)phenyl | S(C═O)OCH₃ |
| A-2103 | 3,4-Di(trifluoromethyl)phenyl | S(C═O)OCH₃ |
| A-2104 | 3,5-Di(trifluoromethyl)phenyl | S(C═O)OCH₃ |
| A-2105 | 2,3-Di(trifluoromethoxy)phenyl | S(C═O)OCH₃ |
| A-2106 | 2,4-Di(trifluoromethoxy)phenyl | S(C═O)OCH₃ |
| A-2107 | 2,5-Di(trifluoromethoxy)phenyl | S(C═O)OCH₃ |
| A-2108 | 2,6-Di(trifluoromethoxy)phenyl | S(C═O)OCH₃ |
| A-2109 | 3,4-Di(trifluoromethoxy)phenyl | S(C═O)OCH₃ |
| A-2110 | 3,5-Di(trifluoromethoxy)phenyl | S(C═O)OCH₃ |
| A-2111 | 2,3-Di(difluoromethoxy)phenyl | S(C═O)OCH₃ |
| A-2112 | 2,4-Di(difluoromethoxy)phenyl | S(C═O)OCH₃ |
| A-2113 | 2,5-Di(difluoromethoxy)phenyl | S(C═O)OCH₃ |
| A-2114 | 2,6-Di(difluoromethoxy)phenyl | S(C═O)OCH₃ |
| A-2115 | 3,4-Di(difluoromethoxy)phenyl | S(C═O)OCH₃ |
| A-2116 | 3,5-Di(difluoromethoxy)phenyl | S(C═O)OCH₃ |
| A-2117 | 2,3-Di(trifluoromethylthio)phenyl | S(C═O)OCH₃ |
| A-2118 | 2,4-Di(trifluoromethylthio)phenyl | S(C═O)OCH₃ |
| A-2119 | 2,5-Di(trifluoromethylthio)phenyl | S(C═O)OCH₃ |
| A-2120 | 2,6-Di(trifluoromethylthio)phenyl | S(C═O)OCH₃ |
| A-2121 | 3,4-Di(trifluoromethylthio)phenyl | S(C═O)OCH₃ |
| A-2122 | 3,5-Di(trifluoromethylthio)phenyl | S(C═O)OCH₃ |
| A-2123 | 2-Fluoro-3-chlorophenyl | S(C═O)OCH₃ |
| A-2124 | 2-Fluoro-4-chlorophenyl | S(C═O)OCH₃ |
| A-2125 | 2-Fluoro-5-chlorophenyl | S(C═O)OCH₃ |
| A-2126 | 2-Fluoro-6-chlorophenyl | S(C═O)OCH₃ |
| A-2127 | 3-Fluoro-4-chlorophenyl | S(C═O)OCH₃ |
| A-2128 | 3-Fluoro-5-chlorophenyl | S(C═O)OCH₃ |
| A-2129 | 2-Chloro-3-fluorophenyl | S(C═O)OCH₃ |
| A-2130 | 2-Chloro-4-fluorophenyl | S(C═O)OCH₃ |
| A-2131 | 2-Chloro-5-fluorophenyl | S(C═O)OCH₃ |
| A-2132 | 3-Chloro-4-fluorophenyl | S(C═O)OCH₃ |
| A-2133 | 2-Methyl-3-chlorophenyl | S(C═O)OCH₃ |
| A-2134 | 2-Methyl-4-chlorophenyl | S(C═O)OCH₃ |
| A-2135 | 2-Methyl-5-chlorophenyl | S(C═O)OCH₃ |
| A-2136 | 2-Methyl-6-chlorophenyl | S(C═O)OCH₃ |
| A-2137 | 3-Methyl-4-chlorophenyl | S(C═O)OCH₃ |
| A-2138 | 3-Methyl-5-chlorophenyl | S(C═O)OCH₃ |
| A-2139 | 2-Chloro-3-methylphenyl | S(C═O)OCH₃ |
| A-2140 | 2-Chloro-4-methylphenyl | S(C═O)OCH₃ |
| A-2141 | 2-Chloro-5-methylphenyl | S(C═O)OCH₃ |
| A-2142 | 3-Chloro-4-methylphenyl | S(C═O)OCH₃ |
| A-2143 | 2-Methyl-3-fluorophenyl | S(C═O)OCH₃ |
| A-2144 | 2-Methyl-4-fluorophenyl | S(C═O)OCH₃ |
| A-2145 | 2-Methyl-5-fluorophenyl | S(C═O)OCH₃ |
| A-2146 | 2-Methyl-6-fluorophenyl | S(C═O)OCH₃ |
| A-2147 | 3-Methyl-4-fluorophenyl | S(C═O)OCH₃ |
| A-2148 | 3-Methyl-5-fluorophenyl | S(C═O)OCH₃ |
| A-2149 | 2-Fluoro-3-methylphenyl | S(C═O)OCH₃ |
| A-2150 | 2-Fluoro-4-methylphenyl | S(C═O)OCH₃ |
| A-2151 | 2-Fluoro-5-methylphenyl | S(C═O)OCH₃ |
| A-2152 | 3-Fluoro-4-methylphenyl | S(C═O)OCH₃ |
| A-2153 | 2-Chloro-3-ethylphenyl | S(C═O)OCH₃ |
| A-2154 | 2-Chloro-4-ethylphenyl | S(C═O)OCH₃ |
| A-2155 | 2-Chloro-5-ethylphenyl | S(C═O)OCH₃ |
| A-2156 | 3-Chloro-4-ethylphenyl | S(C═O)OCH₃ |
| A-2157 | 2-Ethyl-3-chlorophenyl | S(C═O)OCH₃ |
| A-2158 | 2-Ethyl-4-chlorophenyl | S(C═O)OCH₃ |
| A-2159 | 2-Ethyl-5-chlorophenyl | S(C═O)OCH₃ |
| A-2160 | 2-Ethyl-6-chlorophenyl | S(C═O)OCH₃ |
| A-2161 | 2-Ethyl-3-fluorophenyl | S(C═O)OCH₃ |
| A-2162 | 2-Ethyl-4-fluorophenyl | S(C═O)OCH₃ |
| A-2163 | 2-Ethyl-5-fluorophenyl | S(C═O)OCH₃ |
| A-2164 | 2-Ethyl-6-fluorophenyl | S(C═O)OCH₃ |
| A-2165 | 3-Ethyl-4-fluorophenyl | S(C═O)OCH₃ |
| A-2166 | 3-Ethyl-5-fluorophenyl | S(C═O)OCH₃ |
| A-2167 | 2-Fluoro-3-ethylphenyl | S(C═O)OCH₃ |
| A-2168 | 2-Fluoro-4-ethylphenyl | S(C═O)OCH₃ |
| A-2169 | 2-Fluoro-5-ethylphenyl | S(C═O)OCH₃ |
| A-2170 | 3-Fluoro-4-ethylphenyl | S(C═O)OCH₃ |
| A-2171 | 2-Methoxy-3-chlorophenyl | S(C═O)OCH₃ |
| A-2172 | 2-Methoxy-4-chlorophenyl | S(C═O)OCH₃ |
| A-2173 | 2-Methoxy-5-chlorophenyl | S(C═O)OCH₃ |
| A-2174 | 2-Methoxy-6-chlorophenyl | S(C═O)OCH₃ |
| A-2175 | 3-Methoxy-4-chlorophenyl | S(C═O)OCH₃ |
| A-2176 | 3-Methoxy-5-chlorophenyl | S(C═O)OCH₃ |
| A-2177 | 2-Chloro-3-methoxyphenyl | S(C═O)OCH₃ |
| A-2178 | 2-Chloro-4-methoxyphenyl | S(C═O)OCH₃ |
| A-2179 | 2-Chloro-5-methoxyphenyl | S(C═O)OCH₃ |
| A-2180 | 3-Chloro-4-methoxyphenyl | S(C═O)OCH₃ |
| A-2181 | 2-Methoxy-3-fluorophenyl | S(C═O)OCH₃ |
| A-2182 | 2-Methoxy-4-fluorophenyl | S(C═O)OCH₃ |
| A-2183 | 2-Methoxy-5-fluorophenyl | S(C═O)OCH₃ |
| A-2184 | 2-Methoxy-6-fluorophenyl | S(C═O)OCH₃ |
| A-2185 | 3-Methoxy-4-fluorophenyl | S(C═O)OCH₃ |
| A-2186 | 3-Methoxy-5-fluorophenyl | S(C═O)OCH₃ |
| A-2187 | 2-Fluoro-3-methoxyphenyl | S(C═O)OCH₃ |
| A-2188 | 2-Fluoro-4-methoxyphenyl | S(C═O)OCH₃ |
| A-2189 | 2-Fluoro-5-methoxyphenyl | S(C═O)OCH₃ |
| A-2190 | 3-Fluoro-4-methoxyphenyl | S(C═O)OCH₃ |
| A-2191 | 3-Fluoro-5-methoxyphenyl | S(C═O)OCH₃ |
| A-2192 | 2-(Trifluoromethyl)-3-chlorophenyl | S(C═O)OCH₃ |
| A-2193 | 2-(Trifluoromethyl)-4-chlorophenyl | S(C═O)OCH₃ |
| A-2194 | 2-(Trifluoromethyl)-5-chlorophenyl | S(C═O)OCH₃ |
| A-2195 | 2-(Trifluoromethyl)-6-chlorophenyl | S(C═O)OCH₃ |
| A-2196 | 3-(Trifluoromethyl)-4-chlorophenyl | S(C═O)OCH₃ |
| A-2197 | 3-(Trifluoromethyl)-5-chlorophenyl | S(C═O)OCH₃ |
| A-2198 | 2-Chloro-3-(trifluoromethyl)phenyl | S(C═O)OCH₃ |
| A-2199 | 2-Chloro-4-(trifluoromethyl)phenyl | S(C═O)OCH₃ |
| A-2200 | 2-Chloro-5-(trifluoromethyl)phenyl | S(C═O)OCH₃ |
| A-2201 | 3-Chloro-4-(trifluoromethyl)phenyl | S(C═O)OCH₃ |
| A-2202 | 2-(Trifluoromethyl)-3-fluorophenyl | S(C═O)OCH₃ |
| A-2203 | 2-(Trifluoromethyl)-4-fluorophenyl | S(C═O)OCH₃ |
| A-2204 | 2-(Trifluoromethyl)-5-fluorophenyl | S(C═O)OCH₃ |
| A-2205 | 2-(Trifluoromethyl)-6-fluorophenyl | S(C═O)OCH₃ |
| A-2206 | 3-(Trifluoromethyl)-4-fluorophenyl | S(C═O)OCH₃ |
| A-2207 | 3-(Trifluoromethyl)-5-fluorophenyl | S(C═O)OCH₃ |
| A-2208 | 2-Fluoro-3-(trifluoromethyl)phenyl | S(C═O)OCH₃ |
| A-2209 | 2-Fluoro-4-(trifluoromethyl)phenyl | S(C═O)OCH₃ |
| A-2210 | 2-Fluoro-5-(trifluoromethyl)phenyl | S(C═O)OCH₃ |
| A-2211 | 3-Fluoro-4-(trifluoromethyl)phenyl | S(C═O)OCH₃ |
| A-2212 | 2-(Trifluoromethoxy)-3-chlorophenyl | S(C═O)OCH₃ |
| A-2213 | 2-(Trifluoromethoxy)-4-chlorophenyl | S(C═O)OCH₃ |
| A-2214 | 2-(Trifluoromethoxy)-5-chlorophenyl | S(C═O)OCH₃ |
| A-2215 | 2-(Trifluoromethoxy)-6-chlorophenyl | S(C═O)OCH₃ |
| A-2216 | 3-(Trifluoromethoxy)-4-chlorophenyl | S(C═O)OCH₃ |
| A-2217 | 3-(Trifluoromethoxy)-5-chlorophenyl | S(C═O)OCH₃ |
| A-2218 | 2-Chloro-3-(trifluoromethoxy)phenyl | S(C═O)OCH₃ |
| A-2219 | 2-Chloro-4-(trifluoromethoxy)phenyl | S(C═O)OCH₃ |
| A-2220 | 2-Chloro-5-(trifluoromethoxy)phenyl | S(C═O)OCH₃ |
| A-2221 | 3-Chloro-4-(trifluoromethoxy)phenyl | S(C═O)OCH₃ |
| A-2222 | 2-(Trifluoromethoxy)-3-fluorophenyl | S(C═O)OCH₃ |
| A-2223 | 2-(Trifluoromethoxy)-4-fluorophenyl | S(C═O)OCH₃ |
| A-2224 | 2-(Trifluoromethoxy)-5-fluorophenyl | S(C═O)OCH₃ |
| A-2225 | 2-(Trifluoromethoxy)-6-fluorophenyl | S(C═O)OCH₃ |
| A-2226 | 3-(Trifluoromethoxy)-4-fluorophenyl | S(C═O)OCH₃ |
| A-2227 | 3-(Trifluoromethoxy)-5-fluorophenyl | S(C═O)OCH₃ |
| A-2228 | 2-Fluoro-3-(trifluoromethoxy)phenyl | S(C═O)OCH₃ |
| A-2229 | 2-Fluoro-4-(trifluoromethoxy)phenyl | S(C═O)OCH₃ |
| A-2230 | 2-Fluoro-5-(trifluoromethoxy)phenyl | S(C═O)OCH₃ |
| A-2231 | 3-Fluoro-4-(trifluoromethoxy)phenyl | S(C═O)OCH₃ |
| A-2232 | 2-(Difluoromethoxy)-3-chlorophenyl | S(C═O)OCH₃ |
| A-2233 | 2-(Difluoromethoxy)-4-chlorophenyl | S(C═O)OCH₃ |
| A-2234 | 2-(Difluoromethoxy)-5-chlorophenyl | S(C═O)OCH₃ |
| A-2235 | 2-(Difluoromethoxy)-6-chlorophenyl | S(C═O)OCH₃ |
| A-2236 | 3-(Difluoromethoxy)-4-chlorophenyl | S(C═O)OCH₃ |
| A-2237 | 3-(Difluoromethoxy)-5-chlorophenyl | S(C═O)OCH₃ |
| A-2238 | 2-Chloro-3-(difluoromethoxy)phenyl | S(C═O)OCH₃ |
| A-2239 | 2-Chloro-4-(difluoromethoxy)phenyl | S(C═O)OCH₃ |
| A-2240 | 2-Chloro-5-(difluoromethoxy)phenyl | S(C═O)OCH₃ |
| A-2241 | 3-Chloro-4-(difluoromethoxy)phenyl | S(C═O)OCH₃ |
| A-2242 | 2-(Difluoromethoxy)-3-fluorophenyl | S(C═O)OCH₃ |
| A-2243 | 2-(Difluoromethoxy)-4-fluorophenyl | S(C═O)OCH₃ |
| A-2244 | 2-(Difluoromethoxy)-5-fluorophenyl | S(C═O)OCH₃ |
| A-2245 | 2-(Difluoromethoxy)-6-fluorophenyl | S(C═O)OCH₃ |
| A-2246 | 3-(Difluoromethoxy)-4-fluorophenyl | S(C═O)OCH₃ |
| A-2247 | 3-(Difluoromethoxy)-5-fluorophenyl | S(C═O)OCH₃ |
| A-2248 | 2-Fluoro-3-(difluoromethoxy)phenyl | S(C═O)OCH₃ |
| A-2249 | 2-Fluoro-4-(difluoromethoxy)phenyl | S(C═O)OCH₃ |
| A-2250 | 2-Fluoro-5-(difluoromethoxy)phenyl | S(C═O)OCH₃ |
| A-2251 | 3-Fluoro-4-(difluoromethoxy)phenyl | S(C═O)OCH₃ |
| A-2252 | 2-(Trifluoromethylthio)-3-chlorophenyl | S(C═O)OCH₃ |
| A-2253 | 2-(Trifluoromethylthio)-4-chlorophenyl | S(C═O)OCH₃ |
| A-2254 | 2-(Trifluoromethylthio)-5-chlorophenyl | S(C═O)OCH₃ |
| A-2255 | 2-(Trifluoromethylthio)-6-chlorophenyl | S(C═O)OCH₃ |
| A-2256 | 3-(Trifluoromethylthio)-4-chlorophenyl | S(C═O)OCH₃ |
| A-2257 | 3-(Trifluoromethylthio)-5-chlorophenyl | S(C═O)OCH₃ |
| A-2258 | 2-Chloro-3-(trifluoromethylthio)phenyl | S(C═O)OCH₃ |
| A-2259 | 2-Chloro-4-(trifluoromethylthio)phenyl | S(C═O)OCH₃ |
| A-2260 | 2-Chloro-5-(trifluoromethylthio)phenyl | S(C═O)OCH₃ |
| A-2261 | 3-Chloro-4-(trifluoromethylthio)phenyl | S(C═O)OCH₃ |
| A-2262 | 2-(Trifluoromethylthio)-3-fluorophenyl | S(C═O)OCH₃ |
| A-2263 | 2-(Trifluoromethylthio)-4-fluorophenyl | S(C═O)OCH₃ |
| A-2264 | 2-(Trifluoromethylthio)-5-fluorophenyl | S(C═O)OCH₃ |
| A-2265 | 2-(Trifluoromethylthio)-6-fluorophenyl | S(C═O)OCH₃ |
| A-2266 | 3-(Trifluoromethylthio)-4-fluorophenyl | S(C═O)OCH₃ |
| A-2267 | 3-(Trifluoromethylthio)-5-fluorophenyl | S(C═O)OCH₃ |
| A-2268 | 2-Fluoro-3-(trifluoromethylthio)phenyl | S(C═O)OCH₃ |
| A-2269 | 2-Fluoro-4-(trifluoromethylthio)phenyl | S(C═O)OCH₃ |
| A-2270 | 2-Fluoro-5-(trifluoromethylthio)phenyl | S(C═O)OCH₃ |
| A-2271 | 3-Fluoro-4-(trifluoromethylthio)phenyl | S(C═O)OCH₃ |
| A-2272 | 2,3,4-Trichlorophenyl | S(C═O)OCH₃ |
| A-2273 | 2,3,5-Trichlorophenyl | S(C═O)OCH₃ |
| A-2274 | 2,3,6-Trichlorophenyl | S(C═O)OCH₃ |
| A-2275 | 2,4,5-Trichlorophenyl | S(C═O)OCH₃ |
| A-2276 | 2,4,6-Trichlorophenyl | S(C═O)OCH₃ |
| A-2277 | 3,4,5-Trichlorophenyl | S(C═O)OCH₃ |
| A-2278 | 2,3,4-Trifluorophenyl | S(C═O)OCH₃ |
| A-2279 | 2,3,5-Trifluorophenyl | S(C═O)OCH₃ |
| A-2280 | 2,3,6-Trifluorophenyl | S(C═O)OCH₃ |
| A-2281 | 2,4,5-Trifluorophenyl | S(C═O)OCH₃ |
| A-2282 | 2,4,6-Trifluorophenyl | S(C═O)OCH₃ |
| A-2283 | 3,4,5-Trifluorophenyl | S(C═O)OCH₃ |
| A-2284 | 2,3,4-Trimethylphenyl | S(C═O)OCH₃ |
| A-2285 | 2,3,5-Trimethylphenyl | S(C═O)OCH₃ |
| A-2286 | 2,3,6-Trimethylphenyl | S(C═O)OCH₃ |
| A-2287 | 2,4,5-Trimethylphenyl | S(C═O)OCH₃ |
| A-2288 | 2,4,6-Trimethylphenyl | S(C═O)OCH₃ |
| A-2289 | 3,4,5-Trimethylphenyl | S(C═O)OCH₃ |
| A-2290 | 2,3,4-Trimethoxyphenyl | S(C═O)OCH₃ |
| A-2291 | 2,3,5-Trimethoxyphenyl | S(C═O)OCH₃ |
| A-2292 | 2,3,6-Trimethoxyphenyl | S(C═O)OCH₃ |
| A-2293 | 2,4,5-Trimethoxyphenyl | S(C═O)OCH₃ |
| A-2294 | 2,4,6-Trimethoxyphenyl | S(C═O)OCH₃ |
| A-2295 | 3,4,5-Trimethoxyphenyl | S(C═O)OCH₃ |

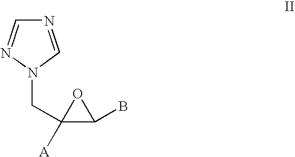

In particular with a view to their use, preference is given to the compounds II compiled in Tables 1c to 162c below. The groups mentioned in the tables for a substituent are furthermore per se, independently of the combination in which they are mentioned, a particularly preferred embodiment of the substituent in question.

Table 1c
Compounds II in which B is 2,3-difluorophenyl and A corresponds in each case to one row of Table B (compounds II.1cB-1 to II.1cB-255)

Table 2c
Compounds II in which B is 2,4-difluorophenyl and A corresponds in each case to one row of Table B (compounds II.2cB-1 to II.2cB-255)

Table 3c
Compounds II in which B is 2,5-difluorophenyl and A corresponds in each case to one row of Table B (compounds II.3cB-1 to II.3cB-255)

Table 4c
Compounds II in which B is 2,6-difluorophenyl and A corresponds in each case to one row of Table B (compounds II.4cB-1 to II.4cB-255)

Table 5c
Compounds II in which B is 2-fluoro-3-chlorophenyl and A corresponds in each case to one row of Table B (compounds II.5cB-1 to II.5cB-255)

Table 6c
Compounds II in which B is 2-fluoro-4-chlorophenyl and A corresponds in each case to one row of Table B (compounds II.6cB-1 to II.6cB-255)

Table 7c
Compounds II in which B is 2-fluoro-5-chlorophenyl and A corresponds in each case to one row of Table B (compounds II.7cB-1 to II.7cB-255)

Table 8c
Compounds II in which B is 2-fluoro-6-chlorophenyl and A corresponds in each case to one row of Table B (compounds II.8cB-1 to II.8cB-255)

Table 9c
Compounds II in which B is 2-chloro-3-fluorophenyl and A corresponds in each case to one row of Table B (compounds II.9cB-1 to II.9cB-255)

Table 10c
Compounds II in which B is 2-chloro-4-fluorophenyl and A corresponds in each case to one row of Table B (compounds II.10cB-1 to II.10cB-255)

Table 11c
Compounds II in which B is 2-chloro-5-fluorophenyl and A corresponds in each case to one row of Table B (compounds II.11cB-1 to II.11cB-255)

Table 12c
Compounds II in which B is 2-methyl-3-fluorophenyl and A corresponds in each case to one row of Table B (compounds II.12cB-1 to II.12cB-255)

Table 13c
Compounds II in which B is 2-methyl-4-fluorophenyl and A corresponds in each case to one row of Table B (compounds II.13cB-1 to II.13cB-255)

Table 14c
Compounds II in which B is 2-methyl-5-fluorophenyl and A corresponds in each case to one row of Table B (compounds II.14cB-1 to II.14cB-255)

Table 15c
Compounds II in which B is 2-methyl-6-fluorophenyl and A corresponds in each case to one row of Table B (compounds II.15cB-1 to II.15cB-255)

Table 16c
Compounds II in which B is 2-fluoro-3-methylphenyl and A corresponds in each case to one row of Table B (compounds II.16cB-1 to II.16cB-255)

Table 17c
Compounds II in which B is 2-fluoro-4-methylphenyl and A corresponds in each case to one row of Table B (compounds II.17cB-1 to II.17cB-255)

Table 18c
Compounds II in which B is 2-fluoro-5-methylphenyl and A corresponds in each case to one row of Table B (compounds II.18cB-1 to II.18cB-255)

Table 19c
Compounds II in which B is 2-ethyl-3-fluorophenyl and A corresponds in each case to one row of Table B (compounds II.19cB-1 to II.19cB-255)

Table 20c
Compounds II in which B is 2-ethyl-4-fluorophenyl and A corresponds in each case to one row of Table B (compounds II.20cB-1 to II.20cB-255)

Table 21c
Compounds II in which B is 2-ethyl-5-fluorophenyl and A corresponds in each case to one row of Table B (compounds II.21cB-1 to II.21cB-255)

Table 22c
Compounds II in which B is 2-ethyl-6-fluorophenyl and A corresponds in each case to one row of Table B (compounds II.22cB-1 to II.22cB-255)

Table 23c
Compounds II in which B is 2-fluoro-3-ethylphenyl and A corresponds in each case to one row of Table B (compounds II.23cB-1 to II.23cB-255)

Table 24c
Compounds II in which B is 2-fluoro-4-ethylphenyl and A corresponds in each case to one row of Table B (compounds II.24cB-1 to II.24cB-255)

Table 25c
Compounds II in which B is 2-methoxy-3-fluorophenyl and A corresponds in each case to one row of Table B (compounds II.25cB-1 to II.25cB-255)

Table 26c
Compounds II in which B is 2-methoxy-4-fluorophenyl and A corresponds in each case to one row of Table B (compounds II.26cB-1 to II.26cB-255)

Table 27c
Compounds II in which B is 2-methoxy-5-fluorophenyl and A corresponds in each case to one row of Table B (compounds II.27cB-1 to II.27cB-255)

Table 28c
Compounds II in which B is 2-methoxy-6-fluorophenyl and A corresponds in each case to one row of Table B (compounds II.28cB-1 to II.28cB-255)

Table 29c
Compounds II in which B is 2-fluoro-3-methoxyphenyl and A corresponds in each case to one row of Table B (compounds II.29cB-1 to II.29cB-255)

Table 30c
Compounds II in which B is 2-fluoro-4-methoxyphenyl and A corresponds in each case to one row of Table B (compounds II.30cB-1 to II.30cB-255)

Table 31c
Compounds II in which B is 2-fluoro-5-methoxyphenyl and A corresponds in each case to one row of Table B (compounds II.31cB-1 to II.31cB-255)

Table 32c
Compounds II in which B is 2-(trifluoromethyl)-3-fluorophenyl and A corresponds in each case to one row of Table B (compounds II.32cB-1 to II.32cB-255)

Table 33c
Compounds II in which B is 2,4,5-trimethoxyphenyl and A corresponds in each case to one row of Table B (compounds II.33cB-1 to II.33cB-255)

Table 34c
Compounds II in which B is 2,4,6-trimethoxyphenyl and A corresponds in each case to one row of Table B (compounds II.34cB-1 to II.34cB-255)

Table 35c
Compounds II in which B is 2-fluoro-5-ethylphenyl and A corresponds in each case to one row of Table B (compounds II.35cB-1 to II.35cB-255)

Table 36c
Compounds II in which B is 2-(trifluoromethyl)-4-fluorophenyl and A corresponds in each case to one row of Table B (compounds II.36cB-1 to II.36cB-255)

Table 37c
Compounds II in which B is 2-(trifluoromethyl)-5-fluorophenyl and A corresponds in each case to one row of Table B (compounds II.37cB-1 to II.37cB-255)

Table 38c
Compounds II in which B is 2-(trifluoromethyl)-6-fluorophenyl and A corresponds in each case to one row of Table B (compounds II.38cB-1 to II.38cB-255)

Table 39c
Compounds II in which B is 2-fluoro-3-(trifluoromethyl)phenyl and A corresponds in each case to one row of Table B (compounds II.39cB-1 to II.39cB-255)

Table 40c
Compounds II in which B is 2-fluoro-4-(trifluoromethyl)phenyl and A corresponds in each case to one row of Table B (compounds II.40cB-1 to II.40cB-255)

Table 41c
Compounds II in which B is 2-fluoro-5-(trifluoromethyl)phenyl and A corresponds in each case to one row of Table B (compounds II.41 cB-1 to II.41 cB-255)

Table 42c
Compounds II in which B is 2-(trifluoromethoxy)-3-fluorophenyl and A corresponds in each case to one row of Table B (compounds II.42cB-1 to II.42cB-255)

Table 43c
Compounds II in which B is 2-(trifluoromethoxy)-4-fluorophenyl and A corresponds in each case to one row of Table B (compounds II.43cB-1 to II.43cB-255)

Table 44c
Compounds II in which B is 2-(trifluoromethoxy)-5-fluorophenyl and A corresponds in each case to one row of Table B (compounds II.44cB-1 to II.44cB-255)

Table 45c
Compounds II in which B is 2-(trifluoromethoxy)-6-fluorophenyl and A corresponds in each case to one row of Table B (compounds II.45cB-1 to II.45cB-255)

Table 46c
Compounds II in which B is 2-fluoro-3-(trifluoromethoxy)phenyl and A corresponds in each case to one row of Table B (compounds II.46cB-1 to II.46cB-255)

Table 47c
Compounds II in which B is 2-fluoro-4-(trifluoromethoxy)phenyl and A corresponds in each case to one row of Table B (compounds II.47cB-1 to II.47cB-255)

Table 48c
Compounds II in which B is 2-fluoro-5-(trifluoromethoxy)phenyl and A corresponds in each case to one row of Table B (compounds II.48cB-1 to II.48cB-255)

Table 49c
Compounds II in which B is 2-(difluoromethoxy)-3-fluorophenyl and A corresponds in each case to one row of Table B (compounds II.49cB-1 to II.49cB-255)

Table 50c
Compounds II in which B is 2-(difluoromethoxy)-4-fluorophenyl and A corresponds in each case to one row of Table B (compounds II.50cB-1 to II.50cB-255)

Table 51c
Compounds II in which B is 2-(difluoromethoxy)-5-fluorophenyl and A corresponds in each case to one row of Table B (compounds II.51cB-1 to II.51cB-255)

Table 52c
Compounds II in which B is 2-(difluoromethoxy)-6-fluorophenyl and A corresponds in each case to one row of Table B (compounds II.52cB-1 to II.52cB-255)

Table 53c
Compounds II in which B is 2-fluoro-3-(difluoromethoxy)phenyl and A corresponds in each case to one row of Table B (compounds II.53cB-1 to II.53cB-255)

Table 54c
Compounds II in which B is 2-fluoro-4-(difluoromethoxy)phenyl and A corresponds in each case to one row of Table B (compounds II.54cB-1 to II.54cB-255)

Table 55c
Compounds II in which B is 2-fluoro-5-(difluoromethoxy)phenyl and A corresponds in each case to one row of Table B (compounds II.55cB-1 to II.55cB-255)

Table 56c
Compounds II in which B is 2-(trifluoromethylthio)-3-fluorophenyl and A corresponds in each case to one row of Table B (compounds II.56cB-1 to II.56cB-255)

Table 57c
Compounds II in which B is 2-(trifluoromethylthio)-4-fluorophenyl and A corresponds in each case to one row of Table B (compounds II.57cB-1 to II.57cB-255)

Table 58c
Compounds II in which B is 2-(trifluoromethylthio)-5-fluorophenyl and A corresponds in each case to one row of Table B (compounds II.58cB-1 to II.58cB-255)

Table 59c
Compounds II in which B is 2-(trifluoromethylthio)-6-fluorophenyl and A corresponds in each case to one row of Table B (compounds II.59cB-1 to II.59cB-255)

Table 60c
Compounds II in which B is 2-fluoro-3-(trifluoromethylthio)phenyl and A corresponds in each case to one row of Table B (compounds II.60cB-1 to II.60cB-255)

Table 61c
Compounds II in which B is 2-fluoro-4-(trifluoromethylthio)phenyl and A corresponds in each case to one row of Table B (compounds II.61cB-1 to II.61cB-255)

Table 62c
Compounds II in which B is 2-fluoro-5-(trifluoromethylthio)phenyl and A corresponds in each case to one row of Table B (compounds II.62cB-1 to II.62cB-255)

Table 63c
Compounds II in which B is 2,3,4-trifluorophenyl and A corresponds in each case to one row of Table B (compounds II.63cB-1 to II.63cB-255)

Table 64c
Compounds II in which B is 2,3,5-trifluorophenyl and A corresponds in each case to one row of Table B (compounds II.64cB-1 to II.64cB-255)

Table 65c
Compounds II in which B is 2,3,6-trifluorophenyl and A corresponds in each case to one row of Table B (compounds II.65cB-1 to II.65cB-255)

Table 66c
Compounds II in which B is 2,4,5-trifluorophenyl and A corresponds in each case to one row of Table B (compounds II.66cB-1 to II.66cB-255)

Table 67c
Compounds II in which B is 2,4,6-trifluorophenyl and A corresponds in each case to one row of Table B (compounds II.67cB-1 to II.67cB-255)

Table 68c
Compounds II in which B is 2,3-dichlorophenyl and A corresponds in each case to one row of Table B (compounds II.68cB-1 to II.68cB-255)

Table 69c
Compounds II in which B is 2,4-dichlorophenyl and A corresponds in each case to one row of Table B (compounds II.69cB-1 to II.69cB-255)

Table 70c
Compounds II in which B is 2,5-dichlorophenyl and A corresponds in each case to one row of Table B (compounds II.70cB-1 to II.70cB-255)

Table 71c
Compounds II in which B is 2,6-dichlorophenyl and A corresponds in each case to one row of Table B (compounds II.71cB-1 to II.71cB-255)

Table 72c
Compounds II in which B is 2,3-dimethylphenyl and A corresponds in each case to one row of Table B (compounds II.72cB-1 to II.72cB-255)

Table 73c
Compounds II in which B is 2,4-dimethylphenyl and A corresponds in each case to one row of Table B (compounds II.73cB-1 to II.73cB-255)

Table 74c
Compounds II in which B is 2,5-dimethylphenyl and A corresponds in each case to one row of Table B (compounds II.74cB-1 to II.74cB-255)

Table 75c
Compounds II in which B is 2,6-dimethylphenyl and A corresponds in each case to one row of Table B (compounds II.75cB-1 to II.75cB-255)

Table 76c
Compounds II in which B is 2,3-diethylphenyl and A corresponds in each case to one row of Table B (compounds II.76cB-1 to II.76cB-255)

Table 77c
Compounds II in which B is 2,4-diethylphenyl and A corresponds in each case to one row of Table B (compounds II.77cB-1 to II.77cB-255)

Table 78c
Compounds II in which B is 2,5-diethylphenyl and A corresponds in each case to one row of Table B (compounds II.78cB-1 to II.78cB-255)

Table 79c
Compounds II in which B is 2,6-diethylphenyl and A corresponds in each case to one row of Table B (compounds II.79cB-1 to II.79cB-255)

Table 80c
Compounds II in which B is 2,3-dimethoxyphenyl and A corresponds in each case to one row of Table B (compounds II.80cB-1 to II.80cB-255)

Table 81c
Compounds II in which B is 2,4-dimethoxyphenyl and A corresponds in each case to one row of Table B (compounds II.81cB-1 to II.81cB-255)

Table 82c
Compounds II in which B is 2,5-dimethoxyphenyl and A corresponds in each case to one row of Table B (compounds II.82cB-1 to II.82cB-255)

Table 83c
Compounds II in which B is 2,6-dimethoxyphenyl and A corresponds in each case to one row of Table B (compounds II.83cB-1 to II.83cB-255)

Table 84c
Compounds II in which B is 2,3-di(trifluoromethyl)phenyl and A corresponds in each case to one row of Table B (compounds II.84cB-1 to II.84cB-255)

Table 85c
Compounds II in which B is 2,4-di(trifluoromethyl)phenyl and A corresponds in each case to one row of Table B (compounds II.85cB-1 to II.85cB-255)

Table 86c
Compounds II in which B is 2,5-di(trifluoromethyl)phenyl and A corresponds in each case to one row of Table B (compounds II.86cB-1 to II.86cB-255)

Table 87c
Compounds II in which B is 2,6-di(trifluoromethyl)phenyl and A corresponds in each case to one row of Table B (compounds II.87cB-1 to II.87cB-255)

Table 88c
Compounds II in which B is 2,3-di(trifluoromethoxy)phenyl and A corresponds in each case to one row of Table B (compounds II.88cB-1 to II.88cB-255)

Table 89c
Compounds II in which B is 2,4-di(trifluoromethoxy)phenyl and A corresponds in each case to one row of Table B (compounds II.89cB-1 to II.89cB-255)

Table 90c
Compounds II in which B is 2,5-di(trifluoromethoxy)phenyl and A corresponds in each case to one row of Table B (compounds II.90cB-1 to II.90cB-255)

Table 91c
Compounds II in which B is 2,6-di(trifluoromethoxy)phenyl and A corresponds in each case to one row of Table B (compounds II.91cB-1 to II.91cB-255)

Table 92c
Compounds II in which B is 2,3-di(difluoromethoxy)phenyl and A corresponds in each case to one row of Table B (compounds II.92cB-1 to II.92cB-255)

Table 93c
Compounds II in which B is 2,4-di(difluoromethoxy)phenyl and A corresponds in each case to one row of Table B (compounds II.93cB-1 to II.93cB-255)

Table 94c
Compounds II in which B is 2,5-di(difluoromethoxy)phenyl and A corresponds in each case to one row of Table B (compounds II.94cB-1 to II.94cB-255)

Table 95c
Compounds II in which B is 2,6-di(difluoromethoxy)phenyl and A corresponds in each case to one row of Table B (compounds II.95cB-1 to II.95cB-255)

Table 96c
Compounds II in which B is 2,3-di(trifluoromethylthio)phenyl and A corresponds in each case to one row of Table B (compounds II.96cB-1 to II.96cB-255)

Table 97c
Compounds II in which B is 2,4-di(trifluoromethylthio)phenyl and A corresponds in each case to one row of Table B (compounds II.97cB-1 to II.97cB-255)

Table 98c
Compounds II in which B is 2,5-di(trifluoromethylthio)phenyl and A corresponds in each case to one row of Table B (compounds II.98cB-1 to II.98cB-255)

Table 99c
Compounds II in which B is 2,6-di(trifluoromethylthio)phenyl and A corresponds in each case to one row of Table B (compounds II.99cB-1 to II.99cB-255)

Table 100c
Compounds II in which B is 2-methyl-3-chlorophenyl and A corresponds in each case to one row of Table B (compounds II.100cB-1 to II.100cB-255)

Table 101c
Compounds II in which B is 2-methyl-4-chlorophenyl and A corresponds in each case to one row of Table B (compounds II.101cB-1 to II.101cB-255)

Table 102c
Compounds II in which B is 2-methyl-5-chlorophenyl and A corresponds in each case to one row of Table B (compounds II.102cB-1 to II.102cB-255)

Table 103c
Compounds II in which B is 2-methyl-6-chlorophenyl and A corresponds in each case to one row of Table B (compounds II.103cB-1 to II.103cB-255)

Table 104c
Compounds II in which B is 2-chloro-3-methylphenyl and A corresponds in each case to one row of Table B (compounds II.104cB-1 to II.104cB-255)

Table 105c
Compounds II in which B is 2-chloro-4-methylphenyl and A corresponds in each case to one row of Table B (compounds II.105cB-1 to II.105cB-255)

Table 106c
Compounds II in which B is 2-chloro-5-methylphenyl and A corresponds in each case to one row of Table B (compounds II.106cB-1 to II.106cB-255)

Table 107c
Compounds II in which B is 2-chloro-3-ethylphenyl and A corresponds in each case to one row of Table B (compounds II.107cB-1 to II.107cB-255)

Table 108c
Compounds II in which B is 2-chloro-4-ethylphenyl and A corresponds in each case to one row of Table B (compounds II.108cB-1 to II.108cB-255)

Table 109c
Compounds II in which B is 2-chloro-5-ethylphenyl and A corresponds in each case to one row of Table B (compounds II.109cB-1 to II.109cB-255)

Table 110c
Compounds II in which B is 2-ethyl-3-chlorophenyl and A corresponds in each case to one row of Table B (compounds II.110cB-1 to II.110cB-255)

### Table 111c

Compounds II in which B is 2-ethyl-4-chlorophenyl and A corresponds in each case to one row of Table B (compounds II.111cB-1 to II.111cB-255)

Table 112c
Compounds II in which B is 2-ethyl-5-chlorophenyl and A corresponds in each case to one row of Table B (compounds II.112cB-1 to II.112cB-255)

Table 113c
Compounds II in which B is 2-ethyl-6-chlorophenyl and A corresponds in each case to one row of Table B (compounds II.113cB-1 to II.113cB-255)

Table 114c
Compounds II in which B is 2-methoxy-3-chlorophenyl and A corresponds in each case to one row of Table B (compounds II.114cB-1 to II.114cB-255)

Table 115c
Compounds II in which B is 2-methoxy-4-chlorophenyl and A corresponds in each case to one row of Table B (compounds II.115cB-1 to II.115cB-255)

Table 116c
Compounds II in which B is 2-methoxy-5-chlorophenyl and A corresponds in each case to one row of Table B (compounds II.116cB-1 to II.116cB-255)

Table 117c
Compounds II in which B is 2-methoxy-6-chlorophenyl and A corresponds in each case to one row of Table B (compounds II.117cB-1 to II.117cB-255)

Table 118c
Compounds II in which B is 2-chloro-3-methoxyphenyl and A corresponds in each case to one row of Table B (compounds II.118cB-1 to II.118cB-255)

Table 119c
Compounds II in which B is 2-chloro-4-methoxyphenyl and A corresponds in each case to one row of Table B (compounds II.119cB-1 to II.119cB-255)

Table 120c
Compounds II in which B is 2-chloro-5-methoxyphenyl and A corresponds in each case to one row of Table B (compounds II.120cB-1 to II.120cB-255)

Table 121c
Compounds II in which B is 2-(trifluoromethyl)-3-chlorophenyl and A corresponds in each case to one row of Table B (compounds II.121cB-1 to II.121 cB-255)

Table 122c
Compounds II in which B is 2-(trifluoromethyl)-4-chlorophenyl and A corresponds in each case to one row of Table B (compounds II.122cB-1 to II.122cB-255)

Table 123c
Compounds II in which B is 2-(trifluoromethyl)-5-chlorophenyl and A corresponds in each case to one row of Table B (compounds II.123cB-1 to II.123cB-255)

Table 124c
Compounds II in which B is 2-(trifluoromethyl)-6-chlorophenyl and A corresponds in each case to one row of Table B (compounds II.124cB-1 to II.124cB-255)

Table 125c
Compounds II in which B is 2-chloro-3-(trifluoromethyl)phenyl and A corresponds in each case to one row of Table B (compounds II.125cB-1 to II.125cB-255)

Table 126c
Compounds II in which B is 2-chloro-4-(trifluoromethyl)phenyl and A corresponds in each case to one row of Table B (compounds II.126cB-1 to II.126cB-255)

Table 127c
Compounds II in which B is 2-chloro-5-(trifluoromethyl)phenyl and A corresponds in each case to one row of Table B (compounds II.127cB-1 to II.127cB-255)

Table 128c
Compounds II in which B is 2-(trifluoromethoxy)-3-chlorophenyl and A corresponds in each case to one row of Table B (compounds II.128cB-1 to II.128cB-255)

Table 129c
Compounds II in which B is 2-(trifluoromethoxy)-4-chlorophenyl and A corresponds in each case to one row of Table B (compounds II.129cB-1 to II.129cB-255)

Table 130c
Compounds II in which B is 2-(trifluoromethoxy)-5-chlorophenyl and A corresponds in each case to one row of Table B (compounds II.130cB-1 to II.130cB-255)

Table 131c
Compounds II in which B is 2-(trifluoromethoxy)-6-chlorophenyl and A corresponds in each case to one row of Table B (compounds II.131cB-1 to II.131cB-255)

Table 132c
Compounds II in which B is 2-chloro-3-(trifluoromethoxy)phenyl and A corresponds in each case to one row of Table B (compounds II.132cB-1 to II.132cB-255)

Table 133c
Compounds II in which B is 2-chloro-4-(trifluoromethoxy)phenyl and A corresponds in each case to one row of Table B (compounds II.133cB-1 to II.133cB-255)

Table 134c
Compounds II in which B is 2-chloro-5-(trifluoromethoxy)phenyl and A corresponds in each case to one row of Table B (compounds II.134cB-1 to II.134cB-255)

Table 135c
Compounds II in which B is 2-(difluoromethoxy)-3-chlorophenyl and A corresponds in each case to one row of Table B (compounds II.135cB-1 to II.135cB-255)

Table 136c
Compounds II in which B is 2-(difluoromethoxy)-4-chlorophenyl and A corresponds in each case to one row of Table B (compounds II.136cB-1 to II.136cB-255)

Table 137c
Compounds II in which B is 2-(difluoromethoxy)-5-chlorophenyl and A corresponds in each case to one row of Table B (compounds II.137cB-1 to II.137cB-255)

Table 138c
Compounds II in which B is 2-(difluoromethoxy)-6-chlorophenyl and A corresponds in each case to one row of Table B (compounds II.138cB-1 to II.138cB-255)

Table 139c
Compounds II in which B is 3-(difluoromethoxy)-4-chlorophenyl and A corresponds in each case to one row of Table B (compounds II.139cB-1 to II.139cB-255)

Table 140c
Compounds II in which B is 2-chloro-3-(difluoromethoxy)phenyl and A corresponds in each case to one row of Table B (compounds II.140cB-1 to II.140cB-255)

Table 141c
Compounds II in which B is 2-chloro-4-(difluoromethoxy)phenyl and A corresponds in each case to one row of Table B (compounds II.141cB-1 to II.141cB-255)

Table 142c
Compounds II in which B is 2-chloro-5-(difluoromethoxy)phenyl and A corresponds in each case to one row of Table B (compounds II.142cB-1 to II.142cB-255)

Table 143c
Compounds II in which B is 2-(trifluoromethylthio)-3-chlorophenyl and A corresponds in each case to one row of Table B (compounds II.143cB-1 to II.143cB-255)

Table 144c
Compounds II in which B is 2-(trifluoromethylthio)-4-chlorophenyl and A corresponds in each case to one row of Table B (compounds II.144cB-1 to II.144cB-255)

Table 145c
Compounds II in which B is 2-(trifluoromethylthio)-5-chlorophenyl and A corresponds in each case to one row of Table B (compounds II.145cB-1 to II.145cB-255)

Table 146c
Compounds II in which B is 2-(trifluoromethylthio)-6-chlorophenyl and A corresponds in each case to one row of Table B (compounds II.146cB-1 to II.146cB-255)

Table 147c
Compounds II in which B is 2-chloro-3-(trifluoromethylthio)phenyl and A corresponds in each case to one row of Table B (compounds II.147cB-1 to II.147cB-255)

Table 148c
Compounds II in which B is 2-chloro-4-(trifluoromethylthio)phenyl and A corresponds in each case to one row of Table B (compounds II.148cB-1 to II.148cB-255)

Table 149c
Compounds II in which B is 2-chloro-5-(trifluoromethylthio)phenyl and A corresponds in each case to one row of Table B (compounds II.149cB-1 to II.149cB-255)

Table 150c
Compounds II in which B is 2,3,4-trichlorophenyl and A corresponds in each case to one row of Table B (compounds II.150cB-1 to II.150cB-255)

Table 151c
Compounds II in which B is 2,3,5-trichlorophenyl and A corresponds in each case to one row of Table B (compounds II.151cB-1 to II.151cB-255)

Table 152c
Compounds II in which B is 2,3,6-trichlorophenyl and A corresponds in each case to one row of Table B (compounds II.152cB-1 to II.152cB-255)

Table 153c
Compounds II in which B is 2,4,5-trichlorophenyl and A corresponds in each case to one row of Table B (compounds II.153cB-1 to II.153cB-255)

Table 154c
Compounds II in which B is 2,4,6-trichlorophenyl and A corresponds in each case to one row of Table B (compounds II.154cB-1 to II.154cB-255)

Table 155c
Compounds II in which B is 2,3,4-trimethylphenyl and A corresponds in each case to one group of one row of Table B (compounds II.155cB-1 to II.155cB-255)

Table 156c
Compounds II in which B is 2,3,5-trimethylphenyl and A corresponds in each case to one group of one row of Table B (compounds II.156cB-1 to II.156cB-255)

Table 162c
Compounds II in which B is 2,3,6-trimethoxyphenyl and A corresponds in each case to one group of one row of Table B (compounds II.162cB-1 to II.162cB-255)

TABLE B

| Row | A |
|---|---|
| B-1 | Phenyl |
| B-2 | 2-Chlorophenyl |
| B-3 | 3-Chlorophenyl |
| B-4 | 4-Chlorophenyl |
| B-5 | 2-Fluorophenyl |
| B-6 | 3-Fluorophenyl |
| B-7 | 4-Fluorophenyl |
| B-8 | 2-Methylphenyl |
| B-9 | 3-Methylphenyl |
| B-10 | 4-Methylphenyl |
| B-11 | 2-Ethylphenyl |
| B-12 | 3-Ethylphenyl |
| B-13 | 4-Ethylphenyl |
| B-14 | 2-Methoxyphenyl |
| B-15 | 3-Methoxyphenyl |
| B-16 | 4-Methoxyphenyl |
| B-17 | 2-Trifluoromethylphenyl |
| B-18 | 3-Trifluoromethylphenyl |
| B-19 | 4-Trifluoromethylphenyl |
| B-20 | 2-Trifluoromethoxyphenyl |
| B-21 | 3-Trifluoromethoxyphenyl |
| B-22 | 4-Trifluoromethoxyphenyl |
| B-23 | 2-Difluoromethoxyphenyl |
| B-24 | 3-Difluoromethoxyphenyl |
| B-25 | 4-Difluoromethoxyphenyl |
| B-26 | 2-Trifluoromethylthiophenyl |
| B-27 | 3-Trifluoromethylthiophenyl |
| B-28 | 4-Trifluoromethylthiophenyl |
| B-29 | 2,3-Dichlorophenyl |
| B-30 | 2,4-Dichlorophenyl |
| B-31 | 2,5-Dichlorophenyl |
| B-32 | 2,6-Dichlorophenyl |
| B-33 | 3,4-Dichlorophenyl |
| B-34 | 3,5-Dichlorophenyl |
| B-35 | 2,3-Difluorophenyl |
| B-36 | 2,4-Difluorophenyl |
| B-37 | 2,5-Difluorophenyl |
| B-38 | 2,6-Difluorophenyl |
| B-39 | 3,4-Difluorophenyl |
| B-40 | 3,5-Difluorophenyl |
| B-41 | 2,3-Dimethylphenyl |
| B-42 | 2,4-Dimethylphenyl |
| B-43 | 2,5-Dimethylphenyl |
| B-44 | 2,6-Dimethylphenyl |
| B-45 | 3,4-Dimethylphenyl |
| B-46 | 3,5-Dimethylphenyl |
| B-47 | 2,3-Diethylphenyl |
| B-48 | 2,4-Diethylphenyl |
| B-49 | 2,5-Diethylphenyl |
| B-50 | 2,6-Diethylphenyl |
| B-51 | 3,5-Diethylphenyl |
| B-52 | 3,4-Diethylphenyl |
| B-53 | 2,3-Dimethoxyphenyl |
| B-54 | 2,4-Dimethoxyphenyl |
| B-55 | 2,5-Dimethoxyphenyl |
| B-56 | 2,6-Dimethoxyphenyl |
| B-57 | 3,4-Dimethoxyphenyl |
| B-58 | 3,5-Dimethoxyphenyl |
| B-59 | 2,3-Di(trifluoromethyl)phenyl |
| B-60 | 2,4-Di(trifluoromethyl)phenyl |
| B-61 | 2,5-Di(trifluoromethyl)phenyl |
| B-62 | 2,6-Di(trifluoromethyl)phenyl |
| B-63 | 3,4-Di(trifluoromethyl)phenyl |
| B-64 | 3,5-Di(trifluoromethyl)phenyl |
| B-65 | 2,3-Di(trifluoromethoxy)phenyl |
| B-66 | 2,4-Di(trifluoromethoxy)phenyl |
| B-67 | 2,5-Di(trifluoromethoxy)phenyl |
| B-68 | 2,6-Di(trifluoromethoxy)phenyl |
| B-69 | 3,4-Di(trifluoromethoxy)phenyl |
| B-70 | 3,5-Di(trifluoromethoxy)phenyl |
| B-71 | 2,3-Di(difluoromethoxy)phenyl |
| B-72 | 2,4-Di(difluoromethoxy)phenyl |
| B-73 | 2,5-Di(difluoromethoxy)phenyl |
| B-74 | 2,6-Di(difluoromethoxy)phenyl |
| B-75 | 3,4-Di(difluoromethoxy)phenyl |
| B-76 | 3,5-Di(difluoromethoxy)phenyl |
| B-77 | 2,3-Di(trifluoromethylthio)phenyl |
| B-78 | 2,4-Di(trifluoromethylthio)phenyl |
| B-79 | 2,5-Di(trifluoromethylthio)phenyl |
| B-80 | 2,6-Di(trifluoromethylthio)phenyl |
| B-81 | 3,4-Di(trifluoromethylthio)phenyl |
| B-82 | 3,5-Di(trifluoromethylthio)phenyl |
| B-83 | 2-Fluoro-3-chlorophenyl |
| B-84 | 2-Fluoro-4-chlorophenyl |
| B-85 | 2-Fluoro-5-chlorophenyl |
| B-86 | 2-Fluoro-6-chlorophenyl |
| B-87 | 3-Fluoro-4-chlorophenyl |
| B-88 | 3-Fluoro-5-chlorophenyl |
| B-89 | 2-Chloro-3-fluorophenyl |
| B-90 | 2-Chloro-4-fluorophenyl |
| B-91 | 2-Chloro-5-fluorophenyl |
| B-92 | 3-Chloro-4-fluorophenyl |
| B-93 | 2-Methyl-3-chlorophenyl |
| B-94 | 2-Methyl-4-chlorophenyl |
| B-95 | 2-Methyl-5-chlorophenyl |
| B-96 | 2-Methyl-6-chlorophenyl |
| B-97 | 3-Methyl-4-chlorophenyl |
| B-98 | 3-Methyl-5-chlorophenyl |
| B-99 | 2-Chloro-3-methylphenyl |
| B-100 | 2-Chloro-4-methylphenyl |
| B-101 | 2-Chloro-5-methylphenyl |
| B-102 | 3-Chloro-4-methylphenyl |
| B-103 | 2-Methyl-3-fluorophenyl |
| B-104 | 2-Methyl-4-fluorophenyl |
| B-105 | 2-Methyl-5-fluorophenyl |
| B-106 | 2-Methyl-6-fluorophenyl |
| B-107 | 3-Methyl-4-fluorophenyl |
| B-108 | 3-Methyl-5-fluorophenyl |
| B-109 | 2-Fluoro-3-methylphenyl |
| B-110 | 2-Fluoro-4-methylphenyl |
| B-111 | 2-Fluoro-5-methylphenyl |
| B-112 | 3-Fluoro-4-methylphenyl |
| B-113 | 2-Chloro-3-ethylphenyl |
| B-114 | 2-Chloro-4-ethylphenyl |
| B-115 | 2-Chloro-5-ethylphenyl |
| B-116 | 3-Chloro-4-ethylphenyl |
| B-117 | 2-Ethyl-3-chlorophenyl |
| B-118 | 2-Ethyl-4-chlorophenyl |
| B-119 | 2-Ethyl-5-chlorophenyl |
| B-120 | 2-Ethyl-6-chlorophenyl |
| B-121 | 2-Ethyl-3-fluorophenyl |
| B-122 | 2-Ethyl-4-fluorophenyl |
| B-123 | 2-Ethyl-5-fluorophenyl |
| B-124 | 2-Ethyl-6-fluorophenyl |
| B-125 | 3-Ethyl-4-fluorophenyl |
| B-126 | 3-Ethyl-5-fluorophenyl |
| B-127 | 2-Fluoro-3-ethylphenyl |
| B-128 | 2-Fluoro-4-ethylphenyl |
| B-129 | 2-Fluoro-5-ethylphenyl |
| B-130 | 3-Fluoro-4-ethylphenyl |
| B-131 | 2-Methoxy-3-chlorophenyl |
| B-132 | 2-Methoxy-4-chlorophenyl |
| B-133 | 2-Methoxy-5-chlorophenyl |
| B-134 | 2-Methoxy-6-chlorophenyl |
| B-135 | 3-Methoxy-4-chlorophenyl |
| B-136 | 3-Methoxy-5-chlorophenyl |
| B-137 | 2-Chloro-3-methoxyphenyl |
| B-138 | 2-Chloro-4-methoxyphenyl |
| B-139 | 2-Chloro-5-methoxyphenyl |
| B-140 | 3-Chloro-4-methoxyphenyl |
| B-141 | 2-Methoxy-3-fluorophenyl |
| B-142 | 2-Methoxy-4-fluorophenyl |
| B-143 | 2-Methoxy-5-fluorophenyl |
| B-144 | 2-Methoxy-6-fluorophenyl |
| B-145 | 3-Methoxy-4-fluorophenyl |
| B-146 | 3-Methoxy-5-fluorophenyl |
| B-147 | 2-Fluoro-3-methoxyphenyl |
| B-148 | 2-Fluoro-4-methoxyphenyl |
| B-149 | 2-Fluoro-5-methoxyphenyl |
| B-150 | 3-Fluoro-4-methoxyphenyl |
| B-151 | 3-Fluoro-5-methoxyphenyl |
| B-152 | 2-(Trifluoromethyl)-3-chlorophenyl |
| B-153 | 2-(Trifluoromethyl)-4-chlorophenyl |
| B-154 | 2-(Trifluoromethyl)-5-chlorophenyl |
| B-155 | 2-(Trifluoromethyl)-6-chlorophenyl |
| B-156 | 3-(Trifluoromethyl)-4-chlorophenyl |
| B-157 | 3-(Trifluoromethyl)-5-chlorophenyl |
| B-158 | 2-Chloro-3-(trifluoromethyl)phenyl |
| B-159 | 2-Chloro-4-(trifluoromethyl)phenyl |
| B-160 | 2-Chloro-5-(trifluoromethyl)phenyl |
| B-161 | 3-Chloro-4-(trifluoromethyl)phenyl |
| B-162 | 2-(Trifluoromethyl)-3-fluorophenyl |
| B-163 | 2-(Trifluoromethyl)-4-fluorophenyl |
| B-164 | 2-(Trifluoromethyl)-5-fluorophenyl |
| B-165 | 2-(Trifluoromethyl)-6-fluorophenyl |
| B-166 | 3-(Trifluoromethyl)-4-fluorophenyl |
| B-167 | 3-(Trifluoromethyl)-5-fluorophenyl |
| B-168 | 2-Fluoro-3-(trifluoromethyl)phenyl |
| B-169 | 2-Fluoro-4-(trifluoromethyl)phenyl |
| B-170 | 2-Fluoro-5-(trifluoromethyl)phenyl |
| B-171 | 3-Fluoro-4-(trifluoromethyl)phenyl |
| B-172 | 2-(Trifluoromethoxy)-3-chlorophenyl |
| B-173 | 2-(Trifluoromethoxy)-4-chlorophenyl |
| B-174 | 2-(Trifluoromethoxy)-5-chlorophenyl |
| B-175 | 2-(Trifluoromethoxy)-6-chlorophenyl |
| B-176 | 3-(Trifluoromethoxy)-4-chlorophenyl |
| B-177 | 3-(Trifluoromethoxy)-5-chlorophenyl |
| B-178 | 2-Chloro-3-(trifluoromethoxy)phenyl |
| B-179 | 2-Chloro-4-(trifluoromethoxy)phenyl |
| B-180 | 2-Chloro-5-(trifluoromethoxy)phenyl |
| B-181 | 3-Chloro-4-(trifluoromethoxy)phenyl |
| B-182 | 2-(Trifluoromethoxy)-3-fluorophenyl |
| B-183 | 2-(Trifluoromethoxy)-4-fluorophenyl |
| B-184 | 2-(Trifluoromethoxy)-5-fluorophenyl |
| B-185 | 2-(Trifluoromethoxy)-6-fluorophenyl |
| B-186 | 3-(Trifluoromethoxy)-4-fluorophenyl |
| B-187 | 3-(Trifluoromethoxy)-5-fluorophenyl |
| B-188 | 2-Fluoro-3-(trifluoromethoxy)phenyl |
| B-189 | 2-Fluoro-4-(trifluoromethoxy)phenyl |
| B-190 | 2-Fluoro-5-(trifluoromethoxy)phenyl |
| B-191 | 3-Fluoro-4-(trifluoromethoxy)phenyl |
| B-192 | 2-(Difluoromethoxy)-3-chlorophenyl |
| B-193 | 2-(Difluoromethoxy)-4-chlorophenyl |
| B-194 | 2-(Difluoromethoxy)-5-chlorophenyl |
| B-195 | 2-(Difluoromethoxy)-6-chlorophenyl |
| B-196 | 3-(Difluoromethoxy)-4-chlorophenyl |
| B-197 | 3-(Difluoromethoxy)-5-chlorophenyl |
| B-198 | 2-Chloro-3-(difluoromethoxy)phenyl |
| B-199 | 2-Chloro-4-(difluoromethoxy)phenyl |
| B-200 | 2-Chloro-5-(difluoromethoxy)phenyl |
| B-201 | 3-Chloro-4-(difluoromethoxy)phenyl |
| B-202 | 2-(Difluoromethoxy)-3-fluorophenyl |
| B-203 | 2-(Difluoromethoxy)-4-fluorophenyl |
| B-204 | 2-(Difluoromethoxy)-5-fluorophenyl |
| B-205 | 2-(Difluoromethoxy)-6-fluorophenyl |
| B-206 | 3-(Difluoromethoxy)-4-fluorophenyl |
| B-207 | 3-(Difluoromethoxy)-5-fluorophenyl |
| B-208 | 2-Fluoro-3-(difluoromethoxy)phenyl |
| B-209 | 2-Fluoro-4-(difluoromethoxy)phenyl |
| B-210 | 2-Fluoro-5-(difluoromethoxy)phenyl |
| B-211 | 3-Fluoro-4-(difluoromethoxy)phenyl |
| B-212 | 2-(Trifluoromethylthio)-3-chlorophenyl |
| B-213 | 2-(Trifluoromethylthio)-4-chlorophenyl |
| B-214 | 2-(Trifluoromethylthio)-5-chlorophenyl |
| B-215 | 2-(Trifluoromethylthio)-6-chlorophenyl |
| B-216 | 3-(Trifluoromethylthio)-4-chlorophenyl |
| B-217 | 3-(Trifluoromethylthio)-5-chlorophenyl |
| B-218 | 2-Chloro-3-(trifluoromethylthio)phenyl |
| B-219 | 2-Chloro-4-(trifluoromethylthio)phenyl |
| B-220 | 2-Chloro-5-(trifluoromethylthio)phenyl |
| B-221 | 3-Chloro-4-(trifluoromethylthio)phenyl |
| B-222 | 2-(Trifluoromethylthio)-3-fluorophenyl |
| B-223 | 2-(Trifluoromethylthio)-4-fluorophenyl |
| B-224 | 2-(Trifluoromethylthio)-5-fluorophenyl |
| B-225 | 2-(Trifluoromethylthio)-6-fluorophenyl |
| B-226 | 3-(Trifluoromethylthio)-4-fluorophenyl |
| B-227 | 3-(Trifluoromethylthio)-5-fluorophenyl |
| B-228 | 2-Fluoro-3-(trifluoromethylthio)phenyl |
| B-229 | 2-Fluoro-4-(trifluoromethylthio)phenyl |
| B-230 | 2-Fluoro-5-(trifluoromethylthio)phenyl |
| B-231 | 3-Fluoro-4-(trifluoromethylthio)phenyl |
| B-232 | 2,3,4-Trichlorophenyl |
| B-233 | 2,3,5-Trichlorophenyl |
| B-234 | 2,3,6-Trichlorophenyl |
| B-235 | 2,4,5-Trichlorophenyl |
| B-236 | 2,4,6-Trichlorophenyl |
| B-237 | 3,4,5-Trichlorophenyl |
| B-238 | 2,3,4-Trifluorophenyl |
| B-239 | 2,3,5-Trifluorophenyl |
| B-240 | 2,3,6-Trifluorophenyl |
| B-241 | 2,4,5-Trifluorophenyl |
| B-242 | 2,4,6-Trifluorophenyl |
| B-243 | 3,4,5-Trifluorophenyl |
| B-244 | 2,3,4-Trimethylphenyl |
| B-245 | 2,3,5-Trimethylphenyl |
| B-246 | 2,3,6-Trimethylphenyl |
| B-247 | 2,4,5-Trimethylphenyl |
| B-248 | 2,4,6-Trimethylphenyl |
| B-249 | 3,4,5-Trimethylphenyl |
| B-250 | 2,3,4-Trimethoxyphenyl |
| B-251 | 2,3,5-Trimethoxyphenyl |
| B-252 | 2,3,6-Trimethoxyphenyl |
| B-253 | 2,4,5-Trimethoxyphenyl |
| B-254 | 2,4,6-Trimethoxyphenyl |
| B-255 | 3,4,5-Trimethoxyphenyl |

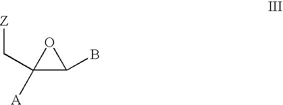

In particular with a view to their use, preference is given to the compounds III compiled in Tables 1d to 162d below. The groups mentioned in the tables for a substituent are furthermore per se, independently of the combination in which they are mentioned, a particularly preferred embodiment of the substituent in question.

Table 1d
Compounds III in which B is 2,3-difluorophenyl and the combination of A and Z corresponds in each case to one row of Table C (compounds III.1dC-1 to III.1dC-1275)

Table 2d
Compounds III in which B is 2,4-difluorophenyl and the combination of A and Z corresponds in each case to one row of Table C (compounds III.2dC-1 to III.2dC-1275)

Table 3d
Compounds III in which B is 2,5-difluorophenyl and the combination of A and Z corresponds in each case to one row of Table C (compounds III.3dC-1 to III.3dC-1275)

Table 4d
Compounds III in which B is 2,6-difluorophenyl and the combination of A and Z corresponds in each case to one row of Table C (compounds III.4dC-1 to III.4dC-1275)

Table 5d
Compounds III in which B is 2-fluoro-3-chlorophenyl and the combination of A and Z corresponds in each case to one row of Table C (compounds III.5dC-1 to III.5dC-1275)

Table 6d
Compounds III in which B is 2-fluoro-4-chlorophenyl and the combination of A and Z corresponds in each case to one row of Table C (compounds III.6dC-1 to III.6dC-1275)

Table 7d
Compounds III in which B is 2-fluoro-5-chlorophenyl and the combination of A and Z corresponds in each case to one row of Table C (compounds III.7dC-1 to III.7dC-1275)

Table 8d
Compounds III in which B is 2-fluoro-6-chlorophenyl and the combination of A and Z corresponds in each case to one row of Table C (compounds III.8dC-1 to III.8dC-1275)

Table 9d
Compounds III in which B is 2-chloro-3-fluorophenyl and the combination of A and Z corresponds in each case to one row of Table C (compounds III.9dC-1 to III.9dC-1275)

Table 10d
Compounds III in which B is 2-chloro-4-fluorophenyl and the combination of A and Z corresponds in each case to one row of Table C (compounds III.10dC-1 to III.10dC-1275)

Table 11d
Compounds III in which B is 2-chloro-5-fluorophenyl and the combination of A and Z corresponds in each case to one row of Table C (compounds III.11dC-1 to III.11dC-1275)

Table 12d
Compounds III in which B is 2-methyl-3-fluorophenyl and the combination of A and Z corresponds in each case to one row of Table C (compounds III.12dC-1 to III.12dC-1275)

Table 13d
Compounds III in which B is 2-methyl-4-fluorophenyl and the combination of A and Z corresponds in each case to one row of Table C (compounds III.13dC-1 to III.13dC-1275)

Table 14d
Compounds III in which B is 2-methyl-5-fluorophenyl and the combination of A and Z corresponds in each case to one row of Table C (compounds III.14dC-1 to III.14dC-1275)

Table 15d
Compounds III in which B is 2-methyl-6-fluorophenyl and the combination of A and Z corresponds in each case to one row of Table C (compounds III.15dC-1 to III.15dC-1275)

Table 16d
Compounds III in which B is 2-fluoro-3-methylphenyl and the combination of A and Z corresponds in each case to one row of Table C (compounds III.16dC-1 to III.16dC-1275)

Table 17d
Compounds III in which B is 2-fluoro-4-methylphenyl and the combination of A and Z corresponds in each case to one row of Table C (compounds III.17dC-1 to III.17dC-1275)

Table 18d
Compounds III in which B is 2-fluoro-5-methylphenyl and the combination of A and Z corresponds in each case to one row of Table C (compounds III.18dC-1 to III.18dC-1275)

Table 19d
Compounds III in which B is 2-ethyl-3-fluorophenyl and the combination of A and Z corresponds in each case to one row of Table C (compounds III.19dC-1 to III.19dC-1275)

Table 20d
Compounds III in which B is 2-ethyl-4-fluorophenyl and the combination of A and Z corresponds in each case to one row of Table C (compounds III.20dC-1 to III.20dC-1275)

Table 21d
Compounds III in which B is 2-ethyl-5-fluorophenyl and the combination of A and Z corresponds in each case to one row of Table C (compounds III.21dC-1 to III.21dC-1275)

Table 22d
Compounds III in which B is 2-ethyl-6-fluorophenyl and the combination of A and Z corresponds in each case to one row of Table C (compounds III.22dC-1 to III.22dC-1275)

Table 23d
Compounds III in which B is 2-fluoro-3-ethylphenyl and the combination of A and Z corresponds in each case to one row of Table C (compounds III.23dC-1 to III.23dC-1275)

Table 24d
Compounds III in which B is 2-fluoro-4-ethylphenyl and the combination of A and Z corresponds in each case to one row of Table C (compounds III.24dC-1 to III.24dC-1275)

Table 25d
Compounds III in which B is 2-methoxy-3-fluorophenyl and the combination of A and Z corresponds in each case to one row of Table C (compounds III.25dC-1 to III.25dC-1275)

Table 26d
Compounds III in which B is 2-methoxy-4-fluorophenyl and the combination of A and Z corresponds in each case to one row of Table C (compounds III.26dC-1 to III.26dC-1275)

Table 27d
Compounds III in which B is 2-methoxy-5-fluorophenyl and the combination of A and Z corresponds in each case to one row of Table C (compounds III.27dC-1 to III.27dC-1275)

Table 28d
Compounds III in which B is 2-methoxy-6-fluorophenyl and the combination of A and Z corresponds in each case to one row of Table C (compounds III.28dC-1 to III.28dC-1275)

Table 29d
Compounds III in which B is 2-fluoro-3-methoxyphenyl and the combination of A and Z corresponds in each case to one row of Table C (compounds III.29dC-1 to III.29dC-1275)

Table 30d
Compounds III in which B is 2-fluoro-4-methoxyphenyl and the combination of A and Z corresponds in each case to one row of Table C (compounds III.30dC-1 to III.30dC-1275)

Table 31d
Compounds III in which B is 2-fluoro-5-methoxyphenyl and the combination of A and Z corresponds in each case to one row of Table C (compounds III.31dC-1 to III.31dC-1275)

Table 32d
Compounds III in which B is 2-(trifluoromethyl)-3-fluorophenyl and the combination of A and Z corresponds in each case to one row of Table C (compounds III.32dC-1 to III.32dC-1275)

Table 33d
Compounds III in which B is 2,4,5-trimethoxyphenyl and the combination of A and Z corresponds in each case to one row of Table C (compounds III.33dC-1 to III.33dC-1275)

Table 34d
Compounds III in which B is 2,4,6-trimethoxyphenyl and the combination of A and Z corresponds in each case to one row of Table C (compounds III.34dC-1 to III.34dC-1275)

Table 35d
Compounds III in which B is 2-fluoro-5-ethylphenyl and the combination of A and Z corresponds in each case to one row of Table C (compounds III.35dC-1 to III.35dC-1275)

Table 36d
Compounds III in which B is 2-(trifluoromethyl)-4-fluorophenyl and the combination of A and Z corresponds in each case to one row of Table C (compounds III.36dC-1 to III.36dC-1275)

Table 37d
Compounds III in which B is 2-(trifluoromethyl)-5-fluorophenyl and the combination of A and Z corresponds in each case to one row of Table C (compounds III.37dC-1 to III.37dC-1275)

Table 38d
Compounds III in which B is 2-(trifluoromethyl)-6-fluorophenyl and the combination of A and Z corresponds in each case to one row of Table C (compounds III.38dC-1 to III.38dC-1275)

Table 39d
Compounds III in which B is 2-fluoro-3-(trifluoromethyl)phenyl and the combination of A and Z corresponds in each case to one row of Table C (compounds III.39dC-1 to III.39dC-1275)

Table 40d
Compounds III in which B is 2-fluoro-4-(trifluoromethyl)phenyl and the combination of A and Z corresponds in each case to one row of Table C (compounds III.40dC-1 to III.40dC-1275)

Table 41d
Compounds III in which B is 2-fluoro-5-(trifluoromethyl)phenyl and the combination of A and Z corresponds in each case to one row of Table C (compounds III.41dC-1 to III.41dC-1275)

Table 42d
Compounds III in which B is 2-(trifluoromethoxy)-3-fluorophenyl and the combination of A and Z corresponds in each case to one row of Table C (compounds III.42dC-1 to III.42dC-1275)

Table 43d
Compounds III in which B is 2-(trifluoromethoxy)-4-fluorophenyl and the combination of A and Z corresponds in each case to one row of Table C (compounds III.43dC-1 to III.43dC-1275)

Table 44d
Compounds III in which B is 2-(trifluoromethoxy)-5-fluorophenyl and the combination of A and Z corresponds in each case to one row of Table C (compounds III.44dC-1 to III.44dC-1275)

Table 45d
Compounds III in which B is 2-(trifluoromethoxy)-6-fluorophenyl and the combination of A and Z corresponds in each case to one row of Table C (compounds III.45dC-1 to III.45dC-1275)

Table 46d
Compounds III in which B is 2-fluoro-3-(trifluoromethoxy)phenyl and the combination of A and Z corresponds in each case to one row of Table C (compounds III.46dC-1 to III.46dC-1275)

Table 47d
Compounds III in which B is 2-fluoro-4-(trifluoromethoxy)phenyl and the combination of A and Z corresponds in each case to one row of Table C (compounds III.47dC-1 to III.47dC-1275)

Table 48d
Compounds III in which B is 2-fluoro-5-(trifluoromethoxy)phenyl and the combination of A and Z corresponds in each case to one row of Table C (compounds III.48dC-1 to III.48dC-1275)

Table 49d
Compounds III in which B is 2-(difluoromethoxy)-3-fluorophenyl and the combination of A and Z corresponds in each case to one row of Table C (compounds III.49dC-1 to III.49dC-1275)

Table 50d
Compounds III in which B is 2-(difluoromethoxy)-4-fluorophenyl and the combination of A and Z corresponds in each case to one row of Table C (compounds III.50dC-1 to III.50dC-1275)

Table 51d
Compounds III in which B is 2-(difluoromethoxy)-5-fluorophenyl and the combination of A and Z corresponds in each case to one row of Table C (compounds III.51dC-1 to III.51dC-1275)

Table 52d
Compounds III in which B is 2-(difluoromethoxy)-6-fluorophenyl and the combination of A and Z corresponds in each case to one row of Table C (compounds III.52dC-1 to III.52dC-1275)

Table 53d
Compounds III in which B is 2-fluoro-3-(difluoromethoxy)phenyl and the combination of A and Z corresponds in each case to one row of Table C (compounds III.53dC-1 to III.53dC-1275)

Table 54d
Compounds III in which B is 2-fluoro-4-(difluoromethoxy)phenyl and the combination of A and Z corresponds in each case to one row of Table C (compounds III.54dC-1 to III.54dC-1275)

Table 55d
Compounds III in which B is 2-fluoro-5-(difluoromethoxy)phenyl and the combination of A and Z corresponds in each case to one row of Table C (compounds III.55dC-1 to III.55dC-1275)

Table 56d
Compounds III in which B is 2-(trifluoromethylthio)-3-fluorophenyl and the combination of A and Z corresponds in each case to one row of Table C (compounds III.56dC-1 to III.56dC-1275)

Table 57d
Compounds III in which B is 2-(trifluoromethylthio)-4-fluorophenyl and the combination of A and Z corresponds in each case to one row of Table C (compounds III.57dC-1 to III.57dC-1275)

Table 58d
Compounds III in which B is 2-(trifluoromethylthio)-5-fluorophenyl and the combination of A and Z corresponds in each case to one row of Table C (compounds III.58dC-1 to III.58dC-1275)

Table 59d
Compounds III in which B is 2-(trifluoromethylthio)-6-fluorophenyl and the combination of A and Z corresponds in each case to one row of Table C (compounds III.59dC-1 to III.59dC-1275)

Table 60d
Compounds III in which B is 2-fluoro-3-(trifluoromethylthio)phenyl and the combination of A and Z corresponds in each case to one row of Table C (compounds III.60dC-1 to III.60dC-1275)

Table 61d
Compounds III in which B is 2-fluoro-4-(trifluoromethylthio)phenyl and the combination of A and Z corresponds in each case to one row of Table C (compounds III.61dC-1 to III.61dC-1275)

Table 62d
Compounds III in which B is 2-fluoro-5-(trifluoromethylthio)phenyl and the combination of A and Z corresponds in each case to one row of Table C (compounds III.62dC-1 to III.62dC-1275)

Table 63d
Compounds III in which B is 2,3,4-trifluorophenyl and the combination of A and Z corresponds in each case to one row of Table C (compounds III.63dC-1 to III.63dC-1275)

Table 64d
Compounds III in which B is 2,3,5-trifluorophenyl and the combination of A and Z corresponds in each case to one row of Table C (compounds III.64dC-1 to III.64dC-1275)

Table 65d
Compounds III in which B is 2,3,6-trifluorophenyl and the combination of A and Z corresponds in each case to one row of Table C (compounds III.65dC-1 to III.65dC-1275)

Table 66d
Compounds III in which B is 2,4,5-trifluorophenyl and the combination of A and Z corresponds in each case to one row of Table C (compounds III.66dC-1 to III.66dC-1275)

Table 67d
Compounds III in which B is 2,4,6-trifluorophenyl and the combination of A and Z corresponds in each case to one row of Table C (compounds III.67dC-1 to III.67dC-1275)

Table 68d
Compounds III in which B is 2,3-dichlorophenyl and the combination of A and Z corresponds in each case to one row of Table C (compounds III.68dC-1 to III.68dC-1275)

Table 69d
Compounds III in which B is 2,4-dichlorophenyl and the combination of A and Z corresponds in each case to one row of Table C (compounds III.69dC-1 to III.69dC-1275)

Table 70d
Compounds III in which B is 2,5-dichlorophenyl and the combination of A and Z corresponds in each case to one row of Table C (compounds III.70dC-1 to III.70dC-1275)

Table 71d
Compounds III in which B is 2,6-dichlorophenyl and the combination of A and Z corresponds in each case to one row of Table C (compounds III.71dC-1 to III.71dC-1275)

Table 72d
Compounds III in which B is 2,3-dimethylphenyl and the combination of A and Z corresponds in each case to one row of Table C (compounds III.72dC-1 to III.72dC-1275)

Table 73d
Compounds III in which B is 2,4-dimethylphenyl and the combination of A and Z corresponds in each case to one row of Table C (compounds III.73dC-1 to III.73dC-1275)

Table 74d
Compounds III in which B is 2,5-dimethylphenyl and the combination of A and Z corresponds in each case to one row of Table C (compounds III.74dC-1 to III.74dC-1275)

Table 75d
Compounds III in which B is 2,6-dimethylphenyl and the combination of A and Z corresponds in each case to one row of Table C (compounds III.75dC-1 to III.75dC-1275)

Table 76d
Compounds III in which B is 2,3-diethylphenyl and the combination of A and Z corresponds in each case to one row of Table C (compounds III.76dC-1 to III.76dC-1275)

Table 77d
Compounds III in which B is 2,4-diethylphenyl and the combination of A and Z corresponds in each case to one row of Table C (compounds III.77dC-1 to III.77dC-1275)

Table 78d
Compounds III in which B is 2,5-diethylphenyl and the combination of A and Z corresponds in each case to one row of Table C (compounds III.78dC-1 to III.78dC-1275)

Table 79d
Compounds III in which B is 2,6-diethylphenyl and the combination of A and Z corresponds in each case to one row of Table C (compounds III.79dC-1 to III.79dC-1275)

Table 80d
Compounds III in which B is 2,3-dimethoxyphenyl and the combination of A and Z corresponds in each case to one row of Table C (compounds III.80dC-1 to III.80dC-1275)

Table 81d
Compounds III in which B is 2,4-dimethoxyphenyl and the combination of A and Z corresponds in each case to one row of Table C (compounds III.81dC-1 to II.81dC-1275)

Table 82d
Compounds III in which B is 2,5-dimethoxyphenyl and the combination of A and Z corresponds in each case to one row of Table C (compounds III.82dC-1 to III.82dC-1275)

Table 83d
Compounds III in which B is 2,6-dimethoxyphenyl and the combination of A and Z corresponds in each case to one row of Table C (compounds III.83dC-1 to III.83dC-1275)

Table 84d
Compounds III in which B is 2,3-di(trifluoromethyl)phenyl and the combination of A and Z corresponds in each case to one row of Table C (compounds III.84dC-1 to III.84dC-1275)

Table 85d
Compounds III in which B is 2,4-di(trifluoromethyl)phenyl and the combination of A and Z corresponds in each case to one row of Table C (compounds III.85dC-1 to III.85dC-1275)

Table 86d
Compounds III in which B is 2,5-di(trifluoromethyl)phenyl and the combination of A and Z corresponds in each case to one row of Table C (compounds III.86dC-1 to III.86dC-1275)

Table 87d
Compounds III in which B is 2,6-di(trifluoromethyl)phenyl and the combination of A and Z corresponds in each case to one row of Table C (compounds III.87dC-1 to III.87dC-1275)

Table 88d
Compounds III in which B is 2,3-di(trifluoromethoxy)phenyl and the combination of A and Z corresponds in each case to one row of Table C (compounds III.88dC-1 to III.88dC-1275)

Table 89d
Compounds III in which B is 2,4-di(trifluoromethoxy)phenyl and the combination of A and Z corresponds in each case to one row of Table C (compounds III.89dC-1 to III.89dC-1275)

Table 90d
Compounds III in which B is 2,5-di(trifluoromethoxy)phenyl and the combination of A and Z corresponds in each case to one row of Table C (compounds III.90dC-1 to III.90dC-1275)

Table 91d
Compounds III in which B is 2,6-di(trifluoromethoxy)phenyl and the combination of A and Z corresponds in each case to one row of Table C (compounds III.91dC-1 to III.91dC-1275)

Table 92d
Compounds III in which B is 2,3-di(difluoromethoxy)phenyl and the combination of A and Z corresponds in each case to one row of Table C (compounds III.92dC-1 to III.92dC-1275)

Table 93d
Compounds III in which B is 2,4-di(difluoromethoxy)phenyl and the combination of A and Z corresponds in each case to one row of Table C (compounds III.93dC-1 to III.93dC-1275)

Table 94d
Compounds III in which B is 2,5-di(difluoromethoxy)phenyl and the combination of A and Z corresponds in each case to one row of Table C (compounds III.94dC-1 to III.94dC-1275)

Table 95d
Compounds III in which B is 2,6-di(difluoromethoxy)phenyl and the combination of A and Z corresponds in each case to one row of Table C (compounds III.95dC-1 to III.95dC-1275)

Table 96d
Compounds III in which B is 2,3-di(trifluoromethylthio)phenyl and the combination of A and Z corresponds in each case to one row of Table C (compounds III.96dC-1 to III.96dC-1275)

Table 97d
Compounds III in which B is 2,4-di(trifluoromethylthio)phenyl and the combination of A and Z corresponds in each case to one row of Table C (compounds III.97dC-1 to III.97dC-1275)

Table 98d
Compounds III in which B is 2,5-di(trifluoromethylthio)phenyl and the combination of A and Z corresponds in each case to one row of Table C (compounds III.98dC-1 to III.98dC-1275)

Table 99d
Compounds III in which B is 2,6-di(trifluoromethylthio)phenyl and the combination of A and Z corresponds in each case to one row of Table C (compounds III.99dC-1 to III.99dC-1275)

Table 100d
Compounds III in which B is 2-methyl-3-chlorophenyl and the combination of A and Z corresponds in each case to one row of Table C (compounds III.100dC-1 to III.100dC-1275)

Table 101d
Compounds III in which B is 2-methyl-4-chlorophenyl and the combination of A and Z corresponds in each case to one row of Table C (compounds III.101dC-1 to III.101dC-1275)

Table 102d
Compounds III in which B is 2-methyl-5-chlorophenyl and the combination of A and Z corresponds in each case to one row of Table C (compounds III.102dC-1 to III.102dC-1275)

Table 103d
Compounds III in which B is 2-methyl-6-chlorophenyl and the combination of A and Z corresponds in each case to one row of Table C (compounds III.103dC-1 to III.103dC-1275)

Table 104d
Compounds III in which B is 2-chloro-3-methylphenyl and the combination of A and Z corresponds in each case to one row of Table C (compounds III.104dC-1 to III.104dC-1275)

Table 105d
Compounds III in which B is 2-chloro-4-methylphenyl and the combination of A and Z corresponds in each case to one row of Table C (compounds III.105dC-1 to III.105dC-1275)

Table 106d
Compounds III in which B is 2-chloro-5-methylphenyl and the combination of A and Z corresponds in each case to one row of Table C (compounds III.106dC-1 to III.106dC-1275)

Table 107d
Compounds III in which B is 2-chloro-3-ethylphenyl and the combination of A and Z corresponds in each case to one row of Table C (compounds III.107dC-1 to III.107dC-1275)

Table 108d
Compounds III in which B is 2-chloro-4-ethylphenyl and the combination of A and Z corresponds in each case to one row of Table C (compounds III.108dC-1 to III.108dC-1275)

Table 109d
Compounds III in which B is 2-chloro-5-ethylphenyl and the combination of A and Z corresponds in each case to one row of Table C (compounds III.109dC-1 to III.109dC-1275)

Table 110d
Compounds III in which B is 2-ethyl-3-chlorophenyl and the combination of A and Z corresponds in each case to one row of Table C (compounds III.110dC-1 to III.110dC-1275)

Table 111d
Compounds III in which B is 2-ethyl-4-chlorophenyl and the combination of A and Z corresponds in each case to one row of Table C (compounds III.111dC-1 to III.111dC-1275)

Table 112d
Compounds III in which B is 2-ethyl-5-chlorophenyl and the combination of A and Z corresponds in each case to one row of Table C (compounds III.112dC-1 to III.112dC-1275)

Table 113d
Compounds III in which B is 2-ethyl-6-chlorophenyl and the combination of A and Z corresponds in each case to one row of Table C (compounds III.113dC-1 to III.113dC-1275)

Table 114d
Compounds III in which B is 2-methoxy-3-chlorophenyl and the combination of A and Z corresponds in each case to one row of Table C (compounds III.114dC-1 to III.114dC-1275)

Table 115d
Compounds III in which B is 2-methoxy-4-chlorophenyl and the combination of A and Z corresponds in each case to one row of Table C (compounds III.115dC-1 to III.115dC-1275)

Table 116d
Compounds III in which B is 2-methoxy-5-chlorophenyl and the combination of A and Z corresponds in each case to one row of Table C (compounds III.116dC-1 to III.116dC-1275)

Table 117d
Compounds III in which B is 2-methoxy-6-chlorophenyl and the combination of A and Z corresponds in each case to one row of Table C (compounds III.117dC-1 to III.117dC-1275)

Table 118d
Compounds III in which B is 2-chloro-3-methoxyphenyl and the combination of A and Z corresponds in each case to one row of Table C (compounds III.118dC-1 to III.118dC-1275)

Table 119d
Compounds III in which B is 2-chloro-4-methoxyphenyl and the combination of A and Z corresponds in each case to one row of Table C (compounds III.119dC-1 to III.119dC-1275)

Table 120d
Compounds III in which B is 2-chloro-5-methoxyphenyl and the combination of A and Z corresponds in each case to one row of Table C (compounds III.120dC-1 to III.120dC-1275)

Table 121d
Compounds III in which B is 2-(trifluoromethyl)-3-chlorophenyl and the combination of A and Z corresponds in each case to one row of Table C (compounds III.121dC-1 to III.121dC-1275)

Table 122d
Compounds III in which B is 2-(trifluoromethyl)-4-chlorophenyl and the combination of A and Z corresponds in each case to one row of Table C (compounds III.122dC-1 to III.122dC-1275)

Table 123d
Compounds III in which B is 2-(trifluoromethyl)-5-chlorophenyl and the combination of A and Z corresponds in each case to one row of Table C (compounds III.123dC-1 to III.123dC-1275)

Table 124d
Compounds III in which B is 2-(trifluoromethyl)-6-chlorophenyl and the combination of A and Z corresponds in each case to one row of Table C (compounds III.124dC-1 to III.124dC-1275)

Table 125d
Compounds III in which B is 2-chloro-3-(trifluoromethyl)phenyl and the combination of A and Z corresponds in each case to one row of Table C (compounds III.125dC-1 to III.125dC-1275)

Table 126d
Compounds III in which B is 2-chloro-4-(trifluoromethyl)phenyl and the combination of A and Z corresponds in each case to one row of Table C (compounds III.126dC-1 to III.126dC-1275)

Table 127d
Compounds III in which B is 2-chloro-5-(trifluoromethyl)phenyl and the combination of A and Z corresponds in each case to one row of Table C (compounds III.127dC-1 to III.127dC-1275)

Table 128d
Compounds III in which B is 2-(trifluoromethoxy)-3-chlorophenyl and the combination of A and Z corresponds in each case to one row of Table C (compounds III.128dC-1 to III.128dC-1275)

Table 129d
Compounds III in which B is 2-(trifluoromethoxy)-4-chlorophenyl and the combination of A and Z corresponds in each case to one row of Table C (compounds III.129dC-1 to III.129dC-1275)

Table 130d
Compounds III in which B is 2-(trifluoromethoxy)-5-chlorophenyl and the combination of A and Z corresponds in each case to one row of Table C (compounds III.130dC-1 to III.130dC-1275)

Table 131d
Compounds III in which B is 2-(trifluoromethoxy)-6-chlorophenyl and the combination of A and Z corresponds in each case to one row of Table C (compounds III.131dC-1 to III.131dC-1275)

Table 132d
Compounds III in which B is 2-chloro-3-(trifluoromethoxy)phenyl and the combination of A and Z corresponds in each case to one row of Table C (compounds III.132dC-1 to III.132dC-1275)

Table 133d
Compounds III in which B is 2-chloro-4-(trifluoromethoxy)phenyl and the combination of A and Z corresponds in each case to one row of Table C (compounds III.133dC-1 to III.133dC-1275)

Table 134d
Compounds III in which B is 2-chloro-5-(trifluoromethoxy)phenyl and the combination of A and Z corresponds in each case to one row of Table C (compounds III.134dC-1 to III.134dC-1275)

Table 135d
Compounds III in which B is 2-(difluoromethoxy)-3-chlorophenyl and the combination of A and Z corresponds in each case to one row of Table C (compounds III.135dC-1 to III.135dC-1275)

Table 136d
Compounds III in which B is 2-(difluoromethoxy)-4-chlorophenyl and the combination of A and Z corresponds in each case to one row of Table C (compounds III.136dC-1 to III.136dC-1275)

Table 137d
Compounds III in which B is 2-(difluoromethoxy)-5-chlorophenyl and the combination of A and Z corresponds in each case to one row of Table C (compounds III.137dC-1 to III.137dC-1275)

Table 138d
Compounds III in which B is 2-(difluoromethoxy)-6-chlorophenyl and the combination of A and Z corresponds in each case to one row of Table C (compounds III.138dC-1 to III.138dC-1275)

Table 139d
Compounds III in which B is 3-(difluoromethoxy)-4-chlorophenyl and the combination of A and Z corresponds in each case to one row of Table C (compounds III.139dC-1 to III.139dC-1275)

Table 140d
Compounds III in which B is 2-chloro-3-(difluoromethoxy)phenyl and the combination of A and Z corresponds in each case to one row of Table C (compounds III.140dC-1 to III.140dC-1275)

Table 141d
Compounds III in which B is 2-chloro-4-(difluoromethoxy)phenyl and the combination of A and Z corresponds in each case to one row of Table C (compounds III.141dC-1 to III.141dC-1275)

Table 142d
Compounds III in which B is 2-chloro-5-(difluoromethoxy)phenyl and the combination of A and Z corresponds in each case to one row of Table C (compounds III.142dC-1 to III.142dC-1275)

Table 143d
Compounds III in which B is 2-(trifluoromethylthio)-3-chlorophenyl and the combination of A and Z corresponds in each case to one row of Table C (compounds III.143dC-1 to III.143dC-1275)

Table 144d
Compounds III in which B is 2-(trifluoromethylthio)-4-chlorophenyl and the combination of A and Z corresponds in each case to one row of Table C (compounds III.144dC-1 to III.144dC-1275)

Table 145d
Compounds III in which B is 2-(trifluoromethylthio)-5-chlorophenyl and the combination of A and Z corresponds in each case to one row of Table C (compounds III.145dC-1 to III.145dC-1275)

Table 146d
Compounds III in which B is 2-(trifluoromethylthio)-6-chlorophenyl and the combination of A and Z corresponds in each case to one row of Table C (compounds III.146dC-1 to III.146dC-1275)

Table 147d
Compounds III in which B is 2-chloro-3-(trifluoromethylthio)phenyl and the combination of A and Z corresponds in each case to one row of Table C (compounds III.147dC-1 to III.147dC-1275)

Table 148d
Compounds III in which B is 2-chloro-4-(trifluoromethylthio)phenyl and the combination of A and Z corresponds in each case to one row of Table C (compounds III.148dC-1 to III.148dC-1275)

Table 149d
Compounds III in which B is 2-chloro-5-(trifluoromethylthio)phenyl and the combination of A and Z corresponds in each case to one row of Table C (compounds III.149dC-1 to III.149dC-1275)

Table 150d
Compounds III in which B is 2,3,4-trichlorophenyl and the combination of A and Z corresponds in each case to one row of Table C (compounds III.150dC-1 to III.150dC-1275)

Table 151d
Compounds III in which B is 2,3,5-trichlorophenyl and the combination of A and Z corresponds in each case to one row of Table C (compounds III.151dC-1 to III.151dC-1275)

Table 152d
Compounds III in which B is 2,3,6-trichlorophenyl and the combination of A and Z corresponds in each case to one row of Table C (compounds III.152dC-1 to III.152dC-1275)

Table 153d
Compounds III in which B is 2,4,5-trichlorophenyl and the combination of A and Z corresponds in each case to one row of Table C (compounds III.153dC-1 to III.153dC-1275)

Table 154d
Compounds III in which B is 2,4,6-trichlorophenyl and the combination of A and Z corresponds in each case to one row of Table C (compounds 111.154dC-1 to III.154dC-1275)

Table 155d
Compounds III in which B is 2,3,4-trimethylphenyl and the combination of A and Z corresponds in each case to one group of one row of Table C (compounds III.155dC-1 to III.155dC-1275)

Table 156d
Compounds III in which B is 2,3,5-trimethylphenyl and the combination of A and Z corresponds in each case to one group of one row of Table C (compounds III.156dC-1 to III.156dC-1275)

Table 157d
Compounds III in which B is 2,3,6-trimethylphenyl and the combination of A and Z corresponds in each case to one group of one row of Table C (compounds III.157dC-1 to III.157dC-1275)

Table 158d
Compounds III in which B is 2,4,5-trimethylphenyl and the combination of A and Z corresponds in each case to one group of one row of Table C (compounds III.158dC-1 to III.158dC-1275)

Table 159d
Compounds III in which B is 2,4,6-trimethylphenyl and the combination of A and Z corresponds in each case to one group of one row of Table C (compounds III.159dC-1 to III.159dC-1275)

Table 160d
Compounds III in which B is 2,3,4-trimethoxyphenyl and the combination of A and Z corresponds in each case to one group of one row of Table C (compounds III.160dC-1 to III.160dC-1275)

Table 161d
Compounds III in which B is 2,3,5-trimethoxyphenyl and the combination of A and Z corresponds in each case to one group of one row of Table C (compounds III.161dC-1 to III.161dC-1275)

Table 162d
Compounds III in which B is 2,3,6-trimethoxyphenyl and the combination of A and Z corresponds in each case to one group of one row of Table C (compounds III.162dC-1 to III.162dC-1275)

TABLE C

| Row | A | Z |
|---|---|---|
| C-1 | Phenyl | Cl |
| C-2 | 2-Chlorophenyl | Cl |
| C-3 | 3-Chlorophenyl | Cl |
| C-4 | 4-Chlorophenyl | Cl |
| C-5 | 2-Fluorophenyl | Cl |
| C-6 | 3-Fluorophenyl | Cl |
| C-7 | 4-Fluorophenyl | Cl |
| C-8 | 2-Methylphenyl | Cl |
| C-9 | 3-Methylphenyl | Cl |
| C-10 | 4-Methylphenyl | Cl |
| C-11 | 2-Ethylphenyl | Cl |
| C-12 | 3-Ethylphenyl | Cl |
| C-13 | 4-Ethylphenyl | Cl |
| C-14 | 2-Methoxyphenyl | Cl |
| C-15 | 3-Methoxyphenyl | Cl |
| C-16 | 4-Methoxyphenyl | Cl |
| C-17 | 2-Trifluoromethylphenyl | Cl |
| C-18 | 3-Trifluoromethylphenyl | Cl |
| C-19 | 4-Trifluoromethylphenyl | Cl |
| C-20 | 2-Trifluoromethoxyphenyl | Cl |
| C-21 | 3-Trifluoromethoxyphenyl | Cl |
| C-22 | 4-Trifluoromethoxyphenyl | Cl |
| C-23 | 2-Difluoromethoxyphenyl | Cl |
| C-24 | 3-Difluoromethoxyphenyl | Cl |
| C-25 | 4-Difluoromethoxyphenyl | Cl |
| C-26 | 2-Trifluoromethylthiophenyl | Cl |
| C-27 | 3-Trifluoromethylthiophenyl | Cl |
| C-28 | 4-Trifluoromethylthiophenyl | Cl |
| C-29 | 2,3-Dichlorophenyl | Cl |
| C-30 | 2,4-Dichlorophenyl | Cl |
| C-31 | 2,5-Dichlorophenyl | Cl |
| C-32 | 2,6-Dichlorophenyl | Cl |
| C-33 | 3,4-Dichlorophenyl | Cl |
| C-34 | 3,5-Dichlorophenyl | Cl |
| C-35 | 2,3-Difluorophenyl | Cl |
| C-36 | 2,4-Difluorophenyl | Cl |
| C-37 | 2,5-Difluorophenyl | Cl |
| C-38 | 2,6-Difluorophenyl | Cl |
| C-39 | 3,4-Difluorophenyl | Cl |
| C-40 | 3,5-Difluorophenyl | Cl |
| C-41 | 2,3-Dimethylphenyl | Cl |
| C-42 | 2,4-Dimethylphenyl | Cl |
| C-43 | 2,5-Dimethylphenyl | Cl |
| C-44 | 2,6-Dimethylphenyl | Cl |
| C-45 | 3,4-Dimethylphenyl | Cl |
| C-46 | 3,5-Dimethylphenyl | Cl |
| C-47 | 2,3-Diethylphenyl | Cl |
| C-48 | 2,4-Diethylphenyl | Cl |
| C-49 | 2,5-Diethylphenyl | Cl |
| C-50 | 2,6-Diethylphenyl | Cl |
| C-51 | 3,5-Diethylphenyl | Cl |
| C-52 | 3,4-Diethylphenyl | Cl |
| C-53 | 2,3-Dimethoxyphenyl | Cl |
| C-54 | 2,4-Dimethoxyphenyl | Cl |
| C-55 | 2,5-Dimethoxyphenyl | Cl |
| C-56 | 2,6-Dimethoxyphenyl | Cl |
| C-57 | 3,4-Dimethoxyphenyl | Cl |
| C-58 | 3,5-Dimethoxyphenyl | Cl |
| C-59 | 2,3-Di(trifluoromethyl)phenyl | Cl |
| C-60 | 2,4-Di(trifluoromethyl)phenyl | Cl |
| C-61 | 2,5-Di(trifluoromethyl)phenyl | Cl |
| C-62 | 2,6-Di(trifluoromethyl)phenyl | Cl |
| C-63 | 3,4-Di(trifluoromethyl)phenyl | Cl |
| C-64 | 3,5-Di(trifluoromethyl)phenyl | Cl |
| C-65 | 2,3-Di(trifluoromethoxy)phenyl | Cl |
| C-66 | 2,4-Di(trifluoromethoxy)phenyl | Cl |
| C-67 | 2,5-Di(trifluoromethoxy)phenyl | Cl |
| C-68 | 2,6-Di(trifluoromethoxy)phenyl | Cl |
| C-69 | 3,4-Di(trifluoromethoxy)phenyl | Cl |
| C-70 | 3,5-Di(trifluoromethoxy)phenyl | Cl |
| C-71 | 2,3-Di(difluoromethoxy)phenyl | Cl |
| C-72 | 2,4-Di(difluoromethoxy)phenyl | Cl |
| C-73 | 2,5-Di(difluoromethoxy)phenyl | Cl |
| C-74 | 2,6-Di(difluoromethoxy)phenyl | Cl |
| C-75 | 3,4-Di(difluoromethoxy)phenyl | Cl |
| C-76 | 3,5-Di(difluoromethoxy)phenyl | Cl |
| C-77 | 2,3-Di(trifluoromethylthio)phenyl | Cl |
| C-78 | 2,4-Di(trifluoromethylthio)phenyl | Cl |
| C-79 | 2,5-Di(trifluoromethylthio)phenyl | Cl |
| C-80 | 2,6-Di(trifluoromethylthio)phenyl | Cl |
| C-81 | 3,4-Di(trifluoromethylthio)phenyl | Cl |
| C-82 | 3,5-Di(trifluoromethylthio)phenyl | Cl |
| C-83 | 2-Fluoro-3-chlorophenyl | Cl |
| C-84 | 2-Fluoro-4-chlorophenyl | Cl |
| C-85 | 2-Fluoro-5-chlorophenyl | Cl |
| C-86 | 2-Fluoro-6-chlorophenyl | Cl |
| C-87 | 3-Fluoro-4-chlorophenyl | Cl |
| C-88 | 3-Fluoro-5-chlorophenyl | Cl |
| C-89 | 2-Chloro-3-fluorophenyl | Cl |
| C-90 | 2-Chloro-4-fluorophenyl | Cl |
| C-91 | 2-Chloro-5-fluorophenyl | Cl |
| C-92 | 3-Chloro-4-fluorophenyl | Cl |
| C-93 | 2-Methyl-3-chlorophenyl | Cl |
| C-94 | 2-Methyl-4-chlorophenyl | Cl |
| C-95 | 2-Methyl-5-chlorophenyl | Cl |
| C-96 | 2-Methyl-6-chlorophenyl | Cl |
| C-97 | 3-Methyl-4-chlorophenyl | Cl |
| C-98 | 3-Methyl-5-chlorophenyl | Cl |
| C-99 | 2-Chloro-3-methylphenyl | Cl |
| C-100 | 2-Chloro-4-methylphenyl | Cl |
| C-101 | 2-Chloro-5-methylphenyl | Cl |
| C-102 | 3-Chloro-4-methylphenyl | Cl |
| C-103 | 2-Methyl-3-fluorophenyl | Cl |
| C-104 | 2-Methyl-4-fluorophenyl | Cl |
| C-105 | 2-Methyl-5-fluorophenyl | Cl |
| C-106 | 2-Methyl-6-fluorophenyl | Cl |
| C-107 | 3-Methyl-4-fluorophenyl | Cl |
| C-108 | 3-Methyl-5-fluorophenyl | Cl |
| C-109 | 2-Fluoro-3-methylphenyl | Cl |
| C-110 | 2-Fluoro-4-methylphenyl | Cl |
| C-111 | 2-Fluoro-5-methylphenyl | Cl |
| C-112 | 3-Fluoro-4-methylphenyl | Cl |
| C-113 | 2-Chloro-3-ethylphenyl | Cl |
| C-114 | 2-Chloro-4-ethylphenyl | Cl |
| C-115 | 2-Chloro-5-ethylphenyl | Cl |
| C-116 | 3-Chloro-4-ethylphenyl | Cl |
| C-117 | 2-Ethyl-3-chlorophenyl | Cl |
| C-118 | 2-Ethyl-4-chlorophenyl | Cl |
| C-119 | 2-Ethyl-5-chlorophenyl | Cl |
| C-120 | 2-Ethyl-6-chlorophenyl | Cl |
| C-121 | 2-Ethyl-3-fluorophenyl | Cl |
| C-122 | 2-Ethyl-4-fluorophenyl | Cl |
| C-123 | 2-Ethyl-5-fluorophenyl | Cl |
| C-124 | 2-Ethyl-6-fluorophenyl | Cl |
| C-125 | 3-Ethyl-4-fluorophenyl | Cl |
| C-126 | 3-Ethyl-5-fluorophenyl | Cl |
| C-127 | 2-Fluoro-3-ethylphenyl | Cl |
| C-128 | 2-Fluoro-4-ethylphenyl | Cl |
| C-129 | 2-Fluoro-5-ethylphenyl | Cl |
| C-130 | 3-Fluoro-4-ethylphenyl | Cl |
| C-131 | 2-Methoxy-3-chlorophenyl | Cl |
| C-132 | 2-Methoxy-4-chlorophenyl | Cl |
| C-133 | 2-Methoxy-5-chlorophenyl | Cl |
| C-134 | 2-Methoxy-6-chlorophenyl | Cl |
| C-135 | 3-Methoxy-4-chlorophenyl | Cl |
| C-136 | 3-Methoxy-5-chlorophenyl | Cl |
| C-137 | 2-Chloro-3-methoxyphenyl | Cl |
| C-138 | 2-Chloro-4-methoxyphenyl | Cl |
| C-139 | 2-Chloro-5-methoxyphenyl | Cl |
| C-140 | 3-Chloro-4-methoxyphenyl | Cl |
| C-141 | 2-Methoxy-3-fluorophenyl | Cl |
| C-142 | 2-Methoxy-4-fluorophenyl | Cl |
| C-143 | 2-Methoxy-5-fluorophenyl | Cl |
| C-144 | 2-Methoxy-6-fluorophenyl | Cl |
| C-145 | 3-Methoxy-4-fluorophenyl | Cl |
| C-146 | 3-Methoxy-5-fluorophenyl | Cl |
| C-147 | 2-Fluoro-3-methoxyphenyl | Cl |
| C-148 | 2-Fluoro-4-methoxyphenyl | Cl |
| C-149 | 2-Fluoro-5-methoxyphenyl | Cl |
| C-150 | 3-Fluoro-4-methoxyphenyl | Cl |
| C-151 | 3-Fluoro-5-methoxyphenyl | Cl |
| C-152 | 2-(Trifluoromethyl)-3-chlorophenyl | Cl |
| C-153 | 2-(Trifluoromethyl)-4-chlorophenyl | Cl |
| C-154 | 2-(Trifluoromethyl)-5-chlorophenyl | Cl |
| C-155 | 2-(Trifluoromethyl)-6-chlorophenyl | Cl |
| C-156 | 3-(Trifluoromethyl)-4-chlorophenyl | Cl |
| C-157 | 3-(Trifluoromethyl)-5-chlorophenyl | Cl |
| C-158 | 2-Chloro-3-(trifluoromethyl)phenyl | Cl |
| C-159 | 2-Chloro-4-(trifluoromethyl)phenyl | Cl |
| C-160 | 2-Chloro-5-(trifluoromethyl)phenyl | Cl |
| C-161 | 3-Chloro-4-(trifluoromethyl)phenyl | Cl |
| C-162 | 2-(Trifluoromethyl)-3-fluorophenyl | Cl |
| C-163 | 2-(Trifluoromethyl)-4-fluorophenyl | Cl |
| C-164 | 2-(Trifluoromethyl)-5-fluorophenyl | Cl |
| C-165 | 2-(Trifluoromethyl)-6-fluorophenyl | Cl |
| C-166 | 3-(Trifluoromethyl)-4-fluorophenyl | Cl |
| C-167 | 3-(Trifluoromethyl)-5-fluorophenyl | Cl |
| C-168 | 2-Fluoro-3-(trifluoromethyl)phenyl | Cl |
| C-169 | 2-Fluoro-4-(trifluoromethyl)phenyl | Cl |
| C-170 | 2-Fluoro-5-(trifluoromethyl)phenyl | Cl |
| C-171 | 3-Fluoro-4-(trifluoromethyl)phenyl | Cl |
| C-172 | 2-(Trifluoromethoxy)-3-chlorophenyl | Cl |
| C-173 | 2-(Trifluoromethoxy)-4-chlorophenyl | Cl |
| C-174 | 2-(Trifluoromethoxy)-5-chlorophenyl | Cl |
| C-175 | 2-(Trifluoromethoxy)-6-chlorophenyl | Cl |
| C-176 | 3-(Trifluoromethoxy)-4-chlorophenyl | Cl |
| C-177 | 3-(Trifluoromethoxy)-5-chlorophenyl | Cl |
| C-178 | 2-Chloro-3-(trifluoromethoxy)phenyl | Cl |
| C-179 | 2-Chloro-4-(trifluoromethoxy)phenyl | Cl |
| C-180 | 2-Chloro-5-(trifluoromethoxy)phenyl | Cl |
| C-181 | 3-Chloro-4-(trifluoromethoxy)phenyl | Cl |
| C-182 | 2-(Trifluoromethoxy)-3-fluorophenyl | Cl |
| C-183 | 2-(Trifluoromethoxy)-4-fluorophenyl | Cl |
| C-184 | 2-(Trifluoromethoxy)-5-fluorophenyl | Cl |
| C-185 | 2-(Trifluoromethoxy)-6-fluorophenyl | Cl |
| C-186 | 3-(Trifluoromethoxy)-4-fluorophenyl | Cl |
| C-187 | 3-(Trifluoromethoxy)-5-fluorophenyl | Cl |
| C-188 | 2-Fluoro-3-(trifluoromethoxy)phenyl | Cl |
| C-189 | 2-Fluoro-4-(trifluoromethoxy)phenyl | Cl |
| C-190 | 2-Fluoro-5-(trifluoromethoxy)phenyl | Cl |
| C-191 | 3-Fluoro-4-(trifluoromethoxy)phenyl | Cl |
| C-192 | 2-(Difluoromethoxy)-3-chlorophenyl | Cl |
| C-193 | 2-(Difluoromethoxy)-4-chlorophenyl | Cl |
| C-194 | 2-(Difluoromethoxy)-5-chlorophenyl | Cl |
| C-195 | 2-(Difluoromethoxy)-6-chlorophenyl | Cl |
| C-196 | 3-(Difluoromethoxy)-4-chlorophenyl | Cl |
| C-197 | 3-(Difluoromethoxy)-5-chlorophenyl | Cl |
| C-198 | 2-Chloro-3-(difluoromethoxy)phenyl | Cl |
| C-199 | 2-Chloro-4-(difluoromethoxy)phenyl | Cl |
| C-200 | 2-Chloro-5-(difluoromethoxy)phenyl | Cl |
| C-201 | 3-Chloro-4-(difluoromethoxy)phenyl | Cl |
| C-202 | 2-(Difluoromethoxy)-3-fluorophenyl | Cl |
| C-203 | 2-(Difluoromethoxy)-4-fluorophenyl | Cl |
| C-204 | 2-(Difluoromethoxy)-5-fluorophenyl | Cl |
| C-205 | 2-(Difluoromethoxy)-6-fluorophenyl | Cl |
| C-206 | 3-(Difluoromethoxy)-4-fluorophenyl | Cl |
| C-207 | 3-(Difluoromethoxy)-5-fluorophenyl | Cl |
| C-208 | 2-Fluoro-3-(difluoromethoxy)phenyl | Cl |
| C-209 | 2-Fluoro-4-(difluoromethoxy)phenyl | Cl |
| C-210 | 2-Fluoro-5-(difluoromethoxy)phenyl | Cl |
| C-211 | 3-Fluoro-4-(difluoromethoxy)phenyl | Cl |
| C-212 | 2-(Trifluoromethylthio)-3-chlorophenyl | Cl |
| C-213 | 2-(Trifluoromethylthio)-4-chlorophenyl | Cl |
| C-214 | 2-(Trifluoromethylthio)-5-chlorophenyl | Cl |
| C-215 | 2-(Trifluoromethylthio)-6-chlorophenyl | Cl |
| C-216 | 3-(Trifluoromethylthio)-4-chlorophenyl | Cl |
| C-217 | 3-(Trifluoromethylthio)-5-chlorophenyl | Cl |
| C-218 | 2-Chloro-3-(trifluoromethylthio)phenyl | Cl |
| C-219 | 2-Chloro-4-(trifluoromethylthio)phenyl | Cl |
| C-220 | 2-Chloro-5-(trifluoromethylthio)phenyl | Cl |
| C-221 | 3-Chloro-4-(trifluoromethylthio)phenyl | Cl |
| C-222 | 2-(Trifluoromethylthio)-3-fluorophenyl | Cl |
| C-223 | 2-(Trifluoromethylthio)-4-fluorophenyl | Cl |
| C-224 | 2-(Trifluoromethylthio)-5-fluorophenyl | Cl |
| C-225 | 2-(Trifluoromethylthio)-6-fluorophenyl | Cl |
| C-226 | 3-(Trifluoromethylthio)-4-fluorophenyl | Cl |
| C-227 | 3-(Trifluoromethylthio)-5-fluorophenyl | Cl |
| C-228 | 2-Fluoro-3-(trifluoromethylthio)phenyl | Cl |
| C-229 | 2-Fluoro-4-(trifluoromethylthio)phenyl | Cl |
| C-230 | 2-Fluoro-5-(trifluoromethylthio)phenyl | Cl |
| C-231 | 3-Fluoro-4-(trifluoromethylthio)phenyl | Cl |
| C-232 | 2,3,4-Trichlorophenyl | Cl |
| C-233 | 2,3,5-Trichlorophenyl | Cl |
| C-234 | 2,3,6-Trichlorophenyl | Cl |
| C-235 | 2,4,5-Trichlorophenyl | Cl |
| C-236 | 2,4,6-Trichlorophenyl | Cl |
| C-237 | 3,4,5-Trichlorophenyl | Cl |
| C-238 | 2,3,4-Trifluorophenyl | Cl |
| C-239 | 2,3,5-Trifluorophenyl | Cl |
| C-240 | 2,3,6-Trifluorophenyl | Cl |
| C-241 | 2,4,5-Trifluorophenyl | Cl |
| C-242 | 2,4,6-Trifluorophenyl | Cl |
| C-243 | 3,4,5-Trifluorophenyl | Cl |
| C-244 | 2,3,4-Trimethylphenyl | Cl |
| C-245 | 2,3,5-Trimethylphenyl | Cl |
| C-246 | 2,3,6-Trimethylphenyl | Cl |
| C-247 | 2,4,5-Trimethylphenyl | Cl |
| C-248 | 2,4,6-Trimethylphenyl | Cl |
| C-249 | 3,4,5-Trimethylphenyl | Cl |
| C-250 | 2,3,4-Trimethoxyphenyl | Cl |
| C-251 | 2,3,5-Trimethoxyphenyl | Cl |
| C-252 | 2,3,6-Trimethoxyphenyl | Cl |
| C-253 | 2,4,5-Trimethoxyphenyl | Cl |
| C-254 | 2,4,6-Trimethoxyphenyl | Cl |
| C-255 | 3,4,5-Trimethoxyphenyl | Cl |
| C-256 | Phenyl | Br |
| C-257 | 2-Chlorophenyl | Br |
| C-258 | 3-Chlorophenyl | Br |
| C-259 | 4-Chlorophenyl | Br |
| C-260 | 2-Fluorophenyl | Br |
| C-261 | 3-Fluorophenyl | Br |
| C-262 | 4-Fluorophenyl | Br |
| C-263 | 2-Methylphenyl | Br |
| C-264 | 3-Methylphenyl | Br |
| C-265 | 4-Methylphenyl | Br |
| C-266 | 2-Ethylphenyl | Br |
| C-267 | 3-Ethylphenyl | Br |
| C-268 | 4-Ethylphenyl | Br |
| C-269 | 2-Methoxyphenyl | Br |
| C-270 | 3-Methoxyphenyl | Br |
| C-271 | 4-Methoxyphenyl | Br |
| C-272 | 2-Trifluoromethylphenyl | Br |
| C-273 | 3-Trifluoromethylphenyl | Br |
| C-274 | 4-Trifluoromethylphenyl | Br |
| C-275 | 2-Trifluoromethoxyphenyl | Br |
| C-276 | 3-Trifluoromethoxyphenyl | Br |
| C-277 | 4-Trifluoromethoxyphenyl | Br |
| C-278 | 2-Difluoromethoxyphenyl | Br |
| C-279 | 3-Difluoromethoxyphenyl | Br |
| C-280 | 4-Difluoromethoxyphenyl | Br |
| C-281 | 2-Trifluoromethylthiophenyl | Br |
| C-282 | 3-Trifluoromethylthiophenyl | Br |
| C-283 | 4-Trifluoromethylthiophenyl | Br |
| C-284 | 2,3-Dichlorophenyl | Br |
| C-285 | 2,4-Dichlorophenyl | Br |
| C-286 | 2,5-Dichlorophenyl | Br |
| C-287 | 2,6-Dichlorophenyl | Br |
| C-288 | 3,4-Dichlorophenyl | Br |
| C-289 | 3,5-Dichlorophenyl | Br |
| C-290 | 2,3-Difluorophenyl | Br |
| C-291 | 2,4-Difluorophenyl | Br |
| C-292 | 2,5-Difluorophenyl | Br |
| C-293 | 2,6-Difluorophenyl | Br |
| C-294 | 3,4-Difluorophenyl | Br |
| C-295 | 3,5-Difluorophenyl | Br |
| C-296 | 2,3-Dimethylphenyl | Br |
| C-297 | 2,4-Dimethylphenyl | Br |
| C-298 | 2,5-Dimethylphenyl | Br |
| C-299 | 2,6-Dimethylphenyl | Br |
| C-300 | 3,4-Dimethylphenyl | Br |
| C-301 | 3,5-Dimethylphenyl | Br |
| C-302 | 2,3-Diethylphenyl | Br |
| C-303 | 2,4-Diethylphenyl | Br |
| C-304 | 2,5-Diethylphenyl | Br |
| C-305 | 2,6-Diethylphenyl | Br |
| C-306 | 3,5-Diethylphenyl | Br |
| C-307 | 3,4-Diethylphenyl | Br |
| C-308 | 2,3-Dimethoxyphenyl | Br |
| C-309 | 2,4-Dimethoxyphenyl | Br |
| C-310 | 2,5-Dimethoxyphenyl | Br |
| C-311 | 2,6-Dimethoxyphenyl | Br |
| C-312 | 3,4-Dimethoxyphenyl | Br |
| C-313 | 3,5-Dimethoxyphenyl | Br |
| C-314 | 2,3-Di(trifluoromethyl)phenyl | Br |
| C-315 | 2,4-Di(trifluoromethyl)phenyl | Br |
| C-316 | 2,5-Di(trifluoromethyl)phenyl | Br |
| C-317 | 2,6-Di(trifluoromethyl)phenyl | Br |
| C-318 | 3,4-Di(trifluoromethyl)phenyl | Br |
| C-319 | 3,5-Di(trifluoromethyl)phenyl | Br |
| C-320 | 2,3-Di(trifluoromethoxy)phenyl | Br |
| C-321 | 2,4-Di(trifluoromethoxy)phenyl | Br |
| C-322 | 2,5-Di(trifluoromethoxy)phenyl | Br |
| C-323 | 2,6-Di(trifluoromethoxy)phenyl | Br |
| C-324 | 3,4-Di(trifluoromethoxy)phenyl | Br |
| C-325 | 3,5-Di(trifluoromethoxy)phenyl | Br |
| C-326 | 2,3-Di(difluoromethoxy)phenyl | Br |
| C-327 | 2,4-Di(difluoromethoxy)phenyl | Br |
| C-328 | 2,5-Di(difluoromethoxy)phenyl | Br |
| C-329 | 2,6-Di(difluoromethoxy)phenyl | Br |
| C-330 | 3,4-Di(difluoromethoxy)phenyl | Br |
| C-331 | 3,5-Di(difluoromethoxy)phenyl | Br |
| C-332 | 2,3-Di(trifluoromethylthio)phenyl | Br |
| C-333 | 2,4-Di(trifluoromethylthio)phenyl | Br |
| C-334 | 2,5-Di(trifluoromethylthio)phenyl | Br |
| C-335 | 2,6-Di(trifluoromethylthio)phenyl | Br |
| C-336 | 3,4-Di(trifluoromethylthio)phenyl | Br |
| C-337 | 3,5-Di(trifluoromethylthio)phenyl | Br |
| C-338 | 2-Fluoro-3-chlorophenyl | Br |
| C-339 | 2-Fluoro-4-chlorophenyl | Br |
| C-340 | 2-Fluoro-5-chlorophenyl | Br |
| C-341 | 2-Fluoro-6-chlorophenyl | Br |
| C-342 | 3-Fluoro-4-chlorophenyl | Br |
| C-343 | 3-Fluoro-5-chlorophenyl | Br |
| C-344 | 2-Chloro-3-fluorophenyl | Br |
| C-345 | 2-Chloro-4-fluorophenyl | Br |
| C-346 | 2-Chloro-5-fluorophenyl | Br |
| C-347 | 3-Chloro-4-fluorophenyl | Br |
| C-348 | 2-Methyl-3-chlorophenyl | Br |
| C-349 | 2-Methyl-4-chlorophenyl | Br |
| C-350 | 2-Methyl-5-chlorophenyl | Br |
| C-351 | 2-Methyl-6-chlorophenyl | Br |
| C-352 | 3-Methyl-4-chlorophenyl | Br |
| C-353 | 3-Methyl-5-chlorophenyl | Br |
| C-354 | 2-Chloro-3-methylphenyl | Br |
| C-355 | 2-Chloro-4-methylphenyl | Br |
| C-356 | 2-Chloro-5-methylphenyl | Br |
| C-357 | 3-Chloro-4-methylphenyl | Br |
| C-358 | 2-Methyl-3-fluorophenyl | Br |
| C-359 | 2-Methyl-4-fluorophenyl | Br |
| C-360 | 2-Methyl-5-fluorophenyl | Br |
| C-361 | 2-Methyl-6-fluorophenyl | Br |
| C-362 | 3-Methyl-4-fluorophenyl | Br |
| C-363 | 3-Methyl-5-fluorophenyl | Br |
| C-364 | 2-Fluoro-3-methylphenyl | Br |
| C-365 | 2-Fluoro-4-methylphenyl | Br |
| C-366 | 2-Fluoro-5-methylphenyl | Br |
| C-367 | 3-Fluoro-4-methylphenyl | Br |
| C-368 | 2-Chloro-3-ethylphenyl | Br |
| C-369 | 2-Chloro-4-ethylphenyl | Br |
| C-370 | 2-Chloro-5-ethylphenyl | Br |
| C-371 | 3-Chloro-4-ethylphenyl | Br |
| C-372 | 2-Ethyl-3-chlorophenyl | Br |
| C-373 | 2-Ethyl-4-chlorophenyl | Br |
| C-374 | 2-Ethyl-5-chlorophenyl | Br |
| C-375 | 2-Ethyl-6-chlorophenyl | Br |
| C-376 | 2-Ethyl-3-fluorophenyl | Br |
| C-377 | 2-Ethyl-4-fluorophenyl | Br |
| C-378 | 2-Ethyl-5-fluorophenyl | Br |
| C-379 | 2-Ethyl-6-fluorophenyl | Br |
| C-380 | 3-Ethyl-4-fluorophenyl | Br |
| C-381 | 3-Ethyl-5-fluorophenyl | Br |
| C-382 | 2-Fluoro-3-ethylphenyl | Br |
| C-383 | 2-Fluoro-4-ethylphenyl | Br |
| C-384 | 2-Fluoro-5-ethylphenyl | Br |
| C-385 | 3-Fluoro-4-ethylphenyl | Br |
| C-386 | 2-Methoxy-3-chlorophenyl | Br |
| C-387 | 2-Methoxy-4-chlorophenyl | Br |
| C-388 | 2-Methoxy-5-chlorophenyl | Br |
| C-389 | 2-Methoxy-6-chlorophenyl | Br |
| C-390 | 3-Methoxy-4-chlorophenyl | Br |
| C-391 | 3-Methoxy-5-chlorophenyl | Br |
| C-392 | 2-Chloro-3-methoxyphenyl | Br |
| C-393 | 2-Chloro-4-methoxyphenyl | Br |
| C-394 | 2-Chloro-5-methoxyphenyl | Br |
| C-395 | 3-Chloro-4-methoxyphenyl | Br |
| C-396 | 2-Methoxy-3-fluorophenyl | Br |
| C-397 | 2-Methoxy-4-fluorophenyl | Br |
| C-398 | 2-Methoxy-5-fluorophenyl | Br |
| C-399 | 2-Methoxy-6-fluorophenyl | Br |
| C-400 | 3-Methoxy-4-fluorophenyl | Br |
| C-401 | 3-Methoxy-5-fluorophenyl | Br |
| C-402 | 2-Fluoro-3-methoxyphenyl | Br |
| C-403 | 2-Fluoro-4-methoxyphenyl | Br |
| C-404 | 2-Fluoro-5-methoxyphenyl | Br |
| C-405 | 3-Fluoro-4-methoxyphenyl | Br |
| C-406 | 3-Fluoro-5-methoxyphenyl | Br |
| C-407 | 2-(Trifluoromethyl)-3-chlorophenyl | Br |
| C-408 | 2-(Trifluoromethyl)-4-chlorophenyl | Br |
| C-409 | 2-(Trifluoromethyl)-5-chlorophenyl | Br |
| C-410 | 2-(Trifluoromethyl)-6-chlorophenyl | Br |
| C-411 | 3-(Trifluoromethyl)-4-chlorophenyl | Br |
| C-412 | 3-(Trifluoromethyl)-5-chlorophenyl | Br |
| C-413 | 2-Chloro-3-(trifluoromethyl)phenyl | Br |
| C-414 | 2-Chloro-4-(trifluoromethyl)phenyl | Br |
| C-415 | 2-Chloro-5-(trifluoromethyl)phenyl | Br |
| C-416 | 3-Chloro-4-(trifluoromethyl)phenyl | Br |
| C-417 | 2-(Trifluoromethyl)-3-fluorophenyl | Br |
| C-418 | 2-(Trifluoromethyl)-4-fluorophenyl | Br |
| C-419 | 2-(Trifluoromethyl)-5-fluorophenyl | Br |
| C-420 | 2-(Trifluoromethyl)-6-fluorophenyl | Br |
| C-421 | 3-(Trifluoromethyl)-4-fluorophenyl | Br |
| C-422 | 3-(Trifluoromethyl)-5-fluorophenyl | Br |
| C-423 | 2-Fluoro-3-(trifluoromethyl)phenyl | Br |
| C-424 | 2-Fluoro-4-(trifluoromethyl)phenyl | Br |
| C-425 | 2-Fluoro-5-(trifluoromethyl)phenyl | Br |
| C-426 | 3-Fluoro-4-(trifluoromethyl)phenyl | Br |
| C-427 | 2-(Trifluoromethoxy)-3-chlorophenyl | Br |
| C-428 | 2-(Trifluoromethoxy)-4-chlorophenyl | Br |
| C-429 | 2-(Trifluoromethoxy)-5-chlorophenyl | Br |
| C-430 | 2-(Trifluoromethoxy)-6-chlorophenyl | Br |
| C-431 | 3-(Trifluoromethoxy)-4-chlorophenyl | Br |
| C-432 | 3-(Trifluoromethoxy)-5-chlorophenyl | Br |
| C-433 | 2-Chloro-3-(trifluoromethoxy)phenyl | Br |
| C-434 | 2-Chloro-4-(trifluoromethoxy)phenyl | Br |
| C-435 | 2-Chloro-5-(trifluoromethoxy)phenyl | Br |
| C-436 | 3-Chloro-4-(trifluoromethoxy)phenyl | Br |
| C-437 | 2-(Trifluoromethoxy)-3-fluorophenyl | Br |
| C-438 | 2-(Trifluoromethoxy)-4-fluorophenyl | Br |
| C-439 | 2-(Trifluoromethoxy)-5-fluorophenyl | Br |
| C-440 | 2-(Trifluoromethoxy)-6-fluorophenyl | Br |
| C-441 | 3-(Trifluoromethoxy)-4-fluorophenyl | Br |
| C-442 | 3-(Trifluoromethoxy)-5-fluorophenyl | Br |
| C-443 | 2-Fluoro-3-(trifluoromethoxy)phenyl | Br |
| C-444 | 2-Fluoro-4-(trifluoromethoxy)phenyl | Br |
| C-445 | 2-Fluoro-5-(trifluoromethoxy)phenyl | Br |
| C-446 | 3-Fluoro-4-(trifluoromethoxy)phenyl | Br |
| C-447 | 2-(Difluoromethoxy)-3-chlorophenyl | Br |
| C-448 | 2-(Difluoromethoxy)-4-chlorophenyl | Br |
| C-449 | 2-(Difluoromethoxy)-5-chlorophenyl | Br |
| C-450 | 2-(Difluoromethoxy)-6-chlorophenyl | Br |
| C-451 | 3-(Difluoromethoxy)-4-chlorophenyl | Br |
| C-452 | 3-(Difluoromethoxy)-5-chlorophenyl | Br |
| C-453 | 2-Chloro-3-(difluoromethoxy)phenyl | Br |
| C-454 | 2-Chloro-4-(difluoromethoxy)phenyl | Br |
| C-455 | 2-Chloro-5-(difluoromethoxy)phenyl | Br |
| C-456 | 3-Chloro-4-(difluoromethoxy)phenyl | Br |
| C-457 | 2-(Difluoromethoxy)-3-fluorophenyl | Br |
| C-458 | 2-(Difluoromethoxy)-4-fluorophenyl | Br |
| C-459 | 2-(Difluoromethoxy)-5-fluorophenyl | Br |
| C-460 | 2-(Difluoromethoxy)-6-fluorophenyl | Br |
| C-461 | 3-(Difluoromethoxy)-4-fluorophenyl | Br |
| C-462 | 3-(Difluoromethoxy)-5-fluorophenyl | Br |
| C-463 | 2-Fluoro-3-(difluoromethoxy)phenyl | Br |
| C-464 | 2-Fluoro-4-(difluoromethoxy)phenyl | Br |
| C-465 | 2-Fluoro-5-(difluoromethoxy)phenyl | Br |
| C-466 | 3-Fluoro-4-(difluoromethoxy)phenyl | Br |
| C-467 | 2-(Trifluoromethylthio)-3-chlorophenyl | Br |
| C-468 | 2-(Trifluoromethylthio)-4-chlorophenyl | Br |
| C-469 | 2-(Trifluoromethylthio)-5-chlorophenyl | Br |
| C-470 | 2-(Trifluoromethylthio)-6-chlorophenyl | Br |
| C-471 | 3-(Trifluoromethylthio)-4-chlorophenyl | Br |
| C-472 | 3-(Trifluoromethylthio)-5-chlorophenyl | Br |
| C-473 | 2-Chloro-3-(trifluoromethylthio)phenyl | Br |
| C-474 | 2-Chloro-4-(trifluoromethylthio)phenyl | Br |
| C-475 | 2-Chloro-5-(trifluoromethylthio)phenyl | Br |
| C-476 | 3-Chloro-4-(trifluoromethylthio)phenyl | Br |
| C-477 | 2-(Trifluoromethylthio)-3-fluorophenyl | Br |
| C-478 | 2-(Trifluoromethylthio)-4-fluorophenyl | Br |
| C-479 | 2-(Trifluoromethylthio)-5-fluorophenyl | Br |
| C-480 | 2-(Trifluoromethylthio)-6-fluorophenyl | Br |
| C-481 | 3-(Trifluoromethylthio)-4-fluorophenyl | Br |
| C-482 | 3-(Trifluoromethylthio)-5-fluorophenyl | Br |
| C-483 | 2-Fluoro-3-(trifluoromethylthio)phenyl | Br |
| C-484 | 2-Fluoro-4-(trifluoromethylthio)phenyl | Br |
| C-485 | 2-Fluoro-5-(trifluoromethylthio)phenyl | Br |
| C-486 | 3-Fluoro-4-(trifluoromethylthio)phenyl | Br |
| C-487 | 2,3,4-Trichlorophenyl | Br |
| C-488 | 2,3,5-Trichlorophenyl | Br |
| C-489 | 2,3,6-Trichlorophenyl | Br |
| C-490 | 2,4,5-Trichlorophenyl | Br |
| C-491 | 2,4,6-Trichlorophenyl | Br |
| C-492 | 3,4,5-Trichlorophenyl | Br |
| C-493 | 2,3,4-Trifluorophenyl | Br |
| C-494 | 2,3,5-Trifluorophenyl | Br |
| C-495 | 2,3,6-Trifluorophenyl | Br |
| C-496 | 2,4,5-Trifluorophenyl | Br |
| C-497 | 2,4,6-Trifluorophenyl | Br |
| C-498 | 3,4,5-Trifluorophenyl | Br |
| C-499 | 2,3,4-Trimethylphenyl | Br |
| C-500 | 2,3,5-Trimethylphenyl | Br |
| C-501 | 2,3,6-Trimethylphenyl | Br |
| C-502 | 2,4,5-Trimethylphenyl | Br |
| C-503 | 2,4,6-Trimethylphenyl | Br |
| C-504 | 3,4,5-Trimethylphenyl | Br |
| C-505 | 2,3,4-Trimethoxyphenyl | Br |
| C-506 | 2,3,5-Trimethoxyphenyl | Br |
| C-507 | 2,3,6-Trimethoxyphenyl | Br |
| C-508 | 2,4,5-Trimethoxyphenyl | Br |
| C-509 | 2,4,6-Trimethoxyphenyl | Br |
| C-510 | 3,4,5-Trimethoxyphenyl | Br |
| C-511 | Phenyl | OTs |
| C-512 | 2-Chlorophenyl | OTs |
| C-513 | 3-Chlorophenyl | OTs |
| C-514 | 4-Chlorophenyl | OTs |
| C-515 | 2-Fluorophenyl | OTs |
| C-516 | 3-Fluorophenyl | OTs |
| C-517 | 4-Fluorophenyl | OTs |
| C-518 | 2-Methylphenyl | OTs |
| C-519 | 3-Methylphenyl | OTs |
| C-520 | 4-Methylphenyl | OTs |
| C-521 | 2-Ethylphenyl | OTs |
| C-522 | 3-Ethylphenyl | OTs |
| C-523 | 4-Ethylphenyl | OTs |
| C-524 | 2-Methoxyphenyl | OTs |
| C-525 | 3-Methoxyphenyl | OTs |
| C-526 | 4-Methoxyphenyl | OTs |
| C-527 | 2-Trifluoromethylphenyl | OTs |
| C-528 | 3-Trifluoromethylphenyl | OTs |
| C-529 | 4-Trifluoromethylphenyl | OTs |
| C-530 | 2-Trifluoromethoxyphenyl | OTs |
| C-531 | 3-Trifluoromethoxyphenyl | OTs |
| C-532 | 4-Trifluoromethoxyphenyl | OTs |
| C-533 | 2-Difluoromethoxyphenyl | OTs |
| C-534 | 3-Difluoromethoxyphenyl | OTs |
| C-535 | 4-Difluoromethoxyphenyl | OTs |
| C-536 | 2-Trifluoromethylthiophenyl | OTs |
| C-537 | 3-Trifluoromethylthiophenyl | OTs |
| C-538 | 4-Trifluoromethylthiophenyl | OTs |
| C-539 | 2,3-Dichlorophenyl | OTs |
| C-540 | 2,4-Dichlorophenyl | OTs |
| C-541 | 2,5-Dichlorophenyl | OTs |
| C-542 | 2,6-Dichlorophenyl | OTs |
| C-543 | 3,4-Dichlorophenyl | OTs |
| C-544 | 3,5-Dichlorophenyl | OTs |
| C-545 | 2,3-Difluorophenyl | OTs |
| C-546 | 2,4-Difluorophenyl | OTs |
| C-547 | 2,5-Difluorophenyl | OTs |
| C-548 | 2,6-Difluorophenyl | OTs |
| C-549 | 3,4-Difluorophenyl | OTs |
| C-550 | 3,5-Difluorophenyl | OTs |
| C-551 | 2,3-Dimethylphenyl | OTs |
| C-552 | 2,4-Dimethylphenyl | OTs |
| C-553 | 2,5-Dimethylphenyl | OTs |
| C-554 | 2,6-Dimethylphenyl | OTs |
| C-555 | 3,4-Dimethylphenyl | OTs |
| C-556 | 3,5-Dimethylphenyl | OTs |
| C-557 | 2,3-Diethylphenyl | OTs |
| C-558 | 2,4-Diethylphenyl | OTs |
| C-559 | 2,5-Diethylphenyl | OTs |
| C-560 | 2,6-Diethylphenyl | OTs |
| C-561 | 3,5-Diethylphenyl | OTs |
| C-562 | 3,4-Diethylphenyl | OTs |
| C-563 | 2,3-Dimethoxyphenyl | OTs |
| C-564 | 2,4-Dimethoxyphenyl | OTs |
| C-565 | 2,5-Dimethoxyphenyl | OTs |
| C-566 | 2,6-Dimethoxyphenyl | OTs |
| C-567 | 3,4-Dimethoxyphenyl | OTs |
| C-568 | 3,5-Dimethoxyphenyl | OTs |
| C-569 | 2,3-Di(trifluoromethyl)phenyl | OTs |
| C-570 | 2,4-Di(trifluoromethyl)phenyl | OTs |
| C-571 | 2,5-Di(trifluoromethyl)phenyl | OTs |
| C-572 | 2,6-Di(trifluoromethyl)phenyl | OTs |
| C-573 | 3,4-Di(trifluoromethyl)phenyl | OTs |
| C-574 | 3,5-Di(trifluoromethyl)phenyl | OTs |
| C-575 | 2,3-Di(trifluoromethoxy)phenyl | OTs |
| C-576 | 2,4-Di(trifluoronnethoxy)phenyl | OTs |
| C-577 | 2,5-Di(trifluoromethoxy)phenyl | OTs |
| C-578 | 2,6-Di(trifluoromethoxy)phenyl | OTs |
| C-579 | 3,4-Di(trifluoromethoxy)phenyl | OTs |
| C-580 | 3,5-Di(trifluoromethoxy)phenyl | OTs |
| C-581 | 2,3-Di(difluoromethoxy)phenyl | OTs |
| C-582 | 2,4-Di(difluoromethoxy)phenyl | OTs |
| C-583 | 2,5-Di(difluoromethoxy)phenyl | OTs |
| C-584 | 2,6-Di(difluoromethoxy)phenyl | OTs |
| C-585 | 3,4-Di(difluoromethoxy)phenyl | OTs |
| C-586 | 3,5-Di(difluoromethoxy)phenyl | OTs |
| C-587 | 2,3-Di(trifluoromethylthio)phenyl | OTs |
| C-588 | 2,4-Di(trifluoromethylthio)phenyl | OTs |
| C-589 | 2,5-Di(trifluoromethylthio)phenyl | OTs |
| C-590 | 2,6-Di(trifluoromethylthio)phenyl | OTs |
| C-591 | 3,4-Di(trifluoromethylthio)phenyl | OTs |
| C-592 | 3,5-Di(trifluoromethylthio)phenyl | OTs |
| C-593 | 2-Fluoro-3-chlorophenyl | OTs |
| C-594 | 2-Fluoro-4-chlorophenyl | OTs |
| C-595 | 2-Fluoro-5-chlorophenyl | OTs |
| C-596 | 2-Fluoro-6-chlorophenyl | OTs |
| C-597 | 3-Fluoro-4-chlorophenyl | OTs |
| C-598 | 3-Fluoro-5-chlorophenyl | OTs |
| C-599 | 2-Chloro-3-fluorophenyl | OTs |
| C-600 | 2-Chloro-4-fluorophenyl | OTs |
| C-601 | 2-Chloro-5-fluorophenyl | OTs |
| C-602 | 3-Chloro-4-fluorophenyl | OTs |
| C-603 | 2-Methyl-3-chlorophenyl | OTs |
| C-604 | 2-Methyl-4-chlorophenyl | OTs |
| C-605 | 2-Methyl-5-chlorophenyl | OTs |
| C-606 | 2-Methyl-6-chlorophenyl | OTs |
| C-607 | 3-Methyl-4-chlorophenyl | OTs |
| C-608 | 3-Methyl-5-chlorophenyl | OTs |
| C-609 | 2-Chloro-3-methylphenyl | OTs |
| C-610 | 2-Chloro-4-methylphenyl | OTs |
| C-611 | 2-Chloro-5-methylphenyl | OTs |
| C-612 | 3-Chloro-4-methylphenyl | OTs |
| C-613 | 2-Methyl-3-fluorophenyl | OTs |
| C-614 | 2-Methyl-4-fluorophenyl | OTs |
| C-615 | 2-Methyl-5-fluorophenyl | OTs |
| C-616 | 2-Methyl-6-fluorophenyl | OTs |
| C-617 | 3-Methyl-4-fluorophenyl | OTs |
| C-618 | 3-Methyl-5-fluorophenyl | OTs |
| C-619 | 2-Fluoro-3-methylphenyl | OTs |
| C-620 | 2-Fluoro-4-methylphenyl | OTs |
| C-621 | 2-Fluoro-5-methylphenyl | OTs |
| C-622 | 3-Fluoro-4-methylphenyl | OTs |
| C-623 | 2-Chloro-3-ethylphenyl | OTs |
| C-624 | 2-Chloro-4-ethylphenyl | OTs |
| C-625 | 2-Chloro-5-ethylphenyl | OTs |
| C-626 | 3-Chloro-4-ethylphenyl | OTs |
| C-627 | 2-Ethyl-3-chlorophenyl | OTs |
| C-628 | 2-Ethyl-4-chlorophenyl | OTs |
| C-629 | 2-Ethyl-5-chlorophenyl | OTs |
| C-630 | 2-Ethyl-6-chlorophenyl | OTs |
| C-631 | 2-Ethyl-3-fluorophenyl | OTs |
| C-632 | 2-Ethyl-4-fluorophenyl | OTs |
| C-633 | 2-Ethyl-5-fluorophenyl | OTs |
| C-634 | 2-Ethyl-6-fluorophenyl | OTs |
| C-635 | 3-Ethyl-4-fluorophenyl | OTs |
| C-636 | 3-Ethyl-5-fluorophenyl | OTs |
| C-637 | 2-Fluoro-3-ethylphenyl | OTs |
| C-638 | 2-Fluoro-4-ethylphenyl | OTs |
| C-639 | 2-Fluoro-5-ethylphenyl | OTs |
| C-640 | 3-Fluoro-4-ethylphenyl | OTs |
| C-641 | 2-Methoxy-3-chlorophenyl | OTs |
| C-642 | 2-Methoxy-4-chlorophenyl | OTs |
| C-643 | 2-Methoxy-5-chlorophenyl | OTs |
| C-644 | 2-Methoxy-6-chlorophenyl | OTs |
| C-645 | 3-Methoxy-4-chlorophenyl | OTs |
| C-646 | 3-Methoxy-5-chlorophenyl | OTs |
| C-647 | 2-Chloro-3-methoxyphenyl | OTs |
| C-648 | 2-Chloro-4-methoxyphenyl | OTs |
| C-649 | 2-Chloro-5-methoxyphenyl | OTs |
| C-650 | 3-Chloro-4-methoxyphenyl | OTs |
| C-651 | 2-Methoxy-3-fluorophenyl | OTs |
| C-652 | 2-Methoxy-4-fluorophenyl | OTs |
| C-653 | 2-Methoxy-5-fluorophenyl | OTs |
| C-654 | 2-Methoxy-6-fluorophenyl | OTs |
| C-655 | 3-Methoxy-4-fluorophenyl | OTs |
| C-656 | 3-Methoxy-5-fluorophenyl | OTs |
| C-657 | 2-Fluoro-3-methoxyphenyl | OTs |
| C-658 | 2-Fluoro-4-methoxyphenyl | OTs |
| C-659 | 2-Fluoro-5-methoxyphenyl | OTs |
| C-660 | 3-Fluoro-4-methoxyphenyl | OTs |
| C-661 | 3-Fluoro-5-methoxyphenyl | OTs |
| C-662 | 2-(Trifluoromethyl)-3-chlorophenyl | OTs |
| C-663 | 2-(Trifluoromethyl)-4-chlorophenyl | OTs |
| C-664 | 2-(Trifluoromethyl)-5-chlorophenyl | OTs |
| C-665 | 2-(Trifluoromethyl)-6-chlorophenyl | OTs |
| C-666 | 3-(Trifluoromethyl)-4-chlorophenyl | OTs |
| C-667 | 3-(Trifluoromethyl)-5-chlorophenyl | OTs |
| C-668 | 2-Chloro-3-(trifluoromethyl)phenyl | OTs |
| C-669 | 2-Chloro-4-(trifluoromethyl)phenyl | OTs |
| C-670 | 2-Chloro-5-(trifluoromethyl)phenyl | OTs |
| C-671 | 3-Chloro-4-(trifluoromethyl)phenyl | OTs |
| C-672 | 2-(Trifluoromethyl)-3-fluorophenyl | OTs |
| C-673 | 2-(Trifluoromethyl)-4-fluorophenyl | OTs |
| C-674 | 2-(Trifluoromethyl)-5-fluorophenyl | OTs |
| C-675 | 2-(Trifluoromethyl)-6-fluorophenyl | OTs |
| C-676 | 3-(Trifluoromethyl)-4-fluorophenyl | OTs |
| C-677 | 3-(Trifluoromethyl)-5-fluorophenyl | OTs |
| C-678 | 2-Fluoro-3-(trifluoromethyl)phenyl | OTs |
| C-679 | 2-Fluoro-4-(trifluoromethyl)phenyl | OTs |
| C-680 | 2-Fluoro-5-(trifluoromethyl)phenyl | OTs |
| C-681 | 3-Fluoro-4-(trifluoromethyl)phenyl | OTs |
| C-682 | 2-(Trifluoromethoxy)-3-chlorophenyl | OTs |
| C-683 | 2-(Trifluoromethoxy)-4-chlorophenyl | OTs |
| C-684 | 2-(Trifluoromethoxy)-5-chlorophenyl | OTs |
| C-685 | 2-(Trifluoromethoxy)-6-chlorophenyl | OTs |
| C-686 | 3-(Trifluoromethoxy)-4-chlorophenyl | OTs |
| C-687 | 3-(Trifluoromethoxy)-5-chlorophenyl | OTs |
| C-688 | 2-Chloro-3-(trifluoromethoxy)phenyl | OTs |
| C-689 | 2-Chloro-4-(trifluoromethoxy)phenyl | OTs |
| C-690 | 2-Chloro-5-(trifluoromethoxy)phenyl | OTs |
| C-691 | 3-Chloro-4-(trifluoromethoxy)phenyl | OTs |
| C-692 | 2-(Trifluoromethoxy)-3-fluorophenyl | OTs |
| C-693 | 2-(Trifluoromethoxy)-4-fluorophenyl | OTs |
| C-694 | 2-(Trifluoromethoxy)-5-fluorophenyl | OTs |
| C-695 | 2-(Trifluoromethoxy)-6-fluorophenyl | OTs |
| C-696 | 3-(Trifluoromethoxy)-4-fluorophenyl | OTs |
| C-697 | 3-(Trifluoromethoxy)-5-fluorophenyl | OTs |
| C-698 | 2-Fluoro-3-(trifluoromethoxy)phenyl | OTs |
| C-699 | 2-Fluoro-4-(trifluoromethoxy)phenyl | OTs |
| C-700 | 2-Fluoro-5-(trifluoromethoxy)phenyl | OTs |
| C-701 | 3-Fluoro-4-(trifluoromethoxy)phenyl | OTs |
| C-702 | 2-(Difluoromethoxy)-3-chlorophenyl | OTs |
| C-703 | 2-(Difluoromethoxy)-4-chlorophenyl | OTs |
| C-704 | 2-(Difluoromethoxy)-5-chlorophenyl | OTs |
| C-705 | 2-(Difluoromethoxy)-6-chlorophenyl | OTs |
| C-706 | 3-(Difluoromethoxy)-4-chlorophenyl | OTs |
| C-707 | 3-(Difluoromethoxy)-5-chlorophenyl | OTs |
| C-708 | 2-Chloro-3-(difluoromethoxy)phenyl | OTs |
| C-709 | 2-Chloro-4-(difluoromethoxy)phenyl | OTs |
| C-710 | 2-Chloro-5-(difluoromethoxy)phenyl | OTs |
| C-711 | 3-Chloro-4-(difluoromethoxy)phenyl | OTs |
| C-712 | 2-(Difluoromethoxy)-3-fluorophenyl | OTs |
| C-713 | 2-(Difluoromethoxy)-4-fluorophenyl | OTs |
| C-714 | 2-(Difluoromethoxy)-5-fluorophenyl | OTs |
| C-715 | 2-(Difluoromethoxy)-6-fluorophenyl | OTs |
| C-716 | 3-(Difluoromethoxy)-4-fluorophenyl | OTs |
| C-717 | 3-(Difluoromethoxy)-5-fluorophenyl | OTs |
| C-718 | 2-Fluoro-3-(difluoromethoxy)phenyl | OTs |
| C-719 | 2-Fluoro-4-(difluoromethoxy)phenyl | OTs |
| C-720 | 2-Fluoro-5-(difluoromethoxy)phenyl | OTs |
| C-721 | 3-Fluoro-4-(difluoromethoxy)phenyl | OTs |
| C-722 | 2-(Trifluoromethylthio)-3-chlorophenyl | OTs |
| C-723 | 2-(Trifluoromethylthio)-4-chlorophenyl | OTs |
| C-724 | 2-(Trifluoromethylthio)-5-chlorophenyl | OTs |
| C-725 | 2-(Trifluoromethylthio)-6-chlorophenyl | OTs |
| C-726 | 3-(Trifluoromethylthio)-4-chlorophenyl | OTs |
| C-727 | 3-(Trifluoromethylthio)-5-chlorophenyl | OTs |
| C-728 | 2-Chloro-3-(trifluoromethylthio)phenyl | OTs |
| C-729 | 2-Chloro-4-(trifluoromethylthio)phenyl | OTs |
| C-730 | 2-Chloro-5-(trifluoromethylthio)phenyl | OTs |
| C-731 | 3-Chloro-4-(trifluoromethylthio)phenyl | OTs |
| C-732 | 2-(Trifluoromethylthio)-3-fluorophenyl | OTs |
| C-733 | 2-(Trifluoromethylthio)-4-fluorophenyl | OTs |
| C-734 | 2-(Trifluoromethylthio)-5-fluorophenyl | OTs |
| C-735 | 2-(Trifluoromethylthio)-6-fluorophenyl | OTs |
| C-736 | 3-(Trifluoromethylthio)-4-fluorophenyl | OTs |
| C-737 | 3-(Trifluoromethylthio)-5-fluorophenyl | OTs |
| C-738 | 2-Fluoro-3-(trifluoromethylthio)phenyl | OTs |
| C-739 | 2-Fluoro-4-(trifluoromethylthio)phenyl | OTs |
| C-740 | 2-Fluoro-5-(trifluoromethylthio)phenyl | OTs |
| C-741 | 3-Fluoro-4-(trifluoromethylthio)phenyl | OTs |
| C-742 | 2,3,4-Trichlorophenyl | OTs |
| C-743 | 2,3,5-Trichlorophenyl | OTs |
| C-744 | 2,3,6-Trichlorophenyl | OTs |
| C-745 | 2,4,5-Trichlorophenyl | OTs |
| C-746 | 2,4,6-Trichlorophenyl | OTs |
| C-747 | 3,4,5-Trichlorophenyl | OTs |
| C-748 | 2,3,4-Trifluorophenyl | OTs |
| C-749 | 2,3,5-Trifluorophenyl | OTs |
| C-750 | 2,3,6-Trifluorophenyl | OTs |
| C-751 | 2,4,5-Trifluorophenyl | OTs |
| C-752 | 2,4,6-Trifluorophenyl | OTs |
| C-753 | 3,4,5-Trifluorophenyl | OTs |
| C-754 | 2,3,4-Trimethylphenyl | OTs |
| C-755 | 2,3,5-Trimethylphenyl | OTs |
| C-756 | 2,3,6-Trimethylphenyl | OTs |
| C-757 | 2,4,5-Trimethylphenyl | OTs |
| C-758 | 2,4,6-Trimethylphenyl | OTs |
| C-759 | 3,4,5-Trimethylphenyl | OTs |
| C-760 | 2,3,4-Trimethoxyphenyl | OTs |
| C-761 | 2,3,5-Trimethoxyphenyl | OTs |
| C-762 | 2,3,6-Trimethoxyphenyl | OTs |
| C-763 | 2,4,5-Trimethoxyphenyl | OTs |
| C-764 | 2,4,6-Trimethoxyphenyl | OTs |
| C-765 | 3,4,5-Trimethoxyphenyl | OTs |
| C-766 | Phenyl | OMs |
| C-767 | 2-Chlorophenyl | OMs |
| C-768 | 3-Chlorophenyl | OMs |
| C-769 | 4-Chlorophenyl | OMs |
| C-770 | 2-Fluorophenyl | OMs |
| C-771 | 3-Fluorophenyl | OMs |
| C-772 | 4-Fluorophenyl | OMs |
| C-773 | 2-Methylphenyl | OMs |
| C-774 | 3-Methylphenyl | OMs |
| C-775 | 4-Methylphenyl | OMs |
| C-776 | 2-Ethylphenyl | OMs |
| C-777 | 3-Ethylphenyl | OMs |
| C-778 | 4-Ethylphenyl | OMs |
| C-779 | 2-Methoxyphenyl | OMs |
| C-780 | 3-Methoxyphenyl | OMs |
| C-781 | 4-Methoxyphenyl | OMs |
| C-782 | 2-Trifluoromethylphenyl | OMs |
| C-783 | 3-Trifluoromethylphenyl | OMs |
| C-784 | 4-Trifluoromethylphenyl | OMs |
| C-785 | 2-Trifluoromethoxyphenyl | OMs |
| C-786 | 3-Trifluoromethoxyphenyl | OMs |
| C-787 | 4-Trifluoromethoxyphenyl | OMs |
| C-788 | 2-Difluoromethoxyphenyl | OMs |
| C-789 | 3-Difluoromethoxyphenyl | OMs |
| C-790 | 4-Difluoromethoxyphenyl | OMs |
| C-791 | 2-Trifluoromethylthiophenyl | OMs |
| C-792 | 3-Trifluoromethylthiophenyl | OMs |
| C-793 | 4-Trifluoromethylthiophenyl | OMs |
| C-794 | 2,3-Dichlorophenyl | OMs |
| C-795 | 2,4-Dichlorophenyl | OMs |
| C-796 | 2,5-Dichlorophenyl | OMs |
| C-797 | 2,6-Dichlorophenyl | OMs |
| C-798 | 3,4-Dichlorophenyl | OMs |
| C-799 | 3,5-Dichlorophenyl | OMs |
| C-800 | 2,3-Difluorophenyl | OMs |
| C-801 | 2,4-Difluorophenyl | OMs |
| C-802 | 2,5-Difluorophenyl | OMs |
| C-803 | 2,6-Difluorophenyl | OMs |
| C-804 | 3,4-Difluorophenyl | OMs |
| C-805 | 3,5-Difluorophenyl | OMs |
| C-806 | 2,3-Dimethylphenyl | OMs |
| C-807 | 2,4-Dimethylphenyl | OMs |
| C-808 | 2,5-Dimethylphenyl | OMs |
| C-809 | 2,6-Dimethylphenyl | OMs |
| C-810 | 3,4-Dimethylphenyl | OMs |
| C-811 | 3,5-Dimethylphenyl | OMs |
| C-812 | 2,3-Diethylphenyl | OMs |
| C-813 | 2,4-Diethylphenyl | OMs |
| C-814 | 2,5-Diethylphenyl | OMs |
| C-815 | 2,6-Diethylphenyl | OMs |
| C-816 | 3,5-Diethylphenyl | OMs |
| C-817 | 3,4-Diethylphenyl | OMs |
| C-818 | 2,3-Dimethoxyphenyl | OMs |
| C-819 | 2,4-Dimethoxyphenyl | OMs |
| C-820 | 2,5-Dimethoxyphenyl | OMs |
| C-821 | 2,6-Dimethoxyphenyl | OMs |
| C-822 | 3,4-Dimethoxyphenyl | OMs |
| C-823 | 3,5-Dimethoxyphenyl | OMs |
| C-824 | 2,3-Di(trifluoromethyl)phenyl | OMs |
| C-825 | 2,4-Di(trifluoromethyl)phenyl | OMs |
| C-826 | 2,5-Di(trifluoromethyl)phenyl | OMs |
| C-827 | 2,6-Di(trifluoromethyl)phenyl | OMs |
| C-828 | 3,4-Di(trifluoromethyl)phenyl | OMs |
| C-829 | 3,5-Di(trifluoromethyl)phenyl | OMs |
| C-830 | 2,3-Di(trifluoromethoxy)phenyl | OMs |
| C-831 | 2,4-Di(trifluoromethoxy)phenyl | OMs |
| C-832 | 2,5-Di(trifluoromethoxy)phenyl | OMs |
| C-833 | 2,6-Di(trifluoromethoxy)phenyl | OMs |
| C-834 | 3,4-Di(trifluoromethoxy)phenyl | OMs |
| C-835 | 3,5-Di(trifluoromethoxy)phenyl | OMs |
| C-836 | 2,3-Di(difluoromethoxy)phenyl | OMs |
| C-837 | 2,4-Di(difluoromethoxy)phenyl | OMs |
| C-838 | 2,5-Di(difluoromethoxy)phenyl | OMs |
| C-839 | 2,6-Di(difluoromethoxy)phenyl | OMs |
| C-840 | 3,4-Di(difluoromethoxy)phenyl | OMs |
| C-841 | 3,5-Di(difluoromethoxy)phenyl | OMs |
| C-842 | 2,3-Di(trifluoromethylthio)phenyl | OMs |
| C-843 | 2,4-Di(trifluoromethylthio)phenyl | OMs |
| C-844 | 2,5-Di(trifluoromethylthio)phenyl | OMs |
| C-845 | 2,6-Di(trifluoromethylthio)phenyl | OMs |
| C-846 | 3,4-Di(trifluoromethylthio)phenyl | OMs |
| C-847 | 3,5-Di(trifluoromethylthio)phenyl | OMs |
| C-848 | 2-Fluoro-3-chlorophenyl | OMs |
| C-849 | 2-Fluoro-4-chlorophenyl | OMs |
| C-850 | 2-Fluoro-5-chlorophenyl | OMs |
| C-851 | 2-Fluoro-6-chlorophenyl | OMs |
| C-852 | 3-Fluoro-4-chlorophenyl | OMs |
| C-853 | 3-Fluoro-5-chlorophenyl | OMs |
| C-854 | 2-Chloro-3-fluorophenyl | OMs |
| C-855 | 2-Chloro-4-fluorophenyl | OMs |
| C-856 | 2-Chloro-5-fluorophenyl | OMs |
| C-857 | 3-Chloro-4-fluorophenyl | OMs |
| C-858 | 2-Methyl-3-chlorophenyl | OMs |
| C-859 | 2-Methyl-4-chlorophenyl | OMs |
| C-860 | 2-Methyl-5-chlorophenyl | OMs |
| C-861 | 2-Methyl-6-chlorophenyl | OMs |
| C-862 | 3-Methyl-4-chlorophenyl | OMs |
| C-863 | 3-Methyl-5-chlorophenyl | OMs |
| C-864 | 2-Chloro-3-methylphenyl | OMs |
| C-865 | 2-Chloro-4-methylphenyl | OMs |
| C-866 | 2-Chloro-5-methylphenyl | OMs |
| C-867 | 3-Chloro-4-methylphenyl | OMs |
| C-868 | 2-Methyl-3-fluorophenyl | OMs |
| C-869 | 2-Methyl-4-fluorophenyl | OMs |
| C-870 | 2-Methyl-5-fluorophenyl | OMs |
| C-871 | 2-Methyl-6-fluorophenyl | OMs |
| C-872 | 3-Methyl-4-fluorophenyl | OMs |
| C-873 | 3-Methyl-5-fluorophenyl | OMs |
| C-874 | 2-Fluoro-3-methylphenyl | OMs |
| C-875 | 2-Fluoro-4-methylphenyl | OMs |
| C-876 | 2-Fluoro-5-methylphenyl | OMs |
| C-877 | 3-Fluoro-4-methylphenyl | OMs |
| C-878 | 2-Chloro-3-ethylphenyl | OMs |
| C-879 | 2-Chloro-4-ethylphenyl | OMs |
| C-880 | 2-Chloro-5-ethylphenyl | OMs |
| C-881 | 3-Chloro-4-ethylphenyl | OMs |
| C-882 | 2-Ethyl-3-chlorophenyl | OMs |
| C-883 | 2-Ethyl-4-chlorophenyl | OMs |
| C-884 | 2-Ethyl-5-chlorophenyl | OMs |
| C-885 | 2-Ethyl-6-chlorophenyl | OMs |
| C-886 | 2-Ethyl-3-fluorophenyl | OMs |
| C-887 | 2-Ethyl-4-fluorophenyl | OMs |
| C-888 | 2-Ethyl-5-fluorophenyl | OMs |
| C-889 | 2-Ethyl-6-fluorophenyl | OMs |
| C-890 | 3-Ethyl-4-fluorophenyl | OMs |
| C-891 | 3-Ethyl-5-fluorophenyl | OMs |
| C-892 | 2-Fluoro-3-ethylphenyl | OMs |
| C-893 | 2-Fluoro-4-ethylphenyl | OMs |
| C-894 | 2-Fluoro-5-ethylphenyl | OMs |
| C-895 | 3-Fluoro-4-ethylphenyl | OMs |
| C-896 | 2-Methoxy-3-chlorophenyl | OMs |
| C-897 | 2-Methoxy-4-chlorophenyl | OMs |
| C-898 | 2-Methoxy-5-chlorophenyl | OMs |
| C-899 | 2-Methoxy-6-chlorophenyl | OMs |
| C-900 | 3-Methoxy-4-chlorophenyl | OMs |
| C-901 | 3-Methoxy-5-chlorophenyl | OMs |
| C-902 | 2-Chloro-3-methoxyphenyl | OMs |
| C-903 | 2-Chloro-4-methoxyphenyl | OMs |
| C-904 | 2-Chloro-5-methoxyphenyl | OMs |
| C-905 | 3-Chloro-4-methoxyphenyl | OMs |
| C-906 | 2-Methoxy-3-fluorophenyl | OMs |
| C-907 | 2-Methoxy-4-fluorophenyl | OMs |
| C-908 | 2-Methoxy-5-fluorophenyl | OMs |
| C-909 | 2-Methoxy-6-fluorophenyl | OMs |
| C-910 | 3-Methoxy-4-fluorophenyl | OMs |
| C-911 | 3-Methoxy-5-fluorophenyl | OMs |
| C-912 | 2-Fluoro-3-methoxyphenyl | OMs |
| C-913 | 2-Fluoro-4-methoxyphenyl | OMs |
| C-914 | 2-Fluoro-5-methoxyphenyl | OMs |
| C-915 | 3-Fluoro-4-methoxyphenyl | OMs |
| C-916 | 3-Fluoro-5-methoxyphenyl | OMs |
| C-917 | 2-(Trifluoromethyl)-3-chlorophenyl | OMs |
| C-918 | 2-(Trifluoromethyl)-4-chlorophenyl | OMs |
| C-919 | 2-(Trifluoromethyl)-5-chlorophenyl | OMs |
| C-920 | 2-(Trifluoromethyl)-6-chlorophenyl | OMs |
| C-921 | 3-(Trifluoromethyl)-4-chlorophenyl | OMs |
| C-922 | 3-(Trifluoromethyl)-5-chlorophenyl | OMs |
| C-923 | 2-Chloro-3-(trifluoromethyl)phenyl | OMs |
| C-924 | 2-Chloro-4-(trifluoromethyl)phenyl | OMs |
| C-925 | 2-Chloro-5-(trifluoromethyl)phenyl | OMs |
| C-926 | 3-Chloro-4-(trifluoromethyl)phenyl | OMs |
| C-927 | 2-(Trifluoromethyl)-3-fluorophenyl | OMs |
| C-928 | 2-(Trifluoromethyl)-4-fluorophenyl | OMs |
| C-929 | 2-(Trifluoromethyl)-5-fluorophenyl | OMs |
| C-930 | 2-(Trifluoromethyl)-6-fluorophenyl | OMs |
| C-931 | 3-(Trifluoromethyl)-4-fluorophenyl | OMs |
| C-932 | 3-(Trifluoromethyl)-5-fluorophenyl | OMs |
| C-933 | 2-Fluoro-3-(trifluoromethyl)phenyl | OMs |
| C-934 | 2-Fluoro-4-(trifluoromethyl)phenyl | OMs |
| C-935 | 2-Fluoro-5-(trifluoromethyl)phenyl | OMs |
| C-936 | 3-Fluoro-4-(trifluoromethyl)phenyl | OMs |
| C-937 | 2-(Trifluoromethoxy)-3-chlorophenyl | OMs |
| C-938 | 2-(Trifluoromethoxy)-4-chlorophenyl | OMs |
| C-939 | 2-(Trifluoromethoxy)-5-chlorophenyl | OMs |
| C-940 | 2-(Trifluoromethoxy)-6-chlorophenyl | OMs |
| C-941 | 3-(Trifluoromethoxy)-4-chlorophenyl | OMs |
| C-942 | 3-(Trifluoromethoxy)-5-chlorophenyl | OMs |
| C-943 | 2-Chloro-3-(trifluoromethoxy)phenyl | OMs |
| C-944 | 2-Chloro-4-(trifluoromethoxy)phenyl | OMs |
| C-945 | 2-Chloro-5-(trifluoromethoxy)phenyl | OMs |
| C-946 | 3-Chloro-4-(trifluoromethoxy)phenyl | OMs |
| C-947 | 2-(Trifluoromethoxy)-3-fluorophenyl | OMs |
| C-948 | 2-(Trifluoromethoxy)-4-fluorophenyl | OMs |
| C-949 | 2-(Trifluoromethoxy)-5-fluorophenyl | OMs |
| C-950 | 2-(Trifluoromethoxy)-6-fluorophenyl | OMs |
| C-951 | 3-(Trifluoromethoxy)-4-fluorophenyl | OMs |
| C-952 | 3-(Trifluoromethoxy)-5-fluorophenyl | OMs |
| C-953 | 2-Fluoro-3-(trifluoromethoxy)phenyl | OMs |
| C-954 | 2-Fluoro-4-(trifluoromethoxy)phenyl | OMs |
| C-955 | 2-Fluoro-5-(trifluoromethoxy)phenyl | OMs |
| C-956 | 3-Fluoro-4-(trifluoromethoxy)phenyl | OMs |
| C-957 | 2-(Difluoromethoxy)-3-chlorophenyl | OMs |
| C-958 | 2-(Difluoromethoxy)-4-chlorophenyl | OMs |
| C-959 | 2-(Difluoromethoxy)-5-chlorophenyl | OMs |
| C-960 | 2-(Difluoromethoxy)-6-chlorophenyl | OMs |
| C-961 | 3-(Difluoromethoxy)-4-chlorophenyl | OMs |
| C-962 | 3-(Difluoromethoxy)-5-chlorophenyl | OMs |
| C-963 | 2-Chloro-3-(difluoromethoxy)phenyl | OMs |
| C-964 | 2-Chloro-4-(difluoromethoxy)phenyl | OMs |
| C-965 | 2-Chloro-5-(difluoromethoxy)phenyl | OMs |
| C-966 | 3-Chloro-4-(difluoromethoxy)phenyl | OMs |
| C-967 | 2-(Difluoromethoxy)-3-fluorophenyl | OMs |
| C-968 | 2-(Difluoromethoxy)-4-fluorophenyl | OMs |
| C-969 | 2-(Difluoromethoxy)-5-fluorophenyl | OMs |
| C-970 | 2-(Difluoromethoxy)-6-fluorophenyl | OMs |
| C-971 | 3-(Difluoromethoxy)-4-fluorophenyl | OMs |
| C-972 | 3-(Difluoromethoxy)-5-fluorophenyl | OMs |
| C-973 | 2-Fluoro-3-(difluoromethoxy)phenyl | OMs |
| C-974 | 2-Fluoro-4-(difluoromethoxy)phenyl | OMs |
| C-975 | 2-Fluoro-5-(difluoromethoxy)phenyl | OMs |
| C-976 | 3-Fluoro-4-(difluoromethoxy)phenyl | OMs |
| C-977 | 2-(Trifluoromethylthio)-3-chlorophenyl | OMs |
| C-978 | 2-(Trifluoromethylthio)-4-chlorophenyl | OMs |
| C-979 | 2-(Trifluoromethylthio)-5-chlorophenyl | OMs |
| C-980 | 2-(Trifluoromethylthio)-6-chlorophenyl | OMs |
| C-981 | 3-(Trifluoromethylthio)-4-chlorophenyl | OMs |
| C-982 | 3-(Trifluoromethylthio)-5-chlorophenyl | OMs |
| C-983 | 2-Chloro-3-(trifluoromethylthio)phenyl | OMs |
| C-984 | 2-Chloro-4-(trifluoromethylthio)phenyl | OMs |
| C-985 | 2-Chloro-5-(trifluoromethylthio)phenyl | OMs |
| C-986 | 3-Chloro-4-(trifluoromethylthio)phenyl | OMs |
| C-987 | 2-(Trifluoromethylthio)-3-fluorophenyl | OMs |
| C-988 | 2-(Trifluoromethylthio)-4-fluorophenyl | OMs |
| C-989 | 2-(Trifluoromethylthio)-5-fluorophenyl | OMs |
| C-990 | 2-(Trifluoromethylthio)-6-fluorophenyl | OMs |
| C-991 | 3-(Trifluoromethylthio)-4-fluorophenyl | OMs |
| C-992 | 3-(Trifluoromethylthio)-5-fluorophenyl | OMs |
| C-993 | 2-Fluoro-3-(trifluoromethylthio)phenyl | OMs |
| C-994 | 2-Fluoro-4-(trifluoromethylthio)phenyl | OMs |
| C-995 | 2-Fluoro-5-(trifluoromethylthio)phenyl | OMs |
| C-996 | 3-Fluoro-4-(trifluoromethylthio)phenyl | OMs |
| C-997 | 2,3,4-Trichlorophenyl | OMs |
| C-998 | 2,3,5-Trichlorophenyl | OMs |
| C-999 | 2,3,6-Trichlorophenyl | OMs |
| C-1000 | 2,4,5-Trichlorophenyl | OMs |
| C-1001 | 2,4,6-Trichlorophenyl | OMs |
| C-1002 | 3,4,5-Trichlorophenyl | OMs |
| C-1003 | 2,3,4-Trifluorophenyl | OMs |
| C-1004 | 2,3,5-Trifluorophenyl | OMs |
| C-1005 | 2,3,6-Trifluorophenyl | OMs |
| C-1006 | 2,4,5-Trifluorophenyl | OMs |
| C-1007 | 2,4,6-Trifluorophenyl | OMs |
| C-1008 | 3,4,5-Trifluorophenyl | OMs |
| C-1009 | 2,3,4-Trimethylphenyl | OMs |
| C-1010 | 2,3,5-Trimethylphenyl | OMs |
| C-1011 | 2,3,6-Trimethylphenyl | OMs |
| C-1012 | 2,4,5-Trimethylphenyl | OMs |
| C-1013 | 2,4,6-Trimethylphenyl | OMs |
| C-1014 | 3,4,5-Trimethylphenyl | OMs |
| C-1015 | 2,3,4-Trimethoxyphenyl | OMs |
| C-1016 | 2,3,5-Trimethoxyphenyl | OMs |
| C-1017 | 2,3,6-Trimethoxyphenyl | OMs |
| C-1018 | 2,4,5-Trimethoxyphenyl | OMs |
| C-1019 | 2,4,6-Trimethoxyphenyl | OMs |
| C-1020 | 3,4,5-Trimethoxyphenyl | OMs |
| C-1021 | Phenyl | OH |
| C-1022 | 2-Chlorophenyl | OH |
| C-1023 | 3-Chlorophenyl | OH |
| C-1024 | 4-Chlorophenyl | OH |
| C-1025 | 2-Fluorophenyl | OH |
| C-1026 | 3-Fluorophenyl | OH |
| C-1027 | 4-Fluorophenyl | OH |
| C-1028 | 2-Methylphenyl | OH |
| C-1029 | 3-Methylphenyl | OH |
| C-1030 | 4-Methylphenyl | OH |
| C-1031 | 2-Ethylphenyl | OH |
| C-1032 | 3-Ethylphenyl | OH |
| C-1033 | 4-Ethylphenyl | OH |
| C-1034 | 2-Methoxyphenyl | OH |
| C-1035 | 3-Methoxyphenyl | OH |
| C-1036 | 4-Methoxyphenyl | OH |
| C-1037 | 2-Trifluoromethylphenyl | OH |
| C-1038 | 3-Trifluoromethylphenyl | OH |
| C-1039 | 4-Trifluoromethylphenyl | OH |
| C-1040 | 2-Trifluoromethoxyphenyl | OH |
| C-1041 | 3-Trifluoromethoxyphenyl | OH |
| C-1042 | 4-Trifluoromethoxyphenyl | OH |
| C-1043 | 2-Difluoromethoxyphenyl | OH |
| C-1044 | 3-Difluoromethoxyphenyl | OH |
| C-1045 | 4-Difluoromethoxyphenyl | OH |
| C-1046 | 2-Trifluoromethylthiophenyl | OH |
| C-1047 | 3-Trifluoromethylthiophenyl | OH |
| C-1048 | 4-Trifluoromethylthiophenyl | OH |
| C-1049 | 2,3-Dichlorophenyl | OH |
| C-1050 | 2,4-Dichlorophenyl | OH |
| C-1051 | 2,5-Dichlorophenyl | OH |
| C-1052 | 2,6-Dichlorophenyl | OH |
| C-1053 | 3,4-Dichlorophenyl | OH |
| C-1054 | 3,5-Dichlorophenyl | OH |
| C-1055 | 2,3-Difluorophenyl | OH |
| C-1056 | 2,4-Difluorophenyl | OH |
| C-1057 | 2,5-Difluorophenyl | OH |
| C-1058 | 2,6-Difluorophenyl | OH |
| C-1059 | 3,4-Difluorophenyl | OH |
| C-1060 | 3,5-Difluorophenyl | OH |
| C-1061 | 2,3-Dimethylphenyl | OH |
| C-1062 | 2,4-Dimethylphenyl | OH |
| C-1063 | 2,5-Dimethylphenyl | OH |
| C-1064 | 2,6-Dimethylphenyl | OH |
| C-1065 | 3,4-Dimethylphenyl | OH |
| C-1066 | 3,5-Dimethylphenyl | OH |
| C-1067 | 2,3-Diethylphenyl | OH |
| C-1068 | 2,4-Diethylphenyl | OH |
| C-1069 | 2,5-Diethylphenyl | OH |
| C-1070 | 2,6-Diethylphenyl | OH |
| C-1071 | 3,5-Diethylphenyl | OH |
| C-1072 | 3,4-Diethylphenyl | OH |
| C-1073 | 2,3-Dimethoxyphenyl | OH |
| C-1074 | 2,4-Dimethoxyphenyl | OH |
| C-1075 | 2,5-Dimethoxyphenyl | OH |
| C-1076 | 2,6-Dimethoxyphenyl | OH |
| C-1077 | 3,4-Dimethoxyphenyl | OH |
| C-1078 | 3,5-Dimethoxyphenyl | OH |
| C-1079 | 2,3-Di(trifluoromethyl)phenyl | OH |
| C-1080 | 2,4-Di(trifluoromethyl)phenyl | OH |
| C-1081 | 2,5-Di(trifluoromethyl)phenyl | OH |
| C-1082 | 2,6-Di(trifluoromethyl)phenyl | OH |
| C-1083 | 3,4-Di(trifluoromethyl)phenyl | OH |
| C-1084 | 3,5-Di(trifluoromethyl)phenyl | OH |
| C-1085 | 2,3-Di(trifluoromethoxy)phenyl | OH |
| C-1086 | 2,4-Di(trifluoromethoxy)phenyl | OH |
| C-1087 | 2,5-Di(trifluoromethoxy)phenyl | OH |
| C-1088 | 2,6-Di(trifluoromethoxy)phenyl | OH |
| C-1089 | 3,4-Di(trifluoromethoxy)phenyl | OH |
| C-1090 | 3,5-Di(trifluoromethoxy)phenyl | OH |
| C-1091 | 2,3-Di(difluoromethoxy)phenyl | OH |
| C-1092 | 2,4-Di(difluoromethoxy)phenyl | OH |
| C-1093 | 2,5-Di(difluoromethoxy)phenyl | OH |
| C-1094 | 2,6-Di(difluoromethoxy)phenyl | OH |
| C-1095 | 3,4-Di(difluoromethoxy)phenyl | OH |
| C-1096 | 3,5-Di(difluoromethoxy)phenyl | OH |
| C-1097 | 2,3-Di(trifluoromethylthio)phenyl | OH |
| C-1098 | 2,4-Di(trifluoromethylthio)phenyl | OH |
| C-1099 | 2,5-Di(trifluoromethylthio)phenyl | OH |
| C-1100 | 2,6-Di(trifluoromethylthio)phenyl | OH |
| C-1101 | 3,4-Di(trifluoromethylthio)phenyl | OH |
| C-1102 | 3,5-Di(trifluoromethylthio)phenyl | OH |
| C-1103 | 2-Fluoro-3-chlorophenyl | OH |
| C-1104 | 2-Fluoro-4-chlorophenyl | OH |
| C-1105 | 2-Fluoro-5-chlorophenyl | OH |
| C-1106 | 2-Fluoro-6-chlorophenyl | OH |
| C-1107 | 3-Fluoro-4-chlorophenyl | OH |
| C-1108 | 3-Fluoro-5-chlorophenyl | OH |
| C-1109 | 2-Chloro-3-fluorophenyl | OH |
| C-1110 | 2-Chloro-4-fluorophenyl | OH |
| C-1111 | 2-Chloro-5-fluorophenyl | OH |
| C-1112 | 3-Chloro-4-fluorophenyl | OH |
| C-1113 | 2-Methyl-3-chlorophenyl | OH |
| C-1114 | 2-Methyl-4-chlorophenyl | OH |
| C-1115 | 2-Methyl-5-chlorophenyl | OH |
| C-1116 | 2-Methyl-6-chlorophenyl | OH |
| C-1117 | 3-Methyl-4-chlorophenyl | OH |
| C-1118 | 3-Methyl-5-chlorophenyl | OH |
| C-1119 | 2-Chloro-3-methylphenyl | OH |
| C-1120 | 2-Chloro-4-methylphenyl | OH |
| C-1121 | 2-Chloro-5-methylphenyl | OH |
| C-1122 | 3-Chloro-4-methylphenyl | OH |
| C-1123 | 2-Methyl-3-fluorophenyl | OH |
| C-1124 | 2-Methyl-4-fluorophenyl | OH |
| C-1125 | 2-Methyl-5-fluorophenyl | OH |
| C-1126 | 2-Methyl-6-fluorophenyl | OH |
| C-1127 | 3-Methyl-4-fluorophenyl | OH |
| C-1128 | 3-Methyl-5-fluorophenyl | OH |
| C-1129 | 2-Fluoro-3-methylphenyl | OH |
| C-1130 | 2-Fluoro-4-methylphenyl | OH |
| C-1131 | 2-Fluoro-5-methylphenyl | OH |
| C-1132 | 3-Fluoro-4-methylphenyl | OH |
| C-1133 | 2-Chloro-3-ethylphenyl | OH |
| C-1134 | 2-Chloro-4-ethylphenyl | OH |
| C-1135 | 2-Chloro-5-ethylphenyl | OH |
| C-1136 | 3-Chloro-4-ethylphenyl | OH |
| C-1137 | 2-Ethyl-3-chlorophenyl | OH |
| C-1138 | 2-Ethyl-4-chlorophenyl | OH |
| C-1139 | 2-Ethyl-5-chlorophenyl | OH |
| C-1140 | 2-Ethyl-6-chlorophenyl | OH |
| C-1141 | 2-Ethyl-3-fluorophenyl | OH |
| C-1142 | 2-Ethyl-4-fluorophenyl | OH |
| C-1143 | 2-Ethyl-5-fluorophenyl | OH |
| C-1144 | 2-Ethyl-6-fluorophenyl | OH |
| C-1145 | 3-Ethyl-4-fluorophenyl | OH |
| C-1146 | 3-Ethyl-5-fluorophenyl | OH |
| C-1147 | 2-Fluoro-3-ethylphenyl | OH |
| C-1148 | 2-Fluoro-4-ethylphenyl | OH |
| C-1149 | 2-Fluoro-5-ethylphenyl | OH |
| C-1150 | 3-Fluoro-4-ethylphenyl | OH |
| C-1151 | 2-Methoxy-3-chlorophenyl | OH |
| C-1152 | 2-Methoxy-4-chlorophenyl | OH |
| C-1153 | 2-Methoxy-5-chlorophenyl | OH |
| C-1154 | 2-Methoxy-6-chlorophenyl | OH |
| C-1155 | 3-Methoxy-4-chlorophenyl | OH |
| C-1156 | 3-Methoxy-5-chlorophenyl | OH |
| C-1157 | 2-Chloro-3-methoxyphenyl | OH |
| C-1158 | 2-Chloro-4-methoxyphenyl | OH |
| C-1159 | 2-Chloro-5-methoxyphenyl | OH |
| C-1160 | 3-Chloro-4-methoxyphenyl | OH |
| C-1161 | 2-Methoxy-3-fluorophenyl | OH |
| C-1162 | 2-Methoxy-4-fluorophenyl | OH |
| C-1163 | 2-Methoxy-5-fluorophenyl | OH |
| C-1164 | 2-Methoxy-6-fluorophenyl | OH |
| C-1165 | 3-Methoxy-4-fluorophenyl | OH |
| C-1166 | 3-Methoxy-5-fluorophenyl | OH |
| C-1167 | 2-Fluoro-3-methoxyphenyl | OH |
| C-1168 | 2-Fluoro-4-methoxyphenyl | OH |
| C-1169 | 2-Fluoro-5-methoxyphenyl | OH |
| C-1170 | 3-Fluoro-4-methoxyphenyl | OH |
| C-1171 | 3-Fluoro-5-methoxyphenyl | OH |
| C-1172 | 2-(Trifluoromethyl)-3-chlorophenyl | OH |
| C-1173 | 2-(Trifluoromethyl)-4-chlorophenyl | OH |
| C-1174 | 2-(Trifluoromethyl)-5-chlorophenyl | OH |
| C-1175 | 2-(Trifluoromethyl)-6-chlorophenyl | OH |
| C-1176 | 3-(Trifluoromethyl)-4-chlorophenyl | OH |
| C-1177 | 3-(Trifluoromethyl)-5-chlorophenyl | OH |
| C-1178 | 2-Chloro-3-(trifluoromethyl)phenyl | OH |
| C-1179 | 2-Chloro-4-(trifluoromethyl)phenyl | OH |
| C-1180 | 2-Chloro-5-(trifluoromethyl)phenyl | OH |
| C-1181 | 3-Chloro-4-(trifluoromethyl)phenyl | OH |
| C-1182 | 2-(Trifluoromethyl)-3-fluorophenyl | OH |
| C-1183 | 2-(Trifluoromethyl)-4-fluorophenyl | OH |
| C-1184 | 2-(Trifluoromethyl)-5-fluorophenyl | OH |
| C-1185 | 2-(Trifluoromethyl)-6-fluorophenyl | OH |
| C-1186 | 3-(Trifluoromethyl)-4-fluorophenyl | OH |
| C-1187 | 3-(Trifluoromethyl)-5-fluorophenyl | OH |
| C-1188 | 2-Fluoro-3-(trifluoromethyl)phenyl | OH |
| C-1189 | 2-Fluoro-4-(trifluoromethyl)phenyl | OH |
| C-1190 | 2-Fluoro-5-(trifluoromethyl)phenyl | OH |
| C-1191 | 3-Fluoro-4-(trifluoromethyl)phenyl | OH |
| C-1192 | 2-(Trifluoromethoxy)-3-chlorophenyl | OH |
| C-1193 | 2-(Trifluoromethoxy)-4-chlorophenyl | OH |
| C-1194 | 2-(Trifluoromethoxy)-5-chlorophenyl | OH |
| C-1195 | 2-(Trifluoromethoxy)-6-chlorophenyl | OH |
| C-1196 | 3-(Trifluoromethoxy)-4-chlorophenyl | OH |
| C-1197 | 3-(Trifluoromethoxy)-5-chlorophenyl | OH |
| C-1198 | 2-Chloro-3-(trifluoromethoxy)phenyl | OH |
| C-1199 | 2-Chloro-4-(trifluoromethoxy)phenyl | OH |
| C-1200 | 2-Chloro-5-(trifluoromethoxy)phenyl | OH |
| C-1201 | 3-Chloro-4-(trifluoromethoxy)phenyl | OH |
| C-1202 | 2-(Trifluoromethoxy)-3-fluorophenyl | OH |
| C-1203 | 2-(Trifluoromethoxy)-4-fluorophenyl | OH |
| C-1204 | 2-(Trifluoromethoxy)-5-fluorophenyl | OH |
| C-1205 | 2-(Trifluoromethoxy)-6-fluorophenyl | OH |
| C-1206 | 3-(Trifluoromethoxy)-4-fluorophenyl | OH |
| C-1207 | 3-(Trifluoromethoxy)-5-fluorophenyl | OH |
| C-1208 | 2-Fluoro-3-(trifluoromethoxy)phenyl | OH |
| C-1209 | 2-Fluoro-4-(trifluoromethoxy)phenyl | OH |
| C-1210 | 2-Fluoro-5-(trifluoromethoxy)phenyl | OH |
| C-1211 | 3-Fluoro-4-(trifluoromethoxy)phenyl | OH |
| C-1212 | 2-(Difluoromethoxy)-3-chlorophenyl | OH |
| C-1213 | 2-(Difluoromethoxy)-4-chlorophenyl | OH |
| C-1214 | 2-(Difluoromethoxy)-5-chlorophenyl | OH |
| C-1215 | 2-(Difluoromethoxy)-6-chlorophenyl | OH |
| C-1216 | 3-(Difluoromethoxy)-4-chlorophenyl | OH |
| C-1217 | 3-(Difluoromethoxy)-5-chlorophenyl | OH |
| C-1218 | 2-Chloro-3-(difluoromethoxy)phenyl | OH |
| C-1219 | 2-Chloro-4-(difluoromethoxy)phenyl | OH |
| C-1220 | 2-Chloro-5-(difluoromethoxy)phenyl | OH |
| C-1221 | 3-Chloro-4-(difluoromethoxy)phenyl | OH |
| C-1222 | 2-(Difluoromethoxy)-3-fluorophenyl | OH |
| C-1223 | 2-(Difluoromethoxy)-4-fluorophenyl | OH |
| C-1224 | 2-(Difluoromethoxy)-5-fluorophenyl | OH |
| C-1225 | 2-(Difluoromethoxy)-6-fluorophenyl | OH |
| C-1226 | 3-(Difluoromethoxy)-4-fluorophenyl | OH |
| C-1227 | 3-(Difluoromethoxy)-5-fluorophenyl | OH |
| C-1228 | 2-Fluoro-3-(difluoromethoxy)phenyl | OH |
| C-1229 | 2-Fluoro-4-(difluoromethoxy)phenyl | OH |
| C-1230 | 2-Fluoro-5-(difluoromethoxy)phenyl | OH |
| C-1231 | 3-Fluoro-4-(difluoromethoxy)phenyl | OH |
| C-1232 | 2-(Trifluoromethylthio)-3-chlorophenyl | OH |
| C-1233 | 2-(Trifluoromethylthio)-4-chlorophenyl | OH |
| C-1234 | 2-(Trifluoromethylthio)-5-chlorophenyl | OH |
| C-1235 | 2-(Trifluoromethylthio)-6-chlorophenyl | OH |
| C-1236 | 3-(Trifluoromethylthio)-4-chlorophenyl | OH |
| C-1237 | 3-(Trifluoromethylthio)-5-chlorophenyl | OH |
| C-1238 | 2-Chloro-3-(trifluoromethylthio)phenyl | OH |
| C-1239 | 2-Chloro-4-(trifluoromethylthio)phenyl | OH |
| C-1240 | 2-Chloro-5-(trifluoromethylthio)phenyl | OH |
| C-1241 | 3-Chloro-4-(trifluoromethylthio)phenyl | OH |
| C-1242 | 2-(Trifluoromethylthio)-3-fluorophenyl | OH |
| C-1243 | 2-(Trifluoromethylthio)-4-fluorophenyl | OH |
| C-1244 | 2-(Trifluoromethylthio)-5-fluorophenyl | OH |
| C-1245 | 2-(Trifluoromethylthio)-6-fluorophenyl | OH |
| C-1246 | 3-(Trifluoromethylthio)-4-fluorophenyl | OH |
| C-1247 | 3-(Trifluoromethylthio)-5-fluorophenyl | OH |
| C-1248 | 2-Fluoro-3-(trifluoromethylthio)phenyl | OH |
| C-1249 | 2-Fluoro-4-(trifluoromethylthio)phenyl | OH |
| C-1250 | 2-Fluoro-5-(trifluoromethylthio)phenyl | OH |
| C-1251 | 3-Fluoro-4-(trifluoromethylthio)phenyl | OH |
| C-1252 | 2,3,4-Trichlorophenyl | OH |
| C-1253 | 2,3,5-Trichlorophenyl | OH |
| C-1254 | 2,3,6-Trichlorophenyl | OH |
| C-1255 | 2,4,5-Trichlorophenyl | OH |
| C-1256 | 2,4,6-Trichlorophenyl | OH |
| C-1257 | 3,4,5-Trichlorophenyl | OH |
| C-1258 | 2,3,4-Trifluorophenyl | OH |
| C-1259 | 2,3,5-Trifluorophenyl | OH |
| C-1260 | 2,3,6-Trifluorophenyl | OH |
| C-1261 | 2,4,5-Trifluorophenyl | OH |
| C-1262 | 2,4,6-Trifluorophenyl | OH |
| C-1263 | 3,4,5-Trifluorophenyl | OH |
| C-1264 | 2,3,4-Trimethylphenyl | OH |
| C-1265 | 2,3,5-Trimethylphenyl | OH |
| C-1266 | 2,3,6-Trimethylphenyl | OH |
| C-1267 | 2,4,5-Trimethylphenyl | OH |
| C-1268 | 2,4,6-Trimethylphenyl | OH |
| C-1269 | 3,4,5-Trimethylphenyl | OH |
| C-1270 | 2,3,4-Trimethoxyphenyl | OH |
| C-1271 | 2,3,5-Trimethoxyphenyl | OH |
| C-1272 | 2,3,6-Trimethoxyphenyl | OH |
| C-1273 | 2,4,5-Trimethoxyphenyl | OH |
| C-1274 | 2,4,6-Trimethoxyphenyl | OH |
| C-1275 | 3,4,5-Trimethoxyphenyl | OH |

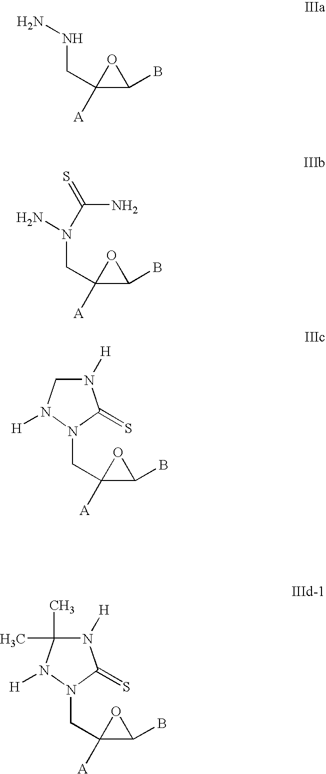

In particular with a view to their use, preference is given to the compounds IIIa, IIIb, IIIc and IIId-1 according to the invention compiled in Tables 1e to 162e, Tables 1f to 162f, Tables 1g to 162g and Tables 1h to 162h, respectively. The groups mentioned in the tables for a substituent are furthermore per se, independently of the combination in which they are mentioned, a particularly preferred embodiment of the substituent in question.

Table 1e
Compounds IIIa in which B is 2,3-difluorophenyl and the combination of A and D corresponds in each case to one row of Table B (compounds IIIa.1eB-1 to IIIa.1eB-255)

Table 2e
Compounds IIIa in which B is 2,4-difluorophenyl and the combination of A and D corresponds in each case to one row of Table B (compounds IIIa.2eB-1 to IIIa.2eB-255)

Table 3e
Compounds IIIa in which B is 2,5-difluorophenyl and the combination of A and D corresponds in each case to one row of Table B (compounds IIIa.3eB-1 to IIIa.3eB-255)

Table 4e
Compounds IIIa in which B is 2,6-difluorophenyl and the combination of A and D corresponds in each case to one row of Table B (compounds IIIa.4eB-1 to IIIa.4eB-255)

Table 5e
Compounds IIIa in which B is 2-fluoro-3-chlorophenyl and the combination of A and D corresponds in each case to one row of Table B (compounds IIIa.5eB-1 to IIIa.5eB-255)

Table 6e
Compounds IIIa in which B is 2-fluoro-4-chlorophenyl and the combination of A and D corresponds in each case to one row of Table B (compounds IIIa.6eB-1 to IIIa.6eB-255)

Table 7e
Compounds IIIa in which B is 2-fluoro-5-chlorophenyl and the combination of A and D corresponds in each case to one row of Table B (compounds IIIa.7eB-1 to IIIa.7eB-255)

Table 8e
Compounds IIIa in which B is 2-fluoro-6-chlorophenyl and the combination of A and D corresponds in each case to one row of Table B (compounds IIIa.8eB-1 to IIIa.8eB-255)

Table 9e
Compounds IIIa in which B is 2-chloro-3-fluorophenyl and the combination of A and D corresponds in each case to one row of Table B (compounds IIIa.9eB-1 to IIIa.9eB-255)

Table 10e
Compounds IIIa in which B is 2-chloro-4-fluorophenyl and the combination of A and D corresponds in each case to one row of Table B (compounds IIIa.10eB-1 to IIIa.10eB-255)

Table 11e
Compounds IIIa in which B is 2-chloro-5-fluorophenyl and the combination of A and D corresponds in each case to one row of Table B (compounds IIIa.11eB-1 to IIIa.11eB-255)

Table 12e
Compounds IIIa in which B is 2-methyl-3-fluorophenyl and the combination of A and D corresponds in each case to one row of Table B (compounds IIIa.12eB-1 to IIIa.12eB-255)

Table 13e
Compounds IIIa in which B is 2-methyl-4-fluorophenyl and the combination of A and D corresponds in each case to one row of Table B (compounds IIIa.13eB-1 to IIIa.13eB-255)

Table 14e
Compounds IIIa in which B is 2-methyl-5-fluorophenyl and the combination of A and D corresponds in each case to one row of Table B (compounds IIIa.14eB-1 to IIIa.14eB-255)

Table 15e
Compounds IIIa in which B is 2-methyl-6-fluorophenyl and the combination of A and D corresponds in each case to one row of Table B (compounds IIIa.15eB-1 to IIIa.15eB-255)

Table 16e
Compounds IIIa in which B is 2-fluoro-3-methylphenyl and the combination of A and D corresponds in each case to one row of Table B (compounds IIIa.16eB-1 to IIIa.16eB-255)

Table 17e
Compounds IIIa in which B is 2-fluoro-4-methylphenyl and the combination of A and D corresponds in each case to one row of Table B (compounds IIIa.17eB-1 to IIIa.17eB-255)

Table 18e
Compounds IIIa in which B is 2-fluoro-5-methylphenyl and the combination of A and D corresponds in each case to one row of Table B (compounds IIIa.18eB-1 to IIIa.18eB-255)

Table 19e
Compounds IIIa in which B is 2-ethyl-3-fluorophenyl and the combination of A and D corresponds in each case to one row of Table B (compounds IIIa.19eB-1 to IIIa.19eB-255)

Table 20e
Compounds IIIa in which B is 2-ethyl-4-fluorophenyl and the combination of A and D corresponds in each case to one row of Table B (compounds IIIa.20eB-1 to IIIa.20eB-255)

Table 21e
Compounds IIIa in which B is 2-ethyl-5-fluorophenyl and the combination of A and D corresponds in each case to one row of Table B (compounds IIIa.21eB-1 to IIIa.21eB-255)

Table 22e
Compounds IIIa in which B is 2-ethyl-6-fluorophenyl and the combination of A and D corresponds in each case to one row of Table B (compounds IIIa.22eB-1 to IIIa.22eB-255)

Table 23e
Compounds IIIa in which B is 2-fluoro-3-ethylphenyl and the combination of A and D corresponds in each case to one row of Table B (compounds IIIa.23eB-1 to IIIa.23eB-255)

Table 24e
Compounds IIIa in which B is 2-fluoro-4-ethylphenyl and the combination of A and D corresponds in each case to one row of Table B (compounds IIIa.24eB-1 to IIIa.24eB-255)

Table 25e
Compounds IIIa in which B is 2-methoxy-3-fluorophenyl and the combination of A and D corresponds in each case to one row of Table B (compounds IIIa.25eB-1 to IIIa.25eB-255)

Table 26e
Compounds IIIa in which B is 2-methoxy-4-fluorophenyl and the combination of A and D corresponds in each case to one row of Table B (compounds IIIa.26eB-1 to IIIa.26eB-255)

Table 27e
Compounds IIIa in which B is 2-methoxy-5-fluorophenyl and the combination of A and D corresponds in each case to one row of Table B (compounds IIIa.27eB-1 to IIIa.27eB-255)

Table 28e
Compounds IIIa in which B is 2-methoxy-6-fluorophenyl and the combination of A and D corresponds in each case to one row of Table B (compounds IIIa.28eB-1 to IIIa.28eB-255)

Table 29e
Compounds IIIa in which B is 2-fluoro-3-methoxyphenyl and the combination of A and D corresponds in each case to one row of Table B (compounds IIIa.29eB-1 to IIIa.29eB-255)

Table 30e
Compounds IIIa in which B is 2-fluoro-4-methoxyphenyl and the combination of A and D corresponds in each case to one row of Table B (compounds IIIa.30eB-1 to IIIa.30eB-255)

Table 31e
Compounds IIIa in which B is 2-fluoro-5-methoxyphenyl and the combination of A and D corresponds in each case to one row of Table B (compounds IIIa.31eB-1 to IIIa.31eB-255)

Table 32e
Compounds IIIa in which B is 2-(trifluoromethyl)-3-fluorophenyl and the combination of A and D corresponds in each case to one row of Table B (compounds IIIa.32eB-1 to IIIa.32eB-255)

Table 33e
Compounds IIIa in which B is 2,4,5-trimethoxyphenyl and the combination of A and D corresponds in each case to one row of Table B (compounds IIIa.33eB-1 to IIIa.33eB-255)

Table 34e
Compounds IIIa in which B is 2,4,6-trimethoxyphenyl and the combination of A and D corresponds in each case to one row of Table B (compounds IIIa.34eB-1 to IIIa.34eB-255)

Table 35e
Compounds IIIa in which B is 2-fluoro-5-ethylphenyl and the combination of A and D corresponds in each case to one row of Table B (compounds IIIa.35eB-1 to IIIa.35eB-255)

Table 36e
Compounds IIIa in which B is 2-(trifluoromethyl)-4-fluorophenyl and the combination of A and D corresponds in each case to one row of Table B (compounds IIIa.36eB-1 to IIIa.36eB-255)

Table 37e
Compounds IIIa in which B is 2-(trifluoromethyl)-5-fluorophenyl and the combination of A and D corresponds in each case to one row of Table B (compounds IIIa.37eB-1 to IIIa.37eB-255)

Table 38e
Compounds IIIa in which B is 2-(trifluoromethyl)-6-fluorophenyl and the combination of A and D corresponds in each case to one row of Table B (compounds IIIa.38eB-1 to IIIa.38eB-255)

Table 39e
Compounds IIIa in which B is 2-fluoro-3-(trifluoromethyl)phenyl and the combination of A and D corresponds in each case to one row of Table B (compounds IIIa.39eB-1 to IIIa.39eB-255)

Table 40e
Compounds IIIa in which B is 2-fluoro-4-(trifluoromethyl)phenyl and the combination of A and D corresponds in each case to one row of Table B (compounds IIIa.40eB-1 to IIIa.40eB-255)

Table 41e
Compounds IIIa in which B is 2-fluoro-5-(trifluoromethyl)phenyl and the combination of A and D corresponds in each case to one row of Table B (compounds IIIa.41eB-1 to IIIa.41eB-255)

Table 42e
Compounds IIIa in which B is 2-(trifluoromethoxy)-3-fluorophenyl and the combination of A and D corresponds in each case to one row of Table B (compounds IIIa.42eB-1 to IIIa.42eB-255)

Table 43e
Compounds IIIa in which B is 2-(trifluoromethoxy)-4-fluorophenyl and the combination of A and D corresponds in each case to one row of Table B (compounds IIIa.43eB-1 to IIIa.43eB-255)

Table 44e
Compounds IIIa in which B is 2-(trifluoromethoxy)-5-fluorophenyl and the combination of A and D corresponds in each case to one row of Table B (compounds IIIa.44eB-1 to IIIa.44eB-255)

Table 45e
Compounds IIIa in which B is 2-(trifluoromethoxy)-6-fluorophenyl and the combination of A and D corresponds in each case to one row of Table B (compounds IIIa.45eB-1 to IIIa.45eB-255)

Table 46e
Compounds IIIa in which B is 2-fluoro-3-(trifluoromethoxy)phenyl and the combination of A and D corresponds in each case to one row of Table B (compounds IIIa.46eB-1 to IIIa.46eB-255)

Table 47e
Compounds IIIa in which B is 2-fluoro-4-(trifluoromethoxy)phenyl and the combination of A and D corresponds in each case to one row of Table B (compounds IIIa.47eB-1 to IIIa.47eB-255)

Table 48e
Compounds IIIa in which B is 2-fluoro-5-(trifluoromethoxy)phenyl and the combination of A and D corresponds in each case to one row of Table B (compounds IIIa.48eB-1 to IIIa.48eB-255)

Table 49e
Compounds IIIa in which B is 2-(difluoromethoxy)-3-fluorophenyl and the combination of A and D corresponds in each case to one row of Table B (compounds IIIa.49eB-1 to IIIa.49eB-255)

Table 50e
Compounds IIIa in which B is 2-(difluoromethoxy)-4-fluorophenyl and the combination of A and D corresponds in each case to one row of Table B (compounds IIIa.50eB-1 to IIIa.50eB-255)

Table 51e
Compounds IIIa in which B is 2-(difluoromethoxy)-5-fluorophenyl and the combination of A and D corresponds in each case to one row of Table B (compounds IIIa.51eB-1 to IIIa.51eB-255)

Table 52e
Compounds IIIa in which B is 2-(difluoromethoxy)-6-fluorophenyl and the combination of A and D corresponds in each case to one row of Table B (compounds IIIa.52eB-1 to IIIa.52eB-255)

Table 53e
Compounds IIIa in which B is 2-fluoro-3-(difluoromethoxy)phenyl and the combination of A and D corresponds in each case to one row of Table B (compounds IIIa.53eB-1 to IIIa.53eB-255)

Table 54e
Compounds IIIa in which B is 2-fluoro-4-(difluoromethoxy)phenyl and the combination of A and D corresponds in each case to one row of Table B (compounds IIIa.54eB-1 to IIIa.54eB-255)

Table 55e
Compounds IIIa in which B is 2-fluoro-5-(difluoromethoxy)phenyl and the combination of A and D corresponds in each case to one row of Table B (compounds IIIa.55eB-1 to IIIa.55eB-255)

Table 56e
Compounds IIIa in which B is 2-(trifluoromethylthio)-3-fluorophenyl and the combination of A and D corresponds in each case to one row of Table B (compounds IIIa.56eB-1 to IIIa.56eB-255)

Table 57e
Compounds IIIa in which B is 2-(trifluoromethylthio)-4-fluorophenyl and the combination of A and D corresponds in each case to one row of Table B (compounds IIIa.57eB-1 to IIIa.57eB-255)

Table 58e
Compounds IIIa in which B is 2-(trifluoromethylthio)-5-fluorophenyl and the combination of A and D corresponds in each case to one row of Table B (compounds IIIa.58eB-1 to IIIa.58eB-255)

Table 59e
Compounds IIIa in which B is 2-(trifluoromethylthio)-6-fluorophenyl and the combination of A and D corresponds in each case to one row of Table B (compounds IIIa.59eB-1 to IIIa.59eB-255)

Table 60e
Compounds IIIa in which B is 2-fluoro-3-(trifluoromethylthio)phenyl and the combination of A and D corresponds in each case to one row of Table B (compounds IIIa.60eB-1 to IIIa.60eB-255)

Table 61e
Compounds IIIa in which B is 2-fluoro-4-(trifluoromethylthio)phenyl and the combination of A and D corresponds in each case to one row of Table B (compounds IIIa.61eB-1 to IIIa.61eB-255)

Table 62e
Compounds IIIa in which B is 2-fluoro-5-(trifluoromethylthio)phenyl and the combination of A and D corresponds in each case to one row of Table B (compounds IIIa.62eB-1 to IIIa.62eB-255)

Table 63e
Compounds IIIa in which B is 2,3,4-trifluorophenyl and the combination of A and D corresponds in each case to one row of Table B (compounds IIIa.63eB-1 to IIIa.63eB-255)

Table 64e
Compounds IIIa in which B is 2,3,5-trifluorophenyl and the combination of A and D corresponds in each case to one row of Table B (compounds IIIa.64eB-1 to IIIa.64eB-255)

Table 65e
Compounds IIIa in which B is 2,3,6-trifluorophenyl and the combination of A and D corresponds in each case to one row of Table B (compounds IIIa.65eB-1 to IIIa.65eB-255)

Table 66e
Compounds IIIa in which B is 2,4,5-trifluorophenyl and the combination of A and D corresponds in each case to one row of Table B (compounds IIIa.66eB-1 to IIIa.66eB-255)

Table 67e
Compounds IIIa in which B is 2,4,6-trifluorophenyl and the combination of A and D corresponds in each case to one row of Table B (compounds IIIa.67eB-1 to IIIa.67eB-255)

Table 68e
Compounds IIIa in which B is 2,3-dichlorophenyl and the combination of A and D corresponds in each case to one row of Table B (compounds IIIa.68eB-1 to IIIa.68eB-255)

Table 69e
Compounds IIIa in which B is 2,4-dichlorophenyl and the combination of A and D corresponds in each case to one row of Table B (compounds IIIa.69eB-1 to IIIa.69eB-255)

Table 70e
Compounds IIIa in which B is 2,5-dichlorophenyl and the combination of A and D corresponds in each case to one row of Table B (compounds IIIa.70eB-1 to IIIa.70eB-255)

Table 71e
Compounds IIIa in which B is 2,6-dichlorophenyl and the combination of A and D corresponds in each case to one row of Table B (compounds IIIa.71eB-1 to IIIa.71eB-255)

Table 72e
Compounds IIIa in which B is 2,3-dimethylphenyl and the combination of A and D corresponds in each case to one row of Table B (compounds IIIa.72eB-1 to IIIa.72eB-255)

Table 73e
Compounds IIIa in which B is 2,4-dimethylphenyl and the combination of A and D corresponds in each case to one row of Table B (compounds IIIa.73eB-1 to IIIa.73eB-255)

Table 74e
Compounds IIIa in which B is 2,5-dimethylphenyl and the combination of A and D corresponds in each case to one row of Table B (compounds IIIa.74eB-1 to IIIa.74eB-255)

Table 75e
Compounds IIIa in which B is 2,6-dimethylphenyl and the combination of A and D corresponds in each case to one row of Table B (compounds IIIa.75eB-1 to IIIa.75eB-255)

Table 76e
Compounds IIIa in which B is 2,3-diethylphenyl and the combination of A and D corresponds in each case to one row of Table B (compounds IIIa.76eB-1 to IIIa.76eB-255)

Table 77e
Compounds IIIa in which B is 2,4-diethylphenyl and the combination of A and D corresponds in each case to one row of Table B (compounds IIIa.77eB-1 to IIIa.77eB-255)

Table 78e
Compounds IIIa in which B is 2,5-diethylphenyl and the combination of A and D corresponds in each case to one row of Table B (compounds IIIa.78eB-1 to IIIa.78eB-255)

Table 79e
Compounds IIIa in which B is 2,6-diethylphenyl and the combination of A and D corresponds in each case to one row of Table B (compounds IIIa.79eB-1 to IIIa.79eB-255)

Table 80e
Compounds IIIa in which B is 2,3-dimethoxyphenyl and the combination of A and D corresponds in each case to one row of Table B (compounds IIIa.80eB-1 to IIIa.80eB-255)

Table 81e
Compounds IIIa in which B is 2,4-dimethoxyphenyl and the combination of A and D corresponds in each case to one row of Table B (compounds IIIa.81eB-1 to IIIa.81 eB-255)

Table 82e
Compounds IIIa in which B is 2,5-dimethoxyphenyl and the combination of A and D corresponds in each case to one row of Table B (compounds IIIa.82eB-1 to IIIa.82eB-255)

Table 83e
Compounds IIIa in which B is 2,6-dimethoxyphenyl and the combination of A and D corresponds in each case to one row of Table B (compounds IIIa.83eB-1 to IIIa.83eB-255)

Table 84e
Compounds IIIa in which B is 2,3-di(trifluoromethyl)phenyl and the combination of A and D corresponds in each case to one row of Table B (compounds IIIa.84eB-1 to IIIa.84eB-255)

Table 85e
Compounds IIIa in which B is 2,4-di(trifluoromethyl)phenyl and the combination of A and D corresponds in each case to one row of Table B (compounds IIIa.85eB-1 to IIIa.85eB-255)

Table 86e
Compounds IIIa in which B is 2,5-di(trifluoromethyl)phenyl and the combination of A and D corresponds in each case to one row of Table B (compounds IIIa.86eB-1 to IIIa.86eB-255)

Table 87e
Compounds IIIa in which B is 2,6-di(trifluoromethyl)phenyl and the combination of A and D corresponds in each case to one row of Table B (compounds IIIa.87eB-1 to IIIa.87eB-255)

Table 88e
Compounds IIIa in which B is 2,3-di(trifluoromethoxy)phenyl and the combination of A and D corresponds in each case to one row of Table B (compounds IIIa.88eB-1 to IIIa.88eB-255)

Table 89e
Compounds IIIa in which B is 2,4-di(trifluoromethoxy)phenyl and the combination of A and D corresponds in each case to one row of Table B (compounds IIIa.89eB-1 to IIIa.89eB-255)

Table 90e
Compounds IIIa in which B is 2,5-di(trifluoromethoxy)phenyl and the combination of A and D corresponds in each case to one row of Table B (compounds IIIa.90eB-1 to IIIa.90eB-255)

Table 91e
Compounds IIIa in which B is 2,6-di(trifluoromethoxy)phenyl and the combination of A and D corresponds in each case to one row of Table B (compounds IIIa.91eB-1 to IIIa.91eB-255)

Table 92e
Compounds IIIa in which B is 2,3-di(difluoromethoxy)phenyl and the combination of A and D corresponds in each case to one row of Table B (compounds IIIa.92eB-1 to IIIa.92eB-255)

Table 93e
Compounds IIIa in which B is 2,4-di(difluoromethoxy)phenyl and the combination of A and D corresponds in each case to one row of Table B (compounds IIIa.93eB-1 to IIIa.93eB-255)

Table 94e
Compounds IIIa in which B is 2,5-di(difluoromethoxy)phenyl and the combination of A and D corresponds in each case to one row of Table B (compounds IIIa.94eB-1 to IIIa.94eB-255)

Table 95e
Compounds IIIa in which B is 2,6-di(difluoromethoxy)phenyl and the combination of A and D corresponds in each case to one row of Table B (compounds IIIa.95eB-1 to IIIa.95eB-255)

Table 96e
Compounds IIIa in which B is 2,3-di(trifluoromethylthio)phenyl and the combination of A and D corresponds in each case to one row of Table B (compounds IIIa.96eB-1 to IIIa.96eB-255)

Table 97e
Compounds IIIa in which B is 2,4-di(trifluoronnethylthio)phenyl and the combination of A and D corresponds in each case to one row of Table B (compounds IIIa.97eB-1 to IIIa.97eB-255)

Table 98e
Compounds IIIa in which B is 2,5-di(trifluoromethylthio)phenyl and the combination of A and D corresponds in each case to one row of Table B (compounds IIIa.98eB-1 to IIIa.98eB-255)

Table 99e
Compounds IIIa in which B is 2,6-di(trifluoromethylthio)phenyl and the combination of A and D corresponds in each case to one row of Table B (compounds IIIa.99eB-1 to IIIa.99eB-255)

Table 100e
Compounds IIIa in which B is 2-methyl-3-chlorophenyl and the combination of A and D corresponds in each case to one row of Table B (compounds IIIa.100eB-1 to IIIa.100eB-255)

Table 101e
Compounds IIIa in which B is 2-methyl-4-chlorophenyl and the combination of A and D corresponds in each case to one row of Table B (compounds IIIa.101eB-1 to IIIa.101eB-255)

Table 102e
Compounds IIIa in which B is 2-methyl-5-chlorophenyl and the combination of A and D corresponds in each case to one row of Table B (compounds IIIa.102eB-1 to IIIa.102eB-255)

Table 103e
Compounds IIIa in which B is 2-methyl-6-chlorophenyl and the combination of A and D corresponds in each case to one row of Table B (compounds IIIa.103eB-1 to IIIa.103eB-255)

Table 104e
Compounds IIIa in which B is 2-chloro-3-methylphenyl and the combination of A and D corresponds in each case to one row of Table B (compounds IIIa.104eB-1 to IIIa.104eB-255)

Table 105e
Compounds IIIa in which B is 2-chloro-4-methylphenyl and the combination of A and D corresponds in each case to one row of Table B (compounds IIIa.105eB-1 to IIIa.105eB-255)

Table 106e
Compounds IIIa in which B is 2-chloro-5-methylphenyl and the combination of A and D corresponds in each case to one row of Table B (compounds IIIa.106eB-1 to IIIa.106eB-255)

Table 107e
Compounds IIIa in which B is 2-chloro-3-ethylphenyl and the combination of A and D corresponds in each case to one row of Table B (compounds IIIa.107eB-1 to IIIa.107eB-255)

Table 108e
Compounds IIIa in which B is 2-chloro-4-ethylphenyl and the combination of A and D corresponds in each case to one row of Table B (compounds IIIa.108eB-1 to IIIa.108eB-255)

Table 109e
Compounds IIIa in which B is 2-chloro-5-ethylphenyl and the combination of A and D corresponds in each case to one row of Table B (compounds IIIa.109eB-1 to IIIa.109eB-255)

Table 110e
Compounds IIIa in which B is 2-ethyl-3-chlorophenyl and the combination of A and D corresponds in each case to one row of Table B (compounds IIIa.110eB-1 to IIIa.110eB-255)

Table 111e
Compounds IIIa in which B is 2-ethyl-4-chlorophenyl and the combination of A and D corresponds in each case to one row of Table B (compounds IIIa.111eB-1 to IIIa.111eB-255)

Table 112e
Compounds IIIa in which B is 2-ethyl-5-chlorophenyl and the combination of A and D corresponds in each case to one row of Table B (compounds IIIa.112eB-1 to IIIa.112eB-255)

Table 113e
Compounds IIIa in which B is 2-ethyl-6-chlorophenyl and the combination of A and D corresponds in each case to one row of Table B (compounds IIIa.113eB-1 to IIIa.113eB-255)

Table 114e
Compounds IIIa in which B is 2-methoxy-3-chlorophenyl and the combination of A and D corresponds in each case to one row of Table B (compounds IIIa.114eB-1 to IIIa.114eB-255)

Table 115e
Compounds IIIa in which B is 2-methoxy-4-chlorophenyl and the combination of A and D corresponds in each case to one row of Table B (compounds IIIa.115eB-1 to IIIa.115eB-255)

Table 116e
Compounds IIIa in which B is 2-methoxy-5-chlorophenyl and the combination of A and D corresponds in each case to one row of Table B (compounds IIIa.116eB-1 to IIIa.116eB-255)

Table 117e
Compounds IIIa in which B is 2-methoxy-6-chlorophenyl and the combination of A and D corresponds in each case to one row of Table B (compounds IIIa.117eB-1 to IIIa.117eB-255)

Table 118e
Compounds IIIa in which B is 2-chloro-3-methoxyphenyl and the combination of A and D corresponds in each case to one row of Table B (compounds IIIa.118eB-1 to IIIa.118eB-255)

Table 119e
Compounds IIIa in which B is 2-chloro-4-methoxyphenyl and the combination of A and D corresponds in each case to one row of Table B (compounds IIIa.119eB-1 to IIIa.119eB-255)

Table 120e
Compounds IIIa in which B is 2-chloro-5-methoxyphenyl and the combination of A and D corresponds in each case to one row of Table B (compounds IIIa.120eB-1 to IIIa.120eB-255)

Table 121e
Compounds IIIa in which B is 2-(trifluoromethyl)-3-chlorophenyl and the combination of A and D corresponds in each case to one row of Table B (compounds IIIa.121eB-1 to IIIa.121eB-255)

Table 122e
Compounds IIIa in which B is 2-(trifluoromethyl)-4-chlorophenyl and the combination of A and D corresponds in each case to one row of Table B (compounds IIIa.122eB-1 to IIIa.122eB-255)

Table 123e
Compounds IIIa in which B is 2-(trifluoromethyl)-5-chlorophenyl and the combination of A and D corresponds in each case to one row of Table B (compounds IIIa.123eB-1 to IIIa.123eB-255)

Table 124e
Compounds IIIa in which B is 2-(trifluoromethyl)-6-chlorophenyl and the combination of A and D corresponds in each case to one row of Table B (compounds IIIa.124eB-1 to IIIa.124eB-255)

Table 125e
Compounds IIIa in which B is 2-chloro-3-(trifluoromethyl)phenyl and the combination of A and D corresponds in each case to one row of Table B (compounds IIIa.125eB-1 to IIIa.125eB-255)

Table 126e
Compounds IIIa in which B is 2-chloro-4-(trifluoromethyl)phenyl and the combination of A and D corresponds in each case to one row of Table B (compounds IIIa.126eB-1 to IIIa.126eB-255)

Table 127e
Compounds IIIa in which B is 2-chloro-5-(trifluoromethyl)phenyl and the combination of A and D corresponds in each case to one row of Table B (compounds IIIa.127eB-1 to IIIa.127eB-255)

Table 128e
Compounds IIIa in which B is 2-(trifluoromethoxy)-3-chlorophenyl and the combination of A and D corresponds in each case to one row of Table B (compounds IIIa.128eB-1 to IIIa.128eB-255)

Table 129e
Compounds IIIa in which B is 2-(trifluoromethoxy)-4-chlorophenyl and the combination of A and D corresponds in each case to one row of Table B (compounds IIIa.129eB-1 to IIIa.129eB-255)

Table 130e
Compounds IIIa in which B is 2-(trifluoromethoxy)-5-chlorophenyl and the combination of A and D corresponds in each case to one row of Table B (compounds IIIa.130eB-1 to IIIa.130eB-255)

Table 131e
Compounds IIIa in which B is 2-(trifluoromethoxy)-6-chlorophenyl and the combination of A and D corresponds in each case to one row of Table B (compounds IIIa.131eB-1 to IIIa.131eB-255)

Table 132e
Compounds IIIa in which B is 2-chloro-3-(trifluoromethoxy)phenyl and the combination of A and D corresponds in each case to one row of Table B (compounds IIIa.132eB-1 to IIIa.132eB-255)

Table 133e
Compounds IIIa in which B is 2-chloro-4-(trifluoromethoxy)phenyl and the combination of A and D corresponds in each case to one row of Table B (compounds IIIa.133eB-1 to IIIa.133eB-255)

Table 134e
Compounds IIIa in which B is 2-chloro-5-(trifluoromethoxy)phenyl and the combination of A and D corresponds in each case to one row of Table B (compounds IIIa.134eB-1 to IIIa.134eB-255)

Table 135e
Compounds IIIa in which B is 2-(difluoromethoxy)-3-chlorophenyl and the combination of A and D corresponds in each case to one row of Table B (compounds IIIa.135eB-1 to IIIa.135eB-255)

Table 136e
Compounds IIIa in which B is 2-(difluoromethoxy)-4-chlorophenyl and the combination of A and D corresponds in each case to one row of Table B (compounds IIIa.136eB-1 to IIIa.136eB-255)

Table 137e
Compounds IIIa in which B is 2-(difluoromethoxy)-5-chlorophenyl and the combination of A and D corresponds in each case to one row of Table B (compounds IIIa.137eB-1 to IIIa.137eB-255)

Table 138e
Compounds IIIa in which B is 2-(difluoromethoxy)-6-chlorophenyl and the combination of A and D corresponds in each case to one row of Table B (compounds IIIa.138eB-1 to IIIa.138eB-255)

Table 139e
Compounds IIIa in which B is 3-(difluoromethoxy)-4-chlorophenyl and the combination of A and D corresponds in each case to one row of Table B (compounds IIIa.139eB-1 to IIIa.139eB-255)

Table 140e
Compounds IIIa in which B is 2-chloro-3-(difluoromethoxy)phenyl and the combination of A and D corresponds in each case to one row of Table B (compounds IIIa.140eB-1 to IIIa.140eB-255)

Table 141e
Compounds IIIa in which B is 2-chloro-4-(difluoromethoxy)phenyl and the combination of A and D corresponds in each case to one row of Table B (compounds IIIa.141eB-1 to IIIa.141eB-255)

Table 142e
Compounds IIIa in which B is 2-chloro-5-(difluoromethoxy)phenyl and the combination of A and D corresponds in each case to one row of Table B (compounds IIIa.142eB-1 to IIIa.142eB-255)

Table 143e
Compounds IIIa in which B is 2-(trifluoromethylthio)-3-chlorophenyl and the combination of A and D corresponds in each case to one row of Table B (compounds IIIa.143eB-1 to IIIa.143eB-255)

Table 144e
Compounds IIIa in which B is 2-(trifluoromethylthio)-4-chlorophenyl and the combination of A and D corresponds in each case to one row of Table B (compounds IIIa.144eB-1 to IIIa.144eB-255)

Table 145e
Compounds IIIa in which B is 2-(trifluoromethylthio)-5-chlorophenyl and the combination of A and D corresponds in each case to one row of Table B (compounds IIIa.145eB-1 to IIIa.145eB-255)

Table 146e
Compounds IIIa in which B is 2-(trifluoromethylthio)-6-chlorophenyl and the combination of A and D corresponds in each case to one row of Table B (compounds IIIa.146eB-1 to IIIa.146eB-255)

Table 147e
Compounds IIIa in which B is 2-chloro-3-(trifluoromethylthio)phenyl and the combination of A and D corresponds in each case to one row of Table B (compounds IIIa.147eB-1 to IIIa.147eB-255)

Table 148e
Compounds IIIa in which B is 2-chloro-4-(trifluoromethylthio)phenyl and the combination of A and D corresponds in each case to one row of Table B (compounds IIIa.148eB-1 to IIIa.148eB-255)

Table 149e
Compounds IIIa in which B is 2-chloro-5-(trifluoromethylthio)phenyl and the combination of A and D corresponds in each case to one row of Table B (compounds IIIa.149eB-1 to IIIa.149eB-255)

Table 150e
Compounds IIIa in which B is 2,3,4-trichlorophenyl and the combination of A and D corresponds in each case to one row of Table B (compounds IIIa.150eB-1 to IIIa.150eB-255)

Table 151e
Compounds IIIa in which B is 2,3,5-trichlorophenyl and the combination of A and D corresponds in each case to one row of Table B (compounds IIIa.151eB-1 to IIIa.151eB-255)

Table 152e
Compounds IIIa in which B is 2,3,6-trichlorophenyl and the combination of A and D corresponds in each case to one row of Table B (compounds IIIa.152eB-1 to IIIa.152eB-255)

Table 153e
Compounds IIIa in which B is 2,4,5-trichlorophenyl and the combination of A and D corresponds in each case to one row of Table B (compounds IIIa.153eB-1 to IIIa.153eB-255)

Table 154e
Compounds IIIa in which B is 2,4,6-trichlorophenyl and the combination of A and D corresponds in each case to one row of Table B (compounds IIIa.154eB-1 to IIIa.154eB-255)

Table 155e
Compounds IIIa in which B is 2,3,4-trimethylphenyl and A corresponds in each case to one group of one row of Table B (compounds IIIa.155eB-1 to IIIa.155eB-255)

Table 156e
Compounds IIIa in which B is 2,3,5-trimethylphenyl and A corresponds in each case to one group of one row of Table B (compounds IIIa.156eB-1 to IIIa.156eB-255)

Table 157e
Compounds IIIa in which B is 2,3,6-trimethylphenyl and A corresponds in each case to one group of one row of Table B (compounds IIIa.157eB-1 to IIIa.157eB-255)

Table 158e
Compounds IIIa in which B is 2,4,5-trimethylphenyl and A corresponds in each case to one group of one row of Table B (compounds IIIa.158eB-1 to IIIa.158eB-255)

Table 159e
Compounds IIIa in which B is 2,4,6-trimethylphenyl and A corresponds in each case to one group of one row of Table B (compounds IIIa.159eB-1 to IIIa.159eB-255)

Table 160e
Compounds IIIa in which B is 2,3,4-trimethoxyphenyl and A corresponds in each case to one group of one row of Table B (compounds IIIa.160eB-1 to IIIa.160eB-255)

Table 161e
Compounds IIIa in which B is 2,3,5-trimethoxyphenyl and A corresponds in each case to one group of one row of Table B (compounds IIIa.161eB-1 to IIIa.161eB-255)

Table 162e
Compounds IIIa in which B is 2,3,6-trimethoxyphenyl and A corresponds in each case to one group of one row of Table B (compounds IIIa.162eB-1 to IIIa.162eB-255)

Table 1f
Compounds IIIb in which B is 2,3-difluorophenyl and the combination of A and D corresponds in each case to one row of Table B (compounds IIIb.1fB-1 to IIIb.1fB-255)

Table 2f
Compounds IIIb in which B is 2,4-difluorophenyl and the combination of A and D corresponds in each case to one row of Table B (compounds IIIb.2fB-1 to IIIb.2fB-255)

Table 3f
Compounds IIIb in which B is 2,5-difluorophenyl and the combination of A and D corresponds in each case to one row of Table B (compounds IIIb.3fB-1 to IIIb.3fB-255)

Table 4f
Compounds IIIb in which B is 2,6-difluorophenyl and the combination of A and D corresponds in each case to one row of Table B (compounds IIIb.4fB-1 to IIIb.4fB-255)

Table 5f
Compounds IIIb in which B is 2-fluoro-3-chlorophenyl and the combination of A and D corresponds in each case to one row of Table B (compounds IIIb.5fB-1 to IIIb.5fB-255)

Table 6f
Compounds IIIb in which B is 2-fluoro-4-chlorophenyl and the combination of A and D corresponds in each case to one row of Table B (compounds IIIb.6fB-1 to IIIb.6fB-255)

Table 7f
Compounds IIIb in which B is 2-fluoro-5-chlorophenyl and the combination of A and D corresponds in each case to one row of Table B (compounds IIIb.7fB-1 to IIIb.7fB-255)

Table 8f
Compounds IIIb in which B is 2-fluoro-6-chlorophenyl and the combination of A and D corresponds in each case to one row of Table B (compounds IIIb.8fB-1 to IIIb.8fB-255)

Table 9f
Compounds IIIb in which B is 2-chloro-3-fluorophenyl and the combination of A and D corresponds in each case to one row of Table B (compounds IIIb.9fB-1 to IIIb.9fB-255)

Table 10f
Compounds IIIb in which B is 2-chloro-4-fluorophenyl and the combination of A and D corresponds in each case to one row of Table B (compounds IIIb.10fB-1 to IIIb.10fB-255)

Table 11f
Compounds IIIb in which B is 2-chloro-5-fluorophenyl and the combination of A and D corresponds in each case to one row of Table B (compounds IIIb.11fB-1 to IIIb.11fB-255)

Table 12f
Compounds IIIb in which B is 2-methyl-3-fluorophenyl and the combination of A and D corresponds in each case to one row of Table B (compounds IIIb.12fB-1 to IIIb.12fB-255)

Table 13f
Compounds IIIb in which B is 2-methyl-4-fluorophenyl and the combination of A and D corresponds in each case to one row of Table B (compounds IIIb.13fB-1 to IIIb.13fB-255)

Table 14f
Compounds IIIb in which B is 2-methyl-5-fluorophenyl and the combination of A and D corresponds in each case to one row of Table B (compounds IIIb.14fB-1 to IIIb.14fB-255)

Table 15f
Compounds IIIb in which B is 2-methyl-6-fluorophenyl and the combination of A and D corresponds in each case to one row of Table B (compounds IIIb.15fB-1 to IIIb.15fB-255)

Table 16f
Compounds IIIb in which B is 2-fluoro-3-methylphenyl and the combination of A and D corresponds in each case to one row of Table B (compounds IIIb.16fB-1 to IIIb.16fB-255)

Table 17f
Compounds IIIb in which B is 2-fluoro-4-methylphenyl and the combination of A and D corresponds in each case to one row of Table B (compounds IIIb.17fB-1 to IIIb.17fB-255)

Table 18f
Compounds IIIb in which B is 2-fluoro-5-methylphenyl and the combination of A and D corresponds in each case to one row of Table B (compounds IIIb.18fB-1 to IIIb.18fB-255)

Table 19f
Compounds IIIb in which B is 2-ethyl-3-fluorophenyl and the combination of A and D corresponds in each case to one row of Table B (compounds IIIb.19fB-1 to IIIb.19fB-255)

Table 20f
Compounds IIIb in which B is 2-ethyl-4-fluorophenyl and the combination of A and D corresponds in each case to one row of Table B (compounds IIIb.20fB-1 to IIIb.20fB-255)

Table 21f
Compounds IIIb in which B is 2-ethyl-5-fluorophenyl and the combination of A and D corresponds in each case to one row of Table B (compounds IIIb.21fB-1 to IIIb.21fB-255)

Table 22f
Compounds IIIb in which B is 2-ethyl-6-fluorophenyl and the combination of A and D corresponds in each case to one row of Table B (compounds IIIb.22fB-1 to IIIb.22fB-255)

Table 23f
Compounds IIIb in which B is 2-fluoro-3-ethylphenyl and the combination of A and D corresponds in each case to one row of Table B (compounds IIIb.23fB-1 to IIIb.23fB-255)

Table 24f
Compounds IIIb in which B is 2-fluoro-4-ethylphenyl and the combination of A and D corresponds in each case to one row of Table B (compounds IIIb.24fB-1 to IIIb.24fB-255)

Table 25f
Compounds IIIb in which B is 2-methoxy-3-fluorophenyl and the combination of A and D corresponds in each case to one row of Table B (compounds IIIb.25fB-1 to IIIb.25fB-255)

Table 26f
Compounds IIIb in which B is 2-methoxy-4-fluorophenyl and the combination of A and D corresponds in each case to one row of Table B (compounds IIIb.26fB-1 to IIIb.26fB-255)

Table 27f
Compounds IIIb in which B is 2-methoxy-5-fluorophenyl and the combination of A and D corresponds in each case to one row of Table B (compounds IIIb.27fB-1 to IIIb.27fB-255)

Table 28f
Compounds IIIb in which B is 2-methoxy-6-fluorophenyl and the combination of A and D corresponds in each case to one row of Table B (compounds IIIb.28fB-1 to IIIb.28fB-255)

Table 29f
Compounds IIIb in which B is 2-fluoro-3-methoxyphenyl and the combination of A and D corresponds in each case to one row of Table B (compounds IIIb.29fB-1 to IIIb.29fB-255)

Table 30f
Compounds IIIb in which B is 2-fluoro-4-methoxyphenyl and the combination of A and D corresponds in each case to one row of Table B (compounds IIIb.30fB-1 to IIIb.30fB-255)

Table 31f
Compounds IIIb in which B is 2-fluoro-5-methoxyphenyl and the combination of A and D corresponds in each case to one row of Table B (compounds IIIb.31fB-1 to IIIb.31fB-255)

Table 32f
Compounds IIIb in which B is 2-(trifluoromethyl)-3-fluorophenyl and the combination of A and D corresponds in each case to one row of Table B (compounds IIIb.32fB-1 to IIIb.32fB-255)

Table 33f
Compounds IIIb in which B is 2,4,5-trimethoxyphenyl and the combination of A and D corresponds in each case to one row of Table B (compounds IIIb.33fB-1 to IIIb.33fB-255)

Table 34f
Compounds IIIb in which B is 2,4,6-trimethoxyphenyl and the combination of A and D corresponds in each case to one row of Table B (compounds IIIb.34fB-1 to IIIb.34fB-255)

Table 35f
Compounds IIIb in which B is 2-fluoro-5-ethylphenyl and the combination of A and D corresponds in each case to one row of Table B (compounds IIIb.35fB-1 to IIIb.35fB-255)

Table 36f
Compounds IIIb in which B is 2-(trifluoromethyl)-4-fluorophenyl and the combination of A and D corresponds in each case to one row of Table B (compounds IIIb.36fB-1 to IIIb.36fB-255)

Table 37f
Compounds IIIb in which B is 2-(trifluoromethyl)-5-fluorophenyl and the combination of A and D corresponds in each case to one row of Table B (compounds IIIb.37fB-1 to IIIb.37fB-255)

Table 38f
Compounds IIIb in which B is 2-(trifluoromethyl)-6-fluorophenyl and the combination of A and D corresponds in each case to one row of Table B (compounds IIIb.38fB-1 to IIIb.38fB-255)

Table 39f
Compounds IIIb in which B is 2-fluoro-3-(trifluoromethyl)phenyl and the combination of A and D corresponds in each case to one row of Table B (compounds IIIb.39fB-1 to IIIb.39fB-255)

Table 40f
Compounds IIIb in which B is 2-fluoro-4-(trifluoromethyl)phenyl and the combination of A and D corresponds in each case to one row of Table B (compounds IIIb.40fB-1 to IIIb.40fB-255)

Table 41f
Compounds IIIb in which B is 2-fluoro-5-(trifluoromethyl)phenyl and the combination of A and D corresponds in each case to one row of Table B (compounds IIIb.41fB-1 to IIIb.41fB-255)

Table 42f
Compounds IIIb in which B is 2-(trifluoromethoxy)-3-fluorophenyl and the combination of A and D corresponds in each case to one row of Table B (compounds IIIb.42fB-1 to IIIb.42fB-255)

Table 43f

Compounds IIIb in which B is 2-(trifluoromethoxy)-4-fluorophenyl and the combination of A and D corresponds in each case to one row of Table B (compounds IIIb.43fB-1 to IIIb.43fB-255)

Table 44f
Compounds IIIb in which B is 2-(trifluoromethoxy)-5-fluorophenyl and the combination of A and D corresponds in each case to one row of Table B (compounds IIIb.44fB-1 to IIIb.44fB-255)

Table 45f
Compounds IIIb in which B is 2-(trifluoromethoxy)-6-fluorophenyl and the combination of A and D corresponds in each case to one row of Table B (compounds IIIb.45fB-1 to IIIb.45fB-255)

Table 46f
Compounds IIIb in which B is 2-fluoro-3-(trifluoromethoxy)phenyl and the combination of A and D corresponds in each case to one row of Table B (compounds IIIb.46fB-1 to IIIb.46fB-255)

Table 47f
Compounds IIIb in which B is 2-fluoro-4-(trifluoromethoxy)phenyl and the combination of A and D corresponds in each case to one row of Table B (compounds IIIb.47fB-1 to IIIb.47fB-255)

Table 48f
Compounds IIIb in which B is 2-fluoro-5-(trifluoromethoxy)phenyl and the combination of A and D corresponds in each case to one row of Table B (compounds IIIb.48fB-1 to IIIb.48fB-255)

Table 49f
Compounds IIIb in which B is 2-(difluoromethoxy)-3-fluorophenyl and the combination of A and D corresponds in each case to one row of Table B (compounds IIIb.49fB-1 to IIIb.49fB-255)

Table 50f
Compounds IIIb in which B is 2-(difluoromethoxy)-4-fluorophenyl and the combination of A and D corresponds in each case to one row of Table B (compounds IIIb.50fB-1 to IIIb.50fB-255)

Table 51f

Compounds IIIb in which B is 2-(difluoromethoxy)-5-fluorophenyl and the combination of A and D corresponds in each case to one row of Table B (compounds IIIb.51fB-1 to IIIb.51fB-255)

Table 52f
Compounds IIIb in which B is 2-(difluoromethoxy)-6-fluorophenyl and the combination of A and D corresponds in each case to one row of Table B (compounds IIIb.52fB-1 to IIIb.52fB-255)

Table 53f
Compounds IIIb in which B is 2-fluoro-3-(difluoromethoxy)phenyl and the combination of A and D corresponds in each case to one row of Table B (compounds IIIb.53fB-1 to IIIb.53fB-255)

Table 54f
Compounds IIIb in which B is 2-fluoro-4-(difluoromethoxy)phenyl and the combination of A and D corresponds in each case to one row of Table B (compounds IIIb.54fB-1 to IIIb.54fB-255)

Table 55f
Compounds IIIb in which B is 2-fluoro-5-(difluoromethoxy)phenyl and the combination of A and D corresponds in each case to one row of Table B (compounds IIIb.55fB-1 to IIIb.55fB-255)

Table 56f
Compounds IIIb in which B is 2-(trifluoromethylthio)-3-fluorophenyl and the combination of A and D corresponds in each case to one row of Table B (compounds IIIb.56fB-1 to IIIb.56fB-255)

Table 57f
Compounds IIIb in which B is 2-(trifluoromethylthio)-4-fluorophenyl and the combination of A and D corresponds in each case to one row of Table B (compounds IIIb.57fB-1 to IIIb.57fB-255)

Table 58f
Compounds IIIb in which B is 2-(trifluoromethylthio)-5-fluorophenyl and the combination of A and D corresponds in each case to one row of Table B (compounds IIIb.58fB-1 to IIIb.58fB-255)

Table 59f
Compounds IIIb in which B is 2-(trifluoromethylthio)-6-fluorophenyl and the combination of A and D corresponds in each case to one row of Table B (compounds IIIb.59fB-1 to IIIb.59fB-255)

Table 60f
Compounds IIIb in which B is 2-fluoro-3-(trifluoromethylthio)phenyl and the combination of A and D corresponds in each case to one row of Table B (compounds IIIb.60fB-1 to IIIb.60fB-255)

Table 61f
Compounds IIIb in which B is 2-fluoro-4-(trifluoromethylthio)phenyl and the combination of A and D corresponds in each case to one row of Table B (compounds IIIb.61fB-1 to IIIb.61fB-255)

Table 62f
Compounds IIIb in which B is 2-fluoro-5-(trifluoromethylthio)phenyl and the combination of A and D corresponds in each case to one row of Table B (compounds IIIb.62fB-1 to IIIb.62fB-255)

Table 63f
Compounds IIIb in which B is 2,3,4-trifluorophenyl and the combination of A and D corresponds in each case to one row of Table B (compounds IIIb.63fB-1 to IIIb.63fB-255)

Table 64f
Compounds IIIb in which B is 2,3,5-trifluorophenyl and the combination of A and D corresponds in each case to one row of Table B (compounds IIIb.64fB-1 to IIIb.64fB-255)

Table 65f
Compounds IIIb in which B is 2,3,6-trifluorophenyl and the combination of A and D corresponds in each case to one row of Table B (compounds IIIb.65fB-1 to IIIb.65fB-255)

Table 66f
Compounds IIIb in which B is 2,4,5-trifluorophenyl and the combination of A and D corresponds in each case to one row of Table B (compounds IIIb.66fB-1 to IIIb.66fB-255)

Table 67f
Compounds IIIb in which B is 2,4,6-trifluorophenyl and the combination of A and D corresponds in each case to one row of Table B (compounds IIIb.67fB-1 to IIIb.67fB-255)

Table 68f
Compounds IIIb in which B is 2,3-dichlorophenyl and the combination of A and D corresponds in each case to one row of Table B (compounds IIIb.68fB-1 to IIIb.68fB-255)

Table 69f
Compounds IIIb in which B is 2,4-dichlorophenyl and the combination of A and D corresponds in each case to one row of Table B (compounds IIIb.69fB-1 to IIIb.69fB-255)

Table 70f
Compounds IIIb in which B is 2,5-dichlorophenyl and the combination of A and D corresponds in each case to one row of Table B (compounds IIIb.70fB-1 to IIIb.70fB-255)

Table 71f
Compounds IIIb in which B is 2,6-dichlorophenyl and the combination of A and D corresponds in each case to one row of Table B (compounds IIIb.71fB-1 to IIIb.71fB-255)

Table 72f
Compounds IIIb in which B is 2,3-dimethylphenyl and the combination of A and D corresponds in each case to one row of Table B (compounds IIIb.72fB-1 to IIIb.72fB-255)

Table 73f
Compounds IIIb in which B is 2,4-dimethylphenyl and the combination of A and D corresponds in each case to one row of Table B (compounds IIIb.73fB-1 to IIIb.73fB-255)

Table 74f
Compounds IIIb in which B is 2,5-dimethylphenyl and the combination of A and D corresponds in each case to one row of Table B (compounds IIIb.74fB-1 to IIIb.74fB-255)

Table 75f
Compounds IIIb in which B is 2,6-dimethylphenyl and the combination of A and D corresponds in each case to one row of Table B (compounds IIIb.75fB-1 to IIIb.75fB-255)

Table 76f
Compounds IIIb in which B is 2,3-diethylphenyl and the combination of A and D corresponds in each case to one row of Table B (compounds IIIb.76fB-1 to IIIb.76fB-255)

Table 77f
Compounds IIIb in which B is 2,4-diethylphenyl and the combination of A and D corresponds in each case to one row of Table B (compounds IIIb.77fB-1 to IIIb.77fB-255)

Table 78f
Compounds IIIb in which B is 2,5-diethylphenyl and the combination of A and D corresponds in each case to one row of Table B (compounds IIIb.78fB-1 to IIIb.78fB-255)

Table 79f
Compounds IIIb in which B is 2,6-diethylphenyl and the combination of A and D corresponds in each case to one row of Table B (compounds IIIb.79fB-1 to IIIb.79fB-255)

Table 80f
Compounds IIIb in which B is 2,3-dimethoxyphenyl and the combination of A and D corresponds in each case to one row of Table B (compounds IIIb.80fB-1 to IIIb.80fB-255)

Table 81f
Compounds IIIb in which B is 2,4-dimethoxyphenyl and the combination of A and D corresponds in each case to one row of Table B (compounds IIIb.81fB-1 to IIIb.81fB-255)

Table 82f
Compounds IIIb in which B is 2,5-dimethoxyphenyl and the combination of A and D corresponds in each case to one row of Table B (compounds IIIb.82fB-1 to IIIb.82fB-255)

Table 83f
Compounds IIIb in which B is 2,6-dimethoxyphenyl and the combination of A and D corresponds in each case to one row of Table B (compounds IIIb.83fB-1 to IIIb.83fB-255)

Table 84f
Compounds IIIb in which B is 2,3-di(trifluoromethyl)phenyl and the combination of A and D corresponds in each case to one row of Table B (compounds IIIb.84fB-1 to IIIb.84fB-255)

Table 85f
Compounds IIIb in which B is 2,4-di(trifluoromethyl)phenyl and the combination of A and D corresponds in each case to one row of Table B (compounds IIIb.85fB-1 to IIIb.85fB-255)

Table 86f
Compounds IIIb in which B is 2,5-di(trifluoromethyl)phenyl and the combination of A and D corresponds in each case to one row of Table B (compounds IIIb.86fB-1 to IIIb.86fB-255)

Table 87f
Compounds IIIb in which B is 2,6-di(trifluoromethyl)phenyl and the combination of A and D corresponds in each case to one row of Table B (compounds IIIb.87fB-1 to IIIb.87fB-255)

Table 88f
Compounds IIIb in which B is 2,3-di(trifluoromethoxy)phenyl and the combination of A and D corresponds in each case to one row of Table B (compounds IIIb.88fB-1 to IIIb.88fB-255)

Table 89f
Compounds IIIb in which B is 2,4-di(trifluoromethoxy)phenyl and the combination of A and D corresponds in each case to one row of Table B (compounds IIIb.89fB-1 to IIIb.89fB-255)

Table 90f
Compounds IIIb in which B is 2,5-di(trifluoromethoxy)phenyl and the combination of A and D corresponds in each case to one row of Table B (compounds IIIb.90fB-1 to IIIb.90fB-255)

Table 91f
Compounds IIIb in which B is 2,6-di(trifluoromethoxy)phenyl and the combination of A and D corresponds in each case to one row of Table B (compounds IIIb.91fB-1 to IIIb.91fB-255)

Table 92f
Compounds IIIb in which B is 2,3-di(difluoromethoxy)phenyl and the combination of A and D corresponds in each case to one row of Table B (compounds IIIb.92fB-1 to IIIb.92fB-255)

Table 93f
Compounds IIIb in which B is 2,4-di(difluoromethoxy)phenyl and the combination of A and D corresponds in each case to one row of Table B (compounds IIIb.93fB-1 to IIIb.93fB-255)

Table 94f
Compounds IIIb in which B is 2,5-di(difluoromethoxy)phenyl and the combination of A and D corresponds in each case to one row of Table B (compounds IIIb.94fB-1 to IIIb.94fB-255)

Table 95f
Compounds IIIb in which B is 2,6-di(difluoromethoxy)phenyl and the combination of A and D corresponds in each case to one row of Table B (compounds IIIb.95fB-1 to IIIb.95fB-255)

Table 96f
Compounds IIIb in which B is 2,3-di(trifluoromethylthio)phenyl and the combination of A and D corresponds in each case to one row of Table B (compounds IIIb.96fB-1 to IIIb.96fB-255)

Table 97f
Compounds IIIb in which B is 2,4-di(trifluoromethylthio)phenyl and the combination of A and D corresponds in each case to one row of Table B (compounds IIIb.97fB-1 to IIIb.97fB-255)

Table 98f
Compounds IIIb in which B is 2,5-di(trifluoromethylthio)phenyl and the combination of A and D corresponds in each case to one row of Table B (compounds IIIb.98fB-1 to IIIb.98fB-255)

Table 99f
Compounds IIIb in which B is 2,6-di(trifluoromethylthio)phenyl and the combination of A and D corresponds in each case to one row of Table B (compounds IIIb.99fB-1 to IIIb.99fB-255)

Table 100f
Compounds IIIb in which B is 2-methyl-3-chlorophenyl and the combination of A and D corresponds in each case to one row of Table B (compounds IIIb.100fB-1 to IIIb.100fB-255)

Table 101f
Compounds IIIb in which B is 2-methyl-4-chlorophenyl and the combination of A and D corresponds in each case to one row of Table B (compounds IIIb.101fB-1 to IIIb.101fB-255)

Table 102f
Compounds IIIb in which B is 2-methyl-5-chlorophenyl and the combination of A and D corresponds in each case to one row of Table B (compounds IIIb.102fB-1 to IIIb.102fB-255)

Table 103f
Compounds IIIb in which B is 2-methyl-6-chlorophenyl and the combination of A and D corresponds in each case to one row of Table B (compounds IIIb.103fB-1 to IIIb.103fB-255)

Table 104f
Compounds IIIb in which B is 2-chloro-3-methylphenyl and the combination of A and D corresponds in each case to one row of Table B (compounds IIIb.104fB-1 to IIIb.104fB-255)

Table 105f
Compounds IIIb in which B is 2-chloro-4-methylphenyl and the combination of A and D corresponds in each case to one row of Table B (compounds IIIb.105fB-1 to IIIb.105fB-255)

Table 106f
Compounds IIIb in which B is 2-chloro-5-methylphenyl and the combination of A and D corresponds in each case to one row of Table B (compounds IIIb.106fB-1 to III b.106fB-255)

Table 107f
Compounds IIIb in which B is 2-chloro-3-ethylphenyl and the combination of A and D corresponds in each case to one row of Table B (compounds IIIb.107fB-1 to IIIb.107fB-255)

Table 108f
Compounds IIIb in which B is 2-chloro-4-ethylphenyl and the combination of A and D corresponds in each case to one row of Table B (compounds IIIb.108fB-1 to IIIb.108fB-255)

Table 109f
Compounds IIIb in which B is 2-chloro-5-ethylphenyl and the combination of A and D corresponds in each case to one row of Table B (compounds IIIb.109fB-1 to IIIb.109fB-255)

Table 110f
Compounds IIIb in which B is 2-ethyl-3-chlorophenyl and the combination of A and D corresponds in each case to one row of Table B (compounds IIIb.110fB-1 to IIIb.110fB-255)

Table 111f
Compounds IIIb in which B is 2-ethyl-4-chlorophenyl and the combination of A and D corresponds in each case to one row of Table B (compounds IIIb.111fB-1 to IIIb.111fB-255)

Table 112f
Compounds IIIb in which B is 2-ethyl-5-chlorophenyl and the combination of A and D corresponds in each case to one row of Table B (compounds IIIb.112fB-1 to IIIb.112fB-255)

Table 113f
Compounds IIIb in which B is 2-ethyl-6-chlorophenyl and the combination of A and D corresponds in each case to one row of Table B (compounds IIIb.113fB-1 to IIIb.113fB-255)

Table 114f
Compounds IIIb in which B is 2-methoxy-3-chlorophenyl and the combination of A and D corresponds in each case to one row of Table B (compounds IIIb.114fB-1 to IIIb.114fB-255)

Table 115f
Compounds IIIb in which B is 2-methoxy-4-chlorophenyl and the combination of A and D corresponds in each case to one row of Table B (compounds IIIb.115fB-1 to IIIb.115fB-255)

Table 116f
Compounds IIIb in which B is 2-methoxy-5-chlorophenyl and the combination of A and D corresponds in each case to one row of Table B (compounds IIIb.116fB-1 to IIIb.116fB-255)

Table 117f
Compounds IIIb in which B is 2-methoxy-6-chlorophenyl and the combination of A and D corresponds in each case to one row of Table B (compounds IIIb.117fB-1 to IIIb.117fB-255)

Table 118f
Compounds IIIb in which B is 2-chloro-3-methoxyphenyl and the combination of A and D corresponds in each case to one row of Table B (compounds IIIb.118fB-1 to IIIb.118fB-255)

Table 119f
Compounds IIIb in which B is 2-chloro-4-methoxyphenyl and the combination of A and D corresponds in each case to one row of Table B (compounds IIIb.119fB-1 to IIIb.119fB-255)

Table 120f
Compounds IIIb in which B is 2-chloro-5-methoxyphenyl and the combination of A and D corresponds in each case to one row of Table B (compounds IIIb.120fB-1 to IIIb.120fB-255)

Table 121f
Compounds IIIb in which B is 2-(trifluoromethyl)-3-chlorophenyl and the combination of A and D corresponds in each case to one row of Table B (compounds IIIb.121fB-1 to IIIb.121fB-255)

Table 122f
Compounds IIIb in which B is 2-(trifluoromethyl)-4-chlorophenyl and the combination of A and D corresponds in each case to one row of Table B (compounds IIIb.122fB-1 to IIIb.122fB-255)

Table 123f
Compounds IIIb in which B is 2-(trifluoromethyl)-5-chlorophenyl and the combination of A and D corresponds in each case to one row of Table B (compounds IIIb.123fB-1 to IIIb.123fB-255)

Table 124f
Compounds IIIb in which B is 2-(trifluoromethyl)-6-chlorophenyl and the combination of A and D corresponds in each case to one row of Table B (compounds IIIb.124fB-1 to IIIb.124fB-255)

Table 125f
Compounds IIIb in which B is 2-chloro-3-(trifluoromethyl)phenyl and the combination of A and D corresponds in each case to one row of Table B (compounds IIIb.125fB-1 to IIIb.125fB-255)

Table 126f
Compounds IIIb in which B is 2-chloro-4-(trifluoromethyl)phenyl and the combination of A and D corresponds in each case to one row of Table B (compounds IIIb.126fB-1 to IIIb.126fB-255)

Table 127f
Compounds IIIb in which B is 2-chloro-5-(trifluoromethyl)phenyl and the combination of A and D corresponds in each case to one row of Table B (compounds IIIb.127fB-1 to IIIb.127fB-255)

Table 128f
Compounds IIIb in which B is 2-(trifluoromethoxy)-3-chlorophenyl and the combination of A and D corresponds in each case to one row of Table B (compounds IIIb.128fB-1 to IIIb.128fB-255)

Table 129f
Compounds IIIb in which B is 2-(trifluoromethoxy)-4-chlorophenyl and the combination of A and D corresponds in each case to one row of Table B (compounds IIIb.129fB-1 to IIIb.129fB-255)

Table 130f
Compounds IIIb in which B is 2-(trifluoromethoxy)-5-chlorophenyl and the combination of A and D corresponds in each case to one row of Table B (compounds IIIb.130fB-1 to IIIb.130fB-255)

Table 131f
Compounds IIIb in which B is 2-(trifluoromethoxy)-6-chlorophenyl and the combination of A and D corresponds in each case to one row of Table B (compounds IIIb.131fB-1 to IIIb.131fB-255)

Table 132f
Compounds IIIb in which B is 2-chloro-3-(trifluoromethoxy)phenyl and the combination of A and D corresponds in each case to one row of Table B (compounds IIIb.132fB-1 to IIIb.132fB-255)

Table 133f
Compounds IIIb in which B is 2-chloro-4-(trifluoromethoxy)phenyl and the combination of A and D corresponds in each case to one row of Table B (compounds IIIb.133fB-1 to IIIb.133fB-255)

Table 134f
Compounds IIIb in which B is 2-chloro-5-(trifluoromethoxy)phenyl and the combination of A and D corresponds in each case to one row of Table B (compounds IIIb.134fB-1 to IIIb.134fB-255)

Table 135f
Compounds IIIb in which B is 2-(difluoromethoxy)-3-chlorophenyl and the combination of A and D corresponds in each case to one row of Table B (compounds IIIb.135fB-1 to IIIb.135fB-255)

Table 136f
Compounds IIIb in which B is 2-(difluoromethoxy)-4-chlorophenyl and the combination of A and D corresponds in each case to one row of Table B (compounds IIIb.136fB-1 to IIIb.136fB-255)

Table 137f
Compounds IIIb in which B is 2-(difluoromethoxy)-5-chlorophenyl and the combination of A and D corresponds in each case to one row of Table B (compounds IIIb.137fB-1 to IIIb.137fB-255)

Table 138f
Compounds IIIb in which B is 2-(difluoromethoxy)-6-chlorophenyl and the combination of A and D corresponds in each case to one row of Table B (compounds IIIb.138fB-1 to IIIb.138fB-255)

Table 139f
Compounds IIIb in which B is 3-(difluoromethoxy)-4-chlorophenyl and the combination of A and D corresponds in each case to one row of Table B (compounds IIIb.139fB-1 to IIIb.139fB-255)

Table 140f
Compounds IIIb in which B is 2-chloro-3-(difluoromethoxy)phenyl and the combination of A and D corresponds in each case to one row of Table B (compounds IIIb.140fB-1 to IIIb.140fB-255)

Table 141f
Compounds IIIb in which B is 2-chloro-4-(difluoromethoxy)phenyl and the combination of A and D corresponds in each case to one row of Table B (compounds IIIb.141fB-1 to IIIb.141fB-255) Table 142f
Compounds IIIb in which B is 2-chloro-5-(difluoromethoxy)phenyl and the combination of A and D corresponds in each case to one row of Table B (compounds IIIb.142fB-1 to IIIb.142fB-255)

Table 143f
Compounds IIIb in which B is 2-(trifluoromethylthio)-3-chlorophenyl and the combination of A and D corresponds in each case to one row of Table B (compounds IIIb.143fB-1 to IIIb.143fB-255)

Table 144f
Compounds IIIb in which B is 2-(trifluoromethylthio)-4-chlorophenyl and the combination of A and D corresponds in each case to one row of Table B (compounds IIIb.144fB-1 to IIIb.144fB-255)

Table 145f
Compounds IIIb in which B is 2-(trifluoromethylthio)-5-chlorophenyl and the combination of A and D corresponds in each case to one row of Table B (compounds IIIb.145fB-1 to IIIb.145fB-255)

Table 146f
Compounds IIIb in which B is 2-(trifluoromethylthio)-6-chlorophenyl and the combination of A and D corresponds in each case to one row of Table B (compounds IIIb.146fB-1 to IIIb.146fB-255)

Table 147f
Compounds IIIb in which B is 2-chloro-3-(trifluoromethylthio)phenyl and the combination of A and D corresponds in each case to one row of Table B (compounds IIIb.147fB-1 to IIIb.147fB-255)

Table 148f
Compounds IIIb in which B is 2-chloro-4-(trifluoromethylthio)phenyl and the combination of A and D corresponds in each case to one row of Table B (compounds IIIb.148fB-1 to IIIb.148fB-255)

Table 149f
Compounds IIIb in which B is 2-chloro-5-(trifluoromethylthio)phenyl and the combination of A and D corresponds in each case to one row of Table B (compounds IIIb.149fB-1 to IIIb.149fB-255)

Table 150f
Compounds IIIb in which B is 2,3,4-trichlorophenyl and the combination of A and D corresponds in each case to one row of Table B (compounds IIIb.150fB-1 to IIIb.150fB-255)

Table 151f
Compounds IIIb in which B is 2,3,5-trichlorophenyl and the combination of A and D corresponds in each case to one row of Table B (compounds IIIb.151fB-1 to IIIb.151fB-255)

Table 152f
Compounds IIIb in which B is 2,3,6-trichlorophenyl and the combination of A and D corresponds in each case to one row of Table B (compounds IIIb.152fB-1 to IIIb.152fB-255)

Table 153f
Compounds IIIb in which B is 2,4,5-trichlorophenyl and the combination of A and D corresponds in each case to one row of Table B (compounds IIIb.153fB-1 to IIIb.153fB-255)

Table 154f
Compounds IIIb in which B is 2,4,6-trichlorophenyl and the combination of A and D corresponds in each case to one row of Table B (compounds IIIb.154fB-1 to IIIb.154fB-255)

Table 155f
Compounds IIIb in which B is 2,3,4-trimethylphenyl and A corresponds in each case to one group of one row of Table B (compounds IIIb.155fB-1 to IIIb.155fB-255)

Table 156f
Compounds IIIb in which B is 2,3,5-trimethylphenyl and A corresponds in each case to one group of one row of Table B (compounds IIIb.156fB-1 to IIIb.156fB-255)

Table 157f
Compounds IIIb in which B is 2,3,6-trimethylphenyl and A corresponds in each case to one group of one row of Table B (compounds IIIb.157fB-1 to IIIb.157fB-255)

Table 158f
Compounds IIIb in which B is 2,4,5-trimethylphenyl and A corresponds in each case to one group of one row of Table B (compounds IIIb.158fB-1 to IIIb.158fB-255)

Table 159f
Compounds IIIb in which B is 2,4,6-trimethylphenyl and A corresponds in each case to one group of one row of Table B (compounds IIIb.159fB-1 to IIIb.159fB-255)

Table 160f
Compounds IIIb in which B is 2,3,4-trimethoxyphenyl and A corresponds in each case to one group of one row of Table B (compounds IIIb.160fB-1 to IIIb.160fB-255)

Table 161f
Compounds IIIb in which B is 2,3,5-trimethoxyphenyl and A corresponds in each case to one group of one row of Table B (compounds IIIb.161fB-1 to IIIb.161fB-255)

Table 162f
Compounds IIIb in which B is 2,3,6-trimethoxyphenyl and A corresponds in each case to one group of one row of Table B (compounds IIIb.162fB-1 to IIIb.162fB-255)

Table 1g
Compounds IIIc in which B is 2,3-difluorophenyl and the combination of A and D corresponds in each case to one row of Table B (compounds IIIc.1gB-1 to IIIc.1gB-255)

Table 2g
Compounds IIIc in which B is 2,4-difluorophenyl and the combination of A and D corresponds in each case to one row of Table B (compoundsIIIc.2gB-1 to IIIc.2gB-255)

Table 3g
Compounds IIIc in which B is 2,5-difluorophenyl and the combination of A and D corresponds in each case to one row of Table B (compounds IIIc.3gB-1 to IIIc.3gB-255)

Table 4g
Compounds IIIc in which B is 2,6-difluorophenyl and the combination of A and D corresponds in each case to one row of Table B (compounds IIIc.4gB-1 to IIIc.4gB-255)

Table 5g
Compounds IIIc in which B is 2-fluoro-3-chlorophenyl and the combination of A and D corresponds in each case to one row of Table B (compounds IIIc.5gB-1 to IIIc.5gB-255)

Table 6g
Compounds IIIc in which B is 2-fluoro-4-chlorophenyl and the combination of A and D corresponds in each case to one row of Table B (compounds IIIc.6gB-1 to IIIc.6gB-255)

Table 7g
Compounds IIIc in which B is 2-fluoro-5-chlorophenyl and the combination of A and D corresponds in each case to one row of Table B (compounds IIIc.7gB-1 to IIIc.7gB-255)

Table 8g
Compounds IIIc in which B is 2-fluoro-6-chlorophenyl and the combination of A and D corresponds in each case to one row of Table B (compounds IIIc.8gB-1 to IIIc.8gB-255)

Table 9g
Compounds IIIc in which B is 2-chloro-3-fluorophenyl and the combination of A and D corresponds in each case to one row of Table B (compounds IIIc.9gB-1 to IIIc.9gB-255)

Table 10g
Compounds IIIc in which B is 2-chloro-4-fluorophenyl and the combination of A and D corresponds in each case to one row of Table B (compounds IIIc.10gB-1 to IIIc.10gB-255)

Table 11g
Compounds IIIc in which B is 2-chloro-5-fluorophenyl and the combination of A and D corresponds in each case to one row of Table B (compounds IIIc.1 1gB-1 to IIIc.11gB-255)

Table 12g
Compounds IIIc in which B is 2-methyl-3-fluorophenyl and the combination of A and D corresponds in each case to one row of Table B (compounds IIIc.12gB-1 to IIIc.12gB-255)

Table 13g
Compounds IIIc in which B is 2-methyl-4-fluorophenyl and the combination of A and D corresponds in each case to one row of Table B (compounds IIIc.13gB-1 to IIIc.13gB-255)

Table 14g
Compounds IIIc in which B is 2-methyl-5-fluorophenyl and the combination of A and D corresponds in each case to one row of Table B (compounds IIIc.14gB-1 to IIIc.14gB-255)

Table 15g
Compounds IIIc in which B is 2-methyl-6-fluorophenyl and the combination of A and D corresponds in each case to one row of Table B (compounds IIIc.15gB-1 to IIIc.15gB-255)

Table 16g
Compounds IIIc in which B is 2-fluoro-3-methylphenyl and the combination of A and D corresponds in each case to one row of Table B (compounds IIIc.16gB-1 to IIIc.16gB-255)

Table 17g
Compounds IIIc in which B is 2-fluoro-4-methylphenyl and the combination of A and D corresponds in each case to one row of Table B (compounds IIIc.17gB-1 to IIIc.17gB-255)

Table 18g
Compounds IIIc in which B is 2-fluoro-5-methylphenyl and the combination of A and D corresponds in each case to one row of Table B (compounds IIIc.18gB-1 to IIIc.18gB-255)

Table 19g
Compounds IIIc in which B is 2-ethyl-3-fluorophenyl and the combination of A and D corresponds in each case to one row of Table B (compounds IIIc.19gB-1 to IIIc.19gB-255)

Table 20g
Compounds IIIc in which B is 2-ethyl-4-fluorophenyl and the combination of A and D corresponds in each case to one row of Table B (compounds IIIc.20gB-1 to IIIc.20gB-255)

Table 21g
Compounds IIIc in which B is 2-ethyl-5-fluorophenyl and the combination of A and D corresponds in each case to one row of Table B (compounds IIIc.21gB-1 to IIIc.21gB-255)

Table 22g
Compounds IIIc in which B is 2-ethyl-6-fluorophenyl and the combination of A and D corresponds in each case to one row of Table B (compounds IIIc.22gB-1 to IIIc.22gB-255)

Table 23g
Compounds IIIc in which B is 2-fluoro-3-ethylphenyl and the combination of A and D corresponds in each case to one row of Table B (compounds IIIc.23gB-1 to IIIc.23gB-255)

Table 24g
Compounds IIIc in which B is 2-fluoro-4-ethylphenyl and the combination of A and D corresponds in each case to one row of Table B (compounds IIIc.24gB-1 to IIIc.24gB-255)

Table 25g
Compounds IIIc in which B is 2-methoxy-3-fluorophenyl and the combination of A and D corresponds in each case to one row of Table B (compounds IIIc.25gB-1 to IIIc.25gB-255)

Table 26g
Compounds IIIc in which B is 2-methoxy-4-fluorophenyl and the combination of A and D corresponds in each case to one row of Table B (compounds IIIc.26gB-1 to IIIc.26gB-255)

Table 27g
Compounds IIIc in which B is 2-methoxy-5-fluorophenyl and the combination of A and D corresponds in each case to one row of Table B (compounds IIIc.27gB-1 to IIIc.27gB-255)

Table 28g
Compounds IIIc in which B is 2-methoxy-6-fluorophenyl and the combination of A and D corresponds in each case to one row of Table B (compounds IIIc.28gB-1 to IIIc.28gB-255)

Table 29g
Compounds IIIc in which B is 2-fluoro-3-methoxyphenyl and the combination of A and D corresponds in each case to one row of Table B (compounds IIIc.29gB-1 to IIIc.29gB-255)

Table 30g
Compounds IIIc in which B is 2-fluoro-4-methoxyphenyl and the combination of A and D corresponds in each case to one row of Table B (compounds IIIc.30gB-1 to IIIc.30gB-255)

Table 31g
Compounds IIIc in which B is 2-fluoro-5-methoxyphenyl and the combination of A and D corresponds in each case to one row of Table B (compounds IIIc.31gB-1 to IIIc.31gB-255)

Table 32g
Compounds IIIc in which B is 2-(trifluoromethyl)-3-fluorophenyl and the combination of A and D corresponds in each case to one row of Table B (compounds IIIc.32gB-1 to IIIc.32gB-255)

Table 33g
Compounds IIIc in which B is 2,4,5-trimethoxyphenyl and the combination of A and D corresponds in each case to one row of Table B (compounds IIIc.33gB-1 to IIIc.33gB-255)

Table 34g
Compounds IIIc in which B is 2,4,6-trimethoxyphenyl and the combination of A and D corresponds in each case to one row of Table B (compounds IIIc.34gB-1 to IIIc.34gB-255)

Table 35g
Compounds IIIc in which B is 2-fluoro-5-ethylphenyl and the combination of A and D corresponds in each case to one row of Table B (compounds IIIc.35gB-1 to IIIc.35gB-255)

Table 36g
Compounds IIIc in which B is 2-(trifluoromethyl)-4-fluorophenyl and the combination of A and D corresponds in each case to one row of Table B (compounds IIIc.36gB-1 to IIIc.36gB-255)

Table 37g
Compounds IIIc in which B is 2-(trifluoromethyl)-5-fluorophenyl and the combination of A and D corresponds in each case to one row of Table B (compounds IIIc.37gB-1 to IIIc.37gB-255)

Table 38g
Compounds IIIc in which B is 2-(trifluoromethyl)-6-fluorophenyl and the combination of A and D corresponds in each case to one row of Table B (compounds IIIc.38gB-1 to IIIc.38gB-255)

Table 39g
Compounds IIIc in which B is 2-fluoro-3-(trifluoromethyl)phenyl and the combination of A and D corresponds in each case to one row of Table B (compounds IIIc.39gB-1 to IIIc.39gB-255)

Table 40g
Compounds IIIc in which B is 2-fluoro-4-(trifluoromethyl)phenyl and the combination of A and D corresponds in each case to one row of Table B (compounds IIIc.40gB-1 to IIIc.40gB-255)

Table 41g
Compounds IIIc in which B is 2-fluoro-5-(trifluoromethyl)phenyl and the combination of A and D corresponds in each case to one row of Table B (compounds IIIc.41gB-1 to IIIc.41gB-255)

Table 42g
Compounds IIIc in which B is 2-(trifluoromethoxy)-3-fluorophenyl and the combination of A and D corresponds in each case to one row of Table B (compounds IIIc.42gB-1 to IIIc.42gB-255)

Table 43g
Compounds IIIc in which B is 2-(trifluoromethoxy)-4-fluorophenyl and the combination of A and D corresponds in each case to one row of Table B (compounds IIIc.43gB-1 to IIIc.43gB-255)

Table 44g
Compounds IIIc in which B is 2-(trifluoromethoxy)-5-fluorophenyl and the combination of A and D corresponds in each case to one row of Table B (compounds IIIc.44gB-1 to IIIc.44gB-255)

Table 45g
Compounds IIIc in which B is 2-(trifluoromethoxy)-6-fluorophenyl and the combination of A and D corresponds in each case to one row of Table B (compounds IIIc.45gB-1 to IIIc.45gB-255)

Table 46g
Compounds IIIc in which B is 2-fluoro-3-(trifluoromethoxy)phenyl and the combination of A and D corresponds in each case to one row of Table B (compounds IIIc.46gB-1 to IIIc.46gB-255)

Table 47g
Compounds IIIc in which B is 2-fluoro-4-(trifluoromethoxy)phenyl and the combination of A and D corresponds in each case to one row of Table B (compounds IIIc.47gB-1 to IIIc.47gB-255)

Table 48g
Compounds IIIc in which B is 2-fluoro-5-(trifluoromethoxy)phenyl and the combination of A and D corresponds in each case to one row of Table B (compounds IIIc.48gB-1 to IIIc.48gB-255)

Table 49g
Compounds IIIc in which B is 2-(difluoromethoxy)-3-fluorophenyl and the combination of A and D corresponds in each case to one row of Table B (compounds IIIc.49gB-1 to IIIc.49gB-255)

Table 50g
Compounds IIIc in which B is 2-(difluoromethoxy)-4-fluorophenyl and the combination of A and D corresponds in each case to one row of Table B (compounds IIIc.50gB-1 to IIIc.50gB-255)

Table 51g
Compounds IIIc in which B is 2-(difluoromethoxy)-5-fluorophenyl and the combination of A and D corresponds in each case to one row of Table B (compounds IIIc.51gB-1 to IIIc.51gB-255)

Table 52g
Compounds IIIc in which B is 2-(difluoromethoxy)-6-fluorophenyl and the combination of A and D corresponds in each case to one row of Table B (compounds IIIc.52gB-1 to IIIc.52gB-255)

Table 53g
Compounds IIIc in which B is 2-fluoro-3-(difluoromethoxy)phenyl and the combination of A and D corresponds in each case to one row of Table B (compounds IIIc.53gB-1 to IIIc.53gB-255)

Table 54g
Compounds IIIc in which B is 2-fluoro-4-(difluoromethoxy)phenyl and the combination of A and D corresponds in each case to one row of Table B (compounds IIIc.54gB-1 to IIIc.54gB-255)

Table 55g
Compounds IIIc in which B is 2-fluoro-5-(difluoromethoxy)phenyl and the combination of A and D corresponds in each case to one row of Table B (compounds IIIc.55gB-1 to IIIc.55gB-255)

Table 56g
Compounds IIIc in which B is 2-(trifluoromethylthio)-3-fluorophenyl and the combination of A and D corresponds in each case to one row of Table B (compounds IIIc.56gB-1 to IIIc.56gB-255)

Table 57g
Compounds IIIc in which B is 2-(trifluoromethylthio)-4-fluorophenyl and the combination of A and D corresponds in each case to one row of Table B (compounds IIIc.57gB-1 to IIIc.57gB-255)

Table 58g
Compounds IIIc in which B is 2-(trifluoromethylthio)-5-fluorophenyl and the combination of A and D corresponds in each case to one row of Table B (compounds IIIc.58gB-1 to IIIc.58gB-255)

Table 59g
Compounds IIIc in which B is 2-(trifluoromethylthio)-6-fluorophenyl and the combination of A and D corresponds in each case to one row of Table B (compounds IIIc.59gB-1 to IIIc.59gB-255)

Table 60g
Compounds IIIc in which B is 2-fluoro-3-(trifluoromethylthio)phenyl and the combination of A and D corresponds in each case to one row of Table B (compounds IIIc.60gB-1 to IIIc.60gB-255)

Table 61g
Compounds IIIc in which B is 2-fluoro-4-(trifluoromethylthio)phenyl and the combination of A and D corresponds in each case to one row of Table B (compounds IIIc.61gB-1 to IIIc.61gB-255)

Table 62g
Compounds IIIc in which B is 2-fluoro-5-(trifluoromethylthio)phenyl and the combination of A and D corresponds in each case to one row of Table B (compounds IIIc.62gB-1 to IIIc.62gB-255)

Table 63g
Compounds IIIc in which B is 2,3,4-trifluorophenyl and the combination of A and D corresponds in each case to one row of Table B (compounds IIIc.63gB-1 to IIIc.63gB-255)

Table 64g
Compounds IIIc in which B is 2,3,5-trifluorophenyl and the combination of A and D corresponds in each case to one row of Table B (compounds IIIc.64gB-1 to IIIc.64gB-255)

Table 65g
Compounds IIIc in which B is 2,3,6-trifluorophenyl and the combination of A and D corresponds in each case to one row of Table B (compounds IIIc.65gB-1 to IIIc.65gB-255)

Table 66g
Compounds IIIc in which B is 2,4,5-trifluorophenyl and the combination of A and D corresponds in each case to one row of Table B (compounds IIIc.66gB-1 to IIIc.66gB-255)

Table 67g
Compounds IIIc in which B is 2,4,6-trifluorophenyl and the combination of A and D corresponds in each case to one row of Table B (compounds IIIc.67gB-1 to IIIc.67gB-255)

Table 68g
Compounds IIIc in which B is 2,3-dichlorophenyl and the combination of A and D corresponds in each case to one row of Table B (compounds IIIc.68gB-1 to IIIc.68gB-255)

Table 69g
Compounds IIIc in which B is 2,4-dichlorophenyl and the combination of A and D corresponds in each case to one row of Table B (compounds IIIc.69gB-1 to IIIc.69gB-255)

Table 70g
Compounds IIIc in which B is 2,5-dichlorophenyl and the combination of A and D corresponds in each case to one row of Table B (compounds IIIc.70gB-1 to IIIc.70gB-255)

Table 71g
Compounds IIIc in which B is 2,6-dichlorophenyl and the combination of A and D corresponds in each case to one row of Table B (compounds IIIc.71gB-1 to IIIc.71gB-255)

Table 72g
Compounds IIIc in which B is 2,3-dimethylphenyl and the combination of A and D corresponds in each case to one row of Table B (compounds IIIc.72gB-1 to IIIc.72gB-255)

Table 73g
Compounds IIIc in which B is 2,4-dimethylphenyl and the combination of A and D corresponds in each case to one row of Table B (compounds IIIc.73gB-1 to IIIc.73gB-255)

Table 74g
Compounds IIIc in which B is 2,5-dimethylphenyl and the combination of A and D corresponds in each case to one row of Table B (compounds IIIc.74gB-1 to IIIc.74gB-255)

Table 75g
Compounds IIIc in which B is 2,6-dimethylphenyl and the combination of A and D corresponds in each case to one row of Table B (compounds IIIc.75gB-1 to IIIc.75gB-255)

Table 76g
Compounds IIIc in which B is 2,3-diethylphenyl and the combination of A and D corresponds in each case to one row of Table B (compounds IIIc.76gB-1 to IIIc.760-255)

Table 77g
Compounds IIIc in which B is 2,4-diethylphenyl and the combination of A and D corresponds in each case to one row of Table B (compounds IIIc.77gB-1 to IIIc.77gB-255)

Table 78g
Compounds IIIc in which B is 2,5-diethylphenyl and the combination of A and D corresponds in each case to one row of Table B (compounds IIIc.78gB-1 to IIIc.78gB-255)

Table 79g
Compounds IIIc in which B is 2,6-diethylphenyl and the combination of A and D corresponds in each case to one row of Table B (compounds IIIc.79gB-1 to IIIc.79gB-255)

Table 80g
Compounds IIIc in which B is 2,3-dimethoxyphenyl and the combination of A and D corresponds in each case to one row of Table B (compounds IIIc.80gB-1 to IIIc.80gB-255)

Table 81g
Compounds IIIc in which B is 2,4-dimethoxyphenyl and the combination of A and D corresponds in each case to one row of Table B (compounds IIIc.81gB-1 to IIIc.81gB-255)

Table 82g
Compounds IIIc in which B is 2,5-dimethoxyphenyl and the combination of A and D corresponds in each case to one row of Table B (compounds IIIc.82gB-1 to IIIc.82gB-255)

Table 83g
Compounds IIIc in which B is 2,6-dimethoxyphenyl and the combination of A and D corresponds in each case to one row of Table B (compounds IIIc.83gB-1 to IIIc.83gB-255)

Table 84g
Compounds IIIc in which B is 2,3-di(trifluoromethyl)phenyl and the combination of A and D corresponds in each case to one row of Table B (compounds IIIc.84gB-1 to IIIc.84gB-255)

Table 85g
Compounds IIIc in which B is 2,4-di(trifluoromethyl)phenyl and the combination of A and D corresponds in each case to one row of Table B (compounds IIIc.85gB-1 to IIIc.85gB-255)

Table 86g
Compounds IIIc in which B is 2,5-di(trifluoromethyl)phenyl and the combination of A and D corresponds in each case to one row of Table B (compounds IIIc.86gB-1 to IIIc.86gB-255)

Table 87g
Compounds IIIc in which B is 2,6-di(trifluoronnethyl)phenyl and the combination of A and D corresponds in each case to one row of Table B (compounds IIIc.87gB-1 to IIIc.87gB-255)

Table 88g
Compounds IIIc in which B is 2,3-di(trifluoromethoxy)phenyl and the combination of A and D corresponds in each case to one row of Table B (compounds IIIc.88gB-1 to IIIc.88gB-255)

Table 89g
Compounds IIIc in which B is 2,4-di(trifluoromethoxy)phenyl and the combination of A and D corresponds in each case to one row of Table B (compounds IIIc.89gB-1 to IIIc.89gB-255)

Table 90g
Compounds IIIc in which B is 2,5-di(trifluoromethoxy)phenyl and the combination of A and D corresponds in each case to one row of Table B (compounds IIIc.90gB-1 to IIIc.90gB-255)

Table 91g
Compounds IIIc in which B is 2,6-di(trifluoromethoxy)phenyl and the combination of A and D corresponds in each case to one row of Table B (compounds IIIc.91gB-1 to IIIc.91gB-255)

Table 92g
Compounds IIIc in which B is 2,3-di(difluoromethoxy)phenyl and the combination of A and D corresponds in each case to one row of Table B (compounds IIIc.92gB-1 to IIIc.92gB-255)

Table 93g
Compounds IIIc in which B is 2,4-di(difluoromethoxy)phenyl and the combination of A and D corresponds in each case to one row of Table B (compounds IIIc.93gB-1 to IIIc.93gB-255)

Table 94g
Compounds IIIc in which B is 2,5-di(difluoromethoxy)phenyl and the combination of A and D corresponds in each case to one row of Table B (compounds IIIc.94gB-1 to IIIc.94gB-255)

Table 95g
Compounds IIIc in which B is 2,6-di(difluoromethoxy)phenyl and the combination of A and D corresponds in each case to one row of Table B (compounds IIIc.95gB-1 to IIIc.95gB-255)

Table 96g
Compounds IIIc in which B is 2,3-di(trifluoromethylthio)phenyl and the combination of A and D corresponds in each case to one row of Table B (compounds IIIc.96gB-1 to IIIc.96gB-255)

Table 97g
Compounds IIIc in which B is 2,4-di(trifluoromethylthio)phenyl and the combination of A and D corresponds in each case to one row of Table B (compounds IIIc.97gB-1 to IIIc.97gB-255)

Table 98g
Compounds IIIc in which B is 2,5-di(trifluoromethylthio)phenyl and the combination of A and D corresponds in each case to one row of Table B (compounds IIIc.98gB-1 to IIIc.98gB-255)

Table 99g
Compounds IIIc in which B is 2,6-di(trifluoromethylthio)phenyl and the combination of A and D corresponds in each case to one row of Table B (compounds IIIc.99gB-1 to IIIc.99gB-255)

Table 100g
Compounds IIIc in which B is 2-methyl-3-chlorophenyl and the combination of A and D corresponds in each case to one row of Table B (compounds IIIc.100gB-1 to IIIc.100gB-255)

Table 101g
Compounds IIIc in which B is 2-methyl-4-chlorophenyl and the combination of A and D corresponds in each case to one row of Table B (compounds IIIc.101gB-1 to IIIc.101gB-255)

Table 102g
Compounds IIIc in which B is 2-methyl-5-chlorophenyl and the combination of A and D corresponds in each case to one row of Table B (compounds IIIc.102gB-1 to IIIc.102gB-255)

Table 103g
Compounds IIIc in which B is 2-methyl-6-chlorophenyl and the combination of A and D corresponds in each case to one row of Table B (compounds IIIc.103gB-1 to IIIc.103gB-255)

Table 104g
Compounds IIIc in which B is 2-chloro-3-methylphenyl and the combination of A and D corresponds in each case to one row of Table B (compounds IIIc.104gB-1 to IIIc.104gB-255)

Table 105g
Compounds IIIc in which B is 2-chloro-4-methylphenyl and the combination of A and D corresponds in each case to one row of Table B (compounds IIIc.105gB-1 to IIIc.105gB-255)

Table 106g
Compounds IIIc in which B is 2-chloro-5-methylphenyl and the combination of A and D corresponds in each case to one row of Table B (compounds IIIc.106gB-1 to IIIc.106gB-255)

Table 107g
Compounds IIIc in which B is 2-chloro-3-ethylphenyl and the combination of A and D corresponds in each case to one row of Table B (compounds IIIc.107gB-1 to IIIc.107gB-255)

Table 108g
Compounds IIIc in which B is 2-chloro-4-ethylphenyl and the combination of A and D corresponds in each case to one row of Table B (compounds IIIc.108gB-1 to IIIc.108gB-255)

Table 109g
Compounds IIIc in which B is 2-chloro-5-ethylphenyl and the combination of A and D corresponds in each case to one row of Table B (compounds IIIc.109gB-1 to IIIc.109gB-255)

Table 110g
Compounds IIIc in which B is 2-ethyl-3-chlorophenyl and the combination of A and D corresponds in each case to one row of Table B (compounds IIIc.110gB-1 to IIIc.110gB-255)

Table 111g
Compounds IIIc in which B is 2-ethyl-4-chlorophenyl and the combination of A and D corresponds in each case to one row of Table B (compounds IIIc.111gB-1 to IIIc.111gB-255)

Table 112g
Compounds IIIc in which B is 2-ethyl-5-chlorophenyl and the combination of A and D corresponds in each case to one row of Table B (compounds IIIc.112gB-1 to IIIc.112gB-255)

Table 113g
Compounds IIIc in which B is 2-ethyl-6-chlorophenyl and the combination of A and D corresponds in each case to one row of Table B (compounds IIIc.113gB-1 to IIIc.113gB-255)

Table 114g
Compounds IIIc in which B is 2-methoxy-3-chlorophenyl and the combination of A and D corresponds in each case to one row of Table B (compounds IIIc.114gB-1 to IIIc.114gB-255)

Table 115g
Compounds IIIc in which B is 2-methoxy-4-chlorophenyl and the combination of A and D corresponds in each case to one row of Table B (compounds IIIc.115gB-1 to IIIc.115gB-255)

Table 116g
Compounds IIIc in which B is 2-methoxy-5-chlorophenyl and the combination of A and D corresponds in each case to one row of Table B (compounds IIIc.116gB-1 to IIIc.116gB-255)

Table 117g
Compounds IIIc in which B is 2-methoxy-6-chlorophenyl and the combination of A and D corresponds in each case to one row of Table B (compounds IIIc.117gB-1 to IIIc.117gB-255)

Table 118g
Compounds IIIc in which B is 2-chloro-3-methoxyphenyl and the combination of A and D corresponds in each case to one row of Table B (compounds IIIc.118gB-1 to IIIc.118gB-255)

Table 119g
Compounds IIIc in which B is 2-chloro-4-methoxyphenyl and the combination of A and D corresponds in each case to one row of Table B (compounds IIIc.119gB-1 to IIIc.119gB-255)

Table 120g
Compounds IIIc in which B is 2-chloro-5-methoxyphenyl and the combination of A and D corresponds in each case to one row of Table B (compounds IIIc.120gB-1 to IIIc.120gB-255)

Table 121g
Compounds IIIc in which B is 2-(trifluoromethyl)-3-chlorophenyl and the combination of A and D corresponds in each case to one row of Table B (compounds IIIc.121gB-1 to IIIc.121gB-255)

Table 122g
Compounds IIIc in which B is 2-(trifluoromethyl)-4-chlorophenyl and the combination of A and D corresponds in each case to one row of Table B (compounds IIIc.122gB-1 to IIIc.122gB-255)

Table 123g
Compounds IIIc in which B is 2-(trifluoromethyl)-5-chlorophenyl and the combination of A and D corresponds in each case to one row of Table B (compounds IIIc.123gB-1 to IIIc.123gB-255)

Table 124g
Compounds IIIc in which B is 2-(trifluoromethyl)-6-chlorophenyl and the combination of A and D corresponds in each case to one row of Table B (compounds IIIc.124gB-1 to IIIc.124gB-255)

Table 125g
Compounds IIIc in which B is 2-chloro-3-(trifluoromethyl)phenyl and the combination of A and D corresponds in each case to one row of Table B (compounds IIIc.125gB-1 to IIIc.125gB-255)

Table 126g
Compounds IIIc in which B is 2-chloro-4-(trifluoromethyl)phenyl and the combination of A and D corresponds in each case to one row of Table B (compounds IIIc.126gB-1 to IIIc.126gB-255)

Table 127g
Compounds IIIc in which B is 2-chloro-5-(trifluoromethyl)phenyl and the combination of A and D corresponds in each case to one row of Table B (compounds IIIc.127gB-1 to IIIc.127gB-255)

Table 128g
Compounds IIIc in which B is 2-(trifluoromethoxy)-3-chlorophenyl and the combination of A and D corresponds in each case to one row of Table B (compounds IIIc.128gB-1 to IIIc.128gB-255)

Table 129g
Compounds IIIc in which B is 2-(trifluoromethoxy)-4-chlorophenyl and the combination of A and D corresponds in each case to one row of Table B (compounds IIIc.129gB-1 to IIIc.129gB-255)

Table 130g
Compounds IIIc in which B is 2-(trifluoromethoxy)-5-chlorophenyl and the combination of A and D corresponds in each case to one row of Table B (compounds IIIc.130gB-1 to IIIc.130gB-255)

Table 131g
Compounds IIIc in which B is 2-(trifluoromethoxy)-6-chlorophenyl and the combination of A and D corresponds in each case to one row of Table B (compounds IIIc.131gB-1 to IIIc.131gB-255)

Table 132g
Compounds IIIc in which B is 2-chloro-3-(trifluoromethoxy)phenyl and the combination of A and D corresponds in each case to one row of Table B (compounds IIIc.132gB-1 to IIIc.132gB-255)

Table 133g
Compounds IIIc in which B is 2-chloro-4-(trifluoromethoxy)phenyl and the combination of A and D corresponds in each case to one row of Table B (compounds IIIc.133gB-1 to IIIc.133gB-255)

Table 134g
Compounds IIIc in which B is 2-chloro-5-(trifluoromethoxy)phenyl and the combination of A and D corresponds in each case to one row of Table B (compounds IIIc.134gB-1 to IIIc.134gB-255)

Table 135g
Compounds IIIc in which B is 2-(difluoromethoxy)-3-chlorophenyl and the combination of A and D corresponds in each case to one row of Table B (compounds IIIc.135gB-1 to IIIc.135gB-255)

Table 136g
Compounds IIIc in which B is 2-(difluoromethoxy)-4-chlorophenyl and the combination of A and D corresponds in each case to one row of Table B (compounds IIIc.136gB-1 to IIIc.136gB-255)

Table 137g
Compounds IIIc in which B is 2-(difluoromethoxy)-5-chlorophenyl and the combination of A and D corresponds in each case to one row of Table B (compounds IIIc.137gB-1 to IIIc.137gB-255)

Table 138g
Compounds IIIc in which B is 2-(difluoromethoxy)-6-chlorophenyl and the combination of A and D corresponds in each case to one row of Table B (compounds IIIc.138gB-1 to IIIc.138gB-255)

Table 139g
Compounds IIIc in which B is 3-(difluoromethoxy)-4-chlorophenyl and the combination of A and D corresponds in each case to one row of Table B (compounds IIIc.139gB-1 to IIIc.139gB-255)

Table 140g
Compounds IIIc in which B is 2-chloro-3-(difluoromethoxy)phenyl and the combination of A and D corresponds in each case to one row of Table B (compounds IIIc.140gB-1 to IIIc.140gB-255)

Table 141g
Compounds IIIc in which B is 2-chloro-4-(difluoromethoxy)phenyl and the combination of A and D corresponds in each case to one row of Table B (compounds IIIc.141gB-1 to IIIc.141gB-255)

Table 142g
Compounds IIIc in which B is 2-chloro-5-(difluoromethoxy)phenyl and the combination of A and D corresponds in each case to one row of Table B (compounds IIIc.142gB-1 to IIIc.142gB-255)

Table 143g
Compounds IIIc in which B is 2-(trifluoromethylthio)-3-chlorophenyl and the combination of A and D corresponds in each case to one row of Table B (compounds IIIc.143gB-1 to IIIc.143gB-255)

Table 144g
Compounds IIIc in which B is 2-(trifluoromethylthio)-4-chlorophenyl and the combination of A and D corresponds in each case to one row of Table B (compounds IIIc.144gB-1 to IIIc.144gB-255)

Table 145g
Compounds IIIc in which B is 2-(trifluoromethylthio)-5-chlorophenyl and the combination of A and D corresponds in each case to one row of Table B (compounds IIIc.145gB-1 to IIIc.145gB-255)

Table 146g
Compounds IIIc in which B is 2-(trifluoromethylthio)-6-chlorophenyl and the combination of A and D corresponds in each case to one row of Table B (compounds IIIc.146gB-1 to IIIc.146gB-255)

Table 147g
Compounds IIIc in which B is 2-chloro-3-(trifluoromethylthio)phenyl and the combination of A and D corresponds in each case to one row of Table B (compounds IIIc.147gB-1 to IIIc.147gB-255)

Table 148g
Compounds IIIc in which B is 2-chloro-4-(trifluoromethylthio)phenyl and the combination of A and D corresponds in each case to one row of Table B (compounds IIIc.148gB-1 to IIIc.148gB-255)

Table 149g
Compounds IIIc in which B is 2-chloro-5-(trifluoromethylthio)phenyl and the combination of A and D corresponds in each case to one row of Table B (compounds IIIc.149gB-1 to IIIc.149gB-255)

Table 150g
Compounds IIIc in which B is 2,3,4-trichlorophenyl and the combination of A and D corresponds in each case to one row of Table B (compounds IIIc.150gB-1 to IIIc.150gB-255)

Table 151g
Compounds IIIc in which B is 2,3,5-trichlorophenyl and the combination of A and D corresponds in each case to one row of Table B (compounds IIIc.151gB-1 to IIIc.151gB-255)

Table 152g
Compounds IIIc in which B is 2,3,6-trichlorophenyl and the combination of A and D corresponds in each case to one row of Table B (compounds IIIc.152gB-1 to IIIc.152gB-255)

Table 153g
Compounds IIIc in which B is 2,4,5-trichlorophenyl and the combination of A and D corresponds in each case to one row of Table B (compounds IIIc.153gB-1 to IIIc.153gB-255)

Table 154g
Compounds IIIc in which B is 2,4,6-trichlorophenyl and the combination of A and D corresponds in each case to one row of Table B (compounds IIIc.154gB-1 to IIIc.154gB-255)

Table 155g
Compounds IIIc in which B is 2,3,4-trimethylphenyl and A corresponds in each case to one group of one row of Table B (compounds IIIc.155gB-1 to IIIc.155gB-255)

Table 156g
Compounds IIIc in which B is 2,3,5-trimethylphenyl and A corresponds in each case to one group of one row of Table B (compounds IIIc.156gB-1 to IIIc.156gB-255)

Table 157g
Compounds IIIc in which B is 2,3,6-trimethylphenyl and A corresponds in each case to one group of one row of Table B (compounds IIIc.157gB-1 to IIIc.157gB-255)

Table 158g
Compounds IIIc in which B is 2,4,5-trimethylphenyl and A corresponds in each case to one group of one row of Table B (compounds IIIc.158gB-1 to IIIc.158gB-255)

Table 159g
Compounds IIIc in which B is 2,4,6-trimethylphenyl and A corresponds in each case to one group of one row of Table B (compounds IIIc.159gB-1 to IIIc.159gB-255)

Table 160g
Compounds IIIc in which B is 2,3,4-trimethoxyphenyl and A corresponds in each case to one group of one row of Table B (compounds IIIc.160gB-1 to IIIc.160gB-255)

Table 161g
Compounds IIIc in which B is 2,3,5-trimethoxyphenyl and A corresponds in each case to one group of one row of Table B (compounds IIIc.161gB-1 to IIIc.161gB-255)

Table 162g
Compounds IIIc in which B is 2,3,6-trimethoxyphenyl and A corresponds in each case to one group of one row of Table B (compounds IIIc.162gB-1 to IIIc.162gB-255)

Table 1h
Compounds IIId-1 in which B is 2,3-difluorophenyl and the combination of A and D corresponds in each case to one row of Table B (compounds IIId-1.1hB-1 to IIId-1.1hB-255)

Table 2h
Compounds IIId-1 in which B is 2,4-difluorophenyl and the combination of A and D corresponds in each case to one row of Table B (compounds IIId-1.2hB-1 to IIId-1.2hB-255)

Table 3h
Compounds IIId-1 in which B is 2,5-difluorophenyl and the combination of A and D corresponds in each case to one row of Table B (compounds IIId-1.3hB-1 to IIId-1.3h B-255)

Table 4h
Compounds IIId-1 in which B is 2,6-difluorophenyl and the combination of A and D corresponds in each case to one row of Table B (compounds IIId-1.4hB-1 to IIId-1.4hB-255)

Table 5h
Compounds IIId-1 in which B is 2-fluoro-3-chlorophenyl and the combination of A and D corresponds in each case to one row of Table B (compounds IIId-1.5hB-1 to IIId-1.5hB-255)

Table 6h
Compounds IIId-1 in which B is 2-fluoro-4-chlorophenyl and the combination of A and D corresponds in each case to one row of Table B (compounds IIId-1.6hB-1 to IIId-1.6hB-255)

Table 7h
Compounds IIId-1 in which B is 2-fluoro-5-chlorophenyl and the combination of A and D corresponds in each case to one row of Table B (compounds IIId-1.7hB-1 to IIId-1.7hB-255)

Table 8h
Compounds IIId-1 in which B is 2-fluoro-6-chlorophenyl and the combination of A and D corresponds in each case to one row of Table B (compounds IIId-1.8hB-1 to IIId-1.8hB-255)

Table 9h
Compounds IIId-1 in which B is 2-chloro-3-fluorophenyl and the combination of A and D corresponds in each case to one row of Table B (compounds IIId-1.9hB-1 to IIId-1.9hB-255)

Table 10h
Compounds IIId-1 in which B is 2-chloro-4-fluorophenyl and the combination of A and D corresponds in each case to one row of Table B (compounds IIId-1.10hB-1 to IIId-1.10hB-255)

Table 11h
Compounds IIId-1 in which B is 2-chloro-5-fluorophenyl and the combination of A and D corresponds in each case to one row of Table B (compounds IIId-1.11hB-1 to IIId-1.11hB-255)

Table 12h
Compounds IIId-1 in which B is 2-methyl-3-fluorophenyl and the combination of A and D corresponds in each case to one row of Table B (compounds IIId-1.12hB-1 to IIId-1.12hB-255)

Table 13h
Compounds IIId-1 in which B is 2-methyl-4-fluorophenyl and the combination of A and D corresponds in each case to one row of Table B (compounds IIId-1.13hB-1 to IIId-1.13hB-255)

Table 14h
Compounds IIId-1 in which B is 2-methyl-5-fluorophenyl and the combination of A and D corresponds in each case to one row of Table B (compounds IIId-1.14hB-1 to IIId-1.14hB-255)

Table 15h
Compounds IIId-1 in which B is 2-methyl-6-fluorophenyl and the combination of A and D corresponds in each case to one row of Table B (compounds IIId-1.15hB-1 to IIId-1.15hB-255)

Table 16h
Compounds IIId-1 in which B is 2-fluoro-3-methylphenyl and the combination of A and D corresponds in each case to one row of Table B (compounds IIId-1.16hB-1 to IIId-1.16hB-255)

Table 17h
Compounds IIId-1 in which B is 2-fluoro-4-methylphenyl and the combination of A and D corresponds in each case to one row of Table B (compounds IIId-1.17hB-1 to IIId-1.17hB-255)

Table 18h
Compounds IIId-1 in which B is 2-fluoro-5-methylphenyl and the combination of A and D corresponds in each case to one row of Table B (compounds IIId-1.18hB-1 to IIId-1.18hB-255)

Table 19h
Compounds IIId-1 in which B is 2-ethyl-3-fluorophenyl and the combination of A and D corresponds in each case to one row of Table B (compounds IIId-1.19hB-1 to IIId-1.19hB-255)

Table 20h
Compounds IIId-1 in which B is 2-ethyl-4-fluorophenyl and the combination of A and D corresponds in each case to one row of Table B (compounds IIId-1.20hB-1 to IIId-1.20hB-255)

Table 21h
Compounds IIId-1 in which B is 2-ethyl-5-fluorophenyl and the combination of A and D corresponds in each case to one row of Table B (compounds IIId-1.21hB-1 to IIId-1.21hB-255)

Table 22h
Compounds IIId-1 in which B is 2-ethyl-6-fluorophenyl and the combination of A and D corresponds in each case to one row of Table B (compounds IIId-1.22hB-1 to IIId-1.22hB-255)

Table 23h
Compounds IIId-1 in which B is 2-fluoro-3-ethylphenyl and the combination of A and D corresponds in each case to one row of Table B (compounds IIId-1.23hB-1 to IIId-1.23hB-255)

Table 24h
Compounds IIId-1 in which B is 2-fluoro-4-ethylphenyl and the combination of A and D corresponds in each case to one row of Table B (compounds IIId-1.24hB-1 to IIId-1.24hB-255)

Table 25h
Compounds IIId-1 in which B is 2-methoxy-3-fluorophenyl and the combination of A and D corresponds in each case to one row of Table B (compounds IIId-1.25hB-1 to IIId-1.25hB-255)

Table 26h
Compounds IIId-1 in which B is 2-methoxy-4-fluorophenyl and the combination of A and D corresponds in each case to one row of Table B (compounds IIId-1.26hB-1 to IIId-1.26hB-255)

Table 27h
Compounds IIId-1 in which B is 2-methoxy-5-fluorophenyl and the combination of A and D corresponds in each case to one row of Table B (compounds IIId-1.27hB-1 to IIId-1.27hB-255)

Table 28h
Compounds IIId-1 in which B is 2-methoxy-6-fluorophenyl and the combination of A and D corresponds in each case to one row of Table B (compounds IIId-1.28hB-1 to IIId-1.28hB-255)

Table 29h
Compounds IIId-1 in which B is 2-fluoro-3-methoxyphenyl and the combination of A and D corresponds in each case to one row of Table B (compounds IIId-1.29hB-1 to IIId-1.29hB-255)

Table 30h
Compounds IIId-1 in which B is 2-fluoro-4-methoxyphenyl and the combination of A and D corresponds in each case to one row of Table B (compounds IIId-1.30hB-1 to IIId-1.30hB-255)

Table 31h
Compounds IIId-1 in which B is 2-fluoro-5-methoxyphenyl and the combination of A and D corresponds in each case to one row of Table B (compounds IIId-1.31hB-1 to IIId-1.31hB-255)

Table 32h
Compounds IIId-1 in which B is 2-(trifluoromethyl)-3-fluorophenyl and the combination of A and D corresponds in each case to one row of Table B (compounds IIId-1.32hB-1 to IIId-1.32hB-255)

Table 33h
Compounds IIId-1 in which B is 2,4,5-trimethoxyphenyl and the combination of A and D corresponds in each case to one row of Table B (compounds IIId-1.33hB-1 to IIId-1.33hB-255)

Table 34h
Compounds IIId-1 in which B is 2,4,6-trimethoxyphenyl and the combination of A and D corresponds in each case to one row of Table B (compounds IIId-1.34hB-1 to IIId-1.34hB-255)

Table 35h
Compounds IIId-1 in which B is 2-fluoro-5-ethylphenyl and the combination of A and D corresponds in each case to one row of Table B (compounds IIId-1.35hB-1 to IIId-1.35hB-255)

Table 36h
Compounds IIId-1 in which B is 2-(trifluoromethyl)-4-fluorophenyl and the combination of A and D corresponds in each case to one row of Table B (compounds IIId-1.36hB-1 to IIId-1.36hB-255)

Table 37h
Compounds IIId-1 in which B is 2-(trifluoromethyl)-5-fluorophenyl and the combination of A and D corresponds in each case to one row of Table B (compounds IIId-1.37hB-1 to IIId-1.37hB-255)

Table 38h
Compounds IIId-1 in which B is 2-(trifluoromethyl)-6-fluorophenyl and the combination of A and D corresponds in each case to one row of Table B (compounds IIId-1.38hB-1 to IIId-1.38hB-255)

Table 39h
Compounds IIId-1 in which B is 2-fluoro-3-(trifluoromethyl)phenyl and the combination of A and D corresponds in each case to one row of Table B (compounds IIId-1.39hB-1 to IIId-1.39hB-255)

Table 40h
Compounds IIId-1 in which B is 2-fluoro-4-(trifluoromethyl)phenyl and the combination of A and D corresponds in each case to one row of Table B (compounds IIId-1.40hB-1 to IIId-1.40hB-255)

Table 41h
Compounds IIId-1 in which B is 2-fluoro-5-(trifluoromethyl)phenyl and the combination of A and D corresponds in each case to one row of Table B (compounds IIId-1.41hB-1 to IIId-1.41hB-255)

Table 42h
Compounds IIId-1 in which B is 2-(trifluoromethoxy)-3-fluorophenyl and the combination of A and D corresponds in each case to one row of Table B (compounds IIId-1.42hB-1 to IIId-1.42hB-255)

Table 43h
Compounds IIId-1 in which B is 2-(trifluoromethoxy)-4-fluorophenyl and the combination of A and D corresponds in each case to one row of Table B (compounds IIId-1.43hB-1 to IIId-1.43hB-255)

Table 44h
Compounds IIId-1 in which B is 2-(trifluoromethoxy)-5-fluorophenyl and the combination of A and D corresponds in each case to one row of Table B (compounds IIId-1.44hB-1 to IIId-1.44hB-255)

Table 45h
Compounds IIId-1 in which B is 2-(trifluoromethoxy)-6-fluorophenyl and the combination of A and D corresponds in each case to one row of Table B (compounds IIId-1.45hB-1 to IIId-1.45hB-255)

Table 46h
Compounds IIId-1 in which B is 2-fluoro-3-(trifluoromethoxy)phenyl and the combination of A and D corresponds in each case to one row of Table B (compounds IIId-1.46hB-1 to IIId-1.46hB-255)

Table 47h
Compounds IIId-1 in which B is 2-fluoro-4-(trifluoromethoxy)phenyl and the combination of A and D corresponds in each case to one row of Table B (compounds IIId-1.47hB-1 to IIId-1.47hB-255)

Table 48h
Compounds IIId-1 in which B is 2-fluoro-5-(trifluoromethoxy)phenyl and the combination of A and D corresponds in each case to one row of Table B (compounds IIId-1.48hB-1 to IIId-1.48hB-255)

Table 49h
Compounds IIId-1 in which B is 2-(difluoromethoxy)-3-fluorophenyl and the combination of A and D corresponds in each case to one row of Table B (compounds IIId-1.49hB-1 to IIId-1.49hB-255)

Table 50h
Compounds IIId-1 in which B is 2-(difluoromethoxy)-4-fluorophenyl and the combination of A and D corresponds in each case to one row of Table B (compounds IIId-1.50hB-1 to IIId-1.50hB-255)

Table 51h
Compounds IIId-1 in which B is 2-(difluoromethoxy)-5-fluorophenyl and the combination of A and D corresponds in each case to one row of Table B (compounds IIId-1.51hB-1 to IIId-1.51hB-255)

Table 52h
Compounds IIId-1 in which B is 2-(difluoromethoxy)-6-fluorophenyl and the combination of A and D corresponds in each case to one row of Table B (compounds IIId-1.52hB-1 to IIId-1.52hB-255)

Table 53h
Compounds IIId-1 in which B is 2-fluoro-3-(difluoromethoxy)phenyl and the combination of A and D corresponds in each case to one row of Table B (compounds IIId-1.53hB-1 to IIId-1.53hB-255)

Table 54h
Compounds IIId-1 in which B is 2-fluoro-4-(difluoromethoxy)phenyl and the combination of A and D corresponds in each case to one row of Table B (compounds IIId-1.54hB-1 to IIId-1.54hB-255)

Table 55h
Compounds IIId-1 in which B is 2-fluoro-5-(difluoromethoxy)phenyl and the combination of A and D corresponds in each case to one row of Table B (compounds IIId-1.55hB-1 to IIId-1.55hB-255)

Table 56h
Compounds IIId-1 in which B is 2-(trifluoromethylthio)-3-fluorophenyl and the combination of A and D corresponds in each case to one row of Table B (compounds IIId-1.56hB-1 to IIId-1.56hB-255)

Table 57h
Compounds IIId-1 in which B is 2-(trifluoromethylthio)-4-fluorophenyl and the combination of A and D corresponds in each case to one row of Table B (compounds IIId-1.57hB-1 to IIId-1.57hB-255)

Table 58h
Compounds IIId-1 in which B is 2-(trifluoromethylthio)-5-fluorophenyl and the combination of A and D corresponds in each case to one row of Table B (compounds IIId-1.58hB-1 to IIId-1.58hB-255)

Table 59h
Compounds IIId-1 in which B is 2-(trifluoromethylthio)-6-fluorophenyl and the combination of A and D corresponds in each case to one row of Table B (compounds IIId-1.59hB-1 to IIId-1.59hB-255) Table 60h
Compounds IIId-1 in which B is 2-fluoro-3-(trifluoromethylthio)phenyl and the combination of A and D corresponds in each case to one row of Table B (compounds IIId-1.60hB-1 to IIId-1.60hB-255)

Table 61h
Compounds IIId-1 in which B is 2-fluoro-4-(trifluoromethylthio)phenyl and the combination of A and D corresponds in each case to one row of Table B (compounds IIId-1.61hB-1 to IIId-1.61hB-255)

Table 62h
Compounds IIId-1 in which B is 2-fluoro-5-(trifluoromethylthio)phenyl and the combination of A and D corresponds in each case to one row of Table B (compounds IIId-1.62hB-1 to IIId-1.62hB-255)

Table 63h
Compounds IIId-1 in which B is 2,3,4-trifluorophenyl and the combination of A and D corresponds in each case to one row of Table B (compounds IIId-1.63hB-1 to IIId-1.63hB-255)

Table 64h
Compounds IIId-1 in which B is 2,3,5-trifluorophenyl and the combination of A and D corresponds in each case to one row of Table B (compounds IIId-1.64hB-1 to IIId-1.64hB-255)

Table 65h
Compounds IIId-1 in which B is 2,3,6-trifluorophenyl and the combination of A and D corresponds in each case to one row of Table B (compounds IIId-1.65hB-1 to IIId-1.65hB-255)

Table 66h
Compounds IIId-1 in which B is 2,4,5-trifluorophenyl and the combination of A and D corresponds in each case to one row of Table B (compounds IIId-1.66hB-1 to IIId-1.66hB-255)

Table 67h
Compounds IIId-1 in which B is 2,4,6-trifluorophenyl and the combination of A and D corresponds in each case to one row of Table B (compounds IIId-1.67hB-1 to IIId-1.67hB-255)

Table 68h
Compounds IIId-1 in which B is 2,3-dichlorophenyl and the combination of A and D corresponds in each case to one row of Table B (compounds IIId-1.68hB-1 to IIId-1.68hB-255)

Table 69h
Compounds IIId-1 in which B is 2,4-dichlorophenyl and the combination of A and D corresponds in each case to one row of Table B (compounds IIId-1.69hB-1 to IIId-1.69hB-255)

Table 70h
Compounds IIId-1 in which B is 2,5-dichlorophenyl and the combination of A and D corresponds in each case to one row of Table B (compounds IIId-1.70hB-1 to IIId-1.70hB-255)

Table 71h
Compounds IIId-1 in which B is 2,6-dichlorophenyl and the combination of A and D corresponds in each case to one row of Table B (compounds IIId-1.71hB-1 to IIId-1.71hB-255)

Table 72h
Compounds IIId-1 in which B is 2,3-dimethylphenyl and the combination of A and D corresponds in each case to one row of Table B (compounds IIId-1.72hB-1 to IIId-1.72hB-255)

Table 73h
Compounds IIId-1 in which B is 2,4-dimethylphenyl and the combination of A and D corresponds in each case to one row of Table B (compounds IIId-1.73hB-1 to IIId-1.73hB-255)

Table 74h
Compounds IIId-1 in which B is 2,5-dimethylphenyl and the combination of A and D corresponds in each case to one row of Table B (compounds IIId-1.74hB-1 to IIId-1.74hB-255)

Table 75h
Compounds IIId-1 in which B is 2,6-dimethylphenyl and the combination of A and D corresponds in each case to one row of Table B (compounds IIId-1.75hB-1 to IIId-1.75hB-255)

Table 76h
Compounds IIId-1 in which B is 2,3-diethylphenyl and the combination of A and D corresponds in each case to one row of Table B (compounds IIId-1.76hB-1 to IIId-1.76hB-255)

Table 77h
Compounds IIId-1 in which B is 2,4-diethylphenyl and the combination of A and D corresponds in each case to one row of Table B (compounds IIId-1.77hB-1 to IIId-1.77hB-255)

Table 78h
Compounds IIId-1 in which B is 2,5-diethylphenyl and the combination of A and D corresponds in each case to one row of Table B (compounds IIId-1.78hB-1 to IIId-1.78hB-255)

Table 79h
Compounds IIId-1 in which B is 2,6-diethylphenyl and the combination of A and D corresponds in each case to one row of Table B (compounds IIId-1.79hB-1 to IIId-1.79hB-255)

Table 80h
Compounds IIId-1 in which B is 2,3-dimethoxyphenyl and the combination of A and D corresponds in each case to one row of Table B (compounds IIId-1.80hB-1 to IIId-1.80hB-255)

Table 81h
Compounds IIId-1 in which B is 2,4-dimethoxyphenyl and the combination of A and D corresponds in each case to one row of Table B (compounds IIId-1.81hB-1 to IIId-1.81hB-255)

Table 82h
Compounds IIId-1 in which B is 2,5-dimethoxyphenyl and the combination of A and D corresponds in each case to one row of Table B (compounds IIId-1.82hB-1 to IIId-1.82hB-255)

Table 83h
Compounds IIId-1 in which B is 2,6-dimethoxyphenyl and the combination of A and D corresponds in each case to one row of Table B (compounds IIId-1.83hB-1 to IIId-1.83hB-255)

Table 84h
Compounds IIId-1 in which B is 2,3-di(trifluoromethyl)phenyl and the combination of A and D corresponds in each case to one row of Table B (compounds IIId-1.84hB-1 to IIId-1.84hB-255)

Table 85h
Compounds IIId-1 in which B is 2,4-di(trifluoromethyl)phenyl and the combination of A and D corresponds in each case to one row of Table B (compounds IIId-1.85hB-1 to IIId-1.85hB-255)

Table 86h
Compounds IIId-1 in which B is 2,5-di(trifluoromethyl)phenyl and the combination of A and D corresponds in each case to one row of Table B (compounds IIId-1.86hB-1 to IIId-1.86hB-255)

Table 87h
Compounds IIId-1 in which B is 2,6-di(trifluoromethyl)phenyl and the combination of A and D corresponds in each case to one row of Table B (compounds IIId-1.87hB-1 to IIId-1.87hB-255)

Table 88h
Compounds IIId-1 in which B is 2,3-di(trifluoromethoxy)phenyl and the combination of A and D corresponds in each case to one row of Table B (compounds IIId-1.88hB-1 to IIId-1.88hB-255)

Table 89h
Compounds IIId-1 in which B is 2,4-di(trifluoromethoxy)phenyl and the combination of A and D corresponds in each case to one row of Table B (compounds IIId-1.89hB-1 to IIId-1.89hB-255)

Table 90h
Compounds IIId-1 in which B is 2,5-di(trifluoromethoxy)phenyl and the combination of A and D corresponds in each case to one row of Table B (compounds IIId-1.90hB-1 to IIId-1.90hB-255)

Table 91h
Compounds IIId-1 in which B is 2,6-di(trifluoromethoxy)phenyl and the combination of A and D corresponds in each case to one row of Table B (compounds IIId-1.91hB-1 to IIId-1.91hB-255)

Table 92h
Compounds IIId-1 in which B is 2,3-di(difluoromethoxy)phenyl and the combination of A and D corresponds in each case to one row of Table B (compounds IIId-1.92hB-1 to IIId-1.92hB-255)

Table 93h
Compounds IIId-1 in which B is 2,4-di(difluoromethoxy)phenyl and the combination of A and D corresponds in each case to one row of Table B (compounds IIId-1.93hB-1 to IIId-1.93hB-255)

Table 94h
Compounds IIId-1 in which B is 2,5-di(difluoromethoxy)phenyl and the combination of A and D corresponds in each case to one row of Table B (compounds IIId-1.94hB-1 to IIId-1.94hB-255)

Table 95h
Compounds IIId-1 in which B is 2,6-di(difluoromethoxy)phenyl and the combination of A and D corresponds in each case to one row of Table B (compounds IIId-1.95hB-1 to IIId-1.95hB-255)

Table 96h
Compounds IIId-1 in which B is 2,3-di(trifluoromethylthio)phenyl and the combination of A and D corresponds in each case to one row of Table B (compounds IIId-1.96hB-1 to IIId-1.96hB-255)

Table 97h
Compounds IIId-1 in which B is 2,4-di(trifluoromethylthio)phenyl and the combination of A and D corresponds in each case to one row of Table B (compounds IIId-1.97hB-1 to IIId-1.97hB-255)

Table 98h
Compounds IIId-1 in which B is 2,5-di(trifluoromethylthio)phenyl and the combination of A and D corresponds in each case to one row of Table B (compounds IIId-1.98hB-1 to IIId-1.98hB-255)

Table 99h
Compounds IIId-1 in which B is 2,6-di(trifluoromethylthio)phenyl and the combination of A and D corresponds in each case to one row of Table B (compounds IIId-1.99hB-1 to IIId-1.99hB-255)

Table 100h
Compounds IIId-1 in which B is 2-methyl-3-chlorophenyl and the combination of A and D corresponds in each case to one row of Table B (compounds IIId-1.100hB-1 to IIId-1.100hB-255)

Table 101h
Compounds IIId-1 in which B is 2-methyl-4-chlorophenyl and the combination of A and D corresponds in each case to one row of Table B (compounds IIId-1.101hB-1 to IIId-1.101hB-255)

Table 102h
Compounds IIId-1 in which B is 2-methyl-5-chlorophenyl and the combination of A and D corresponds in each case to one row of Table B (compounds IIId-1.102hB-1 to IIId-1.102hB-255)

Table 103h
Compounds IIId-1 in which B is 2-methyl-6-chlorophenyl and the combination of A and D corresponds in each case to one row of Table B (compounds IIId-1.103hB-1 to IIId-1.103hB-255)

Table 104h
Compounds IIId-1 in which B is 2-chloro-3-methylphenyl and the combination of A and D corresponds in each case to one row of Table B (compounds IIId-1.104hB-1 to IIId-1.104hB-255)

Table 105h
Compounds IIId-1 in which B is 2-chloro-4-methylphenyl and the combination of A and D corresponds in each case to one row of Table B (compounds IIId-1.105hB-1 to IIId-1.105hB-255)

Table 106h
Compounds IIId-1 in which B is 2-chloro-5-methylphenyl and the combination of A and D corresponds in each case to one row of Table B (compounds IIId-1.106hB-1 to IIId-1.106hB-255)

Table 107h
Compounds IIId-1 in which B is 2-chloro-3-ethylphenyl and the combination of A and D corresponds in each case to one row of Table B (compounds IIId-1.107hB-1 to IIId-1.107hB-255)

Table 108h
Compounds IIId-1 in which B is 2-chloro-4-ethylphenyl and the combination of A and D corresponds in each case to one row of Table B (compounds IIId-1.108hB-1 to IIId-1.108hB-255)

Table 109h
Compounds IIId-1 in which B is 2-chloro-5-ethylphenyl and the combination of A and D corresponds in each case to one row of Table B (compounds IIId-1.109hB-1 to IIId-1.109hB-255)

Table 110h
Compounds IIId-1 in which B is 2-ethyl-3-chlorophenyl and the combination of A and D corresponds in each case to one row of Table B (compounds IIId-1.110hB-1 to IIId-1.110hB-255)

Table 111h
Compounds IIId-1 in which B is 2-ethyl-4-chlorophenyl and the combination of A and D corresponds in each case to one row of Table B (compounds IIId-1.111hB-1 to IIId-1.111hB-255)

Table 112h
Compounds IIId-1 in which B is 2-ethyl-5-chlorophenyl and the combination of A and D corresponds in each case to one row of Table B (compounds IIId-1.112hB-1 to IIId-1.112hB-255)

Table 113h
Compounds IIId-1 in which B is 2-ethyl-6-chlorophenyl and the combination of A and D corresponds in each case to one row of Table B (compounds IIId-1.113hB-1 to IIId-1.113hB-255)

Table 114h
Compounds IIId-1 in which B is 2-methoxy-3-chlorophenyl and the combination of A and D corresponds in each case to one row of Table B (compounds IIId-1.114hB-1 to IIId-1.114hB-255)

Table 115h
Compounds IIId-1 in which B is 2-methoxy-4-chlorophenyl and the combination of A and D corresponds in each case to one row of Table B (compounds IIId-1.115hB-1 to IIId-1.115hB-255)

Table 116h
Compounds IIId-1 in which B is 2-methoxy-5-chlorophenyl and the combination of A and D corresponds in each case to one row of Table B (compounds IIId-1.116hB-1 to IIId-1.116hB-255)

Table 117h
Compounds IIId-1 in which B is 2-methoxy-6-chlorophenyl and the combination of A and D corresponds in each case to one row of Table B (compounds IIId-1.117hB-1 to IIId-1.117hB-255)

Table 118h
Compounds IIId-1 in which B is 2-chloro-3-methoxyphenyl and the combination of A and D corresponds in each case to one row of Table B (compounds IIId-1.118hB-1 to IIId-1.118hB-255)

Table 119h
Compounds in which B is 2-chloro-4-methoxyphenyl and the combination of A and D corresponds in each case to one row of Table B (compounds IIId-1.119hB-1 to IIId-1.119hB-255)

Table 120h
Compounds IIId-1 in which B is 2-chloro-5-methoxyphenyl and the combination of A and D corresponds in each case to one row of Table B (compounds IIId-1.120hB-1 to IIId-1.120hB-255)

Table 121h
Compounds IIId-1 in which B is 2-(trifluoromethyl)-3-chlorophenyl and the combination of A and D corresponds in each case to one row of Table B (compounds IIId-1.121hB-1 to IIId-1.121hB-255)

Table 122h
Compounds IIId-1 in which B is 2-(trifluoromethyl)-4-chlorophenyl and the combination of A and D corresponds in each case to one row of Table B (compounds IIId-1.122hB-1 to IIId-1.122hB-255)

Table 123h
Compounds IIId-1 in which B is 2-(trifluoromethyl)-5-chlorophenyl and the combination of A and D corresponds in each case to one row of Table B (compounds IIId-1.123hB-1 to IIId-1.123hB-255)

Table 124h
Compounds IIId-1 in which B is 2-(trifluoromethyl)-6-chlorophenyl and the combination of A and D corresponds in each case to one row of Table B (compounds IIId-1.124hB-1 to IIId-1.124hB-255)

Table 125h
Compounds IIId-1 in which B is 2-chloro-3-(trifluoromethyl)phenyl and the combination of A and D corresponds in each case to one row of Table B (compounds IIId-1.125hB-1 to IIId-1.125hB-255)

Table 126h
Compounds IIId-1 in which B is 2-chloro-4-(trifluoromethyl)phenyl and the combination of A and D corresponds in each case to one row of Table B (compounds IIId-1.126hB-1 to IIId-1.126hB-255)

Table 127h
Compounds IIId-1 in which B is 2-chloro-5-(trifluoromethyl)phenyl and the combination of A and D corresponds in each case to one row of Table B (compounds IIId-1.127hB-1 to IIId-1.127hB-255)

Table 128h
Compounds IIId-1 in which B is 2-(trifluoromethoxy)-3-chlorophenyl and the combination of A and D corresponds in each case to one row of Table B (compounds IIId-1.128hB-1 to IIId-1.128hB-255)

Table 129h
Compounds IIId-1 in which B is 2-(trifluoromethoxy)-4-chlorophenyl and the combination of A and D corresponds in each case to one row of Table B (compounds IIId-1.129hB-1 to IIId-1.129hB-255)

Table 130h
Compounds IIId-1 in which B is 2-(trifluoromethoxy)-5-chlorophenyl and the combination of A and D corresponds in each case to one row of Table B (compounds IIId-1.130hB-1 to IIId-1.130hB-255)

Table 131h
Compounds IIId-1 in which B is 2-(trifluoromethoxy)-6-chlorophenyl and the combination of A and D corresponds in each case to one row of Table B (compounds IIId-1.131hB-1 to IIId-1.131hB-255)

Table 132h
Compounds IIId-1 in which B is 2-chloro-3-(trifluoromethoxy)phenyl and the combination of A and D corresponds in each case to one row of Table B (compounds IIId-1.132hB-1 to IIId-1.132hB-255)

Table 133h
Compounds IIId-1 in which B is 2-chloro-4-(trifluoromethoxy)phenyl and the combination of A and D corresponds in each case to one row of Table B (compounds IIId-1.133hB-1 to IIId-1.133hB-255)

Table 134h
Compounds IIId-1 in which B is 2-chloro-5-(trifluoromethoxy)phenyl and the combination of A and D corresponds in each case to one row of Table B (compounds IIId-1.134hB-1 to IIId-1.134hB-255)

Table 135h
Compounds IIId-1 in which B is 2-(difluoromethoxy)-3-chlorophenyl and the combination of A and D corresponds in each case to one row of Table B (compounds IIId-1.135hB-1 to IIId-1.135hB-255)

Table 136h
Compounds IIId-1 in which B is 2-(difluoromethoxy)-4-chlorophenyl and the combination of A and D corresponds in each case to one row of Table B (compounds IIId-1.136hB-1 to IIId-1.136hB-255)

Table 137h
Compounds IIId-1 in which B is 2-(difluoromethoxy)-5-chlorophenyl and the combination of A and D corresponds in each case to one row of Table B (compounds IIId-1.137hB-1 to IIId-1.137hB-255)

Table 138h
Compounds IIId-1 in which B is 2-(difluoromethoxy)-6-chlorophenyl and the combination of A and D corresponds in each case to one row of Table B (compounds IIId-1.138hB-1 to IIId-1.138hB-255)

Table 139h
Compounds IIId-1 in which B is 3-(difluoromethoxy)-4-chlorophenyl and the combination of A and D corresponds in each case to one row of Table B (compounds IIId-1.139hB-1 to IIId-1.139hB-255)

Table 140h
Compounds IIId-1 in which B is 2-chloro-3-(difluoromethoxy)phenyl and the combination of A and D corresponds in each case to one row of Table B (compounds IIId-1.140hB-1 to IIId-1.140hB-255)

Table 141h
Compounds IIId-1 in which B is 2-chloro-4-(difluoromethoxy)phenyl and the combination of A and D corresponds in each case to one row of Table B (compounds IIId-1.141hB-1 to IIId-1.141hB-255)

Table 142h
Compounds IIId-1 in which B is 2-chloro-5-(difluoromethoxy)phenyl and the combination of A and D corresponds in each case to one row of Table B (compounds IIId-1.142hB-1 to IIId-1.142hB-255)

Table 143h
Compounds IIId-1 in which B is 2-(trifluoromethylthio)-3-chlorophenyl and the combination of A and D corresponds in each case to one row of Table B (compounds IIId-1.143hB-1 to IIId-1.143hB-255)

Table 144h
Compounds IIId-1 in which B is 2-(trifluoromethylthio)-4-chlorophenyl and the combination of A and D corresponds in each case to one row of Table B (compounds IIId-1.144hB-1 to IIId-1.144hB-255)

Table 145h
Compounds IIId-1 in which B is 2-(trifluoromethylthio)-5-chlorophenyl and the combination of A and D corresponds in each case to one row of Table B (compounds IIId-1.145hB-1 to IIId-1.145hB-255)

Table 146h
Compounds IIId-1 in which B is 2-(trifluoromethylthio)-6-chlorophenyl and the combination of A and D corresponds in each case to one row of Table B (compounds IIId-1.146hB-1 to IIId-1.146hB-255)

Table 147h
Compounds IIId-1 in which B is 2-chloro-3-(trifluoromethylthio)phenyl and the combination of A and D corresponds in each case to one row of Table B (compounds IIId-1.147hB-1 to IIId-1.147hB-255)

Table 148h
Compounds IIId-1 in which B is 2-chloro-4-(trifluoromethylthio)phenyl and the combination of A and D corresponds in each case to one row of Table B (compounds IIId-1.148hB-1 to IIId-1.148hB-255)

Table 149h
Compounds IIId-1 in which B is 2-chloro-5-(trifluoromethylthio)phenyl and the combination of A and D corresponds in each case to one row of Table B (compounds IIId-1.149hB-1 to IIId-1.149hB-255)

Table 150h
Compounds IIId-1 in which B is 2,3,4-trichlorophenyl and the combination of A and D corresponds in each case to one row of Table B (compounds IIId-1.150hB-1 to IIId-1.150hB-255)

Table 151h
Compounds IIId-1 in which B is 2,3,5-trichlorophenyl and the combination of A and D corresponds in each case to one row of Table B (compounds IIId-1.151hB-1 to IIId-1.151hB-255)

Table 152h
Compounds IIId-1 in which B is 2,3,6-trichlorophenyl and the combination of A and D corresponds in each case to one row of Table B (compounds IIId-1.152hB-1 to IIId-1.152hB-255)

Table 153h
Compounds IIId-1 in which B is 2,4,5-trichlorophenyl and the combination of A and D corresponds in each case to one row of Table B (compounds IIId-1.153hB-1 to IIId-1.153hB-255)

Table 154h
Compounds IIId-1 in which B is 2,4,6-trichlorophenyl and the combination of A and D corresponds in each case to one row of Table B (compounds IIId-1.154hB-1 to IIId-1.154hB-255)

Table 155h
Compounds IIId-1 in which B is 2,3,4-trimethylphenyl and A corresponds in each case to one group of one row of Table B (compounds IIId-1.155hB-1 to IIId-1.155hB-255)

Table 156h
Compounds IIId-1 in which B is 2,3,5-trimethylphenyl and A corresponds in each case to one group of one row of Table B (compounds IIId-1.156hB-1 to IIId-1.156hB-255)

Table 157h
Compounds IIId-1 in which B is 2,3,6-trimethylphenyl and A corresponds in each case to one group of one row of Table B (compounds IIId-1.157hB-1 to IIId-1.157hB-255)

Table 158h
Compounds IIId-1 in which B is 2,4,5-trimethylphenyl and A corresponds in each case to one group of one row of Table B (compounds IIId-1.158hB-1 to IIId-1.158hB-255)

Table 159h
Compounds IIId-1 in which B is 2,4,6-trimethylphenyl and A corresponds in each case to one group of one row of Table B (compounds IIId-1.159hB-1 to IIId-1.159hB-255)

Table 160h
Compounds IIId-1 in which B is 2,3,4-trimethoxyphenyl and A corresponds in each case to one group of one row of Table B (compounds IIId-1.160hB-1 to IIId-1.160hB-255)

Table 161h
Compounds IIId-1 in which B is 2,3,5-trimethoxyphenyl and A corresponds in each case to one group of one row of Table B (compounds IIId-1.161hB-1 to IIId-1.161hB-255)

Table 162h
Compounds IIId-1 in which B is 2,3,6-trimethoxyphenyl and A corresponds in each case to one group of one row of Table B (compounds IIId-1.162hB-1 to IIId-1.162hB-255)

From the tables above, the compound names for the individual compounds can be derived as follows: the “compound I-3aA-10” (emphases added), for example, is the compound of the formula I according to the invention in which B is 2,5-difluorophenyl (as stated in Table 3a), A is 4-methylphenyl and D is SH (as stated in row 10 of Table A).

The compounds according to the invention, in particular the compounds of the formulae I and II, and their compositions according to the invention are suitable as fungicides for controlling harmful fungi. They are distinguished by excellent activity against a broad spectrum of phytopathogenic fungi including soilborne pathogens which originate in particular from the classes of the Plasmodiophoramycetes, Peronosporomycetes (syn. Oomycetes), Chytridiomycetes, Zygomycetes, Ascomycetes, Basidiomycetes and Deuteromycetes (syn. Fungi imperfecti). Some of them are systemically active and can be used in crop protection as foliar fungicides, fungicides for seed dressing and soil fungicides. In addition, they are suitable for controlling fungi which, inter alia, attack the wood or the roots of plants.

The compounds according to the invention are of particular importance for the control of a large number of pathogenic fungi on various crop plants such as cereals, for example wheat, rye, barley, triticale, oats or rice; beets, for example sugar beets or fodder beets; pomaceous fruits, stone fruits and soft fruits, for example apples, pears, plums, peaches, almonds, cherries, strawberries, raspberries, currants or gooseberries; leguminous plants, for example beans, lentils, peas, lucerne or soybeans; oil plants, for example oilseed rape, mustard, olives, sunflowers, coconut, cocoa, castor beans, oil palms, peanuts or soybeans; cucurbits, for example pumpkins, cucumbers or melons; fiber plants, for example cotton, flax, hemp or jute; citrus fruits, for example oranges, lemons, grapefruits or mandarins; vegetable plants, for example spinach, lettuce, asparagus, cabbage plants, carrots, onions, tomatoes, potatoes, pumpkins or bell peppers; laurel plants, for example avocados, cinnamon or camphor; energy and raw material plants, for example corn, soybeans, wheat, oilseed rape, sugar cane or oil palms; corn; tobacco; nuts; coffee; tea; bananas; grape vines (grapes for eating and grapes for wine making); hops; grass, for example lawns; rubber plants; ornamental and forest plants, for example flowers, shrubs, deciduous trees and coniferous trees, and also on the propagation material, for example seeds, and on the harvested material of these plants.

Preferably, the compounds I according to the invention and the compositions according to the invention are used for controlling a large number of fungal pathogens in agricultural crops, for example potatoes, sugar beets, tobacco, wheat, rye, barley, oats, rice, corn, cotton, soybeans, oilseed rape, leguminous plants, sunflowers, coffee or sugarcane; fruit plants, grapevines and ornamental plants and vegetables, for example cucumbers, tomatoes, beans and cucurbits and also on the propagation material, for example seeds, and the harvested products of these plants.

The term “plant propagation materials” includes all generative parts of the plant, for example seeds, and vegetative plant parts, such as seedlings and tubers (for example potatoes) which can be utilized for propagating a plant. These include seeds, roots, fruits, tubers, bulbs, rhizomes, shoots and other plant parts including seedlings and young plants which are transplanted after germination or after emergence. The young plants can be protected by partial or complete treatment, for example by immersion or watering, against harmful fungi.

The treatment of plant propagation materials with the compounds or the compositions according to the invention is used for controlling a large number of fungal pathogens in cereal crops, for example wheat, rye, barley or oats; rice, corn, cotton and soybeans.

The term crop plants also includes those plants which have been modified by breeding, mutagenesis or genetic engineering methods including the biotechnological agricultural products which are on the market or under development (see, for example, http://www.bio.org/speeches/pubs/er/agri_products.asp). Genetically modified plants are plants whose genetic material has been modified in a manner which does not occur under natural conditions by crossing, mutations or by natural recombination (that is a recombination of the genetic information). In general, one or more genes are integrated into the genetic material of the plant in order to improve the properties of the plant. Such modifications by genetic engineering include post-translational modifications of proteins, oligopeptides or polypeptides, for example by glycosylation or attachment of polymers such as, for example, prenylated, acetylated or farnesylated radicals or PEG radicals.

By way of example, mention may be made of plants which, by breeding and genetic engineering, are tolerant to certain classes of herbicides, such as hydroxyphenylpyruvate dioxygenase (HPPD) inhibitors, acetolactate synthase (ALS) inhibitors, such as, for example, sulfonylureas (EP-A 257 993, U.S. Pat. No. 5,013,659) or imidazolinones (for example U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073), enolpyruvylshikimate 3-phosphate synthase (EPSPS) inhibitors, such as, for example, glyphosate (see, for example, WO 92/00377), glutamine synthetase (GS) inhibitors, such as, for example, glufosinate (see, for example, EP-A 242 236, EP-A 242 246) or oxynil herbicides (see, for example, U.S. Pat. No. 5,559,024). Clearfield® oilseed rape (BASF SE, Germany), for example, which is tolerant to imidazolinones, for example imazamox, was generated by breeding and mutagenesis. With the aid of genetic engineering methods, crop plants such as soybeans, cotton, corn, beets and oilseed rape were generated which are resistant to glyphosate or glufosinate, and which are obtainable under the trade names RoundupReady® (glyphosate-resistant, Monsanto, U.S.A.) and Liberty Link® (glufosinate-resistant, Bayer CropScience, Germany).

Also included are plants which, owing to interventions by genetic engineering, produce one or more toxins, for example those of the bacterial strain Bacillus. Toxins which are produced by such genetically modified plants include, for example, insecticidal proteins of Bacillus spp., in particular B. thuringiensis, such as the endotoxins Cry1Ab, Cry1Ac, Cry1F, Cry1Fa2, Cry2Ab, Cry3A, Cry3Bb1, Cry9c, Cry34Ab1 or Cry35Ab1; or vegetative insecticidal proteins (VIPs), for example VIP1, VIP2, VIP3, or VIP3A; insecticidal proteins of nematode-colonizing bacteria, for example Photorhabdus spp. or Xenorhabdus spp.; toxins of animal organisms, for example wasp, spider or scorpion toxins; fungal toxins, for example from Streptomycetes; plant lectins, for example from peas or barley; agglutinins; protease inhibitors, for example trypsin inhibitors, serine protease inhibitors, patatin, cystatin or papain inhibitors; ribosome-inactivating proteins (RIPs), for example ricin, corn-RIP, abrin, luffin, saporin or bryodin; steroid-metabolizing enzymes, for example 3-hydroxysteroid oxidase, ecdysteroid-IDP glycosyl transferase, cholesterol oxidase, ecdyson inhibitors, or HMG-CoA reductase; ion channel blockers, for example inhibitors of sodium channels or calcium channels; juvenile hormone esterase; receptors of the diuretic hormone (helicokinin receptors); stilbene synthase, bibenzyl synthase, chitinases and glucanases. In the plants, these toxins may also be produced as pretoxins, hybrid proteins or truncated or otherwise modified proteins. Hybrid proteins are characterized by a novel combination of different protein domains (see, for example, WO 2002/015701). Further examples of such toxins or genetically modified plants which produce these toxins are disclosed in EP-A 374 753, WO 93/07278, WO 95/34656, EP-A 427 529, EP-A 451 878, WO 03/18810 and WO 03/52073. The methods for producing these genetically modified plants are known to the person skilled in the art and disclosed, for example, in the publications mentioned above. Many of the toxins mentioned above bestow, upon the plants by which they are produced, tolerance to pests from all taxonomic classes of arthropods, in particular to beetles (Coeleropta), dipterans (Diptera) and butterflies (Lepidoptera) and to nematodes (Nematoda). Genetically modified plants which produce one or more genes coding for insecticidal toxins are described, for example, in the publications mentioned above, and some of them are commercially available, such as, for example, YieldGard® (corn varieties producing the toxin Cry1Ab), YieldGard® Plus (corn varieties which produce the toxins Cry1Ab and Cry3Bb1), Starlink® (corn varieties which produce the toxin Cry9c), Herculex® RW (corn varieties which produce the toxins Cry34Ab1, Cry35Ab1 and the enzyme phosphinothricin-N-acetyltransferase [PAT]); NuCOTN® 33B (cotton varieties which produce the toxin Cry1Ac), Bollgard® I (cotton varieties which produce the toxin Cry1Ac), Bollgard® II (cotton varieties which produce the toxins Cry1Ac and Cry2Ab2); VIPCOT® (cotton varieties which produce a VIP toxin); NewLeaf® (potato varieties which produce the toxin Cry3A); Bt-Xtra®, NatureGard®, KnockOut®, BiteGard®, Protecta®, Bt11 (for example Agrisure® CB) and Bt176 from Syngenta Seeds SAS, France (corn varieties which produce the toxin Cry1Ab and the PAT enyzme), MIR604 from Syngenta Seeds SAS, France (corn varieties which produce a modified version of the toxin Cry3A, see WO 03/018810), MON 863 from Monsanto Europe S. A., Belgium (corn varieties which produce the toxin Cry3Bb1), IPC 531 from Monsanto Europe S. A., Belgium (cotton varieties which produce a modified version of the toxin Cry1Ac) and 1507 from Pioneer Overseas Corporation, Belgium (corn varieties which produce the toxin Cry1F and the PAT enzyme).

Also included are plants which, with the aid of genetic engineering, produce one or more proteins which are more robust or have increased resistance to bacterial, viral or fungal pathogens, such as, for example, pathogenesis-related proteins (PR proteins, see EP-A 0 392 225), resistance proteins (for example potato varieties producing two resistance genes against Phytophthora infestans from the wild Mexican potato Solanum bulbocastanum) or T4 lysozyme (for example potato varieties which, by producing this protein, are resistant to bacteria such as Erwinia amylvora).

Also included are plants whose productivity has been improved with the aid of genetic engineering methods, for example by enhancing the potential yield (for example biomass, grain yield, starch, oil or protein content), tolerance to drought, salt or other limiting environmental factors or resistance to pests and fungal, bacterial and viral pathogens.

Also included are plants whose ingredients have been modified with the aid of genetic engineering methods in particular for improving human or animal diet, for example oil plants producing health-promoting long-chain omega 3 fatty acids or monounsaturated omega 9 fatty acids (for example Nexera® oilseed rape, DOW Agro Sciences, Canada).

Also included are plants which have been modified with the aid of genetic engineering methods for improving the production of raw materials, for example by increasing the amylopectin content of potatoes (Amflora® potato, BASF SE, Germany).

Accordingly, the present invention also embraces the use of compounds according to the invention or their compositions for treating transgenic plants, in particular transgenic soybean plants or transgenic corn plants. Transgenic plants are plants as described above which have been changed using genetic engineering methods, in particular plants whose properties have been improved using genetic engineering methods. The invention embraces in particular the use of compounds according to the invention or their compositions for treating transgenic plants resistant to glyphosate, glufosinate or glufosinate-ammonium. In a further embodiment, the invention also embraces the use of compounds according to the invention or their compositions for treating herbicide-resistant plants. In a further embodiment, the invention also embraces the use of compounds according to the invention or their compositions for treating herbicide-sensitive plants.

Specifically, the compounds according to the invention and their compositions according to the invention are suitable for controlling the following plant diseases:

Albugo spp. (white rust) on ornamental plants, vegetable crops (for example A. candida) and sunflowers (for example A. tragopogonis); Alternaria spp. (black spot disease, black blotch) on vegetables, oilseed rape (for example A. brassicola or A. brassicae), sugar beet (for example A. tenuis), fruit, rice, soybeans and also on potatoes (for example A. solani or A. alternata) and tomatoes (for example A. solani or A. alternata) and Alternaria spp. (black head) on wheat; Aphanomyces spp. on sugar beet and vegetables; Ascochyta spp. on cereals and vegetables, for example A. tritici (Ascochyta leaf blight) on wheat and A. hordei on barley; Bipolaris and Drechslera spp. (teleomorph: Cochilobolus spp.) for example leaf spot diseases (D. maydis and B. zeicola) on corn, for example glume blotch (B. sorokinfana) on cereals and B. oryzae on rice and on lawn; Blumeria (old name: Elysiphe) graminis (powdery mildew) on cereals (for example wheat or barley); Botryosphaeria spp. (Black Dead Arm Disease') on grapevines (for example B. obtusa); Botrytis cinerea (teleomorph: Botryotinia fuckeliana: gray mold, gray rot) on soft fruit and pome fruit (inter alia strawberries), vegetables (inter alia lettuce, carrots, celeriac and cabbage), oilseed rape, flowers, grapevines, forest crops and wheat (ear mold); Bremia lactucae (downy mildew) on lettuce; Ceratocystis (syn. Ophiostoma) spp. (blue stain fungus) on deciduous trees and coniferous trees, for example C. ulmi (Dutch elm disease) on elms; Cercospora spp. (Cercospora leaf spot) on corn (for example C. zeae-maydis), rice, sugar beet (for example C. beticola), sugar cane, vegetables, coffee, soybeans (for example C. sojina or C. kikuchli) and rice; Cladosporium spp. on tomato (for example C. fulvum: tomato leaf mold) and cereals, for example C. herbarum (ear rot) on wheat; Claviceps purpurea (ergot) on cereals; Cochliobolus (anamorph: Helminthosporium or Bipolaris) spp. (leaf spot) on corn (for example C. carbonum), cereals (for example C. sativus, anamorph: B. sorokiniana: glume blotch) and rice (for example C. miyabeanus, anamorph: H. oryzae); Colletotrichum (teleomorph: Glomerella) spp. (anthracnosis) on cotton (for example C. gossypii), corn (for example C. graminicola: stem rot and anthracnosis), soft fruit, potatoes (for example C. coccodes: wilt disease), beans (for example C. lindemuthianum) and soybeans (for example C. truncatum); Corticium spp., for example C. sasakii (sheath blight) on rice; Corynespora casskola (leaf spot) on soybeans and ornamental plants; Cycloconium spp., for example C. oleaginum on olive; Cylindrocarpon spp. (for example fruit tree cancer or black foot disease of grapevine, teleomorph: Nectria or Neonectria spp.) on fruit trees, grapevines (for example C. kriodendri, teleomorph: Neonectria liriodendri, black foot disease) and many ornamental trees; Dematophora (teleomorph: Rosellinia) necatrix (root/stem rot) on soybeans; Diaporthe spp. for example D. phaseolorum (stem disease) on soybeans; Drechslera (syn. Helminthosporium, teleomorph: Pyrenophora) spp. on corn, cereals, such as barley (for example D. teres, net blotch) and on wheat (for example D. triticirepentis: DTR leaf spot), rice and lawn; esca disease (dieback of grapevine, apoplexia) on grapevines, caused by Formitiporia (syn. Phellinus) punctata, F. mediterranea, Phaeomoniella chlamydospora (old name Phaeoacremonium chlamydosporum), Phaeoacremonium aleophllum and/or Botryosphaeria obtusa; Elsinoe spp. on pome fruit (E. pyri) and soft fruit (E. veneta: anthracnosis) and also grapevines (E. ampelina: anthracnosis); Entyloma oryzae (leaf smut) on rice; Epicoccum spp. (black head) on wheat; Erysiphe spp. (powdery mildew) on sugar beet (E. betae), vegetables (for example E. pisi), such as cucumber species (for example E. cichoracearum) and cabbage species, such as oilseed rape (for example E. cruciferarum); Eutypa lata (Eutypa cancer or dieback, anamorph: Cytosporina lata, syn. Libertella blepharis) on fruit trees, grapevines and many ornamental trees; Exserohilum (syn. Helminthosporium) spp. on corn (for example E. turcicum); Fusarium (teleomorph: Gibberella) spp. (wilt disease, root and stem rot) on various plants, such as for example F. graminearum or F. culmorum (root rot and silver-top) on cereals (for example wheat or barley), F. oxysporum on tomatoes, F solani on soybeans and F. verticillioides on corn; Gaeumannomyces graminis (take-all) on cereals (for example wheat or barley) and corn; Gibberella spp. on cereals (for example G. zeae) and rice (for example G. fujikuroi: bakanae disease); Glomerella cingulata on grapevines, pome fruit and other plants and G. gossypii on cotton; Grainstaining complex on rice; Guignardia (black rot) on grapevines; Gymnosporangium spp. on Rosaceae and juniper, for example G. sabinae (pear rust) on pears; Helminthosporium spp. (syn. Drechslera, teleomorph: Cochliobolus) on corn, cereals and rice; Hemlleia spp., for example H. vastatrix (coffee leaf rust) on coffee; Isariopsis clavispora (syn. Cladosporium vitis) on grapevines; Macrophomina phaseolina (syn. phaseoli) (root/stem rot) on soybeans and cotton; Microdochium (syn. Fusarium) nivale (pink snow mold) on cereals (for example wheat or barley); Microsphaera diffuse (powdery mildew) on soybeans; Morginia spp., for example M. laxa, M. fructicola and M. fructigena (blossom and twig blight) on stone fruit and other Rosaceae; Mycosphaerella spp. on cereals, bananas, soft fruit and peanuts, such as for example M. graminicola (anamorph: Septoria tritici; Septoria leaf blotch) on wheat or M. fijiensis (sigatoka disease) on bananas; Peronospora spp. (downy mildew) on cabbage (for example P. brassicae), oilseed rape (for example P. parasitica), bulbous plants (for example P. destructor), tobacco (P. tabacina) and soybeans (for example P. manshurica); Phakopsora pachyrhizi and P. meibomiae (soybean rust) on soybeans; Phialophora spp. for example on grapevines (for example P. tracheiphila and P. tetraspora) and soybeans (for example P. gregata: stem disease); Phoma lingam (root and stem rot) on oilseed rape and cabbage and P. betae (leaf spot) on sugar beet; Phomopsis spp. on sunflowers, grapevines (for example P. viticola: dead-arm disease) and soybeans (for example stem canker/stem blight: P. phaseoli, teleomorph: Diaporthe phaseolorum); Physoderma maydis (brown spot) on corn; Phytophthora spp. (wilt disease, root, leaf, stem and fruit rot) on various plants, such as on bell peppers and cucumber species (for example P. capsici), soybeans (for example P. megasperma, syn. P. sojae), potatoes and tomatoes (for example P. infestans: late blight and brown rot) and deciduous trees (for example P. ramorum: sudden oak death); Plasmodiophora brassicae (club-root) on cabbage, oilseed rape, radish and other plants; Plasmopara spp., for example P. viticola (peronospora of grapevines, downy mildew) on grapevines and P. halstedii on sunflowers; Podosphaera spp. (powdery mildew) on Rosaceae, hops, pome fruit and soft fruit, for example P. leucotricha on apple; Polymyxa spp., for example on cereals, such as barley and wheat (P. graminis) and sugar beet (P. betae) and the viral diseases transmitted thereby; Pseudocercosporella herpotrichoides (eyespot/stem break, teleomorph: Tapesia yallundae) on cereals, for example wheat or barley; Pseudoperonospora (downy mildew) on various plants, for example P. cubensis on cucumber species or P. humili on hops; Pseudopezicula tracheiphila (angular leaf scorch, anamorph: Phialophora) on grapevines; Puccinia spp. (rust disease) on various plants, for example P. triticina (also brown rust of wheat), P. striiformis (yellow rust), P. hordei (dwarf leaf rust of barley), P. graminis (black rust) or P. recondita (brown rust of rye) on cereals, such as for example wheat, barley or rye, and on asparagus (for example P. asparagi); Pyrenophora (anamorph: Drechslera) tritici-repentis (speckled leaf blotch) on wheat and P. teres (net blotch) on barley; Pyricularia spp., for example P. oryzae (teleomorph: Magnaporthe grisea, rice blast) on rice and P. grisea on lawn and cereals; Pythium spp. (damping-off disease) on lawn, rice, corn, wheat, cotton, oilseed rape, sunflowers, sugar beet, vegetables and other plants (for example P. ultimum or P. aphanidermatum); Ramularia spp., for example R. collo-cygni (Ramularia leaf and awn spot/physiological leaf spot) on barley and R. beticola on sugar beet; Rhizoctonia spp. on cotton, rice, potatoes, lawn, corn, oilseed rape, potatoes, sugar beet, vegetables and on various other plants, for example R. solani (root and stem rot) on soybeans, R. solani (sheath blight) on rice or R. cerealis (sharp eyespot) on wheat or barley; Rhizopus stolonifer (soft rot) on strawberries, carrots, cabbage, grapevines and tomato; Rhynchosporium secalis (leaf spot) on barley, rye and triticale; Sarocladium oryzae and S. attenuatum (sheath rot) on rice; Sclerotinia spp. (stem or white rot) on vegetable and field crops, such as oilseed rape, sunflowers (for example Sclerotinia sclerotiorum) and soybeans (for example S. rolfsii); Septoria spp. on various plants, for example S. glycines (leaf spot) on soybeans, S. tritici(Septoria leaf blotch) on wheat and S. (syn. Stagonospora) nodorum (leaf blotch and glume blotch) on cereals; Uncinula (syn. Erysiphe) necator(powdery mildew, anamorph: Oidium tucker) on grapevines; Setosphaeria spp. (leaf spot) on corn (for example S. turcicum, syn. Helminthosporium turcicum) and lawn; Sphacelotheca spp. (head smut) on corn, (for example S. relliana: kernel smut), millet and sugar cane; Sphaerotheca fuliginea (powdery mildew) on cucumber species; Spongospora subterranea (powdery scab) on potatoes and the viral diseases transmitted thereby; Stagonospora spp. on cereals, for example S. nodorum (leaf blotch and glume blotch, teleomorph: Leptosphaeria [syn. Phaeosphaeria] nodorum) on wheat; Synchytrium endobioticum on potatoes (potato wart disease); Taphrina spp., for example T. deformans (curly-leaf disease) on peach and T. pruni (plum-pocket disease) on plums; Thielaviopsis spp. (black root rot) on tobacco, pome fruit, vegetable crops, soybeans and cotton, for example T. basicola (syn. Chalara elegans); Tilletia spp. (bunt or stinking smut) on cereals, such as for example T. tritici (syn. T. caries, wheat bunt) and T. controversa (dwarf bunt) on wheat; Typhula incarnata (gray snow mold) on barley or wheat; Urocystis spp., for example U. occulta (flag smut) on rye; Uromyces spp. (rust) on vegetable plants, such as beans (for example U. appendiculatus, syn. U. phaseoli) and sugar beet (for example U. betae); Ustilago spp. (loose smut) on cereals (for example U. nuda and U. avaenae), corn (for example U. maydis: corn smut) and sugar cane; Venturia spp. (scab) on apples (for example V. inaequalis) and pears and Verticillium spp. (leaf and shoot wilt) on various plants, such as fruit trees and ornamental trees, grapevines, soft fruit, vegetable and field crops, such as for example V. dahliae on strawberries, oilseed rape, potatoes and tomatoes.

Moreover, the compounds according to the invention and their compositions according to the invention are suitable for controlling harmful fungi in the protection of materials and buildings (for example wood, paper, paint dispersions, fibers or tissues) and in the protection of stored products. In the protection of wood and buildings, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Porta spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of materials to the following yeast fungi: Candida spp. and Saccharomyces cerevisae.

The compounds according to the invention and their compositions according to the invention are suitable for improving plant health. Moreover, the invention relates to a method for improving plant health by treating the plants, the plant propagation material and/or the site at which the plants grow or are intended to grow with an effective amount of the compounds according to the invention or their compositions according to the invention.

The term “plant health” comprises states of a plant and/or its harvested material which are determined by various indicators individually or in combination, such as, for example, yield (for example increased biomass and/or increased content of utilizable ingredients), plant vitality (for example increased plant growth and/or greener leaves (“greening effect”)), quality (for example increased content or composition of certain ingredients) and tolerance to biotic and/or abiotic stress. The indicators mentioned here for a state of plant health may occur independently of one another or may influence each other.

Accordingly, the invention also provides the use of compounds according to the invention and/or their agriculturally acceptable salts for controlling phytopathogenic fungi.

The invention furthermore provides a method for controlling phytopathogenic fungi which comprises treating the fungi or the materials, plants, the soil or seeds to be protected against fungal attack with an effective amount of a compound according to the invention and/or an agriculturally acceptable salt thereof.

The compounds according to the invention are employed as such or in the form of a composition by treating the harmful fungi, their habitat or the plants or plant propagation materials, for example seed materials to be protected against fungal attack, the soil, areas, materials or spaces with a fungicidally effective amount of the compounds according to the invention. The application can be carried out both before and after the infection of the plants, plant propagation materials, for example seed materials, the soil, the areas, materials or spaces by the fungi.

Plant propagation materials can be treated prophylactically during or even before sowing or during or even before transplanting with compounds according to the invention or their compositions according to the invention.

Moreover, the invention relates to preparations and agrochemical compositions comprising a solvent or solid carrier and at least one compound according to the invention, and to the use of these compositions for controlling harmful fungi. The present invention also provides a preparation or an agrochemical composition comprising at least one compound according to the invention and/or an agriculturally acceptable salt thereof for use in crop protection. Such a preparation usually comprises at least one liquid or solid carrier.

Accordingly, the invention also embraces preparations and agrochemical compositions comprising a solid or liquid carrier and a fungicidal compound according to the invention. In the present context, the term “liquid carrier” is synonymous with solvent.

An agrochemical composition comprises a fungicidally effective amount of a compound according to the invention. The term “effective amount” refers to an amount of the agrochemical composition or of the compound according to the invention which is sufficient for controlling harmful fungi on crop plants or in the protection of materials and buildings and does not cause any significant damage to the treated crop plants. Such an amount may vary within a wide range and is influenced by numerous factors, such as, for example, the harmful fungus to be controlled, the respective crop plant or materials treated, the climatic conditions and compounds.

The compounds according to the invention, their N-oxides and their salts can be converted into the types customary for agrochemical compositions, for example solutions, emulsions, suspensions, dusts, powders, pastes and granules. The type of composition depends on the respective intended purpose; in each case, it should ensure a fine and even distribution of the compound according to the invention.

In the present context, the term “preparation” is synonymous with the term “composition”, in particular “agrochemical composition”, and “formulation”.

Here, examples of types of compositions are suspensions (SC, OD, FS), pastes, pastilles, wettable powders or dusts (WP, SP, SS, WS, DP, DS) or granules (GR, FG, GG, MG) which may either be water-soluble or dispersible (wettable), and also gels for treating plant propagation materials such as seed (GF).

In general, the composition types (for example SC, OD, FS, WG, SG, WP, SP, SS, WS, GF) are used in diluted form. Composition types such as DP, DS, GR, FG, GG and MG are generally employed in undiluted form.

The agrochemical compositions are prepared in a known manner (see, for example, U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th edition, McGraw-Hill, New York, 1963, 8-57 and ff., WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (John Wiley & Sons, New York, 1961), Hance et al.: Weed Control Handbook (8th Ed., Blackwell Scientific Publications, Oxford, 1989) and Mollet, H. and Grubemann, A.: Formulation Technology (Wiley VCH Verlag, Weinheim, 2001).

The agrochemical compositions may furthermore also comprise auxiliaries customary for crop protection compositions, the selection of the auxiliaries depending on the use form or the active compound in question.

Examples of suitable auxiliaries are solvents, solid carriers, surfactants (such as further solubilizers, protective colloids, wetting agents and tackifiers), organic and inorganic thickeners, bactericides, antifreeze agents, antifoams, if appropriate colorants and adhesives (for example for the treatment of seed).

Suitable solvents are water, organic solvents, such as mineral oil fractions having a medium to high boiling point, such as kerosene and diesel oil, furthermore coal tar oils, and also oils of vegetable or animal origin, aliphatic, cyclic and aromatic hydrocarbons, for example paraffins, tetrahydronaphthalene, alkylated naphthalenes and derivatives thereof, alkylated benzenes and derivatives thereof, alcohols, such as methanol, ethanol, propanol, butanol and cyclohexanol, glycols, ketones, such as cyclohexanone, gamma-butyrolactone, dimethyl fatty amides, fatty acids and fatty acid esters and strongly polar solvents, for example amines, such as N-methylpyrrolidone. In principle, it is also possible to use solvent mixtures, and also mixtures of the solvents mentioned above and water.

Solid carriers are mineral earths, such as silicic acids, silica gels, silicates, talc, kaolin, limestone, lime, chalk, bole, loess, clay, dolomite, diatomaceous earth, calcium sulfate and magnesium sulfate, magnesium oxide, ground synthetic substances, fertilizers, such as ammonium sulfate, ammonium phosphate, ammonium nitrate, ureas and vegetable products, such as cereal meal, tree bark meal, sawdust and nutshell meal, cellulose powder or other solid carriers.

Suitable surfactants (adjuvants, wetting agents, tackifiers, dispersants or emulsifiers) are the alkali metal, alkaline earth metal and ammonium salts of aromatic sulfonic acids, for example of lignosulfonic acid (Borresperse® types, Borregaard, Norway), phenolsulfonic acid, naphthalenesulfonic acid (Morwet® types, Akzo Nobel, USA) and dibutylnaphthalenesulfonic acid (Nekal® types, BASF, Germany), and also of fatty acids, alkyl- and alkylarylsulfonates, alkyl, lauryl ether and fatty alcohol sulfates, and also salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octyl phenol ether, ethoxylated isooctylphenol, octylphenol or nonylphenol, alkylphenyl polyglycol ether, tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfide waste liquors, and also proteins, denatured proteins, polysaccharides (for example methylcellulose), hydrophobically modified starches, polyvinyl alcohol (Mowiol® types, Clariant, Switzerland), polycarboxylates (Sokalan® types, BASF, Germany), polyalkoxylates, polyvinylamine (Lupamin® types, BASF, Germany), polyethylenimine (Lupasol® types, BASF, Germany), polyvinylpyrrolidone and copolymers thereof.

Examples of thickeners (i.e. compounds which impart modified flow properties to the composition, i.e. high viscosity in the state of rest and low viscosity in motion) are polysaccharides and also organic and inorganic sheet minerals, such as xanthan gum (Kelzan®, CP Kelco, USA), Rhodopol® 23 (Rhodia, France) or Veegum® (R.T. Vanderbilt, USA) or Attaclay® (Engelhard Corp., NJ, USA).

Bactericides can be added for stabilizing the composition. Examples of bactericides are bactericides based on dichlorophen and benzyl alcohol hemiformal (Proxel® from ICI or Acticide® RS from Thor Chemie and Kathon® MK from Rohm & Haas), and also isothiazolinone derivatives, such as alkylisothiazolinones and benzisothiazolinones (Acticide® MBS from Thor Chemie).

Examples of suitable antifreeze agents are ethylene glycol, propylene glycol, urea and glycerol.

Examples of antifoams are silicone emulsions (such as, for example, Silikon® SRE, Wacker, Germany or Rhodorsil®, Rhodia, France), long-chain alcohols, fatty acids, salts of fatty acids, organofluorine compounds and mixtures thereof.

Examples of colorants are both sparingly water-soluble pigments and water-soluble dyes. Examples which may be mentioned are the dyes and pigments known under the names Rhodamin B, C. I. Pigment Red 112 and C. I. Solvent Red 1, Pigment blue 15:4, Pigment blue 15:3, Pigment blue 15:2, Pigment blue 15:1, Pigment blue 80, Pigment yellow 1, Pigment yellow 13, Pigment red 48:2, Pigment red 48:1, Pigment red 57:1, Pigment red 53:1, Pigment orange 43, Pigment orange 34, Pigment orange 5, Pigment green 36, Pigment green 7, Pigment white 6, Pigment brown 25, Basic violet 10, Basic violet 49, Acid red 51, Acid red 52, Acid red 14, Acid blue 9, Acid yellow 23, Basic red 10, Basic red 108.

Examples of adhesives are polyvinylpyrrolidone, polyvinyl acetate, polyvinyl alcohol and cellulose ether (Tylose®, Shin-Etsu, Japan).

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydro-naphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the compounds I and, if present further active compounds with at least one solid carrier.

Granules, for example coated granules, impregnated granules and homogeneous granules, can be prepared by binding the active compounds to at least one solid carrier. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

The following are examples of types of composition:

1. Types of Composition for Dilution with Water

i) Water-Soluble Concentrates (SL, LS)

10 parts by weight of the active compounds are dissolved with 90 parts by weight of water or with a water-soluble solvent. As an alternative, wetters or other auxiliaries are added. The active compound dissolves upon dilution with water. This gives a composition having an active compound content of 10% by weight.

ii) Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight.

iii) Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The composition has an active compound content of 15% by weight.

iv) Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The composition has an active compound content of 25% by weight.

v) Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the composition is 20% by weight.

vi) Water-Dispersible Granules and Water-Soluble Granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The composition has an active compound content of 50% by weight.

vii) Water-Dispersible Powders and Water-Soluble Powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the composition is 75% by weight.

viii) Gels (GF)

20 parts by weight of the active compounds, 10 parts by weight of dispersant, 1 part by weight of gelling agent and 70 parts by weight of water or an organic solvent are ground in a ball mill to give a fine suspension. Dilution with water gives a stable suspension with an active compound content of 20% by weight.

### 2. Types of Composition to be Applied Undiluted

ix) Dusts (DP, DS)

5 parts by weight of the active compounds are ground finely and mixed intimately with 95 parts by weight of finely divided kaolin. This gives a dustable product with an active compound content of 5% by weight.

x) Granules (GR, FG, GG, MG)

0.5 part by weight of the active compounds is ground finely and associated with 99.5 parts by weight of carriers. Current methods are extrusion, spray-drying or the fluidized bed. This gives granules with an active compound content of 0.5% by weight to be applied undiluted.

xi) ULV Solutions (UL)

10 parts by weight of the active compounds are dissolved in 90 parts by weight of an organic solvent, for example xylene. This gives a composition with an active compound content of 10% by weight to be applied undiluted.

In general, the compositions of the compounds according to the invention comprise from 0.1 to 95% by weight, preferably from 0.1 to 90% by weight, preferably from 0.5 to 90% by weight, of active compound (compound according to the invention). The compounds I and II are employed in a purity of from 90% to 100%, preferably 95% to 100% (according to the NMR spectrum).

Water-soluble concentrates (LS), suspensions (FS), dusts (DS), water-dispersible and water-soluble powders (WS, SS), emulsions (ES), emulsifiable concentrates (EC) and gels (GF) are usually used for the treatment of plant propagation materials, in particular seed. These compositions can be applied to the propagation materials, in particular seed, in undiluted or, preferably, diluted form. In this case, the corresponding composition can be diluted 2 to 10 times so that in the compositions used for the seed dressing from 0.01 to 60% by weight, preferably from 0.1 to 40% by weight of active compound are present. The application can be carried out before or during sowing. The treatment of plant propagation material, in particular the treatment of seed, is known to the person skilled in the art and is carried out by dusting, coating, pelleting, dipping or drenching the plant propagation material, the treatment preferably being carried out by pelleting, coating and dusting or by furrow treatment, such that, for example, premature germination of the seed is prevented.

For seed treatment, preference is given to using suspensions. Such compositions usually comprise from 1 to 800g of active compound/I, from 1 to 200g of surfactants/I, from 0 to 200g of antifreeze agent/I, from 0 to 400g of binders/I, from 0 to 200g of colorants/I and solvents, preferably water.

The compounds can be used as such or in the form of their compositions, for example in the form of directly sprayable solutions, powders, suspensions, dispersions, emulsions, oil dispersions, pastes, dustable products, materials for spreading or granules, by means of spraying, atomizing, dusting, spreading, raking in, immersing or pouring. The types of composition depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply compositions comprising over 95% by weight of active compound, or even to apply the active compound without additives.

When used in crop protection, the application rates are from 0.001 to 2.0 kg of active compound per ha, preferably from 0.005 to 2 kg per ha, more preferably from 0.01 to 2.0 kg per ha, particularly preferably from 0.05 to 0.9 kg per ha, especially from 0.1 to 0.75 kg per ha, depending on the nature of the desired effect.

In the treatment of plant propagation materials, for example seed, the amounts of active compound used are generally from 0.1 to 1000g/100 kg of propagation material or seed, preferably from 1 to 1000g/100 kg, particularly preferably from 1 to 100g/100 kg, especially from 5 to 100g/100 kg. Accordingly, the invention furthermore provides seed comprising at least one compound according to the invention and/or an agriculturally acceptable salt thereof in an amount of from 1 to 1000g per 100 kg.

When used in the protection of materials or stored products, the active compound application rate depends on the kind of application area and on the desired effect. Amounts typically applied in the protection of materials are, for example, from 0.001 g to 2 kg, preferably from 0.005 g to 1 kg, of active compound per cubic meter of treated material.

Various types of oils, wetters, adjuvants, herbicides, bactericides, other fungicides and/or pesticides may be added to the compounds according to the invention (active compounds) or the compositions comprising them, if appropriate not until immediately prior to use (tank mix). These compositions can be admixed with the compositions according to the invention in a weight ratio of from 1:100 to 100:1, preferably from 1:10 to 10:1.

The following are particularly suitable as adjuvants in this context: organically modified polysiloxanes, for example Break Thru S 240®; alcohol alkoxylates, for example Atplus 245®, Atplus MBA 1303®, Plurafac LF 300® and Lutensol ON 30®; EO-PO block polymers, for example Pluronic RPE 2035® and Genapol B®; alcohol ethoxylates, for example Lutensol XP 80®; and sodium dioctylsulfosuccinate, for example Leophen RA®.

The compounds according to the invention and their compositions in the application form as fungicides can also be present together with other active compounds, for example with herbicides, insecticides, growth regulators, fungicides or else with fertilizers, as premix or if appropriate also only immediately prior to use (tank mix). When mixing the compounds I or the compositions comprising them with one or more further active compounds, in particular fungicides, it is in many cases possible, for example, to widen the activity spectrum or to prevent the development of resistance. In many cases, synergistic effects are obtained.

Accordingly, the invention also provides a composition for crop protection which comprises a compound according to the invention, in particular a compound I or a compound II, and at least one further fungicidally, insecticidally and/or herbicidally active compound. According to one embodiment, the further active compound is a fungicidally active compound, in particular one selected from the list below. Here, the active compounds are preferably present in synergistic amounts.

A further subject matter of the invention relates to a composition comprising a compound according to the invention and/or an acid addition salt or metal salt thereof. As crop protection composition, this composition furthermore comprises at least one solid or liquid carrier. According to a further embodiment, the compositions mentioned may furthermore comprise at least one further fungicidally, insecticidally and/or herbicidally active compound.

The fungicides are preferably selected from the following groups:

Strobilurins, carboxamides, such as carboxanilides, carboxylic acid morpholides, benzamides, other carboxamides, azoles, such as triazoles, imidazoles, benzimidazoles, others, nitrogenous heterocyclyl compounds, such as pyridines, pyrimidines, pyrroles, morpholines, dicarboximides, other nitrogenous heterocyclyl compounds, thio- and dithiocarbamates, carbamates, guanidines, antibiotics, nitrophenyl derivatives, organometal compounds, sulfur-containing heterocyclyl compounds, organophosphorus compounds, organochlorine compounds, inorganic active compounds, other fungicides.

The following list of active compounds with which the compounds according to the invention can be applied together is meant to illustrate the possible combinations, but not to limit them:

A) strobilurins
azoxystrobin, dimoxystrobin, enestroburin, fluoxastrobin, kresoxim-methyl, methominostrobin, orysastrobin, picoxystrobin, pyraclostrobin, pyribencarb, trifloxystrobin, 2-(2-(6-(3-chloro-2-methylphenoxy)-5-fluoropyrimidin-4-yloxy)phenyl)-2-methoxyimino-N-methylacetamide, methyl 2-(ortho-((2,5-dimethylphenyloxymethylene)phenyl)-3-methoxyacrylate, methyl 3-methoxy-2-(2-(N-(4-methoxyphenyl)cyclopropanecarboximidoylsulfanylmethyl)phenyl)acrylate, 2-(2-(3-(2,6-dichlorophenyl)-1-methylallylideneaminooxymethyl)phenyl-2-methoxyimino-N-methyl acetamide;
B) carboxamides
carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penthiopyrad, sedaxane, tecloftalam, thifluzamide, tiadinil, 2-amino-4-methylthiazole-5-carboxanilide, 2-chloro-N-(1,1,3-trimethylindan-4-yl)-nicotinamide, N-(3′,4′-dichloro-5-fluorobiphenyl-2-yl)-3-difluoromethyl-1-methyl-1H-pyrazole-4-carboxamide, [2-(1,3-dimethylbutyl)phenyl]-5-fluoro-1,3-dimethyl-1H-pyrazole-4-carboxamide, N-(4′-chloro-3′,5-difluorobiphenyl-2-yl)-3-difluoromethyl-1-methyl-1H-pyrazole-4-carboxamide, N-(4′-chloro-3′,5-difluorobiphenyl-2-yl)-3-trifluoromethyl-1-methyl-1H-pyrazole-4-carboxamide, N-(3′,4′-dichloro-4-fluorobiphenyl-2-yl)-3-trifluoromethyl-1-methyl-1H-pyrazole-4-carboxamide, N-(3′,5-difluoro-4′-methylbiphenyl-2-yl)-3-difluoromethyl-1-methyl-1H-pyrazole-4-carboxamide, N-(3′, 5′-difluoro-4′-methylbiphenyl-2-yl)-3-trifluoromethyl-1-methyl-1H-pyrazole-4-carboxamide, N-(2-bicyclopropyl-2-ylphenyl)-3-difluoromethyl-1-methyl-1H-pyrazole-4-carboxamide, N-(cis-2-bicyclopropyl-2-ylphenyl)-3-difluoromethyl-1-methyl-1H-pyrazole-4-carboxamide, N-(trans-2-bicyclopropyl-2-ylphenyl)-3-difluoromethyl-1-methyl-1H-pyrazole-4-carboxamide, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, 3,4-dichloro-N-(2-cyanophenyl)isothiazole-5-carboxamide, N-(2′,4′-difluorobiphenyl-2-yl)-1-methyl-3-trifluoromethyl-1H-pyrazole-4-carboxamide, N-(2′,4′-dichlorobiphenyl-2-yl)-1-methyl-3-trifluoromethyl-1H-pyrazole-4-carboxamide, N-(2′,4′-difluorobiphenyl-2-yl)-3-difluoromethyl-1-methyl-1H-pyrazole-4-carboxamide, N-(2′,4′-dichlorobiphenyl-2-yl)-3-difluoromethyl-1-methyl-1H-pyrazole-4-carboxamide, N-(2′,5′-difluorobiphenyl-2-yl)-1-methyl-3-trifluoronnethyl-1H-pyrazole-4-carboxamide, N-(2′,5′-dichlorobiphenyl-2-yl)-1-methyl-3-trifluoromethyl-1H-pyrazole-4-carboxamide, N-(2′,5′-difluorobiphenyl-2-yl)-3-difluoromethyl-1-methyl-1H-pyrazole-4-carboxamide, N-(2′,5′-dichlorobiphenyl-2-yl)-3-difluoromethyl-1-methyl-1H-pyrazole-4-carboxamide, N-(3′,5′-difluorobiphenyl-2-yl)-1-methyl-3-trifluoromethyl-1H-pyrazole-4-carboxamide, N-(3′,5′-dichlorobiphenyl-2-yl)-1-methyl-3-trifluoromethyl-1H-pyrazole-4-carboxamide, N-(3′,5′-difluorobiphenyl-2-yl)-3-difluoromethyl-1-methyl-1H-pyrazole-4-carboxamide, N-(3′,5′-dichlorobiphenyl-2-yl)-3-difluoromethyl-1-methyl-1H-pyrazole-4-carboxamide, N-(3′-fluorobiphenyl-2-yl)-1-methyl-3-trifluoromethyl-1H-pyrazole-4-carboxamide, N-(3′-chlorobiphenyl-2-yl)-1-methyl-3-trifluoromethyl-1H-pyrazole-4-carboxamide, N-(3′-fluorobiphenyl-2-yl)-3-difluoromethyl-1-methyl-1H-pyrazole-4-carboxamide, N-(3′-chlorobiphenyl-2-yl)-3-difluoromethyl-1-methyl-1H-pyrazole-4-carboxamide, N-(2′-fluorobiphenyl-2-yl)-1-methyl-3-trifluoromethyl-1H-pyrazole-4-carboxamide, N-(2′-chlorobiphenyl-2-yl)-1-methyl-3-trifluoromethyl-1H-pyrazole-4-carboxamide, N-(2′-fluorobiphenyl-2-yl)-3-difluoromethyl-1-methyl-1H-pyrazole-4-carboxamide, N-(2′-chlorobiphenyl-2-yl)-3-difluoromethyl-1-methyl-1H-pyrazole-4-carboxamide, N-(2′-fluoro-4′-chloro-5′-methylbiphenyl-2-yl)-1-methyl-3-trifluoromethyl-1H-pyrazole-4-carboxamide, N-(3′,4′,5′-trifluorobiphenyl-2-yl)-1-methyl-3-trifluoromethyl-1H-pyrazole-4-carboxamide, N-(3′,4′,5′-trifluorobiphenyl-2-yl)-1-methyl-3-difluoromethyl-1H-pyrazole-4-carboxamide, N-(2′,4′,5′-tri-fluorobiphenyl-2-yl)-1-methyl-3-difluoromethyl-1H-pyrazole-4-carboxamide, N-(3′,4′,5′-trifluorobiphenyl-2-yl)-3-chlorofluoromethyl-1-methyl-1H-pyrazole-4-carboxamide, N-[2-(1,1,2,3,3,3-hexafluoropropoxy)phenyl]-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[2-(2-chloro-1,1,2-trifluoroethoxy)phenyl]-1-methyl-3-trifluoromethyl-1H-pyrazole-4-carboxamide, N-[2-(2-chloro-1,1,2-trifluoroethoxy)phenyl]-3-difluoromethyl-1-methyl-1H-pyrazole-4-carboxamide, N-[2-(1,1,2,2-tetrafluoroethoxy)phenyl]-3-difluoromethyl-1-methyl-1H-pyrazole-4-carboxamide, N-[2-(1,1,2,2-tetrafluoroethoxy)phenyl]-1-methyl-3-trifluoromethyl-1H-pyrazole-4-carboxamide, N-(4′-(trifluoromethylthio)biphenyl-2-yl)-3-difluoromethyl-1-methyl-1H-pyrazole-4-carboxamide, N-(4′-(trifluoromethylthio)biphenyl-2-yl)-1-methyl-3-trifluoromethyl-1H-pyrazole-4-carboxamide, [2-(1,2-dimethylpropyl)phenyl]-5-fluoro-1,3-dimethyl-1H-pyrazole-4-carboxamide, N-(2-{4-[3-(4-chlorophenyl)prop-2-ynyloxy]-3-methoxyphenyl}ethyl)-2-methanesulfonylamino-3-methylbutyramide, N-(2-{4-[3-(4-chlorophenyl)prop-2-ynyloxy]-3-methoxyphenyl}ethyl)-2-ethanesulfonylamino-3-methylbutyramide; N-(3′,4′-dichloro-5-fluorobiphenyl-2-yl)-3-trifluoromethyl-1-methyl-1H-pyrazol-4-carboxamide, N-(2-(1,3,3-trimethylbutyl)phenyl)-1,3-dimethyl-5-fluoro-1H-pyrazol-4-carboxamide, N-[1,2,3,4-tetrahydro-9-(1-methylethyl)-1,4-methanonaphthalene-5-yl]-3-(difluoromethyl)-1-methyl-1H-pyrazol-4-carboxamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanylpropoxy)phenyl)-N-ethyl-N-methylformamidine and N′-(5-difluoromethyl-2-methyl-4-(3-trimethylsilanylpropoxy)phenyl)-N-ethyl-N-methylformamidine;
carboxylic acid morpholides: dimethomorph, flumorph, pyrimorph;
benzamides: flumetover, fluopicolide, fluopyram, zoxamide, N-(3-ethyl-3,5,5-trimethylcyclohexyl)-3-formylamino-2-hydroxybenzamide;
other carboxamides: carpropamid, diclocymet, mandipropamid, oxytetracyclin, silthiofam, N-(6-methoxypyridin-3-yl)cyclopropanecarboxamide;
   C) azoles
triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-(4-chlorophenyl)-2-([1,2,4]triazol-1-yl)-cycloheptanol;
imidazoles: cyazofamid, imazalil, imazalil-sulfate, pefurazoate, prochloraz, triflumizole;
benzimidazoles: benomyl, carbendazim, fuberidazole, thiabendazole;
others: ethaboxam, etridiazole, hymexazole, 1-(4-chlorophenyl)-1-(propyn-2-yloxy)-3-(4-(3,4-dimethoxyphenyl)isoxazol-5-yl)propan-2-one, 2-(4-chlorophenyl)-N-[4-(3,4-dimethoxyphenypisoxazol-5-yl]-2-prop-2-inyloxy acetamide;

### D) Nitrogenous Heterocyclyl Compounds

pyridines: fluazinam, pyrifenox, 3-[5-(4-chlorophenyl)-2,3-dimethylisoxazolidin-3-yl]-pyridine, 3-[5-(4-methylphenyl)-2,3-dimethylisoxazolidin-3-yl]-pyridine, 2,3,5,6-tetrachloro-4-methanesulfonylpyridine, 3,4,5-trichloropyridine-2,6-dicarbonitrile, N-(1-(5-bromo-3-chloropyridin-2-yl)ethyl)-2,4-dichloronicotinannide, N-((5-bromo-3-chloropyridin-2-yl)methyl)-2,4-dichloronicotinamide;
pyrimidines: bupirimate, cyprodinil, diflumetorim, fenarimol, ferimzone, mepanipyrim, nitrapyrin, nuarimol, pyrimethanil;
piperazines: triforine;
pyrroles: fludioxonil, fenpiclonil;
morpholines: aldimorph, dodemorph, dodemorph-acetate, fenpropimorph,
tridemorph;
piperidines: fenpropidine;
dicarboximides: fluoroimide, iprodione, procymidone, vinclozolin;
non-aromatic 5-membered heterocycles: famoxadone, fenamidone, flutianil, octhilinon, probenazol, S-allyl 5-amino-2-isopropyl-3-oxo-4-orthotolyl-2,3-dihydropyrazol-1-thiocarboxylate;
others: acibenzolar-5-methyl, amisulbrom, anilazine, blasticidin-S, captafol, captan, quinomethionate, dazomet, debacarb, diclomezine, difenzoquat, difenzoquat-methyl sulfate, fenoxanil, folpet, oxolinic acid, piperalin, proquinazid, pyroquilon, quinoxyfen, triazoxide, tricyclazole, 5-chloro-7-(4-methylpiperidin-1-yl)-6-(2,4,6-trifluorophenyl)--[1,2,4]triazolo[1,5-a]pyrimidine, 6-(4-tert-butylphenyl)-5-methyl-[1,2,4]triazolo[1,5-a]pyrimidin-7-ylamine, 5-methyl-6-(3,5,5-trinnethylhexyl)-[1,2,4]triazolo[1,5-a]pyrimidin-7-ylamine, 5-methyl-6-octyl-[1,2,4]triazolo[1,5-a]pyrimidin-7-ylamine, 6-methyl-5-octyl-[1,2,4]triazolo[1,5-a]pyrimidin-7-ylamine, 6-ethyl-5-octyl-[1,2,4]triazolo[1,5-a]pyrimidin-7-ylamine, 5-ethyl-6-octyl-[1,2,4]triazolo[1,5-a]pyrimidin-7-ylamine, 5-ethyl-6-(3,5,5-trimethylhexyl)-[1,2,4]triazolo[1,5-a]pyrimidin-7-ylamine, 6-octyl-5-propyl-[1,2,4]triazolo[1,5-a]pyrimidin-7-ylamine, 5-methoxymethyl-6-octyl-[1,2,4]triazolo[1,5-a]pyrimidin-7-ylamine, 6-octyl-5-trifluoromethyl[1,2,4]triazolo[1,5-a]pyrimidin-7-ylamine, 5-trifluoromethyl-6-(3,5,5-trimethylhexyl)-[1,2,4]triazolo[1,5-a]pyrimidin-7-ylamine, 2-butoxy-6-iodo-3-propylchromen-4-one, 5-chloro-1-(4,6-dimethoxypyrimidin-2-yl)-2-methyl-1H-benzoimidazole, 6-(3,4-dichlorophenyl)-5-methyl-[1,2,4]triazolo[1,5-a]pyrimidin-7-ylamine;

### E) Carbamates and Dithiocarbamates

thio- and dithiocarbamates: ferbam, mancozeb, maneb, metam, methasulfocarb, metiram, propineb, thiram, zineb, ziram;
carbamates: diethofencarb, benthiavalicarb, iprovalicarb, propamocarb, propamocarb hydrochloride, valiphenal, (4-fluorophenyl) N-(1-(1-(4-cyanophenyl)-ethanesulfonyl)but-2-yl)carbamate;
   F) other Fungicides
guanidines: dodine, dodine-free base, guazatine, guazatine-acetate, iminoctadine, iminoctadine-triacetate, iminoctadine-tris(albesilate);
antibiotics: kasugamycin, kasugamycin-hydrochloride hydrate, polyoxins, streptomycin, validamycin A;
nitrophenyl derivatives:
binapacryl, dicloran, dinobuton, dinocap, nitrothal-isopropyl, tecnazen;
organometallic compounds: fentin salts such as, for example, fentin-acetate, fentin-chloride, fentin-hydroxide;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl;
organochlorine compounds: chlorothalonil, dichlofluanid, dichlorophen, flusulfamide, hexachlorobenzene, pencycuron, pentachlorophenol and salts thereof, phthalide, quintozene, thiophanate-methyl, tolylfluanid, N-(4-chloro-2-nitrophenyl)-N-ethyl-4-methylbenzenesulfonamide;
inorganic active compounds: phosphorous acid and salts thereof, sulfur, Bordeaux mixture, copper salts such as, for example, copper acetate, copper hydroxide, copper oxychloride, basic copper sulfate;
others: biphenyl, bronopol, cyflufenamid, cymoxanil, diphenylamine, metrafenone, mildiomycin, oxine-copper, prohexadione-calcium, spiroxamine, tolylfluanid, N-(cyclopropylmethoxyimino-(6-difluoromethoxy-2,3-difluorophenyl)methyl)-2-phenyl acetamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanyl-propoxy)phenyl)-N-ethyl-N-methylformamidine, N′-(5-difluoromethyl-2-methyl-4-(3-trimethylsilanylpropoxy)phenyl)-N-ethyl-N-methylformamidine, methyl N-(1,2,3,4-tetrahydronaphthalen-1-yl)-2-{1-[2-(5-methyl-3-trifluoromethylpyrazol-1-yl)acetyl]piperidin-4-yl}thiazole-4-carboxamide, methyl (R)—N-(1,2,3,4-tetrahydronaphthalen-1-yl)-2-{1-[2-(5-methyl-3-trifluoromethylpyrazol-1-yl)acetyl]-piperidin-4-yl}thiazole-4-carboxamide, 6-tert-butyl-8-fluoro-2,3-dimethylquinolin-4-yl acetate, 6-tert-butyl-8-fluoro-2,3-dimethylquinolin-4-ylmethoxyacetate;

### G) Growth Regulators

abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfid, indole-3-acetic acid, maleic hydrazide, mefluidide, mepiquat (mepiquat chloride), metconazole, naphthalene acetic acid, N-6-benzyladenine, paclobutrazole, prohexadione (prohexadione-calcium), prohydrojasmon, thidiazuron, triapenthenol, tributyl phosphorotrithioate, 2,3,5-triiodobenzoic acid, trinexapac-ethyl and uniconazole;
H) herbicides
acetamides: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor;
amino acid analogues: bilanafos, glyphosate, glufosinate, sulfosate;
aryloxyphenoxypropionates: clodinafop, cyhalofop-butyl, fenoxaprop, fluazifop, haloxyfop, metamifop, propaquizafop, quizalofop, quizalofop-p-tefuryl;
bipyridyls: diquat, paraquat;
carbamates and thiocarbamates: asulam, butylate, carbetamide, desmedipham, dimepiperate, eptam (EPTC), esprocarb, molinate, orbencarb, phenmedipham, prosulfocarb, pyributicarb, thiobencarb, triallate;
cyclohexanediones: butroxydim, clethodim, cycloxydim, profoxydim, sethoxydim, tepraloxydim, tralkoxydim;
dinitroanilines: benfluralin, ethalfluralin, oryzalin, pendimethalin, prodiamine,
trifluralin;
diphenyl ethers: acifluorfen, aclonifen, bifenox, diclofop, ethoxyfen, fomesafen, lactofen, oxyfluorfen;
hydroxybenzonitriles: bromoxynil, dichlobenil, ioxynil;
imidazolinones: imazamethabenz, imazamox, imazapic, imazapyr, imazaquin,
imazethapyr;
phenoxyacetic acids: clomeprop, 2,4-dichlorophenoxyacetic acid (2,4-D), 2,4-DB, dichlorprop, MCPA, MCPA-thioethyl, MCPB, mecoprop;
pyrazines: chloridazone, flufenpyr-ethyl, fluthiacet, norflurazone, pyridate;
pyridines: aminopyralid, clopyralid, diflufenican, dithiopyr, fluridone, fluoroxypyr, picloram, picolinafen, thiazopyr;
sulfonylureas: amidosulfuron, azimsulfuron, bensulfuron, chlorimuron-ethyl, chlorsulfuron, cinosulfuron, cyclosulfamuron, ethoxysulfuron, flazasulfuron, fluce-tosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosul-furon, pyrazosulfuron, rimsulfuron, sulfometuron, sulfosulfuron, thifensulfuron, tria-sulfuron, tribenuron, trifloxysulfuron, triflusulfuron, tritosulfuron, 14(2-chloro-6-propyl-imidazo[1,2-b]pyridazin-3-yl)sulfonyl)-3-(4,6-dimethoxypyrimidin-2-yl)urea;
triazines: ametryn, atrazine, cyanazine, dimethametryn, ethiozine, hexazinone, meta-mitron, metribuzin, prometryn, simazine, terbuthylazine, terbutryn, triaziflam;
ureas: chlorotoluron, daimuron, diuron, fluometuron, isoproturon, linuron, methabenzthiazuron, tebuthiuron;
other inhibitors of acetolactate synthase: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam;
others: amicarbazone, aminotriazole, anilofos, beflubutamid, benazolin, bencarbazone, benfluresate, benzolenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, fluorochloridone, flurtamone, indanofan, isoxaben, isoxaflutole, lenacil, propanil, propyzamide, quinclorac, quinmerac, mesotrione, methylarsenic acid, naptalam, oxadiargyl, oxadiazon, oxaziclomefone, pentoxazone, pinoxaden, pyraclonil, pyraflufen-ethyl, pyrasulfotol, pyrazoxyfen, pyrazolynate, quinoclamine, saflufenacil, sulcotrion, sulfentrazone, terbacil, tefuryltrione, tembotrione, thiencarbazone, topramezone, 4-hydroxy-3-[2-(2-methoxyethoxymethyl)-6-trifluoromethylpyridine-3-carbonyl]bicyclo[3.2.1]oct-3-ene-2-one, ethyl (3-[2-chloro-4-fluoro-5-(3-methyl-2,6-dioxo-4-trifluoromethyl-3,6-dihydro-2H-pyrimidin-1-yl)phenoxy]pyridin-2-yloxy)acetate, methyl 6-amino-5-chloro-2-cyclopropylpyrimidine-4-carboxylate, 6-chloro-3-(2-cyclopropyl-6-methylphenoxy)-pyridazin-4-ol, 4-amino-3-chloro-6-(4-chlorophenyl)-5-fluoropyridine-2-carboxylic acid, methyl 4-amino-3-chloro-6-(4-chloro-2-fluoro-3-methoxyphenyl)pyridine-2-carboxylate and methyl 4-amino-3-chloro-6-(4-chloro-3-dimethylamino-2-fluorophenyl)pyridine-2-carboxylate;

### I) Insecticides

organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, bifenthrin, cyfluthrin, cyhalothrin, cyphenothrin, cypermethrin, alpha-cypermethrin, beta-cypermethrin, zeta-cypermethrin, deltannethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin,
dimefluthrin,
inhibitors of insect growth: a) chitin synthesis inhibitors: benzoylureas: chlorfluazuron, cyromazine, diflubenzuron, flucycloxuron, flufenoxuron, hexaflumuron, lufenuron, novaluron, teflubenzuron, triflumuron; buprofezin, diofenolan, hexythiazox, etoxazole, clofentazin; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramate;
nicotine receptor agonists/antagonists: clothianidin, dinotefuran, imidacloprid, thiamethoxam, nitenpyram, acetamiprid, thiacloprid, 1-(2-chlorothiazol-5-ylmethyl)-2-nitrinnino-3,5-dimethyl-[1,3,5]triazinane;
GABA antagonists: endosulfan, ethiprol, fipronil, vaniliprol, pyrafluprol, pyriprol, 5-amino-1-(2,6-dichloro-4-methylphenyl)-4-sulfinamoyl-1H-pyrazole-3-thiocarboxamide;
macrocyclic lactones: abamectin, emamectin, milbemectin, lepimectin, spinosad, spinetoram;
mitochondrial electron transport chain inhibitors (METI) I acaricides: fenazaquin, pyridaben, tebufenpyrad, tolfenpyrad, flufenerim;
METI II and III substances: acequinocyl, fluacyprim, hydramethylnon;
decouplers: chlorfenapyr;
inhibitors of oxidative phosphorylation: cyhexatin, diafenthiuron, fenbutatin oxide,
propargite;
insect molting inhibitors: cryomazine;
mixed function oxidase inhibitors: piperonyl butoxide;
sodium channel blockers: indoxacarb, metaflumizone;
others: benclothiaz, bifenazate, cartap, flonicamid, pyridalyl, pymetrozine, sulfur, thiocyclam, flubendiamid, chlorantraniliprol, cyazypyr (HGW86); cyenopyrafen, flupyrazofos, cyflumetofen, amidoflumet, imicyafos, bistrifluoron, and pyrifluquinazon.

The present invention relates in particular also to fungicidal compositions which comprise at least one compound of the general formula I and at least one further crop protection agent, selected, for example, from the active compounds of groups A) to I) mentioned above, in particular at least one fungicidally active compound, in particular selected from the active compounds of groups A) to F) mentioned above, and, if appropriate, one or more agriculturally suitable carriers. With a view to reducing the application rates, these mixtures are of interest, since many show, at a reduced total amount of active compounds applied, an improved activity against harmful fungi, in particular for certain indications. By simultaneous joint or separate application of compound(s) I with at least one active compound of groups A) to I), the fungicidal activity can be increased in a superadditive manner.

In the sense of the present application, joint application means that the at least one compound I and the at least one further active compound are present simultaneously at the site of action (i.e. the plant-damaging fungi to be controlled and their habitat, such as infected plants, plant propagation materials, in particular seed, soils, materials or spaces and also plants, plant propagation materials, in particular seed, soils, materials or spaces to be protected against fungal attack) in an amount sufficient for an effective control of fungal growth. This can be achieved by applying the compounds I and at least one further active compound jointly in a joint active compound preparation or in at least two separate active compound preparations simultaneously, or by applying the active compounds successively to the site of action, the interval between the individual active compound applications being chosen such that the active compound applied first is, at the time of application of the further active compound(s), present at the site of action in a sufficient amount. The order in which the active compounds are applied is of minor importance.

In binary mixtures, i.e. compositions according to the invention comprising a compound I and a further active compound, for example an active compound of groups A) to I), the weight ratio of compound I to further active compound depends on the properties of the active compounds in question; usually, it is in the range of from 1:100 to 100:1, frequently in the range of from 1:50 to 50:1, preferably in the range of from 1:20 to 20:1, particularly preferably in the range of from 1:10 to 10:1, especially in the range of from 1:3 to 3:1.

The components of the composition according to the invention can be packaged and used individually or as a ready-mix or as a kit of parts.

In one embodiment of the invention, the kits may comprise one or more, and even all, components used for preparing an agrochemical composition according to the invention. For example, these kits may comprise one or more fungicide components and/or an adjuvant component and/or an insecticide component and/or a growth regulator component and/or a herbicide. One or more components may be present combined or preformulated with one another. In the embodiments where more than two components are provided in a kit, the components can be combined with one another and be packaged in a single container, such as a vessel, a bottle, a tin, a bag, a sack or canister. In other embodiments, two or more components of a kit may be packaged separately, i.e. not preformulated or mixed. Kits may comprise one or more separate containers, such as vessels, bottles, tins, bags, sacks or canisters, each container comprising a separate component of the agrochemical composition. The components of the composition according to the invention may be packaged and used individually or as a ready-mix or as a kit of parts. In both forms, a component may be used separately or together with the other components or as a part of a kit of parts according to the invention for preparing the mixture according to the invention.

The user uses the composition according to the invention usually for use in a predosage device, a knapsack sprayer, a spray tank or a spray plane. Here, the agrochemical composition is diluted with water and/or buffer to the desired application concentration, with further auxiliaries being added, if appropriate, thus giving the ready-to-use spray liquor or the agrochemical composition according to the invention. Usually, from 50 to 500 liters of the ready-to-use spray liquor are applied per hectare of agricultural utilized area, preferably from 100 to 400 liters.

According to one embodiment, the user may himself mix individual components, such as, for example, parts of a kit or a mixture of the composition according to the invention in a spray tank and, if appropriate, add further auxiliaries (tank mix).

In a further embodiment, the user may mix both individual components of the composition according to the invention and partially pre-mixed components, for example components comprising compounds I and/or active compounds from groups A) to I), in a spray tank and, if appropriate, add further auxiliaries (tank mix).

In a further embodiment, the user may use both individual components of the composition according to the invention and partially pre-mixed components, for example components comprising compounds I and/or active compounds from groups A) to I), jointly (for example as a tank mix) or in succession.

Preference is given to compositions of a compound I (component 1) with at least one active compound from group A) (component 2) of the strobilurins and in particular selected from the group consisting of azoxystrobin, dimoxystrobin, fluoxastrobin, kresoxim-methyl, orysastrobin, picoxystrobin, pyraclostrobin and trifloxystrobin. Preference is also given to compositions of a compound I (component I) with at least one active compound selected from group B) (component 2) of the carboxamides and in particular selected from the group consisting of bixafen, boscalid, sedaxane, fenhexamid, metalaxyl, mefenoxam, ofurace, dimethomorph, flumorph, fluopicolid (picobenzamid), zoxamide, carpropamid and mandipropamid.

Preference is also given to compositions of a compound I (component I) with at least one active compound selected from group C) (component 2) of the azoles and in particular selected from the group consisting of cyproconazole, difenoconazole, epoxiconazole, fluquinconazole, flusilazole, flutriafole, metconazole, myclobutanil, penconazole, propiconazole, prothioconazole, triadimefon, triadimenol, tebuconazole, tetraconazole, triticonazole, prochloraz, cyazofamid, benomyl, carbendazim and ethaboxam.

Preference is also given to compositions of a compound I (component I) with at least one active compound selected from group D) (component 2) of the nitrogenous heterocyclyl compounds and in particular selected from the group consisting of fluazinam, cyprodinil, fenarimol, mepanipyrim, pyrimethanil, triforin, fludioxonil, fodemorph, fenpropimorph, tridemorph, fenpropidin, iprodion, vinclozolin, famoxadone, fenamidone, probenazole, proquinazid, acibenzolar-s-methyl, captafol, folpet, fenoxanil and quinoxyfen.

Preference is also given to compositions of a compound I (component I) with at least one active compound selected from group E) (component 2) of the carbamates and in particular selected from the group consisting of mancozeb, metiram, propineb, thiram, iprovalicarb, benthiavalicarb and propamocarb.

Preference is also given to compositions of a compound I (component I) with at least one active compound selected from the fungicides of group F) (component 2) and in particular selected from the group consisting of dithianon, fentin salts, such as fentin acetate, fosetyl, fosetyl-aluminum, H₃PO₃ and salts thereof, chlorothalonil, dichlofluanid, thiophanate-methyl, copper acetate, copper hydroxide, copper oxychloride, copper sulfate, sulfur, cymoxanil, metrafenone, spiroxamine and 5-chloro-7-(4-methylpiperidin-1-yl)-6-(2,4,6-trifluorophenyl)-[1,2,4]triazolo[1,5-a]pyrimidine.

Accordingly, the present invention furthermore relates to compositions of a compound I (component 1) with a further active compound (component 2), the latter being selected from rows D-1 to D-416 in the column “component 2” of Table D.

A further embodiment of the invention relates to the compositions D-1 to D-416 listed in Table D, each line of Table D corresponding to an agrochemical composition comprising a compound of the formula I (component 1), which is preferably one of the compounds described herein as being preferred, and comprising the further active compound from groups A to I indicated in each case in the line in question (component 2). According to one embodiment of the invention, component 1 in each line of Table D is in each case one of the compounds of the formula I that are specifically individualized in Tables 1a to 162a. The active compounds in the compositions described are in each case preferably present in synergistically effective amounts.

TABLE D
Active compound composition, comprising an individualized compound I and
a further active compound from groups A) to I).

| Row | Component 1 | Component 2 |
|---|---|---|
| D-1 | a compound of the formula I | azoxystrobin |
| D-2 | a compound of the formula I | dimoxystrobin |
| D-3 | a compound of the formula I | enestroburin |
| D-4 | a compound of the formula I | fluoxastrobin |
| D-5 | a compound of the formula I | kresoxim-methyl |
| D-6 | a compound of the formula I | metominostrobin |
| D-7 | a compound of the formula I | orysastrobin |
| D-8 | a compound of the formula I | picoxystrobin |
| D-9 | a compound of the formula I | pyraclostrobin |
| D-10 | a compound of the formula I | pyribencarb |
| D-11 | a compound of the formula I | trifloxystrobin |
| D-12 | a compound of the formula I | 2-(2-(6-(3-chloro-2-methylphenoxy)- |
| | | 5-fluoropyrimidin-4-yloxy)phenyl)- |
| | | 2-methoxyimino-N-methylacetamide |
| D-13 | a compound of the formula I | 2-(ortho-((2,5-dimethylphenyloxy- |
| | | methylene)phenyl)-3-methoxyacrylic acid methyl |
| | | ester |
| D-14 | a compound of the formula I | 3-methoxy-2-(2-(N-(4-methoxyphenyl)- |
| | | cyclopropanecarboximidoylsulfanyl- |
| | | methyl)phenyl)acrylic acid methyl ester |
| D-15 | a compound of the formula I | benalaxyl |
| D-16 | a compound of the formula I | benalaxyl-M |
| D-17 | a compound of the formula I | benodanil |
| D-18 | a compound of the formula I | bixafen |
| D-19 | a compound of the formula I | boscalid |
| D-20 | a compound of the formula I | carboxin |
| D-21 | a compound of the formula I | fenfuram |
| D-22 | a compound of the formula I | fenhexamid |
| D-23 | a compound of the formula I | flutolanil |
| D-24 | a compound of the formula I | furametpyr |
| D-25 | a compound of the formula I | isotianil |
| D-26 | a compound of the formula I | kiralaxyl |
| D-27 | a compound of the formula I | mepronil |
| D-28 | a compound of the formula I | metalaxyl |
| D-29 | a compound of the formula I | ofurace |
| D-30 | a compound of the formula I | oxadixyl |
| D-31 | a compound of the formula I | oxycarboxin |
| D-32 | a compound of the formula I | penthiopyrad |
| D-33 | a compound of the formula I | sedaxane |
| D-34 | a compound of the formula I | thifluzamide |
| D-35 | a compound of the formula I | tecloftalam |
| D-36 | a compound of the formula I | tiadinil |
| D-37 | a compound of the formula I | 2-amino-4-methylthiazole-5-carboxanilide |
| D-38 | a compound of the formula I | 2-chloro-N-(1,1,3-trimethylindan-4-yl)- |
| | | nicotinamide |
| D-39 | a compound of the formula I | N-(3′,4′-dichloro-5-fluorobiphenyl-2-yl)- |
| | | 3-difluoromethyl-1-methyl-1H-pyrazole- |
| | | 4-carboxamide |
| D-40 | a compound of the formula I | 5-fluoro-1,3-dimethyl-1H-pyrazole- |
| | | 4-carboxylic acid [2-(1,3-dimethylbutyl)- |
| | | phenyl]amide |
| D-41 | a compound of the formula I | N-(4′-chloro-3′,5-difluorobiphenyl-2-yl)- |
| | | 3-difluoromethyl-1-methyl-1H-pyrazole- |
| | | 4-carboxamide |
| D-42 | a compound of the formula I | N-(4′-chloro-3′,5-difluorobiphenyl-2-yl)- |
| | | 3-trifluoromethyl-1-methyl-1H-pyrazole- |
| | | 4-carboxamide |
| D-43 | a compound of the formula I | N-(3′,4′-dichloro-4-fluorobiphenyl-2-yl)- |
| | | 3-trifluoromethyl-1-methyl-1H-pyrazole- |
| | | 4-carboxamide |
| D-44 | a compound of the formula I | N-(3′,5-difluoro-4′-methylbiphenyl-2-yl)- |
| | | 3-difluoromethyl-1-methyl-1H-pyrazole- |
| | | 4-carboxamide |
| D-45 | a compound of the formula I | N-(3′,5-difluoro-4′-methylbiphenyl-2-yl)- |
| | | 3-trifluoromethyl-1-methyl-1H-pyrazole- |
| | | 4-carboxamide |
| D-46 | a compound of the formula I | N-(2-bicyclopropyl-2-ylphenyl)-3-difluoro- |
| | | methyl-1-methyl-1H-pyrazole- |
| | | 4-carboxamide |
| D-47 | a compound of the formula I | N-(cis-2-bicyclopropyl-2-ylphenyl)- |
| | | 3-difluoromethyl-1-methyl-1H-pyrazole- |
| | | 4-carboxamide |
| D-48 | a compound of the formula I | N-(trans-2-bicyclopropyl-2-ylphenyl)-3- |
| | | difluoromethyl-1-methyl-1H-pyrazole- |
| | | 4-carboxamide |
| D-49 | a compound of the formula I | N-(4′-bromobiphenyl-2-yl)-4- |
| | | difluoromethyl-2-methylthiazole-5- |
| | | carboxamide |
| D-50 | a compound of the formula I | N-(4′-trifluoromethylbiphenyl-2-yl)-4- |
| | | difluoromethyl-2-methylthiazole-5- |
| | | carboxamide |
| D-51 | a compound of the formula I | N-(4′-chloro-3′-fluorobiphenyl-2-yl)-4- |
| | | difluoromethyl-2-methylthiazole-5- |
| | | carboxamide |
| D-52 | a compound of the formula I | 3,4-dichloro-N-(2-cyanophenyl)isothiazole- |
| | | 5-carboxamide |
| D-53 | a compound of the formula I | N-(2′,4′-difluorobiphenyl-2-yl)-1-methyl-3- |
| | | trifluoromethyl-1H-pyrazole-4-carboxamide |
| D-54 | a compound of the formula I | N-(2′,4′-dichlorobiphenyl-2-yl)-1-methyl-3- |
| | | trifluoromethyl-1H-pyrazole-4-carboxamide |
| D-55 | a compound of the formula I | N-(2′,4′-difluorobiphenyl-2-yl)-3- |
| | | difluoromethyl-1-methyl-1H-pyrazole-4- |
| | | carboxamide |
| D-56 | a compound of the formula I | N-(2′,4′-dichlorobiphenyl-2-yl)-3- |
| | | difluoromethyl-1-methyl-1H-pyrazole-4- |
| | | carboxamide |
| D-57 | a compound of the formula I | N-(2′,5′-difluorobiphenyl-2-yl)-1-methyl-3- |
| | | trifluoromethyl-1H-pyrazole-4-carboxamide |
| D-58 | a compound of the formula I | N-(2′,5′-dichlorobiphenyl-2-yl)-1-methyl-3- |
| | | trifluoromethyl-1H-pyrazole-4-carboxamide |
| D-59 | a compound of the formula I | N-(2′,5′-difluorobiphenyl-2-yl)-3- |
| | | difluoromethyl-1-methyl-1H-pyrazole-4- |
| | | carboxamide |
| D-60 | a compound of the formula I | N-(2′,5′-dichlorobiphenyl-2-yl)-3- |
| | | difluoromethyl-1-methyl-1H-pyrazole-4- |
| | | carboxamide |
| D-61 | a compound of the formula I | N-(3′,5′-difluorobiphenyl-2-yl)-1-methyl-3- |
| | | trifluoromethyl-1H-pyrazole-4-carboxamide |
| D-62 | a compound of the formula I | N-(3′,5′-dichlorobiphenyl-2-yl)-1-methyl-3- |
| | | trifluoromethyl-1H-pyrazole-4-carboxamide |
| D-63 | a compound of the formula I | N-(3′,5′-difluorobiphenyl-2-yl)-3- |
| | | difluoromethyl-1-methyl-1H-pyrazole-4- |
| | | carboxamide |
| D-64 | a compound of the formula I | N-(3′,5′-dichlorobiphenyl-2-yl)-3- |
| | | difluoromethyl-1-methyl-1H-pyrazole-4- |
| | | carboxamide |
| D-65 | a compound of the formula I | N-(3′-fluorobiphenyl-2-yl)-1-methyl-3- |
| | | trifluoromethyl-1H-pyrazole-4-carboxamide |
| D-66 | a compound of the formula I | N-(3′-chlorobiphenyl-2-yl)-1-methyl-3- |
| | | trifluoromethyl-1H-pyrazole-4-carboxamide |
| D-67 | a compound of the formula I | N-(3′-fluorobiphenyl-2-yl)-3-difluoromethyl- |
| | | 1-methyl-1H-pyrazole-4-carboxamide |
| D-68 | a compound of the formula I | N-(3′-chlorobiphenyl-2-yl)-3-difluoromethyl- |
| | | 1-methyl-1H-pyrazole-4-carboxamide |
| D-69 | a compound of the formula I | N-(2′-fluorobiphenyl-2-yl)-1-methyl-3- |
| | | trifluoromethyl-1H-pyrazole-4-carboxamide |
| D-70 | a compound of the formula I | N-(2′-chlorobiphenyl-2-yl)-1-methyl-3- |
| | | trifluoromethyl-1H-pyrazole-4-carboxamide |
| D-71 | a compound of the formula I | N-(2′-fluorobiphenyl-2-yl)-3-difluoromethyl- |
| | | 1-methyl-1H-pyrazole-4-carboxamide |
| D-72 | a compound of the formula I | N-(2′-chlorobiphenyl-2-yl)-3-difluoromethyl- |
| | | 1-methyl-1H-pyrazole-4-carboxamide |
| D-73 | a compound of the formula I | N-(2′-fluoro-4′-chloro-5′-methylbiphenyl-2- |
| | | yl)-1-methyl-3-trifluoromethyl-1H-pyrazole- |
| | | 4-carboxamide |
| D-74 | a compound of the formula I | N-(3′,4′,5′-trifluorobiphenyl-2-yl)-1-methyl- |
| | | 3-trifluoromethyl-1H-pyrazole-4- |
| | | carboxamide |
| D-75 | a compound of the formula I | N-(3′,4′,5′-trifluorobiphenyl-2-yl)-1-methyl- |
| | | 3-difluoromethyl-1H-pyrazole-4- |
| | | carboxamide |
| D-76 | a compound of the formula I | N-(2′,4′,5′-trifluorobiphenyl-2-yl)-1-methyl- |
| | | 3-difluoromethyl-1H-pyrazole-4- |
| | | carboxamide |
| D-77 | a compound of the formula I | N-(3′,4′,5′-trifluorobiphenyl-2-yl)-3- |
| | | chlorofluoromethyl-1-methyl-1H-pyrazole- |
| | | 4-carboxamide |
| D-78 | a compound of the formula I | N-[2-(1,1,2,3,3,3- |
| | | hexafluoropropoxy)phenyl]-1-methyl-3- |
| | | trifluoromethyl-1H-pyrazole-4-carboxamide |
| D-79 | a compound of the formula I | N-[2-(1,1,2,3,3,3-hexafluoropropoxy)- |
| | | phenyl]-3-difluoromethyl-1-methyl-1H- |
| | | pyrazole-4-carboxamide |
| D-80 | a compound of the formula I | N-[2-(2-chloro-1,1,2-trifluoroethoxy)- |
| | | phenyl]-1-methyl-3-trifluoromethyl-1H- |
| | | pyrazole-4-carboxamide |
| D-81 | a compound of the formula I | N-[2-(2-chloro-1,1,2- |
| | | trifluoroethoxy)phenyl]-3-difluoromethyl-1- |
| | | methyl-1H-pyrazole-4-carboxamide |
| D-82 | a compound of the formula I | N-[2-(1,1,2,2-tetrafluoroethoxy)phenyl]-3- |
| | | difluoromethyl-1-methyl-1H-pyrazole-4- |
| | | carboxamide |
| D-83 | a compound of the formula I | N-[2-(1,1,2,2-tetrafluorethoxy)phenyl]-1- |
| | | methyl-3-trifluoromethyl-1H-pyrazole-4- |
| | | carboxamide |
| D-84 | a compound of the formula I | N-(4′-(trifluoromethylthio)biphenyl-2-yl)-3- |
| | | difluoromethyl-1-methyl-1H-pyrazole-4- |
| | | carboxamide |
| D-85 | a compound of the formula I | N-(4′-(trifluoromethylthio)biphenyl-2-yl)-1- |
| | | methyl-3-trifluoromethyl-1H-pyrazole-4- |
| | | carboxamide |
| D-86 | a compound of the formula I | [2-(1,2-dimethylpropyl)phenyl]-5-fluoro- |
| | | 1,3-dimethyl-1H-pyrazole-4-carboxamide |
| D-87 | a compound of the formula I | N-(2-{4-[3-(4-chlorophenyl)prop-2- |
| | | ynyloxy]-3-methoxyphenyl}ethyl)-2- |
| | | methanesulfonylamino-3- |
| | | methylbutyramide |
| D-88 | a compound of the formula I | N-(2-{4-[3-(4-chlorophenyl)prop-2- |
| | | ynyloxy]-3-methoxyphenyl}ethyl)-2- |
| | | ethanesulfonylamino-3-methylbutyramide |
| D-89 | a compound of the formula I | dimethomorph |
| D-90 | a compound of the formula I | flumorph |
| D-91 | a compound of the formula I | flumetover |
| D-92 | a compound of the formula I | fluopicolide (picobenzamid) |
| D-93 | a compound of the formula I | fluopyram |
| D-94 | a compound of the formula I | zoxamide |
| D-95 | a compound of the formula I | N-(3-ethyl-3,5,5-trimethylcyclohexyl)- |
| | | 3-formylamino-2-hydroxybenzamide |
| D-96 | a compound of the formula I | carpropamid |
| D-97 | a compound of the formula I | diclocymet |
| D-98 | a compound of the formula I | mandipropamid |
| D-99 | a compound of the formula I | oxytetracyclin |
| D-100 | a compound of the formula I | silthiofam |
| D-101 | a compound of the formula I | N-(6-methoxypyridin-3-yl)cyclopropane- |
| | | carboxamide |
| D-102 | a compound of the formula I | azaconazole |
| D-103 | a compound of the formula I | bitertanol |
| D-104 | a compound of the formula I | bromuconazole |
| D-105 | a compound of the formula I | cyproconazole |
| D-106 | a compound of the formula I | difenoconazole |
| D-107 | a compound of the formula I | diniconazole |
| D-108 | a compound of the formula I | diniconazole-M |
| D-109 | a compound of the formula I | enilconazole |
| D-110 | a compound of the formula I | epoxiconazole |
| D-111 | a compound of the formula I | fenbuconazole |
| D-112 | a compound of the formula I | flusilazole |
| D-113 | a compound of the formula I | fluquinconazole |
| D-114 | a compound of the formula I | flutriafol |
| D-115 | a compound of the formula I | hexaconazole |
| D-116 | a compound of the formula I | imibenconazole |
| D-117 | a compound of the formula I | ipconazole |
| D-118 | a compound of the formula I | metconazole |
| D-119 | a compound of the formula I | myclobutanil |
| D-120 | a compound of the formula I | oxpoconazole |
| D-121 | a compound of the formula I | paclobutrazole |
| D-122 | a compound of the formula I | penconazole |
| D-123 | a compound of the formula I | propiconazole |
| D-124 | a compound of the formula I | prothioconazole |
| D-125 | a compound of the formula I | simeconazole |
| D-126 | a compound of the formula I | tebuconazole |
| D-127 | a compound of the formula I | tetraconazole |
| D-128 | a compound of the formula I | triadimenol |
| D-129 | a compound of the formula I | triadimefon |
| D-130 | a compound of the formula I | triticonazole |
| D-131 | a compound of the formula I | uniconazole |
| D-132 | a compound of the formula I | 1-(4-chlorophenyl)-2-([1,2,4]triazol-1-yl)- |
| | | cycloheptanol |
| D-133 | a compound of the formula I | cyazofamid |
| D-134 | a compound of the formula I | imazalil |
| D-135 | a compound of the formula I | imazalil-sulfate |
| D-136 | a compound of the formula I | pefurazoate |
| D-137 | a compound of the formula I | prochloraz |
| D-138 | a compound of the formula I | triflumizole |
| D-139 | a compound of the formula I | benomyl |
| D-140 | a compound of the formula I | carbendazim |
| D-141 | a compound of the formula I | fuberidazole |
| D-142 | a compound of the formula I | thiabendazole |
| D-143 | a compound of the formula I | ethaboxam |
| D-144 | a compound of the formula I | etridiazole |
| D-145 | a compound of the formula I | hymexazole |
| D-146 | a compound of the formula I | fluazinam |
| D-147 | a compound of the formula I | pyrifenox |
| D-148 | a compound of the formula I | 1-(4-chlorophenyl)-1-(propyn-2-yloxy)- |
| | | 3-(4-(3,4-dimethoxyphenyl)isoxazol-5-yl)- |
| | | propan-2-one |
| D-149 | a compound of the formula I | 3-[5-(4-chlorophenyl)-2,3-dimethyl- |
| | | isoxazolidin-3-yl]pyridine |
| D-150 | a compound of the formula I | 2,3,5,6-tetrachloro-4-methanesulfonyl- |
| | | pyridine |
| D-151 | a compound of the formula I | 3,4,5-trichloropyridine-2,6-dicarbonitrile |
| D-152 | a compound of the formula I | N-(1-(5-bromo-3-chloropyridin-2-yl)ethyl)- |
| | | 2,4-dichloronicotinamide |
| D-153 | a compound of the formula I | N-((5-bromo-3-chloropyridin-2-yl)methyl)- |
| | | 2,4-dichloronicotinamide |
| D-154 | a compound of the formula I | bupirimate |
| D-155 | a compound of the formula I | cyprodinil |
| D-156 | a compound of the formula I | diflumetorim |
| D-157 | a compound of the formula I | ferimzone |
| D-158 | a compound of the formula I | fenarimol |
| D-159 | a compound of the formula I | mepanipyrim |
| D-160 | a compound of the formula I | nitrapyrin |
| D-161 | a compound of the formula I | nuarimol |
| D-162 | a compound of the formula I | pyrimethanil |
| D-163 | a compound of the formula I | fludioxonil |
| D-164 | a compound of the formula I | fenpiclonil |
| D-165 | a compound of the formula I | aldimorph |
| D-166 | a compound of the formula I | dodemorph |
| D-167 | a compound of the formula I | dodemorph acetate |
| D-168 | a compound of the formula I | fenpropimorph |
| D-169 | a compound of the formula I | tridemorph |
| D-170 | a compound of the formula I | fluoroimid |
| D-171 | a compound of the formula I | iprodione |
| D-172 | a compound of the formula I | procymidone |
| D-173 | a compound of the formula I | vinclozolin |
| D-174 | a compound of the formula I | acibenzolar-S-methyl |
| D-175 | a compound of the formula I | amisulbrom |
| D-176 | a compound of the formula I | anilazin |
| D-177 | a compound of the formula I | blasticidin-S |
| D-178 | a compound of the formula I | captan |
| D-179 | a compound of the formula I | captafol |
| D-180 | a compound of the formula I | quinomethionate |
| D-181 | a compound of the formula I | dazomet |
| D-182 | a compound of the formula I | debacarb |
| D-183 | a compound of the formula I | diclomezine |
| D-184 | a compound of the formula I | difenzoquat |
| D-185 | a compound of the formula I | difenzoquat methylsulfate |
| D-186 | a compound of the formula I | famoxadone |
| D-187 | a compound of the formula I | fenamidone |
| D-188 | a compound of the formula I | fenoxanil |
| D-189 | a compound of the formula I | fenpropidin |
| D-190 | a compound of the formula I | folpet |
| D-191 | a compound of the formula I | octhilinone |
| D-192 | a compound of the formula I | oxolinic acid |
| D-193 | a compound of the formula I | piperalin |
| D-194 | a compound of the formula I | probenazole |
| D-195 | a compound of the formula I | proquinazid |
| D-196 | a compound of the formula I | pyroquilon |
| D-197 | a compound of the formula I | quinoxyfen |
| D-198 | a compound of the formula I | triazoxid |
| D-199 | a compound of the formula I | tricyclazole |
| D-200 | a compound of the formula I | triforine |
| D-201 | a compound of the formula I | 5-chloro-7-(4-methylpiperidin-1-yl)- |
| | | 6-(2,4,6-trifluorophenyl)-[1,2,4]triazolo- |
| | | [1,5-a]pyrimidine |
| D-202 | a compound of the formula I | 6-(4-tert-butylphenyl)-5-methyl- |
| | | [1,2,4]triazolo[1,5-a]pyrimidin-7-ylamine |
| D-203 | a compound of the formula I | 5-methyl-6-(3,5,5-trimethylhexyl)- |
| | | [1,2,4]triazolo[1,5-a]pyrimidin-7-ylamine |
| D-204 | a compound of the formula I | 5-methyl-6-octyl-[1,2,4]triazolo[1,5-a]- |
| | | pyrimidin-7-ylamine |
| D-205 | a compound of the formula I | 6-methyl-5-octyl-[1,2,4]triazolo[1,5-a]- |
| | | pyrimidin-7-ylamine |
| D-206 | a compound of the formula I | 6-ethyl-5-octyl-[1,2,4]triazolo[1,5-a]- |
| | | pyrimidin-7-ylamine |
| D-207 | a compound of the formula I | 5-ethyl-6-octyl-[1,2,4]triazolo[1,5- |
| | | a]pyrimidin-7-ylamine |
| D-208 | a compound of the formula I | 5-ethyl-6-(3,5,5-trimethylhexyl)- |
| | | [1,2,4]triazolo[1,5-a]pyrimidin-7-ylamine |
| D-209 | a compound of the formula I | 6-octyl-5-propyl-[1,2,4]triazolo[1,5-a]- |
| | | pyrimidin-7-ylamine |
| D-210 | a compound of the formula I | 5-methoxymethyl-6-octyl- |
| | | [1,2,4]triazolo[1,5-a]pyrimidin-7-ylamine |
| D-211 | a compound of the formula I | 6-octyl-5-trifluoromethyl[1,2,4]triazolo[1,5- |
| | | a]pyrimidin-7-ylamine |
| D-212 | a compound of the formula I | 5-trifluoromethyl-6-(3,5,5-trimethylhexyl)- |
| | | [1,2,4]triazolo[1,5-a]pyrimidin-7-ylamine |
| D-213 | a compound of the formula I | 2-butoxy-6-iodo-3-propylchromen-4-one |
| D-214 | a compound of the formula I | ferbam |
| D-215 | a compound of the formula I | mancozeb |
| D-216 | a compound of the formula I | maneb |
| D-217 | a compound of the formula I | metiram |
| D-218 | a compound of the formula I | metam |
| D-219 | a compound of the formula I | methasulphocarb |
| D-220 | a compound of the formula I | propineb |
| D-221 | a compound of the formula I | thiram |
| D-222 | a compound of the formula I | zineb |
| D-223 | a compound of the formula I | ziram |
| D-224 | a compound of the formula I | diethofencarb |
| D-225 | a compound of the formula I | benthiavalicarb |
| D-226 | a compound of the formula I | iprovalicarb |
| D-227 | a compound of the formula I | propamocarb |
| D-228 | a compound of the formula I | propamocarb hydrochloride |
| D-229 | a compound of the formula I | 3-(4-chlorophenyl)-3-(2-isopropoxy- |
| | | carbonylamino-3-methylbutyrylamino)- |
| | | propionic acid methyl ester |
| D-230 | a compound of the formula I | valiphenal |
| D-231 | a compound of the formula I | 4-fluorophenyl N-(1-(1-(4-cyanophenyl)- |
| | | ethanesulfonyl)but-2-yl)carbamate |
| D-232 | a compound of the formula I | dodine |
| D-233 | a compound of the formula I | dodine free base |
| D-234 | a compound of the formula I | iminoctadine |
| D-235 | a compound of the formula I | iminoctadine triacetate |
| D-236 | a compound of the formula I | iminoctadine tris(albesilate) |
| D-237 | a compound of the formula I | guazatine |
| D-238 | a compound of the formula I | guazatine acetate |
| D-239 | a compound of the formula I | kasugamycin |
| D-240 | a compound of the formula I | kasugamycin hydrochloride hydrate |
| D-241 | a compound of the formula I | polyoxine |
| D-242 | a compound of the formula I | streptomycin |
| D-243 | a compound of the formula I | validamycin A |
| D-244 | a compound of the formula I | binapacryl |
| D-245 | a compound of the formula I | dicloran |
| D-246 | a compound of the formula I | dinobuton |
| D-247 | a compound of the formula I | dinocap |
| D-248 | a compound of the formula I | nitrothal-isopropyl |
| D-249 | a compound of the formula I | tecnazen |
| D-250 | a compound of the formula I | fentin acetate |
| D-251 | a compound of the formula I | fentin chloride |
| D-252 | a compound of the formula I | fentin hydroxide |
| D-253 | a compound of the formula I | isoprothiolane |
| D-254 | a compound of the formula I | dithianon |
| D-255 | a compound of the formula I | edifenphos |
| D-256 | a compound of the formula I | fosetyl |
| D-257 | a compound of the formula I | fosetyl aluminum |
| D-258 | a compound of the formula I | iprobenfos |
| D-259 | a compound of the formula I | pyrazophos |
| D-260 | a compound of the formula I | tolclofos-methyl |
| D-261 | a compound of the formula I | chlorothalonil |
| D-262 | a compound of the formula I | dichlofluanid |
| D-263 | a compound of the formula I | dichlorophen |
| D-264 | a compound of the formula I | flusulfamide |
| D-265 | a compound of the formula I | hexachlorobenzene |
| D-266 | a compound of the formula I | pencycuron |
| D-267 | a compound of the formula I | pentachlorophenol and salts thereof |
| D-268 | a compound of the formula I | phthalide |
| D-269 | a compound of the formula I | quintozene |
| D-270 | a compound of the formula I | thiophanate methyl |
| D-271 | a compound of the formula I | tolylfluanid |
| D-272 | a compound of the formula I | N-(4-chloro-2-nitrophenyl)-N-ethyl- |
| | | 4-methylbenzenesulfonamide |
| D-273 | a compound of the formula I | phosphorous acid and its salts |
| D-274 | a compound of the formula I | sulfur |
| D-275 | a compound of the formula I | Bordeaux mixture |
| D-276 | a compound of the formula I | copper acetate |
| D-277 | a compound of the formula I | copper hydroxide |
| D-278 | a compound of the formula I | copper oxychloride |
| D-279 | a compound of the formula I | basic copper sulfate |
| D-280 | a compound of the formula I | biphenyl |
| D-281 | a compound of the formula I | bronopol |
| D-282 | a compound of the formula I | cyflufenamid |
| D-283 | a compound of the formula I | cymoxanil |
| D-284 | a compound of the formula I | diphenylamine |
| D-285 | a compound of the formula I | metrafenon |
| D-286 | a compound of the formula I | mildiomycin |
| D-287 | a compound of the formula I | oxine-copper |
| D-288 | a compound of the formula I | prohexadione-calcium |
| D-289 | a compound of the formula I | spiroxamine |
| D-290 | a compound of the formula I | tolylfluanid |
| D-291 | a compound of the formula I | N-(cyclopropylmethoxyimino-(6-difluoro- |
| | | methoxy-2,3-difluorophenyl)methyl)- |
| | | 2-phenylacetamide |
| D-292 | a compound of the formula I | N′-(4-(4-chloro-3-trifluoromethylphenoxy)- |
| | | 2,5-dimethylphenyl)-N-ethyl-N-methyl- |
| | | formamidine |
| D-293 | a compound of the formula I | N′-(4-(4-fluoro-3-trifluoromethylphenoxy)- |
| | | 2,5-dimethylphenyl)-N-ethyl-N-methyl- |
| | | formamidine |
| D-294 | a compound of the formula I | N′-(2-methyl-5-trifluoromethyl-4-(3-tri- |
| | | methylsilanylpropoxy)phenyl)-N-ethyl- |
| | | N-methylformamidine |
| D-295 | a compound of the formula I | N′-(5-difluoromethyl-2-methyl-4-(3-tri- |
| | | methylsilanylpropoxy)phenyl)-N-ethyl- |
| | | N-methylformamidine |
| D-296 | a compound of the formula I | 2-(2-(3-(2,6-dichlorophenyl)-1-methyl- |
| | | allylideneaminooxymethyl)phenyl)-2- |
| | | methoxyimino-N-methylacetamide |
| D-297 | a compound of the formula I | isopyrazam |
| D-298 | a compound of the formula I | metalaxyl-M (mefenoxam) |
| D-299 | a compound of the formula I | N-(3′,4′-dichloro-5-fluorobiphenyl-2-yl)-3- |
| | | trifluoromethyl-1-methyl-1H-pyrazol-4- |
| | | carboxamide |
| D-300 | a compound of the formula I | N-(2-(1,3,3-trimethylbutyl)phenyl)-1,3- |
| | | dimethyl-5-fluoro-1H-pyrazole-4- |
| | | carboxamide |
| D-301 | a compound of the formula I | N-[1,2,3,4-tetrahydro-9-(1-methylethyl)- |
| | | 1,4-methanonaphthalene-5-yl]-3- |
| | | (difluoromethyl)-1-methyl-1H-pyrazole-4- |
| | | carboxamide |
| D-302 | a compound of the formula I | N′-(4-(4-chloro-3-trifluoromethylphenoxy)- |
| | | 2,5-dimethylphenyl)-N-ethyl-N- |
| | | methylformamidine |
| D-303 | a compound of the formula I | N′-(4-(4-fluoro-3-trifluoromethylphenoxy)- |
| | | 2,5-dimethylphenyl)-N-ethyl-N- |
| | | methylformamidine |
| D-304 | a compound of the formula I | N′-(2-methyl-5-trifluoromethyl-4-(3- |
| | | trimethylsilanylpropoxy)phenyl)-N-ethyl-N- |
| | | methylformamidine |
| D-305 | a compound of the formula I | N′-(5-difluoromethyl-2-methyl-4-(3- |
| | | trimethylsilanylpropoxy)phenyl)-N-ethyl-N- |
| | | methylformamidine |
| D-306 | a compound of the formula I | 2-(4-chlorophenyl)-N-[4-(3,4- |
| | | dimethoxyphenyl)isoxazol-5-yl]-2-prop-2- |
| | | inyloxyacetamide |
| D-307 | a compound of the formula I | 3-[5-(4-methylphenyl)-2,3- |
| | | dimethylisoxazolidin-3-yl]-pyridine |
| D-308 | a compound of the formula I | flutianil |
| D-309 | a compound of the formula I | S-allyl 5-amino-2-isopropyl-3-oxo-4-ortho- |
| | | tolyl-2,3-dihydropyrazol-1-thiocarboxylate |
| D-310 | a compound of the formula I | 5-chloro-1-(4,6-dimethoxypyrimidin-2-yl)-2- |
| | | methyl-1H-benzoimidazol |
| D-311 | a compound of the formula I | 6-(3,4-dichlorophenyl)-5-methyl- |
| | | [1,2,4]triazolo[1,5-a]pyrimidin-7-ylamine |
| D-312 | a compound of the formula I | methyl N-(1,2,3,4-tetrahydronaphthalene- |
| | | 1-yl)-2-{1-[2-(5-methyl-3- |
| | | trifluoromethylpyrazol-1-yl)acetyl]piperidin- |
| | | 4-yl}thiazole-4-carboxamide |
| D-313 | a compound of the formula I | methyl (R)—N-(1,2,3,4- |
| | | tetrahydronaphthalene-1-yl)-2-{1-[2-(5- |
| | | methyl-3-trifluoromethylpyrazol-1- |
| | | yl)acetyl]piperidin-4-yl}thiazole-4- |
| | | carboxamide |
| D-314 | a compound of the formula I | 6-tert-butyl-8-fluoro-2,3-dimethylquinolin-4- |
| | | yl acetate |
| D-315 | a compound of the formula I | 6-tert-butyl-8-fluoro-2,3-dimethylquinolin-4- |
| | | yl methoxy acetate |
| D-316 | a compound of the formula I | carbaryl |
| D-317 | a compound of the formula I | carbofuran |
| D-318 | a compound of the formula I | carbosulfan |
| D-319 | a compound of the formula I | methomylthiodicarb |
| D-320 | a compound of the formula I | bifenthrin |
| D-321 | a compound of the formula I | cyfluthrin |
| D-322 | a compound of the formula I | cypermethrin |
| D-323 | a compound of the formula I | alpha-cypermethrin |
| D-324 | a compound of the formula I | zeta-cypermethrin |
| D-325 | a compound of the formula I | deltamethrin |
| D-326 | a compound of the formula I | esfenvalerate |
| D-327 | a compound of the formula I | lambda-cyhalothrin |
| D-328 | a compound of the formula I | permethrin |
| D-329 | a compound of the formula I | tefluthrin |
| D-330 | a compound of the formula I | diflubenzuron |
| D-331 | a compound of the formula I | flufenoxuron |
| D-332 | a compound of the formula I | lufenuron |
| D-333 | a compound of the formula I | teflubenzuron |
| D-334 | a compound of the formula I | spirotetramate |
| D-335 | a compound of the formula I | clothianidin |
| D-336 | a compound of the formula I | dinotefuran |
| D-337 | a compound of the formula I | imidacloprid |
| D-338 | a compound of the formula I | thiamethoxam |
| D-339 | a compound of the formula I | acetamiprid |
| D-340 | a compound of the formula I | thiacloprid |
| D-341 | a compound of the formula I | endosulfan |
| D-342 | a compound of the formula I | fipronil |
| D-343 | a compound of the formula I | abamectin |
| D-344 | a compound of the formula I | emamectin |
| D-345 | a compound of the formula I | spinosad |
| D-346 | a compound of the formula I | spinetoram |
| D-347 | a compound of the formula I | hydramethylnon |
| D-348 | a compound of the formula I | chlorfenapyr |
| D-349 | a compound of the formula I | fenbutatin oxide |
| D-350 | a compound of the formula I | indoxacarb |
| D-351 | a compound of the formula I | metaflumizone |
| D-352 | a compound of the formula I | flonicamid |
| D-353 | a compound of the formula I | lubendiamid |
| D-354 | a compound of the formula I | chlorantraniliprol |
| D-355 | a compound of the formula I | cyazypyr (HGW86) |
| D-356 | a compound of the formula I | cyflumetofen |
| D-357 | a compound of the formula I | acetochlor |
| D-358 | a compound of the formula I | dimethenamid |
| D-359 | a compound of the formula I | metolachlor |
| D-360 | a compound of the formula I | metazachlor |
| D-361 | a compound of the formula I | glyphosate |
| D-362 | a compound of the formula I | glufosinate |
| D-363 | a compound of the formula I | sulfosate |
| D-364 | a compound of the formula I | clodinafop |
| D-365 | a compound of the formula I | fenoxaprop |
| D-366 | a compound of the formula I | fluazifop |
| D-367 | a compound of the formula I | haloxyfop |
| D-368 | a compound of the formula I | paraquat |
| D-369 | a compound of the formula I | phenmedipham |
| D-370 | a compound of the formula I | clethodim |
| D-371 | a compound of the formula I | cycloxydim |
| D-372 | a compound of the formula I | profoxydim |
| D-373 | a compound of the formula I | sethoxydim |
| D-374 | a compound of the formula I | tepraloxydim |
| D-375 | a compound of the formula I | pendimethalin |
| D-376 | a compound of the formula I | prodiamine |
| D-377 | a compound of the formula I | trifluralin |
| D-378 | a compound of the formula I | acifluorfen |
| D-379 | a compound of the formula I | bromoxynil |
| D-380 | a compound of the formula I | imazamethabenz |
| D-381 | a compound of the formula I | imazamox |
| D-382 | a compound of the formula I | imazapic |
| D-383 | a compound of the formula I | imazapyr |
| D-384 | a compound of the formula I | imazaquin |
| D-385 | a compound of the formula I | imazethapyr |
| D-386 | a compound of the formula I | 2,4-dichlorophenoxy acetic acid (2,4-D) |
| D-387 | a compound of the formula I | chloridazon |
| D-388 | a compound of the formula I | clopyralid |
| D-389 | a compound of the formula I | fluroxypyr |
| D-390 | a compound of the formula I | picloram |
| D-391 | a compound of the formula I | picolinafen |
| D-392 | a compound of the formula I | bensulfuron |
| D-393 | a compound of the formula I | chlorimuron-ethyl |
| D-394 | a compound of the formula I | cyclosulfamuron |
| D-395 | a compound of the formula I | iodosulfuron |
| D-396 | a compound of the formula I | mesosulfuron |
| D-397 | a compound of the formula I | metsulfuron-methyl |
| D-398 | a compound of the formula I | nicosulfuron |
| D-399 | a compound of the formula I | rimsulfuron |
| D-400 | a compound of the formula I | triflusulfuron |
| D-401 | a compound of the formula I | atrazine |
| D-402 | a compound of the formula I | hexazinone |
| D-403 | a compound of the formula I | diuron |
| D-404 | a compound of the formula I | florasulam |
| D-405 | a compound of the formula I | pyroxasulfon |
| D-406 | a compound of the formula I | bentazone |
| D-407 | a compound of the formula I | cinidon-ethlyl |
| D-408 | a compound of the formula I | cinmethylin |
| D-409 | a compound of the formula I | dicamba |
| D-410 | a compound of the formula I | diflufenzopyr |
| D-411 | a compound of the formula I | quinclorac |
| D-412 | a compound of the formula I | quinmerac |
| D-413 | a compound of the formula I | mesotrione |
| D-414 | a compound of the formula I | saflufenacil |
| D-415 | a compound of the formula I | topramezone |
| D-416 | a compound of the formula I | pyrimorph |

The active compounds specified above as component 2, their preparation, and their effect against fungal pathogens are known (cf.: http://www.hcIrss.demon.co.uk/index.html; http://www.alanwood.net/pesticides/); they are available commercially. The compounds with IUPAC nomenclature, their preparation, and their fungicidal activity are likewise known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 05/120234; WO 05/123689; WO 05/123690; WO 05/63721; WO 05/87772; WO 05/87773; WO 06/15866; WO 06/87325; WO 06/87343; WO 07/82098; WO 07/90624).

The preparation of the compositions for mixtures of active compounds is carried out in a manner known per se in the form of compositions comprising, in addition to the active compounds, a solvent or solid carrier, for example as stated for compositions of the compounds I.

With respect to the customary ingredients of such compositions, reference is made to what was said about the compositions comprising the compounds I. The compositions for mixtures of active compounds are suitable as fungicides for controlling harmful fungi. They are distinguished by excellent efficacy against a broad spectrum of phytopathogenic fungi including soil-borne pathogens originating, in particular, from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Reference is furthermore made to what was said about the efficacy of the compounds I and the compositions comprising the compounds I.

The present invention also provides the use of compounds I and their pharmaceutically acceptable salts for treating diseases, in particular to the use of the compounds I as antimycotic. Thus, one embodiment of the invention relates to a medicament comprising at least one compound of the formula I and/or a pharmaceutically acceptable salt thereof. A further embodiment relates to the use of a compound I and/or a pharmaceutically effective salt thereof for preparing an antimycotic.

The present invention furthermore provides the use of compounds II and their pharmaceutically acceptable salts for treating diseases, in particular to the use of the compounds II as antimycotic. Thus, one embodiment of the invention relates to a medicament comprising at least one compound of the formula II and/or a pharmaceutically acceptable salt thereof. A further embodiment relates to the use of a compound II and/or a pharmaceutically effective salt thereof for preparing an antimycotic.

The present invention furthermore provides the use of compounds I and their pharmaceutically acceptable salts for treating tumors in mammals such as, for example, in humans. Thus, one embodiment of the invention relates to the use of a compound I and/or a pharmaceutically active salt thereof for preparing a composition which inhibits the growth of tumors and cancer in mammals. “Cancer” refers in particular to malignant tumors, for example breast cancer, prostate cancer, lung cancer, cancer of the CNS, melanocarcinomas, ovarial carcinomas or renal cancer, in particular in humans.

The present invention furthermore provides the use of compounds I and their pharmaceutically acceptable salts for treating virus infections, in particular virus infections causing diseases in warm-blooded animals. Thus, one embodiment of the invention relates to the use of a compound I and/or a pharmaceutically active salt thereof for preparing a composition for treating virus infections. The virus diseases to be treated include retrovirus diseases, such as, for example: HIV and HTLV, influenza virus, rhino virus diseases, herpes and the like.

### SYNTHESIS EXAMPLES

TABLE E
Compounds of the formula I

| No. | A | B | D | HPCL-MS* |
|---|---|---|---|---|
| I.1 | 4-F-Phenyl | 2,4-Dimethylphenyl | SH | 3.481 |
| I.2 | 2-Cl-Phenyl | 2,4-Dichlorophenyl | SH | 3.582 |
| I.3 | 4-F-Phenyl | 2-F-3-Cl-Phenyl | SH | 3.029 |
| I.4 | Phenyl | 2,3,4-Trichlorophenyl | SH | 3.260 |

*HPLC column: RP-18 column (Chromolith Speed ROD from Merck KgaA, Germany), 50 mm × 4.6 mm; mobile phase: acetonitrile + 0.1% trifluoroacetic acid (TFA)/water + 0.1% TFA in a gradient of from 5:95 to 95:5 over 5 minutes at 40° C., flow rate 1.8 ml/min
MS: Quadrupol electrospray ionization, 80 V (positive mode)

### Synthesis Examples

With appropriate modification of the starting materials, the procedures given in the synthesis examples below were used to obtain further compounds of the formula I or precursors thereof:

Melting points were determined on a MeI-Temp II instrument and are uncorrected. 1H-NMR spectra measured on a Bruker AC 300 spectrometer at 300 MHz and are based on tetramethylsilane as internal standard (from Aldrich or Cambridge Isotope Laboratories).

ESI mass spectra were measured on a Shimadzu LCMS-2010 EV mass spectrometer. APCI mass spectra were measured on a Shimadzu LCMS-2010 EV mass spectrometer.

### Example 1

### Synthesis of 1-[rel-(2R,3S)-3-(2,4-dimethylphenyl)-2-(4-fluorophenyl)-oxiranylmethyl]-1H-[1,2,4]triazole-3-thiol (Compound 1.1)

At −78° C. 3.1 ml of a 1.6M solution of n-butyllithium in hexane were added dropwise to a solution of 1g of 1-[rel-(2R,3S)-3-(2,4-dimethylphenyl)-2-(4-fluorophenyl)oxiranyl-methyl]-1H-[1,2,4]triazole in 30 ml of anhydrous THF. 2.4 g of sulfur were then added, and the mixture was stirred at −78° C. for another 90 minutes. At −78° C., saturated ammonium chloride solution was added to the solution and, after thawing to room temperature, the solution was extracted three times with ethyl acetate. The combined organic phases were washed with saturated sodium chloride solution, dried and concentrated. The residue was repeatedly taken up in a methyl ted-butyl ether/ethyl acetate mixture unit the excess sulfur had been removed. Finally, the product was crystallized from ethyl acetate/diisopropyl ether/cyclohexane. This gave 246 mg of 1-[rel-(2R,3S)-3-(2,4-dimethylphenyl)-2-(4-fluorophenyl)oxiranylnnethyl]-1H-[1,2,4]triazole-3-thiol in the form of a white powder (HPLC retention time: 3.647).

### Example 2

### Synthesis of 1-[rel-(2S,3R)-2-(2-chlorophenyl)-3-(2,4-dichlorophenyl)oxiran-2-ylmethyl]-1H-1,2,4-triazole-5(4H-6-thione

At −78° C., lithium diisopropylamide (LDA, 8.6 ml, 17.2 mmol, 2.0 M in THF) was added dropwise to a solution of 1-[rel-(2S,3R)-2-(2-chlorophenyl)-3-(2,4-dichlorophenyl)oxiran-2-ylmethyl]-1H-1,2,4-triazole (5.0 g, 13.2 mmol) in dry tetrahydrofuran (THF, 100 ml). After 20 minutes, sulfur (844 mg, 26.4 mmol) was added. The reaction mixture was stirred for 4 hours, during which time it was allowed to warm from −70° C. to −40° C. Saturated ammonium chloride solution (30 ml) was added, and the reaction mixture was then extracted with ethyl acetate (50 ml). The organic phase was washed with saturated sodium chloride solution (three times in each case 40 ml), dried with sodium sulfate and concentrated. The residue was purified by column chromatography (silica gel, 3:2 hexane/ethyl acetate) and recrystallized from hexane/methylene chloride. This gave the target compound (2.72 g, 50%) as a white solid (melting point 196-197° C.).

### Example 3

### Synthesis of 1-[rel-(2S,3R)-3-(3-chloro-2-fluorophenyl)-2-(4-fluorophenyl)oxiran-2-ylmethyl]-1H-1,2,4-triazole-5(4M-thione

At −78° C., lithium diisopropylamide (LDA, 3.5 ml, 7.0 mmol, 2.0 M in THF/heptane/ethylbenzene) was added dropwise to a solution of 1-[rel-(2S,3R)-3-(3-chloro-2-fluorophenyl)-2-(4-fluorophenyl)oxiran-2-ylmethyl]-1H-1,2,4-triazole (2.0 g, 5.8 mmol) in dry tetrahydrofuran (THF, 20 ml). After 15 minutes, sulfur (0.39 g, 12.1 mmol) was added. The reaction mixture was stirred at −78° C. for 5.5 hours. Saturated ammonium chloride solution (30 ml) was added, and the reaction mixture was then extracted with ethyl acetate (50 ml). The organic phase was washed with saturated sodium chloride solution (three times in each case 40 ml), dried with sodium sulfate and concentrated. The residue was purified by column chromatography (silica gel, 9:1 to 5:1 methylene chloride/ethyl acetate) and recrystallized from hexane/methylene chloride. This gave the target compound (540 mg, 10%) as a beige solid (melting point 164-167° C.).

### Example 4

### Synthesis of 1-[rel-(2S,3R)-2-phenyl-3-(2,3,4-trichlorophenyl)oxiran-2-ylmethyl]-1H-1,2,4-triazole-5(4H)-thione

At −70° C., lithium diisopropylamide (LDA, 8.4 ml, 12.6 mmol, 1.5 M in THF/heptane/ethylbenzene) was added dropwise to a solution of 1-[rel-(2S,3R)-2-phenyl-3-(2,3,4-trichlorophenyl)oxiran-2-ylmethyl]-1H-1,2,4-triazole (4.0 g, 10.5 mmol) in dry tetrahydrofuran (THF, 40 ml). After 15 minutes, sulfur (0.69 g, 21.5 mmol) was added. The reaction mixture was stirred at −78° C. for 5 hours. Saturated ammonium chloride solution (30 ml) was added, and the reaction mixture was then extracted with ethyl acetate (50 ml). The organic phase was washed with saturated sodium chloride solution (three times in each case 40 ml), dried with sodium sulfate and concentrated. The residue was purified by column chromatography (silica gel, 19:1 to 9:1 methylene chloride/ethyl acetate) and recrystallized from hexane/methylene chloride. This gave the target compound (340 mg, 8%) as a pale-yellow solid (melting point 168-171° C.).

### Example 5

### Synthesis of 1-[rel-(2S,3R)-2-(2-chlorophenyl)-3-(2,4-dichlorophenyl) oxiran-2-ylmethyl]-5-(methylthio)-1H-1,2,4-triazole

At −70° C., LDA (0.85 ml, 1.7 mmol, 2.0 M in THF) was added dropwise to a solution of 1-[rel-(2S,38)-2-(2-chlorophenyl)-3-(2,4-dichlorophenyl)oxiran-2-ylmethyl]-1H-1,2,4-triazole (500 mg, 1.3 mmol) in dry tetrahydrofuran (THF, 5 ml). After 20 minutes, dimethyl disulfide (153 μl, 1.7 mmol) was added, and the solution was stirred at −70° C. for 3 hours. Saturated ammonium chloride solution (15 ml) was added, and the reaction mixture was then extracted with ethyl acetate (20 ml). The organic phase was washed with saturated sodium chloride solution (three times in each case 20 ml), dried with sodium sulfate and freed from the solvent. The residue was purified by column chromatography (silica gel, 3:1 hexane/ethyl acetate). This gave the target compound (400 mg, 71%) as a yellow oil.

1H NMR (300 MHz, CDCl₃) δ 7.79 (s, 1H), 7.65-7.14 (m, 7H), 4.76 (d, 1H), 4.33 (s, 1H), 4.17 (d, 1H), 2.42 (s, 3H).

### Example 6

### Synthesis of 1-[rel-(2S,3R)-2-phenyl-3-(2,3,4-trichlorophenyl)oxiran-2-ylmethyl]-5-(methylthio)-1H-1,2,4-triazole

At −75° C., LDA (0.3 ml, 0.60 mmol, 2.0 M in THF) was added dropwise to a solution of 1-[rel-(2S,3R)-2-phenyl-3-(2,3,4-trichlorophenyl)oxiran-2-ylmethyl]-1H-1,2,4-triazole (185 mg, 0.49 mmol) in dry tetrahydrofuran (THF, 5 ml). After 15 minutes, dimethyl disulfide (60 μl, 0.66 mmol) was added, and the solution was stirred at −75° C. for 4.8 hours. Saturated ammonium chloride solution (15 ml) was added, and the reaction mixture was then extracted with ethyl acetate (20 ml). The organic phase was washed with saturated sodium chloride solution (three times in each case 20 ml), dried with sodium sulfate and freed from the solvent. The residue was purified by column chromatography (silica gel, 9:1 to 2:1 hexane/ethyl acetate). This gave the target compound (56 mg, 27%) as a pale-yellow oil.

1H NMR (300 MHz, CDCl₃) δ 7.70 (s, 1H), 7.50-7.29 (m, 7H), 4.62 (d, 1H), 4.15 (s, 1H), 3.92 (d, 1H).

### Example 7

### Synthesis of 1-[rel-(2S,3R)-3-(3-chloro-2-fluorophenyl)-2-(4-fluorophenyl)oxiran-2-ylmethyl]-5-(methylthio)-1,2,4-triazole

At −78° C., LDA (1.4 ml, 2.8 mmol, 2.0 M in THF) was added dropwise to a solution of 1-[rel-(2S,3R)-3-(3-chloro-2-fluorophenyl)-2-(4-fluorophenyl)oxiran-2-ylmethyl]-1/1-1,2,4-triazole (804 mg, 2.3 mmol) in dry tetrahydrofuran (THF, 10 ml). After 20 minutes, dimethyl disulfide (250 μl, 2.8 mmol) was added, and the solution was stirred at −78° C. for 2 hours. Saturated ammonium chloride solution (15 ml) was added, and the reaction mixture was then extracted with ethyl acetate (20 ml). The organic phase was washed with saturated sodium chloride solution (three times in each case 20 ml), dried with sodium sulfate and freed from the solvent. The residue was purified by column chromatography (silica gel, 9:1 to 7:3 hexane/ethyl acetate) and recrystallized from hexane/methylene chloride. This gave the target compound (238 mg, 26%) as a beige solid (melting point 88° C.).

### Example 8

### Synthesis of 1-[rel-(2S,3R)-2-(2-chlorophenyl)-3-(2,4-dichlorophenyl)-oxiran-2-ylmethyl]-5-(methylsulfonyl)-1H-1,2,4-triazole

At room temperature, a mixture of 1-[rel-(2S,3R)-2-(2-chlorophenyl)-3-(2,4-dichlorophenyl)oxiran-2-ylmethyl]-5-(methylthio)-1H-1,2,4-triazole (280 mg, 0.66 mmol) and m-CPBA (444 mg, 1.98 mmol) in methylene chloride CH₂Cl₂ (20 ml) was stirred for 24 hours. The reaction mixture was washed with 1N sodium hydroxide solution (two times in each case 20 ml) and saturated sodium chloride solution (two times in each case 20 ml), dried with sodium sulfate and freed from the solvent. The residue was purified by column chromatography (silica gel, 5:1 hexane/ethyl acetate). This gave the target compound (125 mg, 42%) as a white solid (melting point 129-133° C.).

### Example 9

### Synthesis of S-1-[rel-(2S,3R)-2-(2-chlorophenyl)-3-(2,4-dichorophenyl)oxiran-2-ylmethyl]-1H-1,2,4-triazole-5-ylethanethioate

At 0° C., acetyl chloride (26 μl, 0.36 mmol) was added dropwise to a mixture of 1-[rel-(2S,3R)-2-(2-chlorophenyl)-3-(2,4-dichlorophenyl)oxiran-2-ylmethyl]-1H-1,2,4-triazole-5(4H)-thione (100 mg, 0.24 mmol) and triethylamine (66 μl, 0.48 mmol) in dry tetrahydrofuran (THF, 3 ml), and the mixture was stirred at this temperature for 3 hours. The mixture was filtered off and concentrated. This gave the target compound (120 mg, 100%) as a yellow solid.

1H NMR (300 MHz, CDCl₃) δ 8.28 (s, 1H), 7.75-7.16 (m, 7H), 4.80 (d, 1H), 4.38 (d, 1H) 4.32 (s, 1H), 2.88 (s, 3H).

### Example 10

### Synthesis of S-1-[rel-(2S,3R)-2-phenyl)-3-(2,3,4-trichlorophenyl)oxiran-2-ylmethyl]-1H-1,2,4-triazole-5-yl ethanethioate

At 0° C., acetyl chloride (18 μl, 0.25 mmol) was added dropwise to a mixture of 1-[rel-(2S,3R)-2-phenyl)-3-(2,3,4-trichlorophenypoxiran-2-ylmethyl]-1H-1,2,4-triazole-5(4H)thione (88 mg, 0.21 mmol) and triethylamine (36 μl, 0.26 mmol) in dry tetrahydrofuran (THF, 2 ml), and the mixture was stirred at this temperature for 3.3 hours. The mixture was filtered off and concentrated. This gave the target compound (83 mg, 84%) as a pale-yellow oil.

1H NMR (300 MHz, CDCl₃) δ 8.22 (s, 1H), 7.57-7.30 (m, 7H), 5.11 (d, 1H), 4.10 (s, 1H), 3.89 (d, 1H), 2.89 (s, 3H).

### Example 11

### Synthesis of S-{2-[2-(2-chlorophenyl)-3-(2,4-dichlorophenyl)oxiranylmethyl]-2H-[1,2,4]triazole-3-yl}methyl rel-(2S,3R)-thiocarbonate

Methyl chloroformate (42 μl, 0.55 mmol) was added dropwise to a mixture of 2-[rel-(2S,3F)-2-(2-chlorophenyl)-3-(2,4-dichlorophenyl)oxiranylmethyl]-2H-[1,2,4]triazole-3-thiole (150 mg, 0.36 mmol) and triethylamine (100 μl, 0.72 mmol) in dry tetrahydrofuran (THF, 5 ml). The mixture was stirred for 3 hours, during which time the temperature was allowed to rise from 0° C. to room temperature. Ethyl acetate (20 ml) was added. The organic phase was washed with saturated sodium chloride solution (three times in each case 20 ml), dried with sodium sulfate and freed from the solvent. The crude product was purified by column chromatography (silica gel, 10:1 methylene chloride/ethyl acetate). This gave the target compound (65 mg, 38%) as a white solid (melting point 68-70° C.).

### Example 12

### Synthesis of 1-[rel-(2S,3R)-2-(2-chlorophenyl)-3-(2,4-dichlorophenyl)oxiran-2-ylmethyl]-5-thlocyanato-1H-1,2,4-triazole

A mixture of 1-[rel-(2S,3R)-2-(2-chlorophenyl)-3-(2,4-dichlorophenyl)oxiranylmethyl]-1/4-[1,2,4]triazole-5(4H)-thione (200 mg, 0.48 mmol), triethylamine (130 μl, 0.96 mmol) and cyanogen bromide BrCN (76 mg, 0.72 mmol) in dry tetrahydrofuran (THF, 5 ml) was stirred for 3 hours, during which time the temperature was allowed to rise from 0° C. to room temperature. Ethyl acetate (20 ml) was added, and the mixture was washed with saturated sodium chloride solution (three times in each case 20 ml), dried with sodium sulfate and freed from the solvent. The residue was purified by column chromatography (silica gel, 10:1 hexane/ethyl acetate). This gave the target compound (170 mg, 80%) as a white solid (melting point 79-83° C.).

### Example 13

### Synthesis of sodium rel-(2S,3R)-2-(2-chlorophenyl)-3-(2,4-dichlorophenyl)oxiran-2-ylethyl]-1H-[1,2,4]triazole-5-thiolate

300 mg of 1-[rel-(2S,3R)-2-(2-chlorophenyl)-3-(2,4-dichlorophenyl)oxiran-2-ylmethyl]-1H-1,2,4-triazole-5(4/1)-thione and sodium methoxide (13 mg, 0.24 mmol) were stirred in dry methanol (5 ml) at room temperature for 3 hours. The mixture was concentrated. This gave 110 mg of the desired product (100%, melting point 164-166° C.) as a beige solid.

### Example 14

### Synthesis of 1,2-bis[1-rel-(2S,3R)-2-(2-chlorophenyl)-3-(2,4-dichlorophenyl)oxiran-2-ylmethyl-1H-1,2,4-triazole-5-yl]disulfane

A mixture of 1-[ref-(2S,3M-2-(2-chlorophenyl)-3-(2,4-dichlorophenyl)oxiranylmethyl]-1H-[1,2,4]triazole-5(4H)-thione (150 mg, 0.36 mmol) and iodine (92 mg, 0.36 mmol) in dry tetrahydrofuran (THF, 20 ml) was stirred at room temperature for 2 hours. The reaction mixture was purified by column chromatography (silica gel, pure hexane to 8:1 hexane/ethyl acetate). This gave the target compound (110 mg, 74%) as a yellow solid (melting point 99-102° C.).

### Biological Experiments

### Greenhouse

### Preparation of Active Compound

The active compounds were prepared separately or jointly as a stock solution comprising 25 mg of active compound which was made up to 10 ml using a mixture of acetone and/or DMSO and the emulsifier Uniperolt EL (wetting agent having emulsifying and dispersing action based on ethoxylated alkyl phenols) in a volume ratio of solvent/emulsifier of 99:1. The mixture was then made up to 100 ml with water. This stock solution was diluted with the solvent/emulsifier/water mixture described to give the concentration of active compounds stated below. Alternatively, the active compounds were used as a commercial finished formulation and diluted with water to the concentration of active compounds stated.

### Use Example 1—Curative Activity Against Soybean Rust Caused by Phakopsora Pachyrhizi (Phakpa K1)

Leaves of potted soybean seedlings were inoculated with a spore suspension of soybean rust (Phakpsora pachyrhizi). The pots were then placed in a chamber with high atmospheric humidity (90 to 95%) and at 23 to 27° C. for 24 hours. During this time, the spores germinated and the germ tubes penetrated into the leaf tissue. The infected plants were then sprayed to runoff point with the active compound solution described above at the active compound concentration stated below. After the spray coating had dried on, the test plants were cultivated in a greenhouse at temperatures between 23 and 27° C. and 60 to 80% relative atmospheric humidity for 14 days. The extent of the rust fungus development on the leaves was then determined visually in percent infection.

Use Example 2—Protective Activity Against Septoria Leaf Spot of Wheat Caused by Septoria tritici(Septtr P2)

Leaves of potted wheat seedlings were sprayed to runoff point with an aqueous suspension having the active compound concentration stated below. 2 days after the spray coating had dried on, they were inoculated with an aqueous spore suspension of Septoria tritici. The test plants were then placed in a greenhouse at temperatures between 20 and 24° C. and a relative atmospheric humidity close to 100% for 4 days and then at temperatures between 18 and 22° C. and an atmospheric humidity of about 70%. After 21 days, the extent of the development of the disease was determined visually in % infection of the total leaf area.

| | | Infection in | Infection in | Infection in |
|---|---|---|---|---|
| | | % at | % at | % at |
| | | 150 ppm | 150 ppm | 600 ppm |
| Compound | | for Phakpa | for Septtr | for Phakpa |
| No. | Structure | K1 | P2 | K1 |
| 7 | 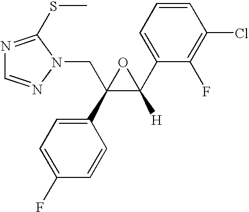 | | | 0 |
| 4 | 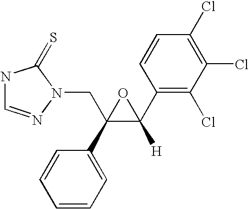 | 0 | 15 | |
| 12 | 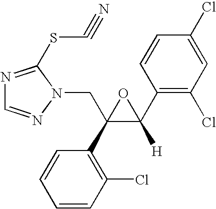 | 0 | 10 | |
| 2 | 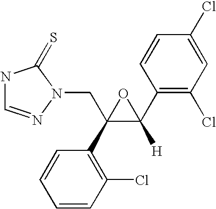 | 3 | 10 | |
| Untreated | | 90 | 90 | 90 |

### Microtest

The active compounds were formulated separately or jointly as a stock solution having a concentration of 10 000 ppm in DMSO.

Use example No. 3—Activity Against the Septoria Leaf Spot Pathogen Septoria tritici in the Microtiter Test (Septtr)

The stock solution was pipetted into a microtiter plate (MTP) and diluted with water to the stated active compound concentration. An aqueous malt-based spore suspension of Septoria tritici was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant (100%) and the fungus- and active compound-free blank value to determine the relative growth in % of the pathogens in the individual active compounds.

| Compound | | Growth in % at 31 ppm |
|---|---|---|
| No. | Structure | for Septtr |
| 5 | 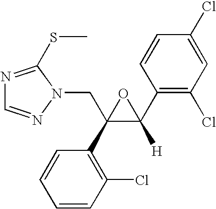 | 0 |
| 14 | 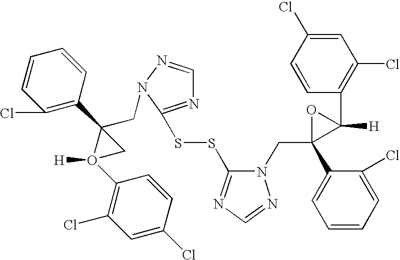 | 4 |
| 10 | 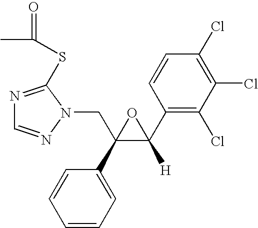 | 5 |

Use Example No. 4—Activity Against the Rice Blast Pathogen Pyricularia oryzae in the Microtiter Test (Pyrior)

The stock solution was pipetted into a microtiter plate (MTP) and diluted with water to the stated active compound concentration. An aqueous malt-based spore suspension of Pyricularia oryzae was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant (100%) and the fungus- and active compound-free blank value to determine the relative growth in % of the pathogens in the individual active compounds.

| Compound | | Growth in % at 31 |
|---|---|---|
| No. | Structure | ppm for Pyrior |
| 13 | 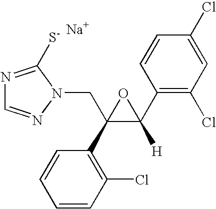 | 19 |
| 2 | 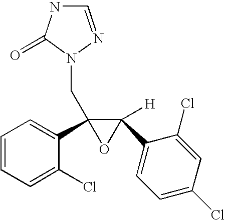 | 5 |
| 8 | 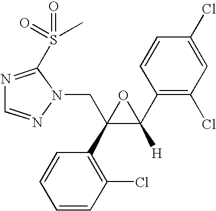 | 8 |
| 9 | 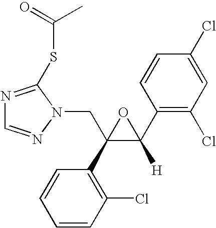 | 1 |
| 11 | 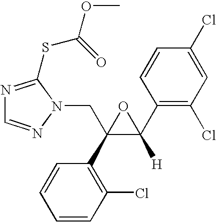 | 3 |

### Comparative Experiments

| | | Growth in | Growth in |
|---|---|---|---|
| | | % at | % at |
| | | 31 ppm for | 31 ppm for |
| Compound No. | Structure | Pyrior | Leptno |
| 13 | 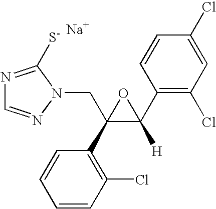 | | 19 |
| WO9741107, p. 50 (5th compound) | 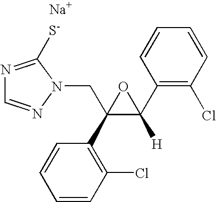 | | 51 |
| 5 | 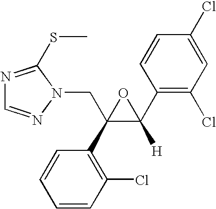 | 0 | |
| WO9638440, p. 14 (5th compound) | 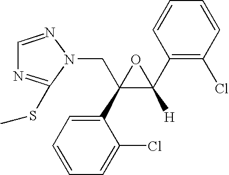 | 83 | |

1-28. (canceled)

29. An azolylmethyloxirane of the formula I 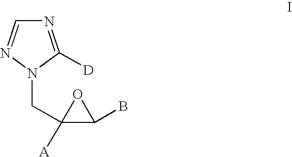
in which the variables have the following meanings:
A is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, naphthyl, or phenyl; where A is unsubstituted or substituted by one, two, three or four identical or different substituents L;
B is phenyl which comprises a substituent L in the 2-position and one, two or three further independently selected substituents L; where L is as defined below: L is halogen, cyano, nitro, cyanato (OCN), C₁-C₈-alkyl, C₁-C₈-haloalkyl, phenyl-C₁-C₆-alkyloxy, C₂-C₈-alkenyl, C₂-C₈-haloalkenyl, C₂-C₈-alkynyl, C₂-C₈-haloalkynyl, C₄-C₁₀-alkadienyl, C₄-C₁₀-haloalkadienyl, C₁-C₈*alkoxy, C₁-C₈-haloalkoxy, C₁-C₈-alkylcarbonyloxy, C₁-C₈-alkylsulfonyloxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-halocycloalkenyl, C₃-C₈-cycloalkoxy, C₃-C₆-cycloalkenyloxy, hydroxyimino-C₁-C₈-alkyl, C₁-C₆-alkylene, oxy-C₂-C₄-alkylene, oxy-C₁-C₃-alkyleneoxy, C₁-C₈-alkoximino-C₁-C₈-alkyl, C₂-C₈-alkenyloximino-C₁-C₈-alkyl, C₂-C₈-alkynyloximino-C₁-C₈-alkyl, S(═O)ₙA¹, C(═O)A², C(═S)A², NA³A⁴, phenyl, phenyloxy or a five- or six-membered saturated, partially unsaturated or aromatic heterocycle which comprises one, two, three or four heteroatoms selected from the group consisting of O, N and S; where n, A¹, A², A³, A⁴ are as defined below:
n is 0, 1 or 2;
A is hydrogen, hydroxyl, C₁-C₈-alkyl, C₁-C₈-haloalkyl, amino, C₁-C₈-alkylamino or di-C₁-C₈-alkylamino,
A² is one of the groups mentioned for A¹ or C₂-C₈-alkenyl, C₂-C₈-haloalkenyl, C₂-C₈-alkynyl, C₂-C₈-haloalkynyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyl-oxy, C₃-C₈-cycloalkyl, C₃-C₈-halocycloalkyl, C₃-C₈-cycloalkoxy or C₃-C₈-halocycloalkoxy;
A³,A⁴ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₃-C₈-cycloalkenyl or C₃-C₈-halo-cycloalkenyl;
where the aliphatic and/or alicyclic and/or aromatic groups of the radical definitions of L and L* for their part may carry one, two, three or four identical or different groups R^{L}:
R^{L} is halogen, cyano, nitro, C₁-C₈-alkyl, C₁-C₈-haloalkyl, C₁-C₈-alkoxy, C₁-C₈-haloalkoxy, C₃-C₈-cycloalkyl, C₃-C₈-halocycloalkyl, C₃-C₈-cycloalkenyl, C₃-C₈-cycloalkoxy, C₃-Cₛ-halocycloalkoxy, C₁-C₈-alkylcarbonyl, C₁-C₈-alkylcarbonyloxy, C₁-C₈alkoxycarbonyl, amino, C₁-C₈-alkylamino, or di-C₁-C₈-alkylamino;
D is S—R, where
R is hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C(═O)R³, C(═S)R³, SO₂R⁴ or CN; where
R³ is C₁-C₈-alkyl, C₁-C₈-haloalkyl, C₁-C₈alkoxy, C₁-C₈-haloalkoxy or NA³A⁴; and
is C₁-C₈-alkyl, phenyl-C₁-C₈-alkyl or phenyl, where the phenyl groups are in each case unsubstituted or substituted by one, two or three groups independently selected from the group consisting of halogen and C₁-C₈ alkyl;
is a group DI 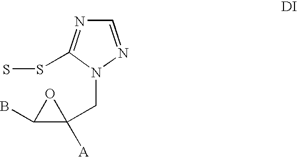
where A and B are as defined above;
is a group DII 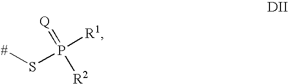
where # is the point of attachment to the triazolyl ring and Q, R¹ and R² are as defined below:
Q is O or S;
R¹, R² independently of one another are C₁-C₈-alkyl, C₁-C₈-haloalkyl, C₁-C₈-alkoxy, C₁-C₈-alkoxy-C₁-C₈-alkoxy, C₁-C₈-haloalkoxy, C₁-C₈-alkoxy-C₁-C₈-alkyl, C₁-C₈-alkylthio, C₂-C₈-alkenylthio, C₂-C₈-alkynylthio, C₃-C₈-cycloalkyl, C₃-C₈-cycloalkylthio, phenyl, phenyl-C₁-C₄-alkyl, phenoxy, phenylthio, phenyl-C₁-C₄-alkoxy or NR⁵R⁶, where R⁵ is H or C₁-C₈-alkyl and R⁶ is C₁-C₈-alkyl, phenyl-C₁-C₄-alkyl or phenyl or R⁵ and R⁶ together are an alkylene chain having four or five carbon atoms or form a radical of the formula —CH₂—CH₂—O—CH₂—CH₂— or —CH₂—CH₂—NR⁷—CH₂—CH₂— in which R⁷ is hydrogen or C₁-C₄-alkyl; where the aromatic groups in the radicals mentioned above are in each case independently of one another unsubstituted or substituted by one, two or three groups selected from the group consisting of halogen and C₁-C₄-alkyl;
or
is a group SM, where M is as defined below:
M is an alkali metal cation, an equivalent of an alkaline earth metal cation, an equivalent of a copper, zinc, iron or nickel cation or an ammonium cation of the formula (E) 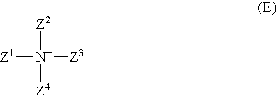
in which
Z¹ and Z² independently are hydrogen or C₁-C₈-alkyl; Z³ and Z⁴ independently are hydrogen, C₁-C₈-alkyl, benzyl or phenyl; where the phenyl groups are in each case unsubstituted or substituted by one, two or three groups independently selected from the group consisting of halogen and C₁-C₄-alkyl;
and an agriculturally acceptable salt thereof.

30. The compound according to claim 29, where A is C₁-C₄-alkyl, C₁-C₄-haloalkyl, C₃-C₆-cycloalkyl or C₃-C₆-halocycloalkyl.

31. The compound according to claim 29, where A is phenyl which comprises one, two, three or four identical or different substituents L.

32. The compound according to claim 30, where A is phenyl which comprises one substituent in the 4-position and zero, one, or two further substituents independently selected from L.

33. The compound according to claim 29 where B is phenyl which comprises a substituent selected from the group consisting of F, Cl, CH₃, OCH₃, CF₃, CHF₂, OCF₃ and OCHF₂ in the 2-position and which contains (1) either a substituent L* in the 4-position or (2) a substituent L in the 3-, 5- or 6-position or (3) two further substituents independently selected from L.

34. The compound according to claim 29, where L is in each case independently selected from the group consisting of halogen, C₁-C₄-alkyl, C₁-C₄-haloalkyl, C₁-C₄-alkoxy, C₁-C₄-haloalkoxy and C₁-C₄-haloalkylthio, and L* is selected from fluorine, bromine, iodine, C₁-C₄-alkyl, C₁-C₄-haloalkyl, C₁-C₄-alkoxy, C₁-C₄-haloalkoxy and C₁-C₄-haloalkylthio.

35. The compound according to claim 34 where L is in each case independently selected from the group consisting of F, Cl, CH₃, C₂H₅, CF₃, OCH₃, OC₂H₅, OCF₃, OCHF₂ and SCF₃ and L* is selected from the group consisting of F, CH₃, C₂H₅, CF₃, OCH₃, OC₂H₅, OCF₃, OCHF₂ and SCF₃.

36. The compound according to claim 29 where D is SH or S—C₁-C₄-alkyl.

37. A composition comprising a compound of the formula I according to claim 29 and/or a salt thereof.

38. The composition according to claim 38 which furthermore comprises at least one solid or liquid carrier.

39. The composition according to claim 38 which further comprises at least one further fungicidally, insecticidally and/or herbicidally active compound.

40. A seed comprising at least one compound of the formula I according to claim 29 and/or an agriculturally acceptable salt thereof.

41. A method for controlling phytopathogenic fungi which comprises treating the fungi or the materials, plants, the soil or seed to be protected against fungal attack with an effective amount of a compound of the formula I according to claim 29 or an agriculturally acceptable salt thereof.

42. A process for preparing an antimycotic which comprises mixing at least one compound of the formula I according to claim 29 and/or a pharmaceutically acceptable salt thereof with a pharmaceutically acceptable carrier.

43. A process for preparing compounds of the formula I according to claim 29 in which D is SR where R is hydrogen, C₁-C₈-alkyl, C₁-C₈-haloalkyl, C₂-C₈-alkenyl, C₂-C₈-haloalkenyl, C₂-C₈-alkynyl or C₂-C₈-haloalkynyl, which comprises
(a1) reacting a compound Mb 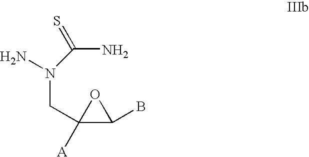
with formic acid to give compounds I-1 (D=SH); 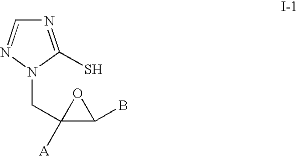
in which D is SH;
and, if desired, to obtain compounds I in which D is SR where R is C₁-C₃-alkyl, C₁-C₈-haloalkyl, C₂-C₈-alkenyl, C₂-C₈-haloalkenyl, C₂-C₈-alkynyl or C₂-C₈-haloalkynyl:
(b1) reacting compounds I-1 with R—X where X is halogen or tri-fluoro(C₁-C₈)alkylsulfonate.

44. A process for preparing compounds of the formula IIIb 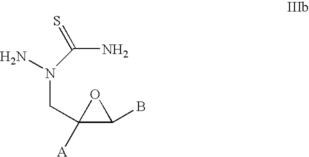
wherein A and B are as defined in claim 29.
which comprises
(a2) reacting a compound of the formula Ma 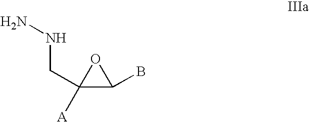
with a thiocyanate YSCN in which Y is an alkali metal or ammonium.

45. A process for preparing compounds I according to claim 29 in which D is SR where R is hydrogen, C₁-C₈-alkyl, C₂-C₈-alkenyl, C₂-C₈-haloalkenyl, C₂-C₈-alkynyl or C₂-C₈-haloalkynyl, which comprises
(a3) oxidizing a compound of the formula IIIc 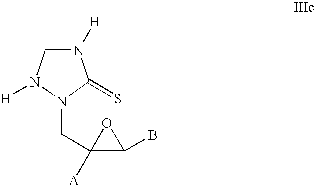
to give a compound I-1; 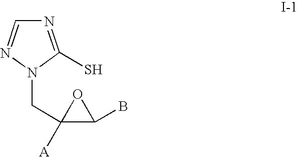
and, if desired, to obtain compounds I in which D is SR where R is C₁-C₈-alkyl, C₁-C₈-haloalkyl, C₂-C₈-alkenyl, C₂-C₈-haloalkenyl, C₂-C₈-alkynyl or C₂-C₈-haloalkynyl:
(b3) reacting compounds I-1 with R—X where X is halogen or trifluoro(C₁-C₈)alkylsulfonate.

46. A process for preparing compounds I according to claim 29 in which D is SR where R=hydrogen, C₁-C₈-alkyl, C₂-C₈-alkenyl, C₂-C₈-haloalkenyl, C₂-C₈-alkynyl or C₂-C₈-haloalkynyl, which comprises
(a4) reacting a compound IIId 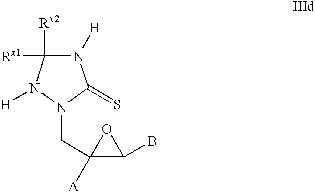
where R^{x1}═C₁-C₄-alkyl or phenyl, R^{x2}=hydrogen or C₁-C₄-alkyl, or R′^{d} and R^{x2} together form a —(CH₂)₅— chain with formic acid to obtain compounds I-1; 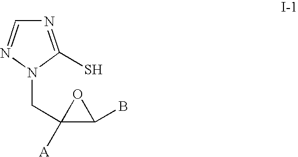
in which D is SH,
and, if desired, to obtain compounds I in which D is SR where R is C₁-C₈-alkyl, C₁-C₈-haloalkyl, C₂-C₈-alkenyl, C₂-C₈-haloalkenyl, C₂-C₈-alkynyl or C₂-C₈-haloalkynyl:
(b4) reacting compounds I-1 with R—X where X is halogen or trifluoro(C₁-C₈)alkylsulfonate.

47. A compound of formula Ma, 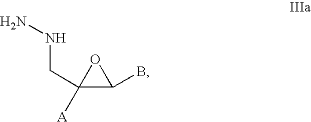
formula IIIb, 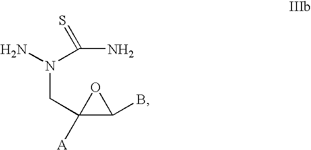
formula IIIc 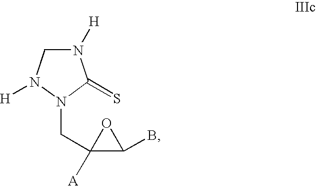
or
formula IIId 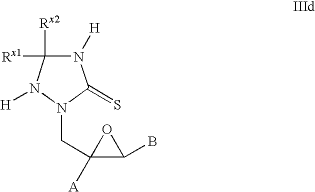
wherein A and B are as defined in claim 29 and is C₁-C₄-alkyl or phenyl, R^{x2} is hydrogen or C₁-C₄-alkyl, or R^{x1} and R^{x2} together form a —(CH₂)₅— chain,
or an agriculturally acceptable salt thereof.

48. A process for preparing compounds I according to claim 29 in which D is SR where R=hydrogen, C₁-C₈-alkyl, C₁-C₈-haloalkyl, C₂-C₈-alkenyl, C₂-C₈-haloalkenyl, C₂-C₈-alkynyl or C₂-C₈-haloalkynyl, which comprises
(a5) reacting of a compound of the formula II 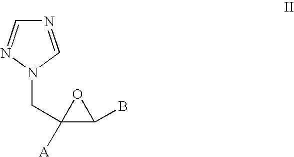
with a strong base and sulfur powder to give a compound I-1; 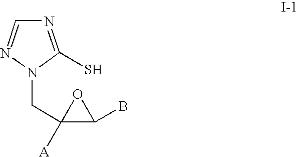
in which D is SH,
and, if desired to obtain compounds I in which D is SR where R is C₁-C₈-alkyl, C₁-C₈-haloalkyl, C₂-C₈-alkenyl, C₂-C₈-haloalkenyl, C₂-C₈-alkynyl or C₂-C₈-haloalkynyl,
(b5) reacting compounds I-1 with R—X where X is halogen or tri-fluoro(C₁-C₈)alkylsulfonate.

49. A process for preparing compounds of the formula II, 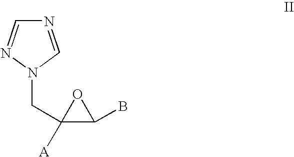
wherein A and B are as defined in claim 29,
which comprises
(a6) reacting a compound of the formula III 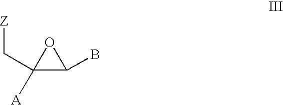
in which Z is a leaving group with 1,2,4-triazole and a base.

50. The compound of the formula III 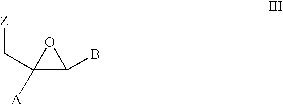
in which A and B are as defined in claim 29, and Z is a leaving group, except for the compounds:
2-bromomethyl-2-(4-chlorophenyl)-3-(2-chloro-4-fluorophenyl)oxirane, 2-bromomethyl-2-(phenyl)-3-(2-chloro-4-fluorophenyl)oxirane, 2-bromomethyl-2-(4-fluorophenyl)-3-(2-chloro-4-fluorophenyl)oxirane, 2-bromomethyl-2-(4-chlorophenyl)-3-(2-chloro-6-fluorophenypoxirane, 2-bromomethyl-2-(phenyl)-3-(2-chloro-6-fluorophenyl)oxirane, and 2-bromomethyl-2-(phenyl)-3-(2,4-dichlorophenyl)oxirane.
